(19) United States
(12) Patent Application Publication
(10) Pub. No.: US 2024/0376123 A1
(43) Pub. Date: Nov. 14, 2024

(54) KRAS G12D INHIBITORS AND USES THEREOF

(71) Applicant: SUZHOU ZANRONG PHARMA LIMITED, Suzhou, Jiangsu (CN)

(72) Inventors: Ding ZHOU, Shanghai (CN); Zheng WANG, Shanghai (CN); Ziqiang CHENG, Shanghai (CN); Shuai CHEN, Shanghai (CN)

(21) Appl. No.: 18/290,798

(22) PCT Filed: Jul. 19, 2022
(86) PCT No.: PCT/CN2022/106447

§ 371 Date: Jan. 21, 2024

(30) Foreign Application Priority Data
Jul. 23, 2021 (WO) PCT/CN2021/108280
Dec. 30, 2021 (WO) PCT/CN2021/143176

(51) Int. Cl. C07D 498/22; A61K 31/4985; A61K 31/551; A61K 31/553; A61K 31/554; A61K 45/06; A61P 35/00; C07D 471/22; C07D 491/22; C07D 513/22; C07D 519/00

(52) U.S. Cl. CPC C07D 498/22; A61K 31/4985; A61K 31/551; A61K 31/553; A61K 31/554; A61K 45/06; A61P 35/00; C07D 471/22; C07D 491/22; C07D 513/22; C07D 519/00

(57) ABSTRACT

Provided novel compounds useful as KRAS G12D inhibitors, as well as pharmaceutical compositions comprising these compounds and methods of treatment by administration of these compounds or the pharmaceutical compositions.

### FIELD OF THE DISCLOSURE

The present disclosure generally relates to novel compounds useful as inhibitors of the KRAS G12D, as well as pharmaceutical compositions comprising these compounds and methods of treatment by administration of these compounds or the pharmaceutical compositions.

### BACKGROUND OF THE DISCLOSURE

RAS is one of the most well-known proto-oncogenes. Its gain-of-function mutations occur in approximately 30% of all human cancers. As the most frequently mutated RAS isoform, KRAS (Kirsten-rat sarcoma viral oncogene homolog) is intensively studied in the past years. KRAS and the highly related NRAS and HRAS GTPases hydrolyze guanosine triphosphate (GTP) to guanosine diphosphate (GDP). They control diverse cellular functions by cycling between an active, GTP-bound and an inactive, GDP-bound conformation (Hobbs, G. A., et al. J. Cell Sci. 129, 1287-1292. (2016)).

KRAS is a prominent oncogene that has been proven to drive tumorigenesis (G G Jinesh, et al. Oncogene volume 37, pages 839-846 (2018)). KRAS also modulates numerous genetic regulatory mechanisms and forms a large tumorigenesis network. KRAS gene encodes a 21 kDa protein, called KRAS, part of the RAS/MAPK pathway. The KRAS protein is a GTPase, which means it binds to guanine nucleotides GDP and guanosine-triphosphate (GTP) with high affinity and can hydrolyze GTP to GDP (Dhirendra K. Simanshu, et al. Cell. 2017 Jun. 29; 170 (1): 17-33). GDP/GTP cycling is tightly regulated by a diverse family of multi-domain proteins: guanine nucleotide exchange-factors (GEFs) and GTPase-activating proteins (GAPs). GEFs stimulate the dissociation of GDP and subsequent association of GTP, activating RAS proteins, while GAPs act to accelerate intrinsic GTP hydrolysis, converting RAS to its inactive state (Dhirendra K. Simanshu, et al. Cell. 2017 Jun. 29; 170 (1): 17-33). The GTP bound form of KRAS is considered the active form, and downstream signaling effectors specifically bind to the GTP-bound form of KRAS. The KRAS protein is turned off (inactivated) when the protein is bound to GDP and does not relay signals to the cell's nucleus.

The cancer-promoting KRAS mutations most commonly occur at codon 12, 13, or 61 (Jozsef Timar, et al. Cancer and Metastasis Reviews volume 39, pages 1029-1038 (2020)). Among these mutation sites, G12 is the most frequently mutated residue (89%) and it most often mutates to aspartate (G12D, 36%) followed by valine (G12V, 23%) and cysteine (G12C, 14%). G12 is located at the protein active site, which consists of a phosphate binding loop (P-loop, residues 10-17) and two switch regions (Switch-I (SI), residues 25-40, and Switch-II (SII), residues 60-74) (Prior, I. A., et al. Cancer Res 72, 2457-2467, (2012)). The residues in the active site bind to the phosphate groups of GTP and are responsible for the GTPase function of KRAS. The switch regions SI and SII are additionally responsible for controlling binding to effector and regulator proteins. The mutation of glycine at position 12 to aspartate (G12D) in the P-loop leads to impair GTP hydrolysis and freeze KRAS in its active (GTP-bound) state, which causes uncontrollable cellular growth and evasion of apoptotic signals (Malumbres, M. & Barbacid, M. Nat Rev Cancer 3, 459-465, (2003)). The G12D mutation causes a shift in the population of local conformational states of KRAS, especially in Switch-II (SII) and α3-helix regions, in favor of a conformation that is associated with a catalytically impaired state through structural changes; it also causes SII motions to anti-correlate with other regions (Sezen Vatansever, et al. Sci Rep. 2019 Aug. 13; 9 (1): 11730).

KRAS mutations are present in up to 25% of cancers, the oncogenic variants have different prevalence rates in different cancers. In pancreatic ductal adenocarcinoma cases, the most common KRAS alteration is the G12D substitution. The G12D variant is also the focus of drug discovery efforts by Mirati, which plans to bring its lead compound, MRTX1133 to clinical trials. Based on epidemiology data reported in Globocan 2022 (accessed November 2019) and frequencies by mutation, KRAS G12D mutation is present in an estimated around 36% of Pancreatic cancer, in 4% colorectal cancer, in around 6% endometrial cancer and in around 4% NSCLC. This significant patient population with high unmet need.

Therefore, KRAS G12D is very commonly observed in pancreatic cancer, which can be considered a representative of the various intractable cancers. KRAS G12D is one of the most important chemotherapy drug targets. To investigate highly selective and potent small molecule inhibitor of KRAS G12D designed to treat patients with high unmet need.

### SUMMARY OF THE DISCLOSURE

Disclosed herein are novel compounds that are capable of inhibiting KRAS G12D proteins. As a result, the compounds of the present disclosure are useful in the treatment of KRAS G12D-associated diseases such as cancers.

In one aspect, the present disclosure provides a compound having Formula (I) or Formula (II):

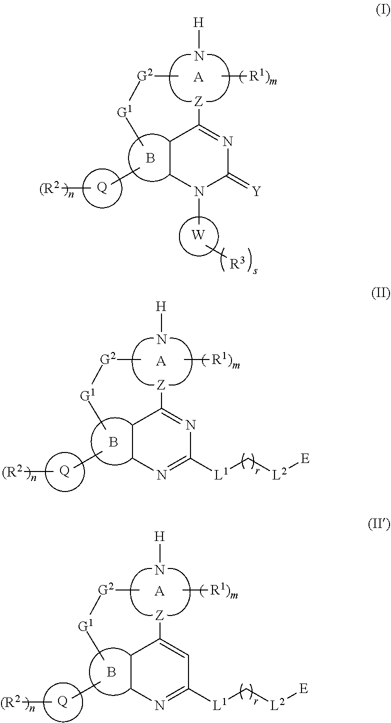

or a pharmaceutically acceptable salt thereof,
wherein
   Y is O or S;
   Ring A is heterocyclyl or heteroaryl,
   each R¹ is independently selected from the group consisting of oxo, hydroxyl, halogen, cyano, alkyl, alkenyl, alkynyl, heteroalkyl, heteroaryl, —C(O)R*, —C(O)OR*, —C(O)N(R^{a})₂, —N(R^{a})₂, —P(O)OR*OR**, and —C(O)OC(R^{a})₂—Z¹—Z², wherein the alkyl, alkenyl, alkynyl and heteroaryl are optionally substituted with one or more groups independently selected from cyano, hydroxyl, halogen, —ORD, or —N(R^{b})₂;
   each R^{a} and R^{b} is independently hydrogen, alkyl, alkenyl or alkynyl;
   R* is selected from hydrogen, alkyl, alkylaryl or aryl;
   R** is selected from hydrogen, alkyl, alkenyl or alkynyl; or
   R* and R** together with the oxygen atoms to which they are attached form a heterocyclyl optionally substituted with aryl or haloaryl;
   Z¹ is —OC(O)-#, —OP(═O)(OR**)O-#, or —OP(═O)(OR*)N(R^{a})-#, wherein # end is connected to Z²;
   Z² is hydrogen or alkyl optionally substituted with —C(O)OR^{a};
   R*** is independently selected from hydrogen, alkyl, alkenyl or alkynyl; or
   R*** and Z² together with the oxygen atoms to which they are attached form a heterocyclyl optionally substituted with aryl or haloaryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   Ring W is selected from cycloalkyl, heterocyclyl, aryl and heteroaryl;
   each R³ is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   G¹ is a bond, —O—, —S(O)ₚ—, —S—S—, —N(R^{c})—, or —C(R⁴)═C(R^{d})—;
   G² is a bond, —[C(R^{d})₂]ᵤ—, —C(O)— or —C(O)C(R^{d})₂—;
   R^{c} is selected from the group consisting of hydrogen, alkyl, haloalkyl, hydroxyalkyl, heteroalkyl, cycloalkyl and heterocyclyl;
   each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of hydroxyl, halogen, cyano, amino, nitro, alkyl, alkoxy, haloalkyl, and hydroxyalkyl; or
   two R^{d} together with the carbon atom to which they are both attached form cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
   Z is C(R^{e}) or N;
   R^{e} is absent or hydrogen;
   L¹ is selected from a bond, —O—, —S—, —N(R^{a})—, —C(O)N(R^{a})—, alkenyl, alkynyl or cycloalkyl;

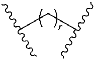

is optionally substituted with hydroxyl, halogen, cyano or amino;
L² is a bond, alkyl, cycloalkyl, heterocyclyl, aryl or heteroaryl, wherein the cycloalkyl, heterocyclyl, aryl and heteroaryl are optionally substituted with one or more groups independently selected from hydroxyl, halogen, cyano, amino, alkyl, hydroxyalkyl or heteroaryl;
E is selected from the group consisting of hydrogen, hydroxyl, halogen, —N(R^{a})₂, alkyl, haloalkyl, heteroalkyl, cycloalkyl, heterocyclyl, aryl, heteroaryl, —COOH, —CH₂OC(O)-heterocyclyl, —CH₂OC(O)N(R^{a})₂, —NHC(═NH)NH₂, —C(O)N(R^{a})₂, —OR^{a}, —(CH₂OR^{a})(CH₂)ₚOR^{a}, —N(R^{a})C(O)-aryl and —(CH₂)ᵤ-heterocyclyl, wherein the cycloalkyl, heterocyclyl, aryl, and heteroaryl are optionally substituted with one or more R″, and the aryl portion in —N(R^{a})C(O)-aryl and the heterocyclyl portion in —(CH₂)ᵤ-heterocyclyl and —CH₂OC(O)-heterocyclyl is optionally substituted with one or more R′″;
each R″ is independently selected from hydroxyl, halogen, —C(O)H, alkyl, alkoxy, haloalkyl, hydroxyalkyl, or —N(R^{a})₂;
each R′″ is independently selected from oxo, hydroxyl, halogen, alkyl, heteroalkyl, hydroxyalkyl, haloalkyl, alkoxy, -T-phenyl, -T-phenylSO₂F, —N(R^{a})₂, —SO₂F, —C(O)(alkyl), or —C(O)(haloalkyl), wherein the alkyl, heteroalkyl, hydroxyalkyl, haloalkyl, and alkoxy are optionally substituted with one or more groups independently selected from aryl, heteroaryl, or tert-butyldimethylsilyloxy;
T is a bond, —O—, or —NHC(O)—;
m is an integer from 0 to 6;
n is an integer from 0 to 5;
r is an integer from 0 to 4;
s is an integer from 0 to 5;
p is an integer from 0 to 2; and
u is an integer from 0 to 4.

In another aspect, the present disclosure provides a compound having Formula (III) or Formula (IV):

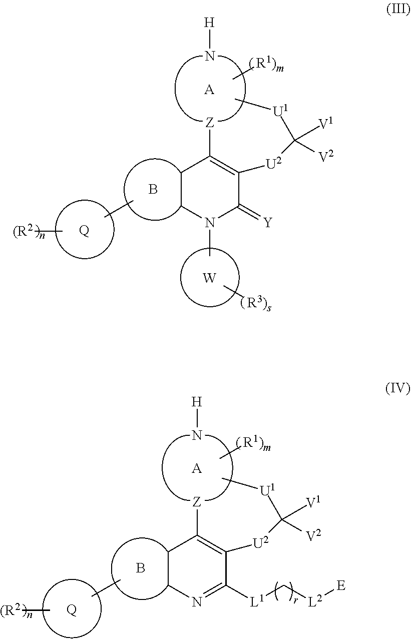

or a pharmaceutically acceptable salt thereof,
wherein
   Y is O or S;
   Ring A is heterocyclyl or heteroaryl,
   each R¹ is independently selected from the group consisting of oxo, hydroxyl, halogen, cyano, alkyl, alkenyl, alkynyl, heteroalkyl, heteroaryl, —C(O)R*, —C(O)OR*, —C(O)N(R^{a})₂, —N(R^{a})₂, —P(O)OR*OR**, and —C(O)OC(R^{a})—Z¹—Z², wherein the alkyl, alkenyl, alkynyl and heteroaryl are optionally substituted with one or more groups independently selected from cyano, hydroxyl, halogen, —OR^{b}, or —N(R^{b})₂;
   each R^{a} and R^{b} is independently hydrogen, alkyl, alkenyl or alkynyl;
   R* is selected from hydrogen, alkyl, alkylaryl or aryl;
   R** is selected from hydrogen, alkyl, alkenyl or alkynyl; or
   R* and R** together with the oxygen atoms to which they are attached form a heterocyclyl optionally substituted with aryl or haloaryl;
   Z¹ is —OC(O)-#, —OP(═O)(OR***)O-#, or —OP(═O)(OR*)N(R^{a})-#, wherein # end is connected to Z²;
   Z² is hydrogen or alkyl optionally substituted with —C(O)OR^{a};
   R*** is independently selected from hydrogen, alkyl, alkenyl or alkynyl; or
   R*** and Z² together with the oxygen atoms to which they are attached form a heterocyclyl optionally substituted with aryl or haloaryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   Ring W is selected from cycloalkyl, heterocyclyl, aryl and heteroaryl;
   each R³ is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   U¹ is selected from a bond, —(CH₂)ₜ—, —O(CH₂)ₜ—, —N(R^{j})—, —C(O)— or —C(O)C(R^{j})₂—;
   U² is selected from —O—, —S(O)ₚ—, —[C(R¹)₂]ₜ—, —C(R^{j})═C(R^{j})ₜ—, —N(R^{j})—, —C(O)— or —C(O)C(R^{j})₂—;
   R^{j} is selected from the group consisting of hydrogen, cyano, alkyl, haloalkyl, hydroxyalkyl, heteroalkyl, cycloalkyl and heterocyclyl, wherein the alkyl, heteroalkyl, cycloalkyl and heterocyclyl are optionally substituted with —N(R^{a})₂ or heterocyclyl;
   V¹ and V² are each independently selected from the group consisting of hydrogen, alkyl, alkenyl, alkynyl, alkoxy, heteroalkyl, cycloalkyl and heterocyclyl, wherein the alkyl, alkenyl, alkynyl, alkoxy, heteroalkyl, cycloalkyl and heterocyclyl are optionally substituted with one or more groups independently selected from hydroxyl, halogen, cyano, amino, alkyl, or haloalkyl; or
   V¹ and V² together with the carbon atom to which they are both attached form oxo, cycloalkyl, heterocyclyl or heteroaryl, wherein cycloalkyl, heterocyclyl, and heteroaryl are optionally substituted with one or more groups independently selected from the group consisting of cyano, halogen, hydroxyl, amino, and alkyl optionally substituted with heterocyclyl or —N(R^{a})₂;
   Z is C(R^{e}) or N;
   R^{e} is absent or hydrogen;
   L¹ is selected from a bond, —O—, —S—, —N(R^{a})—, alkenyl, alkynyl or cycloalkyl;

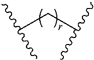

is optionally substituted with hydroxyl, halogen, cyano or amino;
L² is a bond, alkyl, cycloalkyl, heterocyclyl, aryl or heteroaryl, wherein the cycloalkyl, heterocyclyl, aryl and heteroaryl are optionally substituted with one or more groups independently selected from hydroxyl, halogen, cyano, amino, alkyl, hydroxyalkyl or heteroaryl;
E is selected from the group consisting of hydrogen, hydroxyl, halogen, —N(R^{a})₂, alkyl, haloalkyl, heteroalkyl, cycloalkyl, heterocyclyl, aryl, heteroaryl, —COOH, —CH₂OC(O)-heterocyclyl, —NHC(═NH)NH₂, —C(O)N(R^{a})₂, —OR^{a}, —(CH₂OR^{a})(CH₂)ₚOR^{a}, —N(R^{a})C(O)-aryl and —(CH₂)ᵤ-heterocyclyl, wherein the cycloalkyl, heterocyclyl, aryl, and heteroaryl are optionally substituted with one or more R″, and the aryl portion in —N(R^{a})C(O)-aryl and the heterocyclyl portion in —(CH₂)ᵤ-heterocyclyl and —CH₂OC(O)-heterocyclyl is optionally substituted with one or more R′″;
each R″ is independently selected from hydroxyl, halogen, —C(O)H, alkyl, alkoxy, haloalkyl, hydroxyalkyl, or —N(R^{a})₂;
each R′″ is independently selected from oxo, hydroxyl, halogen, alkyl, heteroalkyl, hydroxyalkyl, haloalkyl, alkoxy, -T-phenyl, -T-phenylSO₂F, —N(R^{a})₂, —SO₂F, —C(O)(alkyl), or —C(O)(haloalkyl), wherein the alkyl, heteroalkyl, hydroxyalkyl, haloalkyl, and alkoxy are optionally substituted with one or more groups independently selected from aryl, heteroaryl, or tert-butyldimethylsilyloxy;
T is a bond, —O—, or —NHC(O)—;
m is an integer from 0 to 6;
n is an integer from 0 to 5;
r is an integer from 0 to 4;
s is an integer from 0 to 5;
p is an integer from 0 to 2;
t is an integer from 0 to 3; and
u is an integer from 0 to 4.

In another aspect, the present disclosure provides a compound having Formula (Ia) or Formula (Ib):

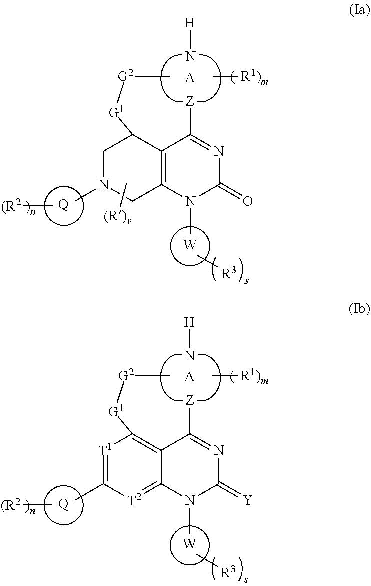

or a pharmaceutically acceptable salt thereof,
wherein
   T¹ is N or C(R′);
   T² is N or C(R′);
   v is an integer from 0 to 4.

In another aspect, the present disclosure provides a compound having a formula selected from the group consisting of:

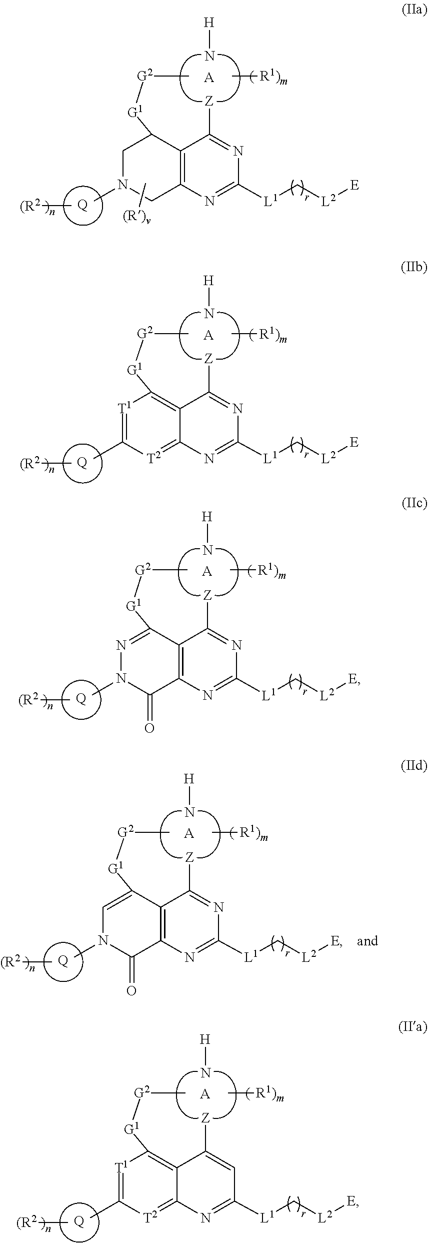

or a pharmaceutically acceptable salt thereof,
wherein
   T¹ is N or C(R′);
   T² is N or C(R′);
   v is an integer from 0 to 4.

In another aspect, the present disclosure provides a compound having Formula (IIIa) or Formula (IIIb):

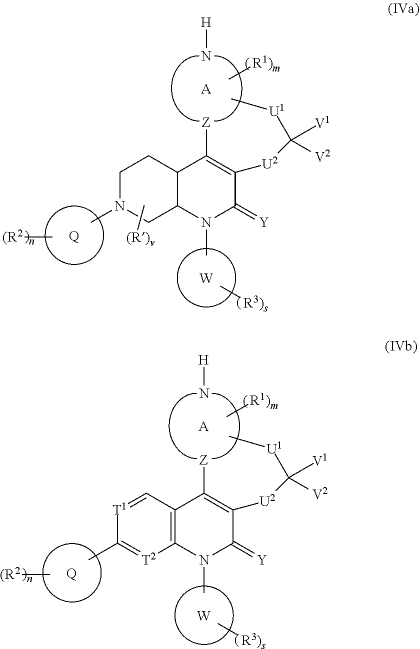

or a pharmaceutically acceptable salt thereof,
wherein
   T¹ is N or C(R′);
   T² is N or C(R′);
   v is an integer from 0 to 4.

In another aspect, the present disclosure provides a compound having Formula (IIIc), Formula (IIId) or Formula (IIIe):

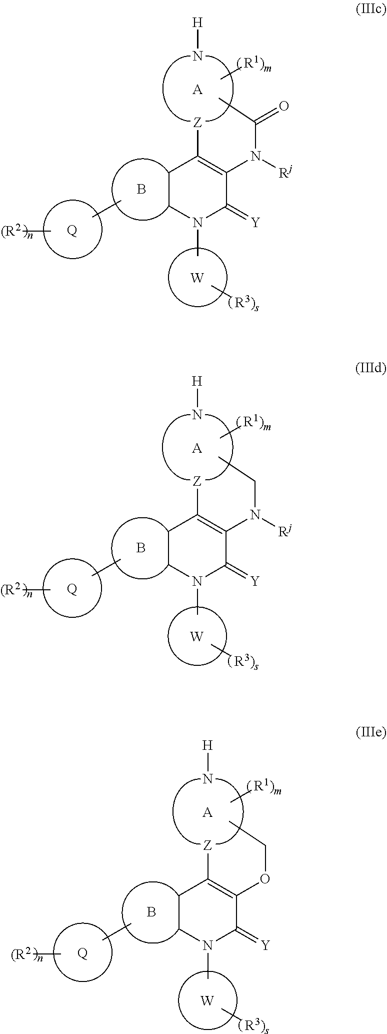

or a pharmaceutically acceptable salt thereof.

In another aspect, the present disclosure provides a compound having Formula (IVa) or Formula (IVb):

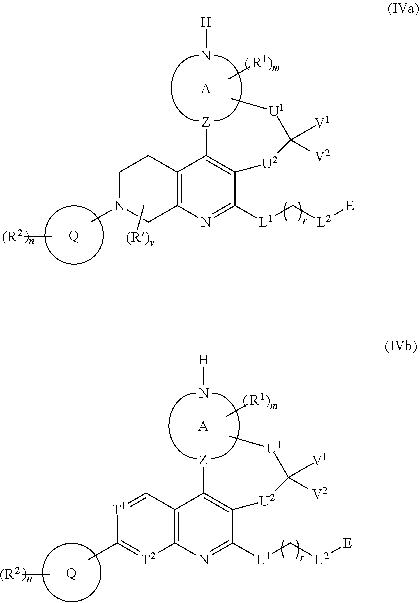

or a pharmaceutically acceptable salt thereof,
wherein
   T¹ is N or C(R′);
   T² is N or C(R′);
   v is an integer from 0 to 4.

In another aspect, the present disclosure provides a compound having Formula (IVc), Formula (IVd) or Formula (IVe):

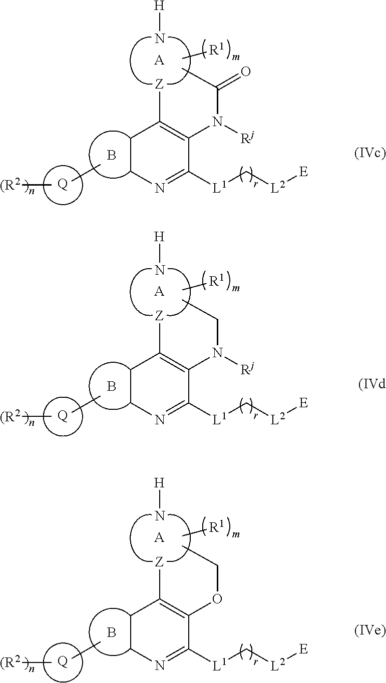

or a pharmaceutically acceptable salt thereof.

In another aspect, the present disclosure provides a pharmaceutical composition comprising the compound of the present disclosure or a pharmaceutically acceptable salt thereof, and a pharmaceutically acceptable excipient.

In a further aspect, the present disclosure provides a method for inhibiting KRas G12D activity in a subject in need thereof, comprising administering an effective amount of a compound of the present disclosure or a pharmaceutically acceptable salt thereof or the pharmaceutical composition of the present disclosure to the subject.

In a further aspect, the present disclosure provides a method for treating a KRas G12D-associated cancer comprising administering an effective amount of a compound of the present disclosure or a pharmaceutically acceptable salt thereof or the pharmaceutical composition of the present disclosure to a subject in need thereof.

In a further aspect, the present disclosure provides a method for treating cancer in a subject in need thereof, the method comprising:
(a) acquiring the knowledge that the cancer is associated with a KRas G12D mutation; and
(b) administering to the subject an effective amount of a compound of the present disclosure or a pharmaceutically acceptable salt thereof or the pharmaceutical composition of the present disclosure.

In another aspect, the present disclosure provides use of the compound of the present disclosure or a pharmaceutically acceptable salt thereof or the pharmaceutical composition of the present disclosure in the manufacture of a medicament for treating cancer.

In another aspect, the present disclosure provides a compound of present disclosure or a pharmaceutically acceptable salt thereof or the pharmaceutical composition of the present disclosure, for use in the treatment of cancer.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. It is specifically intended that each linking substituent includes both the forward and backward forms of the linking substituent. For example, —NR(CR′R″)— includes both —NR(CR′R″)— and —(CR′R″)NR—. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

As used herein, a dash “-” at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “compounds provided herein”, or “compounds disclosed herein” or “compounds of the present disclosure” refers to the compounds of Formula (I), Formula (II), Formula (III), Formula (IV), Formula (Ia), Formula (Ib), Formula (IIa), Formula (IIb), Formula (IIIa), Formula (IIIb), Formula (IIIc), Formula (IIId), Formula (IIIe), Formula (IVa), Formula (IVb), Formula (IVc), Formula (IVd), Formula (IVe) as well as the specific compounds disclosed herein.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxy”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxy” include, but are not limited to, methoxy, ethoxy, propoxy (e.g. n-propoxy and isopropoxy), t-butoxy, neopentoxy, n-hexoxy, and the like.

As used herein, the term “amino” refers to —NH₂ group. Amino groups may also be substituted with one or more groups such as alkyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cyano” refers to —CN.

As used herein, the term “cyanoalkyl” refers to an alkyl, as defined above, substituted with one or more cyano.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. The fused, spiro and bridged ring systems are also included within the scope of this definition. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “halogen” refers to an atom selected from fluorine (or fluoro), chlorine (or chloro), bromine (or bromo) and iodine (or iodo).

As used herein, the term “haloalkyl” refers to an alkyl, as defined above, that is substituted by one or more halogens, as defined above. Examples of haloalkyl include, but are not limited to, trifluoromethyl, difluoromethyl, trichloromethyl, 2,2,2-trifluoroethyl, 1,2-difluoroethyl, 3-bromo-2-fluoropropyl, 1,2-dibromoethyl, and the like.

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxy and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, hexahydro-1H-pyrrolizinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” or “hydroxy” refers to —OH.

As used herein, the term “hydroxyalkyl” refers to an alkyl, as defined above, substituted with one or more hydroxyl.

As used herein, the term “oxo” refers to ═O substituent.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### COMPOUNDS

In one aspect, the present disclosure provides a compound having Formula (I) or Formula (II):

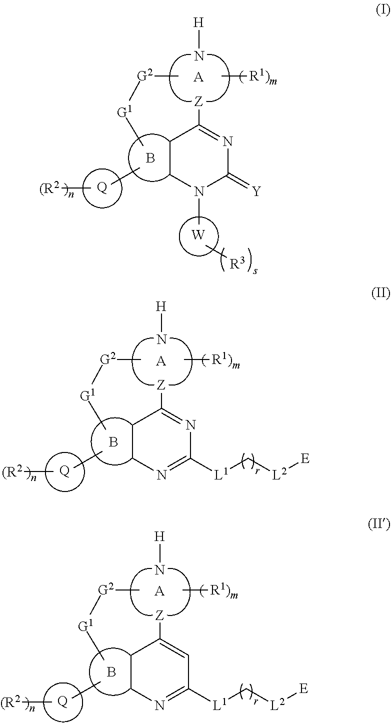

or a pharmaceutically acceptable salt thereof,
wherein
   Y is O or S;
   Ring A is heterocyclyl or heteroaryl,
   each R¹ is independently selected from the group consisting of oxo, hydroxyl, halogen, cyano, alkyl, alkenyl, alkynyl, heteroalkyl, heteroaryl, —C(O)R*, —C(O)OR*, —C(O)N(R^{a})₂, —N(R^{a})₂, —P(O)OR*OR**, and —C(O)OC(R^{a})—Z¹—Z², wherein the alkyl, alkenyl, alkynyl and heteroaryl are optionally substituted with one or more groups independently selected from cyano, hydroxyl, halogen, —OR^{b}, or —N(R^{b})₂;
   each R^{a} and R^{b} is independently hydrogen, alkyl, alkenyl or alkynyl;
   R* is selected from hydrogen, alkyl, alkylaryl or aryl;
   R** is selected from hydrogen, alkyl, alkenyl or alkynyl; or
   R* and R** together with the oxygen atoms to which they are attached form a heterocyclyl optionally substituted with aryl or haloaryl;
   Z¹ is —OC(O)-#, —OP(═O)(OR***)O-#, or —OP(═O)(OR*)N(R^{a})-#, wherein # end is connected to Z²;
   Z² is hydrogen or alkyl optionally substituted with —C(O)OR^{a};
   R*** is independently selected from hydrogen, alkyl, alkenyl or alkynyl; or
   R*** and Z² together with the oxygen atoms to which they are attached form a heterocyclyl optionally substituted with aryl or haloaryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   Ring W is selected from cycloalkyl, heterocyclyl, aryl and heteroaryl;
   each R³ is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   G¹ is a bond, —O—, —S(O)ₚ—, —S—S—, —N(R^{c})—, or —C(R^{d})═C(R^{d})—;
   G² is a bond, —[C(R⁴)₂]ᵤ—, —C(O)— or —C(O)C(R^{d})₂—;
   R^{e} is selected from the group consisting of hydrogen, alkyl, haloalkyl, hydroxyalkyl, heteroalkyl, cycloalkyl and heterocyclyl;
   each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of hydroxyl, halogen, cyano, amino, nitro, alkyl, alkoxy, haloalkyl, and hydroxyalkyl; or
   two R^{d} together with the carbon atom to which they are both attached form cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
   Z is C(R^{e}) or N;
   R^{e} is absent or hydrogen;
   L¹ is selected from a bond, —O—, —S—, —N(R^{a})—, —C(O)N(R^{a})—, alkenyl, alkynyl or cycloalkyl;

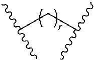

is optionally substituted with hydroxyl, halogen, cyano or amino;
L² is a bond, alkyl, cycloalkyl, heterocyclyl, aryl or heteroaryl, wherein the cycloalkyl, heterocyclyl, aryl and heteroaryl are optionally substituted with one or more groups independently selected from hydroxyl, halogen, cyano, amino, alkyl, hydroxyalkyl or heteroaryl;
E is selected from the group consisting of hydrogen, hydroxyl, halogen, —N(R^{a})₂, alkyl, haloalkyl, heteroalkyl, cycloalkyl, heterocyclyl, aryl, heteroaryl, —COOH, —CH₂OC(O)-heterocyclyl, —CH₂OC(O)N(R^{a})₂, —NHC(═NH)NH₂, —C(O)N(R^{a})₂, —OR^{a}, —(CH₂OR^{a})(CH₂)ₚOR^{a}, —N(R^{a})C(O)-aryl and —(CH₂)ᵤ-heterocyclyl, wherein the cycloalkyl, heterocyclyl, aryl, and heteroaryl are optionally substituted with one or more R″, and the aryl portion in —N(R^{a})C(O)-aryl and the heterocyclyl portion in —(CH₂)ᵤ-heterocyclyl and —CH₂OC(O)-heterocyclyl is optionally substituted with one or more R′″;
each R″ is independently selected from hydroxyl, halogen, —C(O)H, alkyl, alkoxy, haloalkyl, hydroxyalkyl, or —N(R^{a})₂;
each R′″ is independently selected from oxo, hydroxyl, halogen, alkyl, heteroalkyl, hydroxyalkyl, haloalkyl, alkoxy, -T-phenyl, -T-phenylSO₂F, —N(R^{a})₂, —SO₂F, —C(O)(alkyl), or —C(O)(haloalkyl), wherein the alkyl, heteroalkyl, hydroxyalkyl, haloalkyl, and alkoxy are optionally substituted with one or more groups independently selected from aryl, heteroaryl, or tert-butyldimethylsilyloxy;
T is a bond, —O—, or —NHC(O)—;
m is an integer from 0 to 6;
n is an integer from 0 to 5;
r is an integer from 0 to 4;
s is an integer from 0 to 5;
p is an integer from 0 to 2; and
u is an integer from 0 to 4.

In another aspect, the present disclosure provides a compound having Formula (III) or Formula (IV):

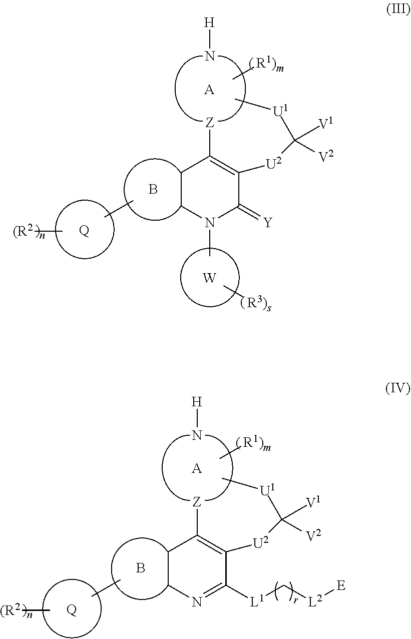

or a pharmaceutically acceptable salt thereof,
wherein
   Y is O or S;
   Ring A is heterocyclyl or heteroaryl,
   each R¹ is independently selected from the group consisting of oxo, hydroxyl, halogen, cyano, alkyl, alkenyl, alkynyl, heteroalkyl, heteroaryl, —C(O)R*, —C(O)OR*, —C(O)N(R^{a})₂, —N(R^{a})₂, —P(O)OR*OR**, and —C(O)OC(R^{a})ₜ—Z¹—Z², wherein the alkyl, alkenyl, alkynyl and heteroaryl are optionally substituted with one or more groups independently selected from cyano, hydroxyl, halogen, —OR^{b}, or —N(R^{b})₂;
   each R^{a} and R^{b} is independently hydrogen, alkyl, alkenyl or alkynyl;
   R* is selected from hydrogen, alkyl, alkylaryl or aryl;
   R** is selected from hydrogen, alkyl, alkenyl or alkynyl; or
   R* and R** together with the oxygen atoms to which they are attached form a heterocyclyl optionally substituted with aryl or haloaryl;
   Z¹ is —OC(O)-#, —OP(═O)(OR***)O-#, or —OP(═O)(OR*)N(R^{a})-#, wherein # end is connected to Z²;
   Z² is hydrogen or alkyl optionally substituted with —C(O)OR^{a};
   R*** is independently selected from hydrogen, alkyl, alkenyl or alkynyl; or
   R*** and Z² together with the oxygen atoms to which they are attached form a heterocyclyl optionally substituted with aryl or haloaryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   Ring W is selected from cycloalkyl, heterocyclyl, aryl and heteroaryl;
   each R³ is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
   U¹ is selected from a bond, —(CH₂)ₜ—, —O(CH₂)ₜ—, —N(R^{j})—, —C(O)— or —C(O)C(R^{j})₂—;
   U² is selected from —O—, —S(O)ₚ—, —[C(R^{j})₂]ₜ—, —C(R^{j})═C(R^{j})—, —N(R^{j})—, —C(O)— or —C(O)C(R^{j})₂—;
   R^{j} is selected from the group consisting of hydrogen, cyano, alkyl, haloalkyl, hydroxyalkyl, heteroalkyl, cycloalkyl and heterocyclyl, wherein the alkyl, heteroalkyl, cycloalkyl and heterocyclyl are optionally substituted with —N(R^{a})₂ or heterocyclyl;
   V¹ and V² are each independently selected from the group consisting of hydrogen, alkyl, alkenyl, alkynyl, alkoxy, heteroalkyl, cycloalkyl and heterocyclyl, wherein the alkyl, alkenyl, alkynyl, alkoxy, heteroalkyl, cycloalkyl and heterocyclyl are optionally substituted with one or more groups independently selected from hydroxyl, halogen, cyano, amino, alkyl, or haloalkyl; or
   V¹ and V² together with the carbon atom to which they are both attached form oxo, cycloalkyl, heterocyclyl or heteroaryl, wherein cycloalkyl, heterocyclyl, and heteroaryl are optionally substituted with one or more groups independently selected from the group consisting of cyano, halogen, hydroxyl, amino, and alkyl optionally substituted with heterocyclyl or —N(R^{a})₂;
   Z is C(R^{e}) or N;
   R^{e} is absent or hydrogen;
   L¹ is selected from a bond, —O—, —S—, —N(R^{a})—, alkenyl, alkynyl or cycloalkyl;

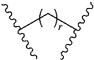

is optionally substituted with hydroxyl, halogen, cyano or amino;
L² is a bond, alkyl, cycloalkyl, heterocyclyl, aryl or heteroaryl, wherein the cycloalkyl, heterocyclyl, aryl and heteroaryl are optionally substituted with one or more groups independently selected from hydroxyl, halogen, cyano, amino, alkyl, hydroxyalkyl or heteroaryl;
E is selected from the group consisting of hydrogen, hydroxyl, halogen, —N(R^{a})₂, alkyl, haloalkyl, heteroalkyl, cycloalkyl, heterocyclyl, aryl, heteroaryl, —COOH, —CH₂OC(O)-heterocyclyl, —NHC(═NH)NH₂, —C(O)N(R^{a})₂, —OR^{a}, —(CH₂OR^{a})(CH₂)ₚOR^{a}, —N(R^{a})C(O)-aryl and —(CH₂)ᵤ-heterocyclyl, wherein the cycloalkyl, heterocyclyl, aryl, and heteroaryl are optionally substituted with one or more R″, and the aryl portion in —N(R^{a})C(O)-aryl and the heterocyclyl portion in —(CH₂)ᵤ-heterocyclyl and —CH₂OC(O)-heterocyclyl is optionally substituted with one or more R′″;
each R″ is independently selected from hydroxyl, halogen, —C(O)H, alkyl, alkoxy, haloalkyl, hydroxyalkyl, or —N(R^{a})₂;
each R′″ is independently selected from oxo, hydroxyl, halogen, alkyl, heteroalkyl, hydroxyalkyl, haloalkyl, alkoxy, -T-phenyl, -T-phenylSO₂F, —N(R^{a})₂, —SO₂F, —C(O)(alkyl), or —C(O)(haloalkyl), wherein the alkyl, heteroalkyl, hydroxyalkyl, haloalkyl, and alkoxy are optionally substituted with one or more groups independently selected from aryl, heteroaryl, or tert-butyldimethylsilyloxy;
T is a bond, —O—, or —NHC(O)—;
m is an integer from 0 to 6;
n is an integer from 0 to 5;
r is an integer from 0 to 4;
s is an integer from 0 to 5;
p is an integer from 0 to 2;
t is an integer from 0 to 3; and
u is an integer from 0 to 4.

In some embodiments, Z is C(R^{e}). In certain embodiments, R^{e} is absent. In certain embodiments, R^{e} is hydrogen.

In some embodiments, Z is N.

In some embodiments, Ring A is heterocyclyl. In certain embodiments, Ring A is a 6- to 12-membered heterocyclyl. In certain embodiments, Ring A is a 6- to 10-membered heterocyclyl. In certain embodiments, Ring A is a 8- to 10-membered heterocyclyl.

In some embodiments, Ring A is heteroaryl. In certain embodiments, Ring A is a 6- to 12-membered heteroaryl. In certain embodiments, Ring A is a 6- to 10-membered heteroaryl. In certain embodiments, Ring A is a 8- to 10-membered heteroaryl.

In some embodiments, Ring A is a bridged heterocyclyl optionally containing at least one further heteroatom selected from the group consisting of N, S and O. In certain embodiments, Ring A is a 6- to 12-membered bridged heterocyclyl optionally containing at least one further heteroatom selected from the group consisting of N, S and O. In certain embodiments, Ring A is a 6- to 10-membered bridged heterocyclyl optionally containing at least one further heteroatom selected from the group consisting of N, S and O. In certain embodiments, Ring A is a 8- to 10-membered bridged heterocyclyl optionally containing at least one further heteroatom selected from the group consisting of N, S and O.

In certain embodiments, Ring A is selected from the group consisting of:

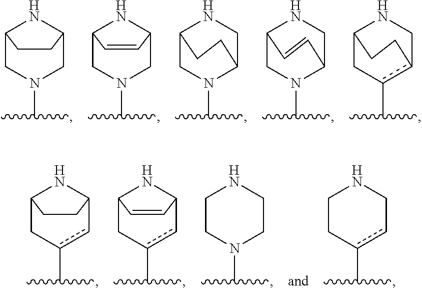

wherein [symbol] represents a single bond or a double bond.

In some embodiments, Ring A is a spiro or fused ring optionally containing at least one further heteroatom selected from the group consisting of N, S and O.

In certain embodiments, Ring A is selected from the group consisting of:

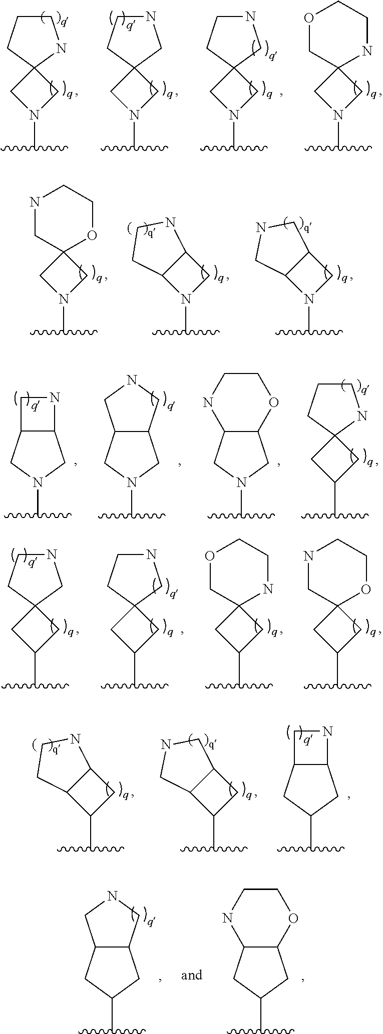

wherein q is an integer from 1 to 4, and q′ is an integer from 0 to 4.

In some embodiments, Ring B is cycloalkyl optionally substituted with one or more R′. In certain embodiments, Ring B is C₅₋₁₂ cycloalkyl optionally substituted with one or more R′. In certain embodiments, Ring B is C₅₋₁₀ cycloalkyl optionally substituted with one or more R′. In certain embodiments, Ring Bis C₅₋₈ cycloalkyl optionally substituted with one or more R′. In certain embodiments, Ring B is C₅₋₇ cycloalkyl optionally substituted with one or more R′. In certain embodiments, Ring B is C₅₋₆ cycloalkyl optionally substituted with one or more R′.

In some embodiments, Ring B is heterocyclyl optionally substituted with one or more R′. In certain embodiments, Ring B is 5- to 12-membered heterocyclyl optionally substituted with one or more R′. In certain embodiments, Ring B is 5- to 10-membered heterocyclyl optionally substituted with one or more R′. In certain embodiments, Ring B is 5- to 8-membered heterocyclyl optionally substituted with one or more R′. In certain embodiments, Ring B is 5- to 7-membered heterocyclyl optionally substituted with one or more R′. In certain embodiments, Ring B is 5- to 6-membered heterocyclyl optionally substituted with one or more R′.

In certain embodiments, Ring B is 1,2,3,6-tetrahydropyridinyl or piperidinyl, each optionally substituted with one or more R′ independently selected from oxo, alkyl, alkynyl, heteroalkyl, or cyano.

In some embodiments, Ring B is aryl optionally substituted with one or more R′. In certain embodiments, Ring B is C₅₋₁₂ aryl optionally substituted with one or more R′. In certain embodiments, Ring B is C₅₋₁₀ aryl optionally substituted with one or more R′. In certain embodiments, Ring B is C₅₋₈ aryl optionally substituted with one or more R′. In certain embodiments, Ring B is C₅₋₇ aryl optionally substituted with one or more R′. In certain embodiments, Ring B is C₅₋₆ aryl optionally substituted with one or more R′.

In certain embodiments, Ring B is phenyl optionally substituted with one or more R′.

In some embodiments, Ring B is heteroaryl optionally substituted with one or more R′. In certain embodiments, Ring B is 5- to 12-membered heteroaryl optionally substituted with one or more R′. In certain embodiments, Ring B is 5- to 10-membered heteroaryl optionally substituted with one or more R′. In certain embodiments, Ring B is 5- to 8-membered heteroaryl optionally substituted with one or more R′. In certain embodiments, Ring B is 5- to 7-membered heteroaryl optionally substituted with one or more R′. In certain embodiments, Ring B is 5- to 6-membered heteroaryl optionally substituted with one or more R′.

In certain embodiments, Ring B is pyridinyl or pyrimidinyl, each optionally substituted with one or more R′.

In some embodiments, Ring Q is cycloalkyl. In certain embodiments, Ring Q is C₅₋₁₂ cycloalkyl. In certain embodiments, Ring Q is C₅₋₁₀ cycloalkyl. In certain embodiments, Ring Q is C₅₋₈ cycloalkyl. In certain embodiments, Ring Q is C₅₋₇ cycloalkyl. In certain embodiments, Ring Q is C₅₋₆ cycloalkyl.

In some embodiments, Ring Q is heterocyclyl. In certain embodiments, Ring Q is 5- to 12-membered heterocyclyl. In certain embodiments, Ring Q is 5- to 10-membered heterocyclyl. In certain embodiments, Ring Q is 5- to 8-membered heterocyclyl. In certain embodiments, Ring Q is 5- to 7-membered heterocyclyl. In certain embodiments, Ring Q is 5- to 6-membered heterocyclyl.

In some embodiments, Ring Q is aryl. In certain embodiments, Ring Q is C₅₋₁₂ aryl. In certain embodiments, Ring Q is C₅₋₁₀ aryl. In certain embodiments, Ring Q is C₅₋₈ aryl. In certain embodiments, Ring Q is C₅₋₇ aryl. In certain embodiments, Ring Q is C₅₋₆ aryl.

In certain embodiments, Ring Q is phenyl or naphthalenyl.

In some embodiments, Ring Q is heteroaryl. In certain embodiments, Ring Q is 5- to 12-membered heteroaryl. In certain embodiments, Ring Q is 5- to 10-membered heteroaryl. In certain embodiments, Ring Q is 5- to 8-membered heteroaryl. In certain embodiments, Ring Q is 5- to 7-membered heteroaryl. In certain embodiments, Ring Q is 5- to 6-membered heteroaryl.

In certain embodiments, Ring Q is selected from benzothiophenyl, benzoimidazolyl, quinazolinyl, benzotriazolyl, thiophenyl, thienopyridinyl, isoquinolinyl, indolyl, or indazolyl.

In some embodiments, Ring W is cycloalkyl or heterocyclyl.

In certain embodiments, Ring W is cyclopropyl, cyclobutyl, cyclopentyl, or cyclohexyl.

In some embodiments, Ring W is heterocyclyl.

In certain embodiments, Ring W is tetrahydrofuranyl, pyrrolidinyl, tetrahydro-2H-pyranyl, piperidinyl, or piperazinyl.

In some embodiments, Ring W is aryl. In certain embodiments, Ring Q is C₅₋₁₂ aryl. In certain embodiments, Ring Q is C₅₋₁₀ aryl. In certain embodiments, Ring Q is C₅₋₈ aryl. In certain embodiments, Ring Q is C₅₋₇ aryl. In certain embodiments, Ring Q is C₅₋₆ aryl.

In certain embodiments, Ring W is phenyl or naphthalenyl.

In some embodiments, Ring W is heteroaryl. In certain embodiments, Ring W is 5- to 12-membered heteroaryl. In certain embodiments, Ring W is 5- to 10-membered heteroaryl. In certain embodiments, Ring W is 5- to 8-membered heteroaryl. In certain embodiments, Ring W is 5- to 7-membered heteroaryl. In certain embodiments, Ring W is 5- to 6-membered heteroaryl.

In certain embodiments, Ring W is selected from the group consisting of pyridinyl, pyrimidinyl pyridazinyl, pyrazinyl, thienyl, thiazolyl, isothiazolyl, oxazolyl, isoxazolyl, imidazolyl, pyrazolyl, 1,2,3-triazolyl, 1,2,4-triazolyl, benzofuranyl, benzothienyl, indolyl, benzimidazolyl, benzopyrazolyl, purinyl, quinolinyl, isoquinolinyl, isoquinoline-1 (2H)-one group, isoindolin-1-one group, benzo[d]oxazole-2 (H)-one group and 1,3-dihydro-2H-benzo[d]imidazol-2-one group.

In some embodiments, G¹ is a bond.

In some embodiments, G¹ is —O—.

In some embodiments, G¹ is —S(O)ₚ—.

In some embodiments, G¹ is —N(R^{c})—. In certain embodiments, R^{e} is hydrogen. In certain embodiments, R^{e} is alkyl. In certain embodiments, R^{e} is C₁₋₆ alkyl, C₁₋₅ alkyl, C₁₋₄ alkyl, C₁₋₃ alkyl. In certain embodiments, R^{e} is methyl. In some embodiments, G¹ is —C(O)—.

In some embodiments, G¹ is —C(R^{d})═C(R^{d})—. In certain embodiments, each R^{d} is independently hydrogen or alkyl. In certain embodiments, both R^{d} are hydrogen. In certain embodiments, both R^{d} are alkyl, such as C₁₋₆ alkyl, C₁₋₅ alkyl, C₁₋₄ alkyl, or C₁₋₃ alkyl. In certain embodiments, one R^{d} is hydrogen and the other is alkyl. In certain embodiments, one R^{d} is hydrogen and the other is methyl.

In some embodiments, G² is a bond.

In some embodiments, G² is —[C(R^{d})₂]ᵤ—. In certain embodiments, each R^{d} is independently hydrogen, hydroxyl or alkyl. In certain embodiments, each R^{d} is independently hydrogen, hydroxyl, C₁₋₆ alkyl, C₁₋₅ alkyl, C₁₋₄ alkyl, or C₁₋₃ alkyl. In certain embodiments, each R^{d} is independently hydrogen, hydroxyl or methyl.

In some embodiments, G² is —C(O)—.

In some embodiments, G² is —C(O)C(R^{d})₂—. In certain embodiments, each R^{d} is independently hydrogen or alkyl. In certain embodiments, both R^{d} are hydrogen. In certain embodiments, both R^{d} are alkyl, such as C₁₋₆ alkyl, C₁₋₅ alkyl, C₁₋₄ alkyl, or C₁₋₃ alkyl. In certain embodiments, one R^{d} is hydrogen and the other is alkyl. In certain embodiments, one R^{d} is hydrogen and the other is methyl.

In some embodiments, G¹ is —O—, and G² is —[C(R^{d})₂]ᵤ— wherein u is 1 or 2. In certain embodiments, each R^{d} is independently hydrogen or alkyl. In certain embodiments, each R^{d} is independently hydrogen, C₁₋₆ alkyl, C₁₋₅ alkyl, C₁₋₄ alkyl, or C₁₋₃ alkyl. In certain embodiments, each R^{d} is independently hydrogen or methyl.

In some embodiments, G¹ is —S(O)ₚ—, and G² is —[C(R^{d})₂]ᵤ— wherein u is 1. In certain embodiments, each R^{d} is independently hydrogen or alkyl. In certain embodiments, each R^{d} is independently hydrogen, C₁₋₆ alkyl, C₁₋₅ alkyl, C₁₋₄ alkyl, or C₁₋₃ alkyl. In certain embodiments, each R^{d} is independently hydrogen or methyl. In certain embodiments, both R^{d} are hydrogen.

In some embodiments, G1 is —N(R^{c})—, and G² is —C(O)—, —C(O)C(R^{d})₂— or —[C(R^{d})₂]ᵤ— wherein u is 1 or 2. In certain embodiments, each R^{d} is independently hydrogen or alkyl. In certain embodiments, each R^{d} is independently hydrogen, C₁₋₆ alkyl, C₁₋₅ alkyl, C₁₋₄ alkyl, or C₁₋₃ alkyl. In certain embodiments, each R^{d} is independently hydrogen or methyl.

In some embodiments, G¹ is —C(R^{d})═C(R^{d})—, G² is —C(O)— or —[C(R^{d})₂]ᵤ— wherein u is 1. In certain embodiments, each R^{d} is independently hydrogen, hydroxyl, C₁₋₆ alkyl, C₁₋₅ alkyl, C₁₋₄ alkyl, or C₁₋₃ alkyl. In certain embodiments, each R^{d} is independently hydrogen, hydroxyl or methyl.

In some embodiments, m is 0.

In some embodiments, m is an integer from 1 to 3, and each R¹ is independently alkyl, such as C₁₋₆ alkyl, C₁₋₅ alkyl, C₁₋₄ alkyl, or C₁₋₃ alkyl.

In some embodiments, m is 1, and R¹ is —C(O)R* or —C(O)OR*, wherein R* is alkyl or alkylaryl. In certain embodiments, m is 1 and R¹ is substituted at the —NH— position in Ring A. In certain embodiments, R^{a} is C₁₋₆ alkyl, C₁₋₅ alkyl, C₁₋₄ alkyl, or C₁₋₃ alkyl. In certain embodiments, R^{a} is benzyl. In certain embodiments, m is 1, and R¹ is selected from

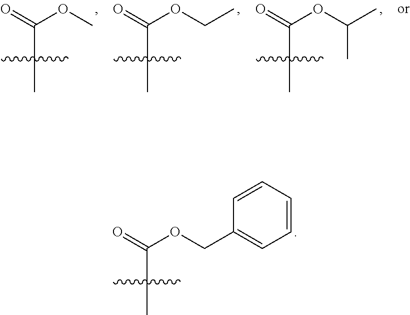

In some embodiments, m is 1, and R¹ is —P(O)OR*OR**. In certain embodiments, m is 1 and R¹ is substituted at the —NH— position in Ring A. In certain embodiments, m is 1, R¹ is —P(O)OR*OR**, and R* and R** together with the oxygen atoms to which they are attached form a heterocyclyl optionally substituted with aryl or haloaryl. In certain embodiments, m is 1, R¹ is

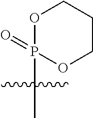 [SMILES: *P1(=O)OCCCO1]

optionally substituted with aryl or haloaryl. In certain embodiments, m is 1, R¹ is

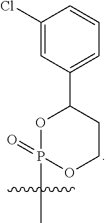 [SMILES: *P1(=O)OCCC(c2cccc(Cl)c2)O1]

In some embodiments, m is 1, R¹ is —C(O)OC(R^{a})₂—Z¹—Z², Z¹ is —OC(O)-# and Z² is alkyl optionally substituted with aryl. In certain embodiments, m is 1 and R¹ is substituted at the —NH— position in Ring A. In certain embodiments, Z² is C₁₋₆ alkyl, C₁₋₅ alkyl, C₁₋₄ alkyl, or C₁₋₃ alkyl, each optionally substituted with aryl (such as phenyl). In certain embodiments, m is 1, R¹ is

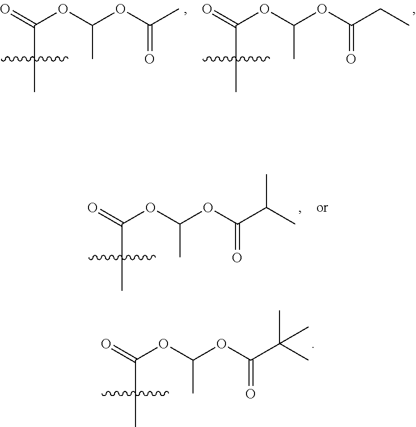

In some embodiments, m is 1, R¹ is —C(O)OC(R^{a})₂—Z¹—Z², Z¹ is —OP(═O)(OR***) N(R^{a})-#, Z² is alkyl substituted with —OC(O)R^{a}, and R*** is aryl. In certain embodiments, m is 1 and R¹ is substituted at the —NH— position in Ring A. In certain embodiments, R* is phenyl. In certain embodiments, m is 1, R¹ is

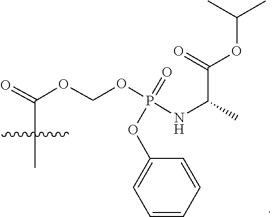 [SMILES: *C(=O)OCOP(=O)(N[C@@H](C)C(=O)OC(C)C)Oc1ccccc1]

In some embodiments, m is 1, R¹ is —C(O)OC(R^{a})₂—Z¹—Z², Z¹ is —OP(═O)(OR***)O-#, Z² is hydrogen or alkyl optionally substituted with aryl, and R*** is hydrogen, alkyl or alkylaryl. In certain embodiments, m is 1 and R¹ is substituted at the —NH— position in Ring A. In certain embodiments, Z² is C₁₋₆ alkyl, C₁₋₅ alkyl, C₁₋₄ alkyl, or C₁₋₃ alkyl, each optionally substituted with aryl.

In some embodiments, m is 1, R¹ is —C(O)OC(R^{a})₂—Z¹—Z², Z¹ is —OP(═O)(OR***)O-#, and R*** and Z² together with the oxygen atoms to which they are attached form a heterocyclyl optionally substituted with aryl or haloaryl. In certain embodiments, m is 1 and R¹ is substituted at the —NH— position in Ring A. In certain embodiments, —Z¹—Z² is

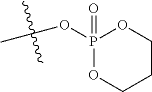 [SMILES: *OP1(=O)OCCCO1]

optionally substituted with aryl or haloaryl. In certain embodiments, —Z¹—Z² is

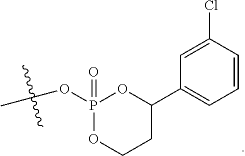 [SMILES: *OP1(=O)OCCC(c2cccc(Cl)c2)O1]

In certain embodiments, m is 1 and R¹ is

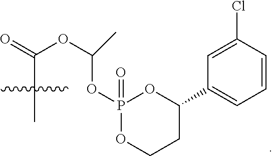 [SMILES: *C(=O)OC(C)OP1(=O)OCC[C@@H](c2cccc(Cl)c2)O1]

In some embodiments, n is an integer from 1 to 4, and each R² is independently selected from hydroxyl, halogen, cyano, amino, alkyl, alkenyl, alkynyl, or cycloalkyl, wherein alkyl, alkenyl, alkynyl, and cycloalkyl are optionally substituted with one or more groups independently selected from cyano, hydroxyl, halogen, or alkyl.

In certain embodiments, n is an integer from 1 to 4, and each R² is independently selected from hydroxyl, halogen, amino, C₁₋₃ alkyl, C₂₋₄ alkynyl, C₁₋₃ haloalkyl, or C₃₋₆ cycloalkyl.

In some embodiments, s is an integer from 1 to 4, and each R³ is independently selected from hydroxyl, halogen, cyano, amino, alkyl, alkenyl, alkynyl, or cycloalkyl, wherein alkyl, alkenyl, alkynyl, and cycloalkyl are optionally substituted with one or more groups independently selected from cyano, hydroxyl, halogen, or alkyl.

In certain embodiments, s is an integer from 1 to 4, and each R³ is independently selected from hydroxyl, halogen, amino, C₁₋₃ alkyl, or C₃₋₆ cycloalkyl.

In some embodiments, L¹ is a bond.

In some embodiments, L¹ is —O—.

In some embodiments, L¹ is —S—.

In some embodiments, L¹ is —N(R^{a})—. In certain embodiments, R^{a} is hydrogen. In certain embodiments, R^{a} is alkyl, such as C₁₋₆ alkyl, C₁₋₅ alkyl, C₁₋₄ alkyl, or C₁₋₃ alkyl.

In some embodiments, L¹ is alkenyl. In certain embodiments, L¹ is ethenyl.

In some embodiments, L¹ is alkynyl. In certain embodiments, L¹ is ethynyl.

In some embodiments, L¹ is cycloalkyl. In certain embodiments, L¹ is cyclopropyl.

In some embodiments, L² is a bond.

In some embodiments, L² is alkyl, cycloalkyl, heterocyclyl, or heteroaryl, each optionally substituted with one or more of halogen or alkyl.

In certain embodiments, L² is selected from hexahydro-1H-pyrrolizinyl, azetidinyl, pyrrolidinyl or pyridinyl.

In some embodiments, E is selected from hydrogen, hydroxyl, halogen, haloalkyl, heteroalkyl, or —CH₂OC(O)-heterocyclyl.

In some embodiments, L¹ is —O— or —N(R^{a})—, and L² is heterocyclyl or heteroaryl.

In some embodiments, L¹ is a bond or alkynyl, and L² is heterocyclyl.

In some embodiments, E is selected from hydrogen, hydroxyl, halogen, haloalkyl, heteroalkyl, —N(R^{a})₂, or —CH₂OC(O)-heterocyclyl.

In a further aspect, the present disclosure provides a compound having having Formula (Ia) or Formula (Ib):

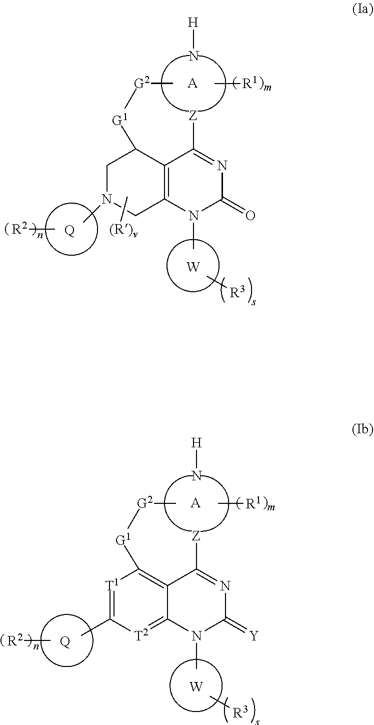

or a pharmaceutically acceptable salt thereof,
wherein
   T′ is N or C(R′);
   T² is N or C(R′);
   v is an integer from 0 to 4.

In a further aspect, the present disclosure provides a compound having a formula selected from the group consisting of:

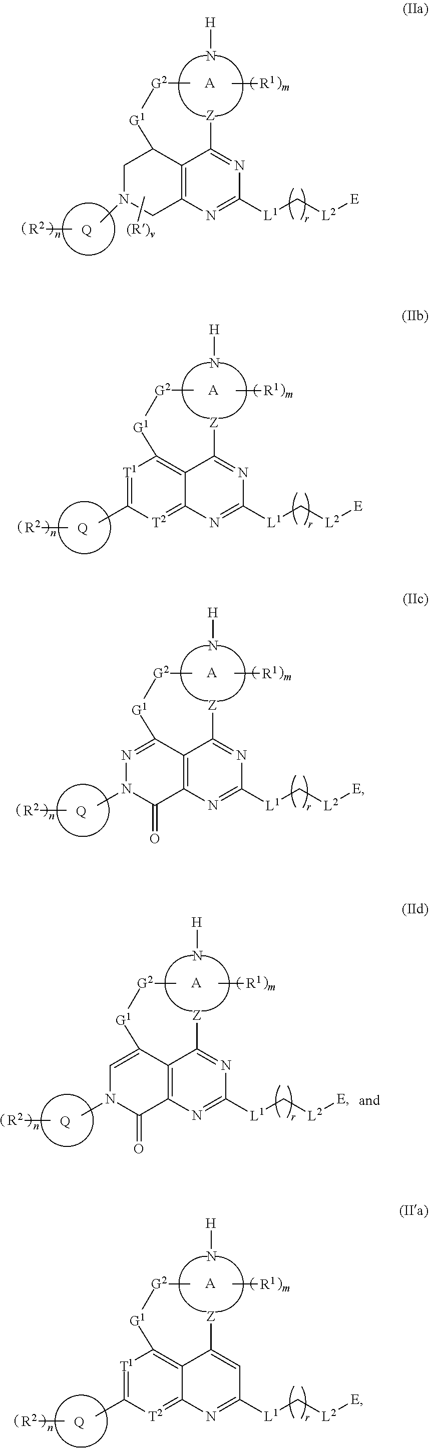

or a pharmaceutically acceptable salt thereof,
wherein
   T¹ is N or C(R′);
T² is N or C(R′);
v is an integer from 0 to 4.

In some embodiments, T² is C(R′), wherein R′ is hydrogen, hydroxyl or halogen.

In some embodiments, G¹ is —O—.

In some embodiments, G² is —[C(R^{d})₂]ᵤ—.

In some embodiments, G¹ is —O—, and G² is —[C(R^{d})₂]ᵤ— wherein u is 1 or 2. In certain embodiments, each R^{d} is independently hydrogen or alkyl. In certain embodiments, each R^{d} is hydrogen.

In some embodiments, L¹ is —O—.

In some embodiments, T¹ is N or C(R′); T² is C(R′) wherein R′ is hydrogen, hydroxyl or halogen; G¹ is —O—; G² is —[C(R^{d})₂]ᵤ— wherein u is 1 or 2; and L¹ is —O—.

In a further aspect, the present disclosure provides a compound having having Formula (IIIa) or Formula (IIIb):

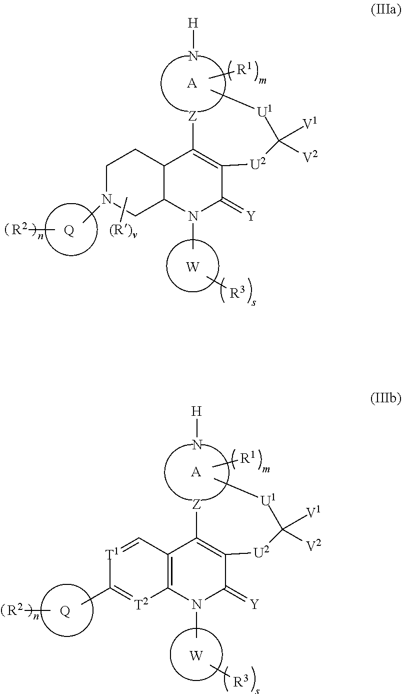

or a pharmaceutically acceptable salt thereof,
wherein
   T¹ is N or C(R′);
   T² is N or C(R′);
   v is an integer from 0 to 4.

In a further aspect, the present disclosure provides a compound having having Formula (IIIc), (IIId) or Formula (IIIe):

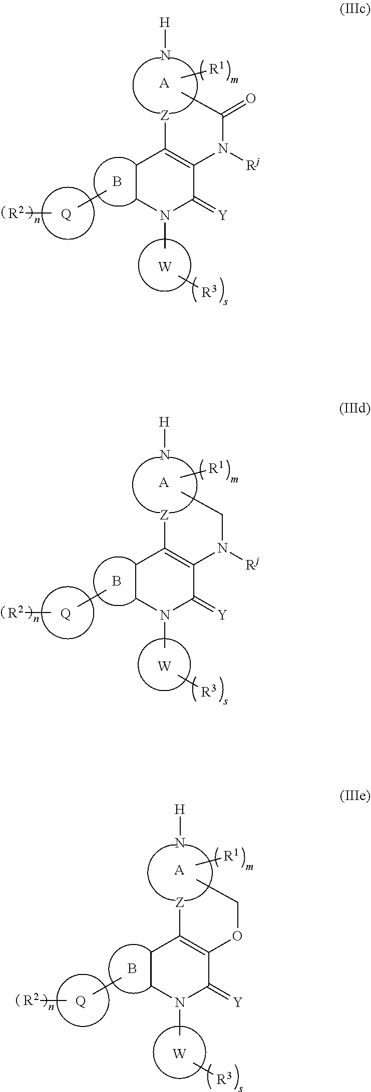

or a pharmaceutically acceptable salt thereof.

In a further aspect, the present disclosure provides a compound having having Formula (IVa) or Formula (IVb):

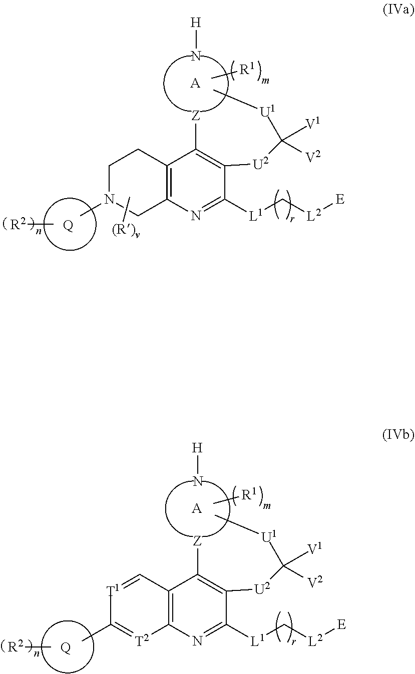

or a pharmaceutically acceptable salt thereof,
wherein
   T¹ is N or C(R′);
   T² is N or C(R′);
   v is an integer from 0 to 4.

In a further aspect, the present disclosure provides a compound having having Formula (IVc), (IVd) or Formula (IVe):

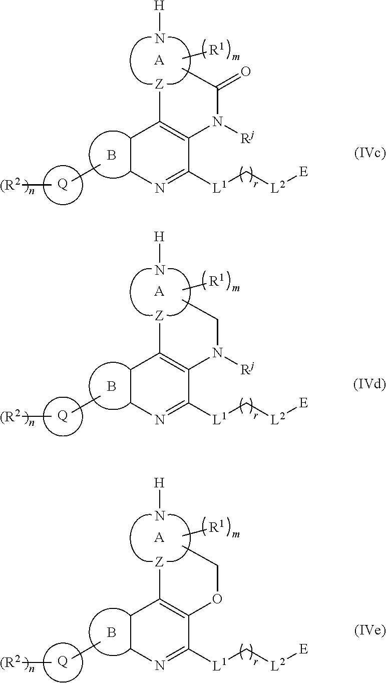

or a pharmaceutically acceptable salt thereof.

In some embodiments, the present disclosure provides a compound having a formula selected from the group consisting of:

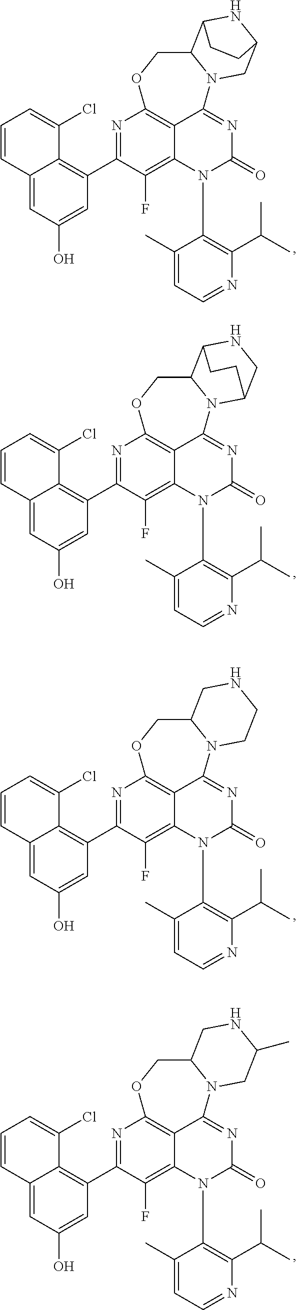

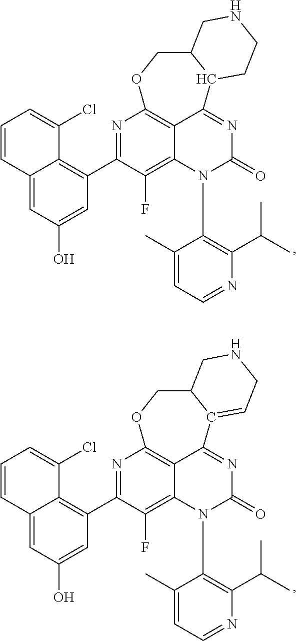 [SMILES: Cc1ccnc(C(C)C)c1-n1c(=O)nc2c3c(nc(-c4cc(O)cc5cccc(Cl)c45)c(F)c31)OCC1CNCCC21 ; "," ; Cc1ccnc(C(C)C)c1-n1c(=O)nc2c3c(nc(-c4cc(O)cc5cccc(Cl)c45)c(F)c31)OCC1CNCC=C21 ; ","]

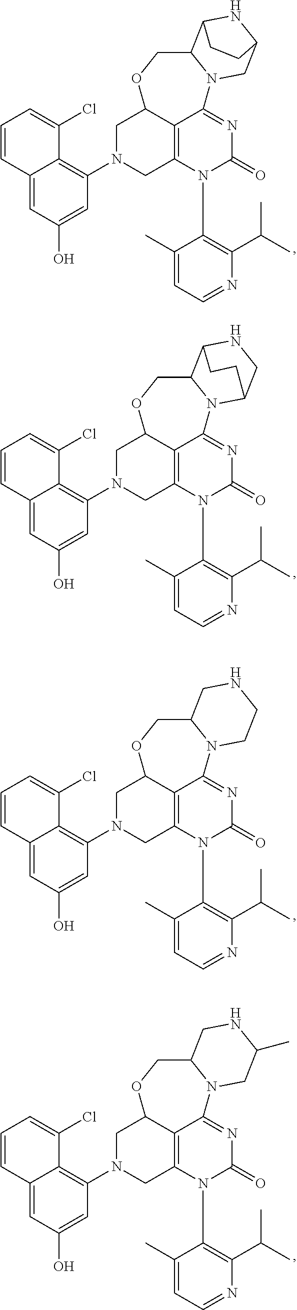

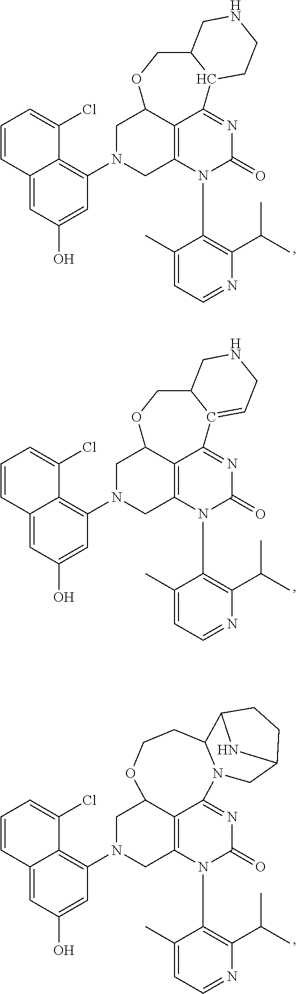 [SMILES: Cc1ccnc(C(C)C)c1-n1c2c3c(nc1=O)C1CCNCC1COC3CN(c1cc(O)cc3cccc(Cl)c13)C2 ; "," ; Cc1ccnc(C(C)C)c1-n1c2c3c(nc1=O)C1=CCNCC1COC3CN(c1cc(O)cc3cccc(Cl)c13)C2 ; "," ; Cc1ccnc(C(C)C)c1-n1c2c3c(nc1=O)N1CC4CCC(N4)C1CCOC3CN(c1cc(O)cc3cccc(Cl)c13)C2 ; ","]

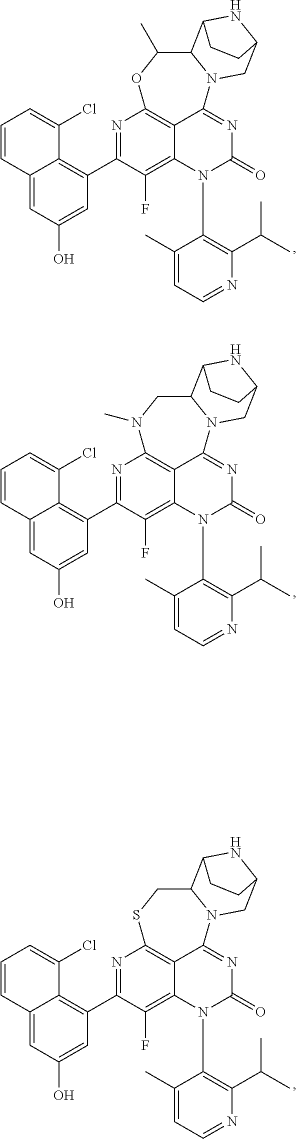 [SMILES: Cc1ccnc(C(C)C)c1-n1c(=O)nc2c3c(nc(-c4cc(O)cc5cccc(Cl)c45)c(F)c31)OC(C)C1C3CCC(CN21)N3 ; "," ; Cc1ccnc(C(C)C)c1-n1c(=O)nc2c3c(nc(-c4cc(O)cc5cccc(Cl)c45)c(F)c31)N(C)CC1C3CCC(CN21)N3 ; "," ; Cc1ccnc(C(C)C)c1-n1c(=O)nc2c3c(nc(-c4cc(O)cc5cccc(Cl)c45)c(F)c31)SCC1C3CCC(CN21)N3 ; ","]

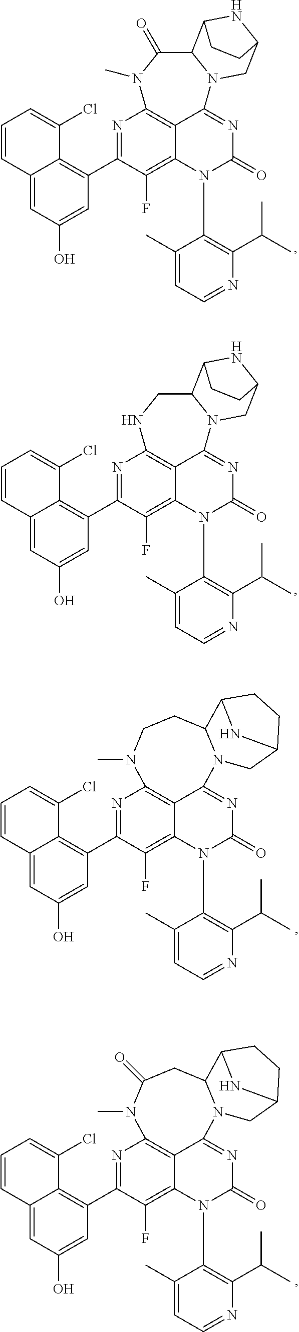

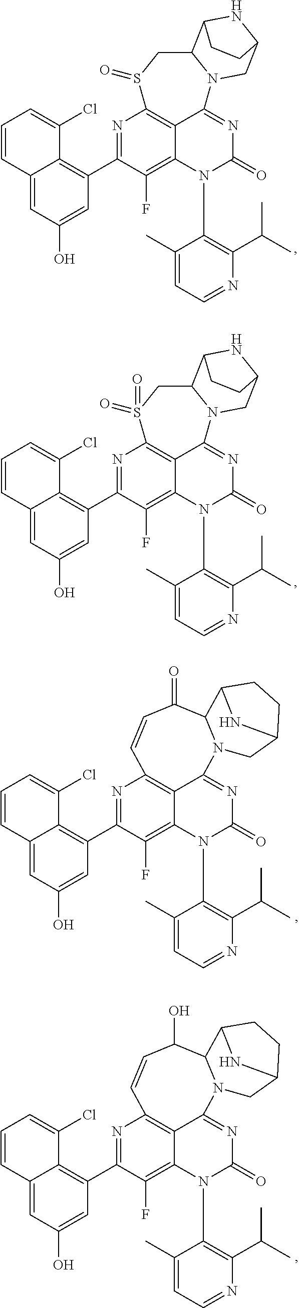

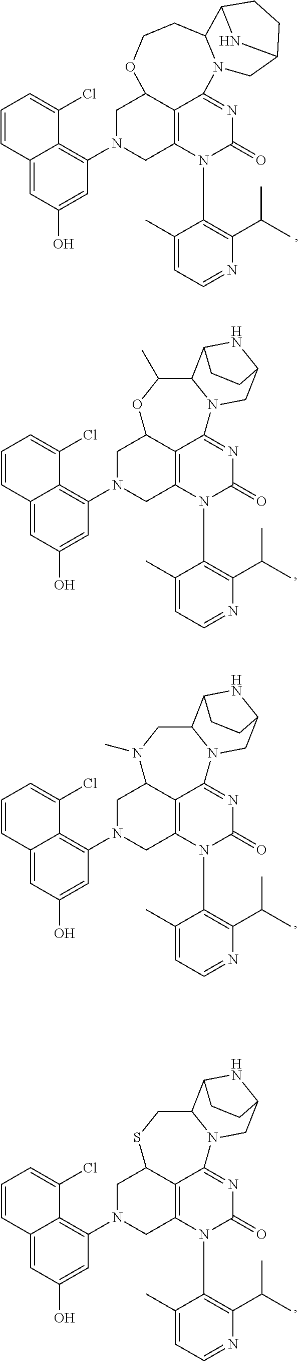

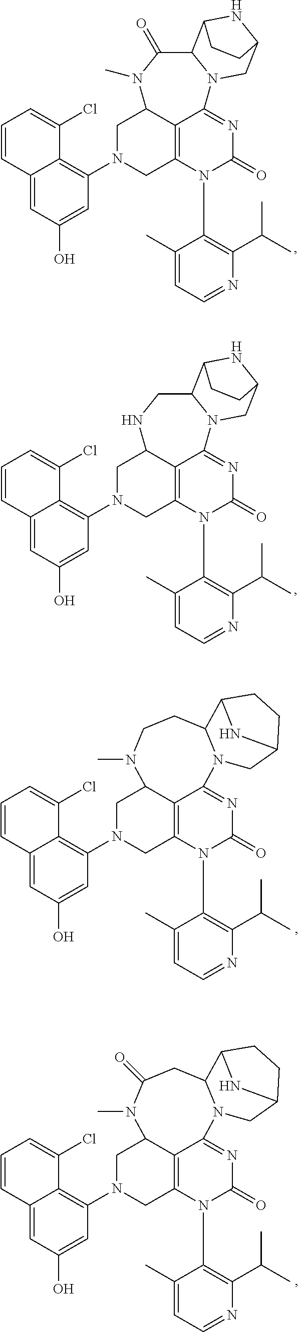

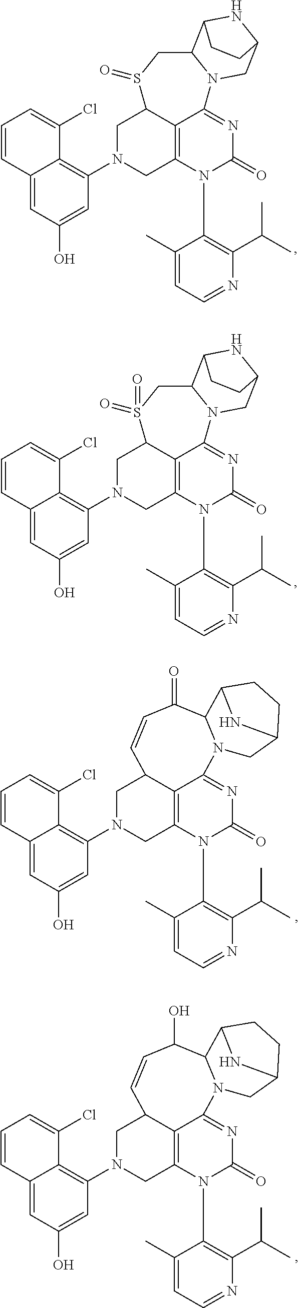

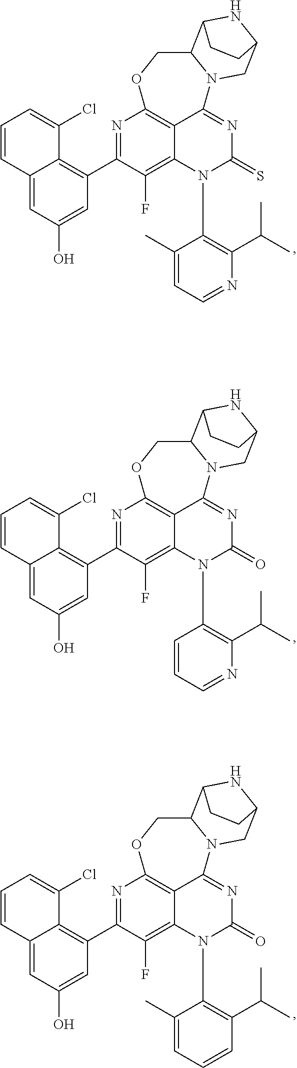 [SMILES: Cc1ccnc(C(C)C)c1-n1c(=S)nc2c3c(nc(-c4cc(O)cc5cccc(Cl)c45)c(F)c31)OCC1C3CCC(CN21)N3 ; "," ; CC(C)c1ncccc1-n1c(=O)nc2c3c(nc(-c4cc(O)cc5cccc(Cl)c45)c(F)c31)OCC1C3CCC(CN21)N3 ; "," ; Cc1cccc(C(C)C)c1-n1c(=O)nc2c3c(nc(-c4cc(O)cc5cccc(Cl)c45)c(F)c31)OCC1C3CCC(CN21)N3 ; ","]

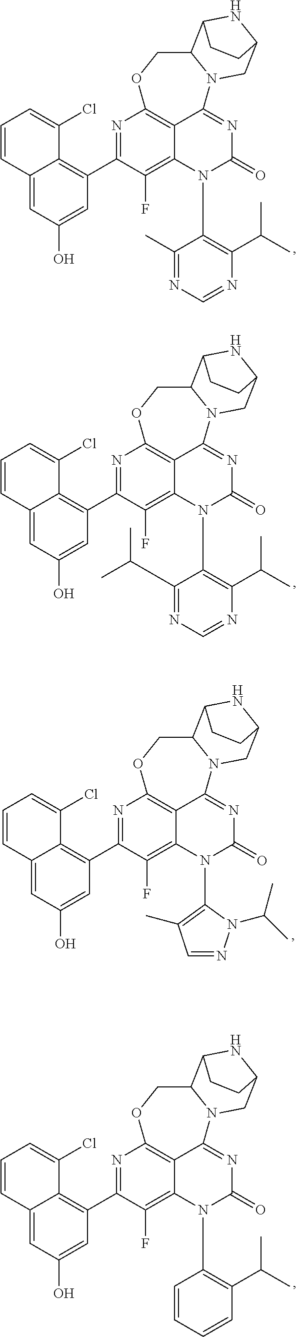

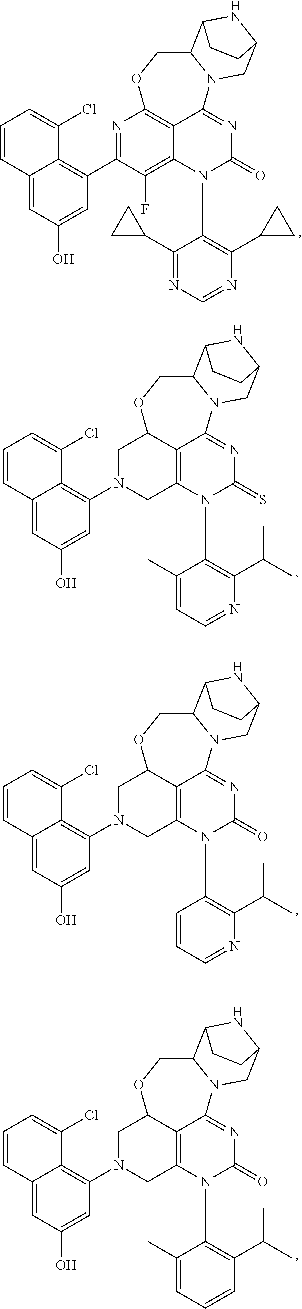

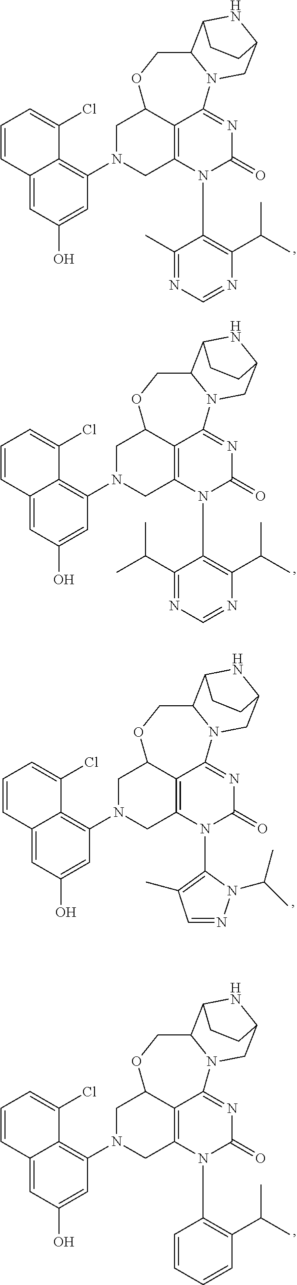

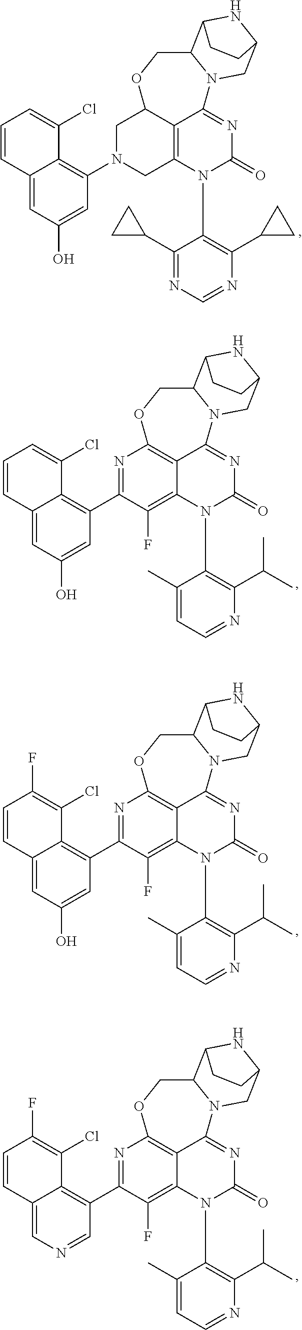

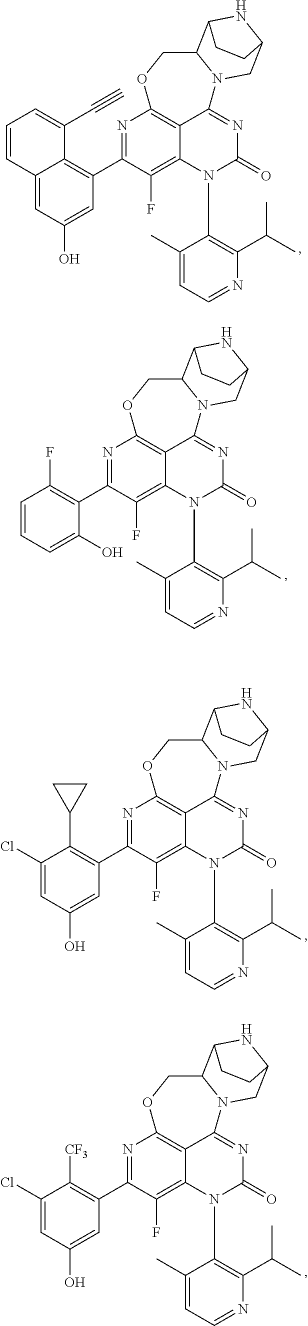

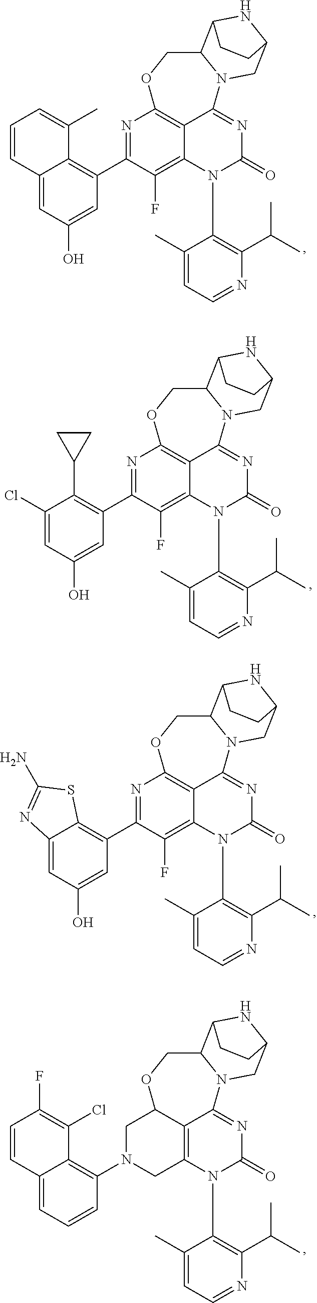

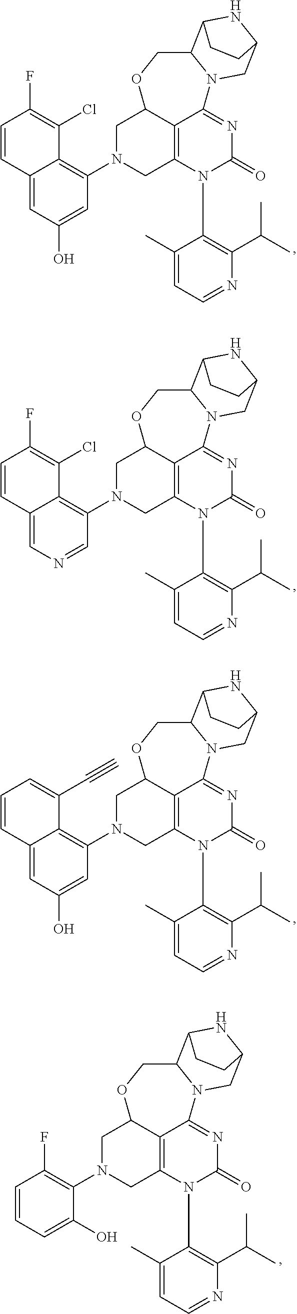

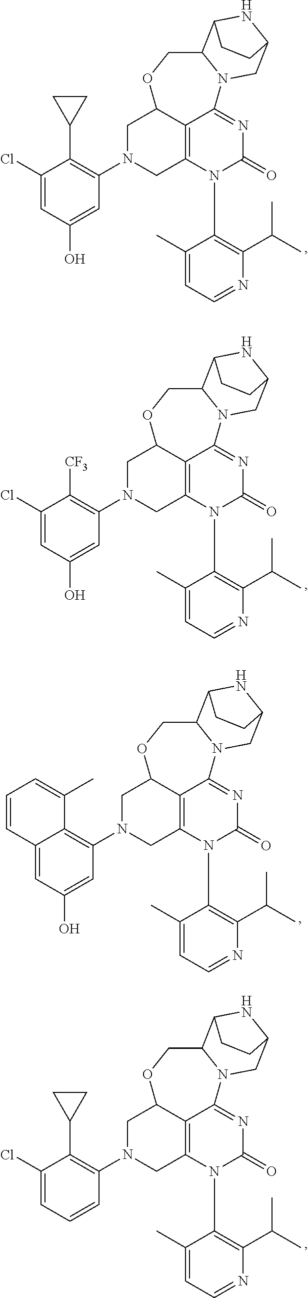

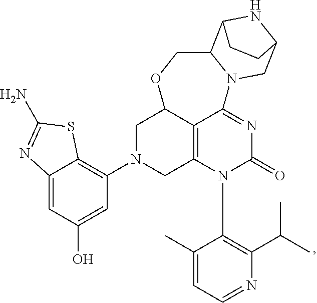 [SMILES: Cc1ccnc(C(C)C)c1-n1c2c3c(nc1=O)N1CC4CCC(N4)C1COC3CN(c1cc(O)cc3nc(N)sc13)C2 ; ","]

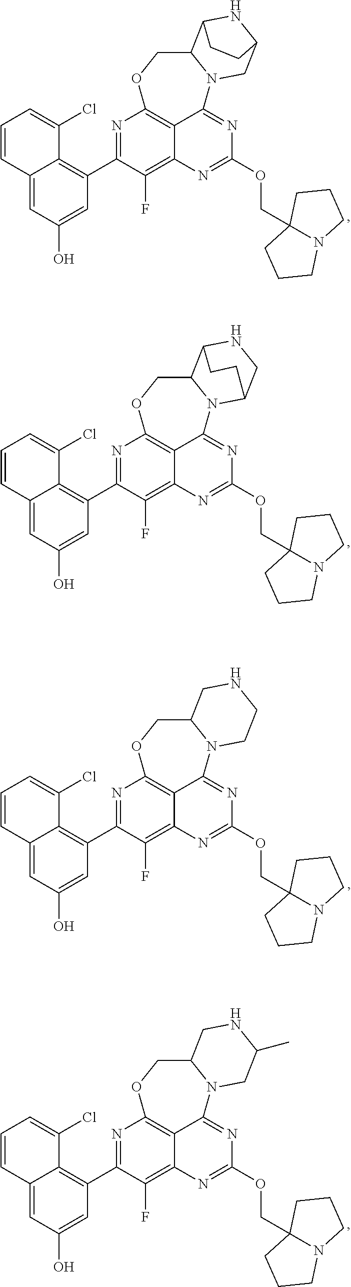

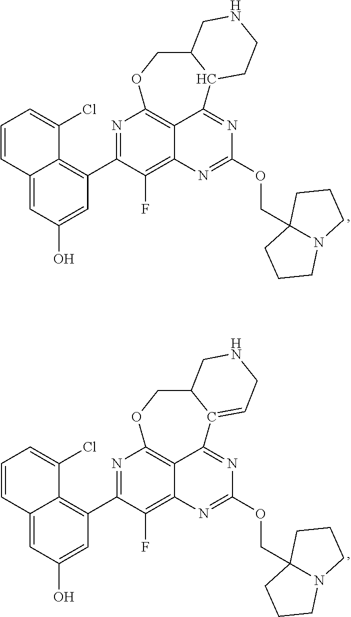 [SMILES: Oc1cc(-c2nc3c4c(nc(OCC56CCCN5CCC6)nc4c2F)C2CCNCC2CO3)c2c(Cl)cccc2c1 ; "," ; Oc1cc(-c2nc3c4c(nc(OCC56CCCN5CCC6)nc4c2F)C2=CCNCC2CO3)c2c(Cl)cccc2c1 ; ","]

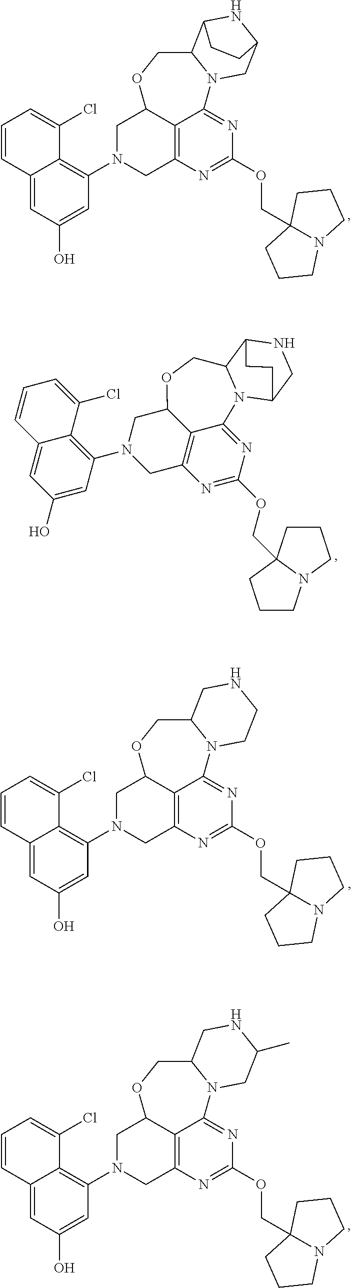

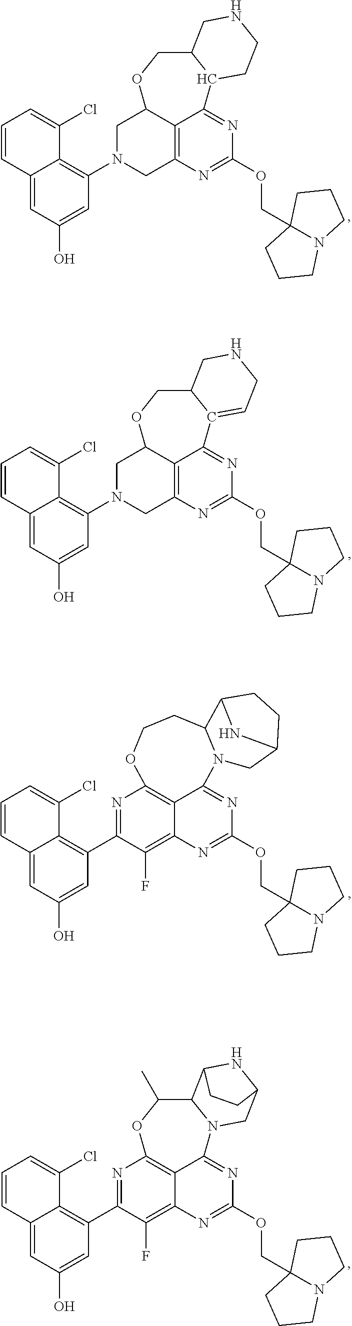

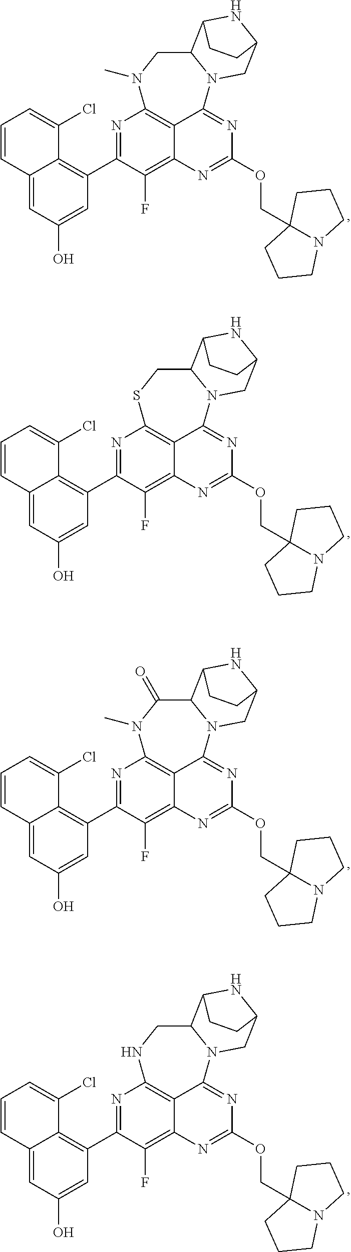

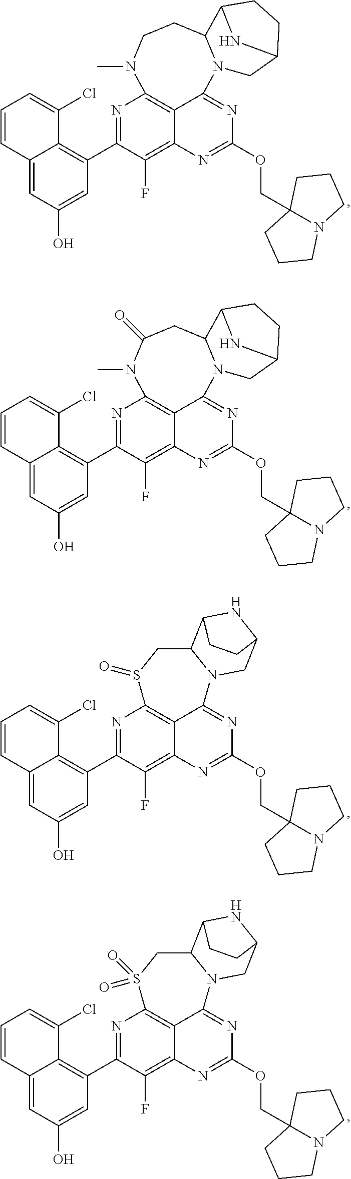

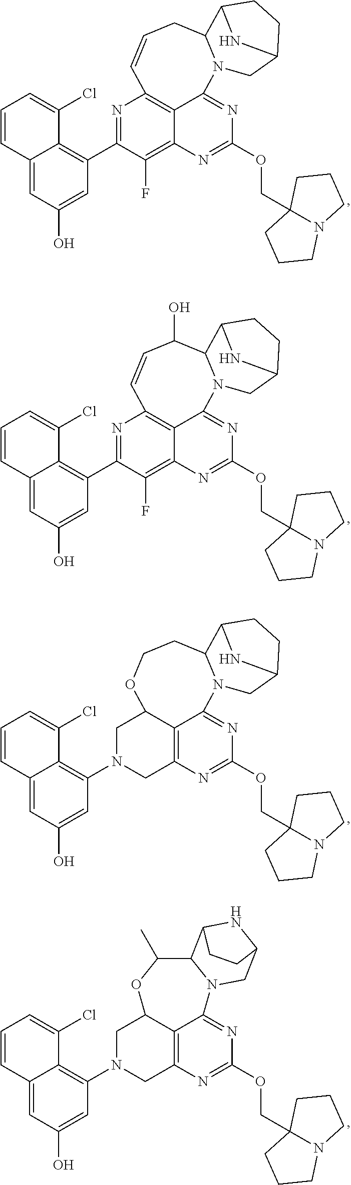

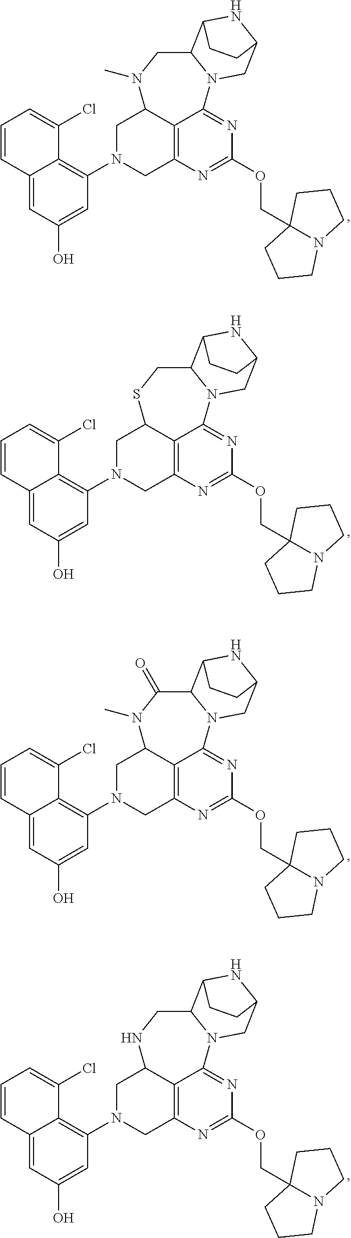

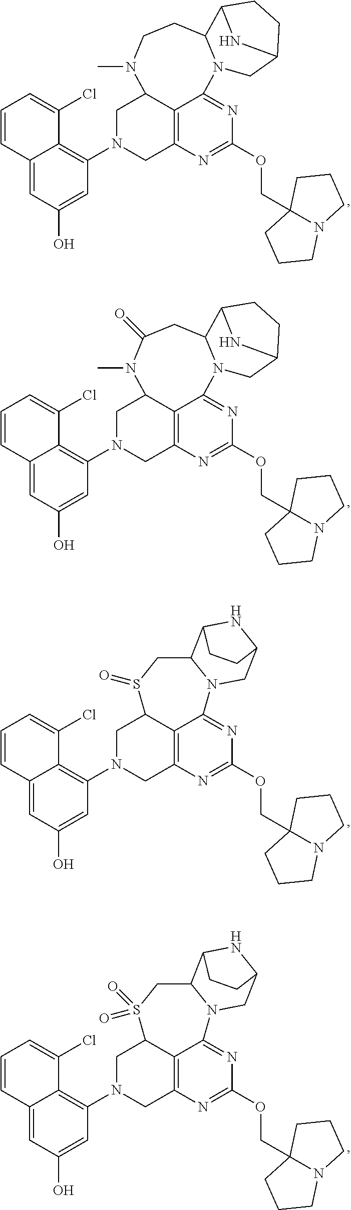

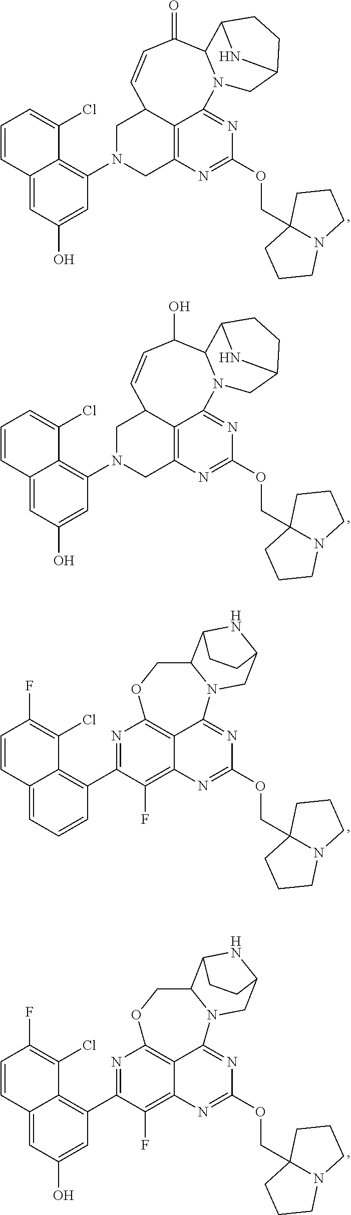

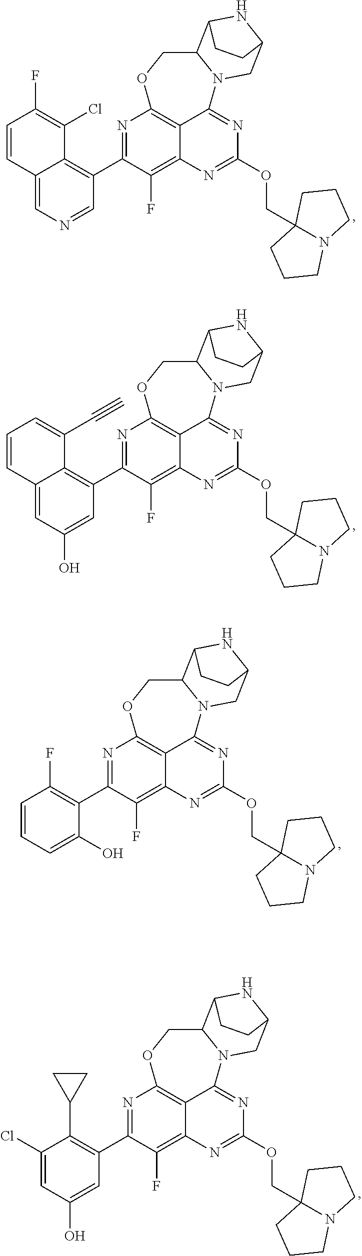

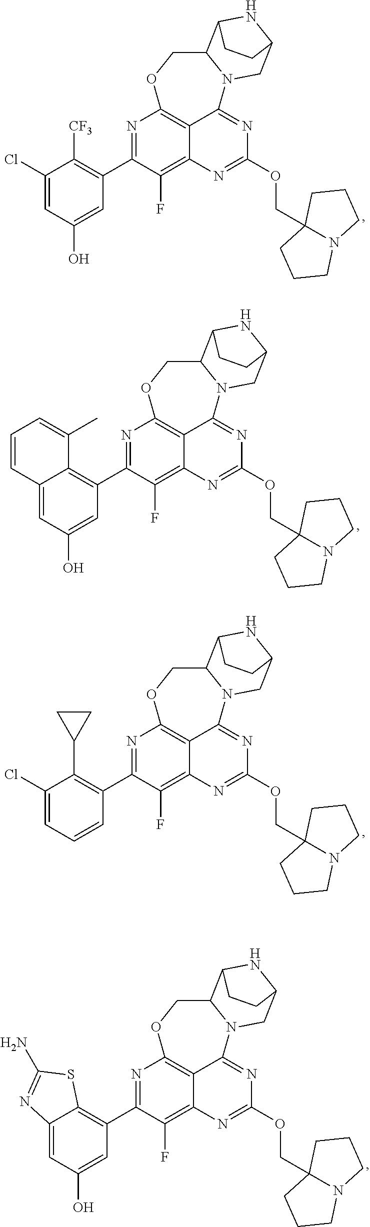

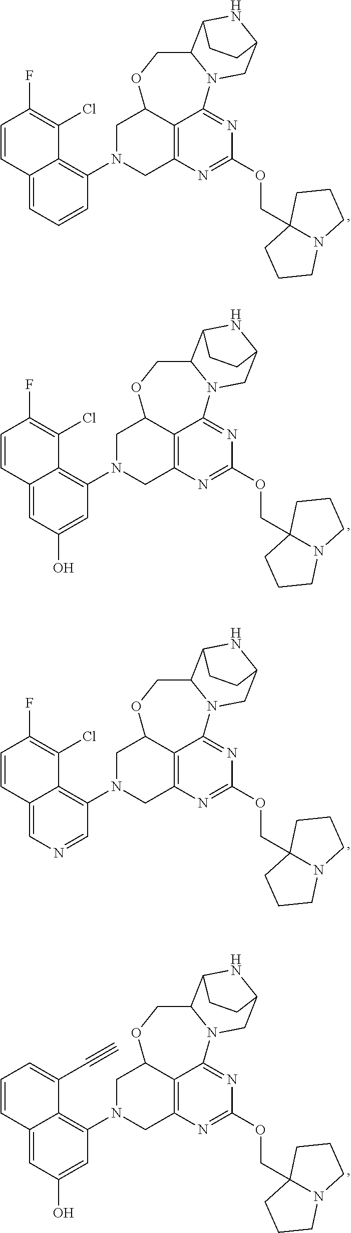

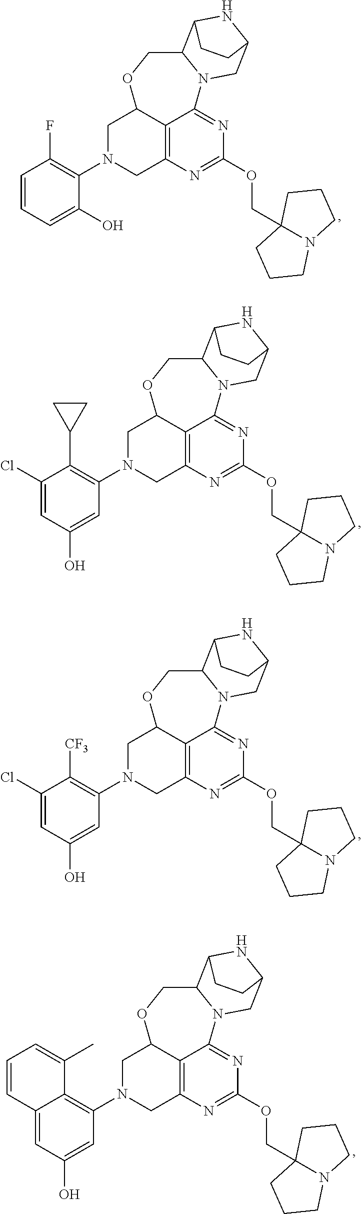

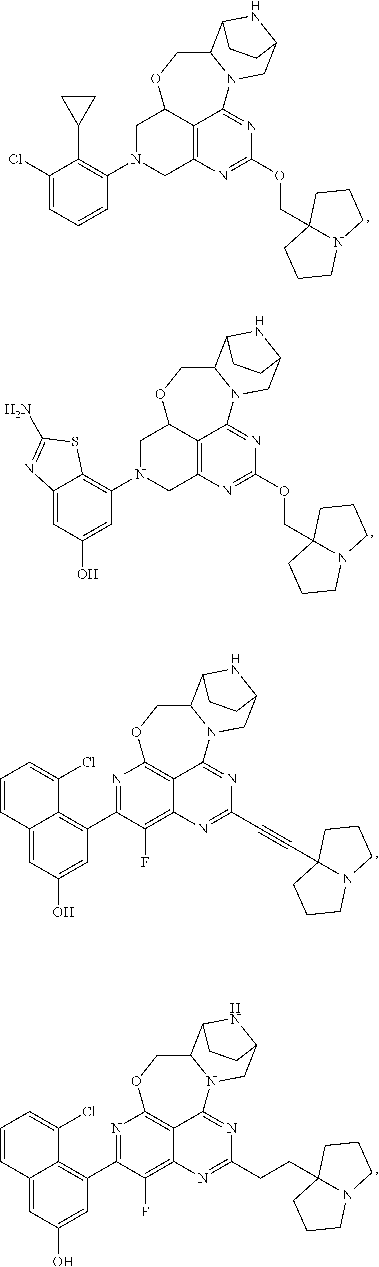

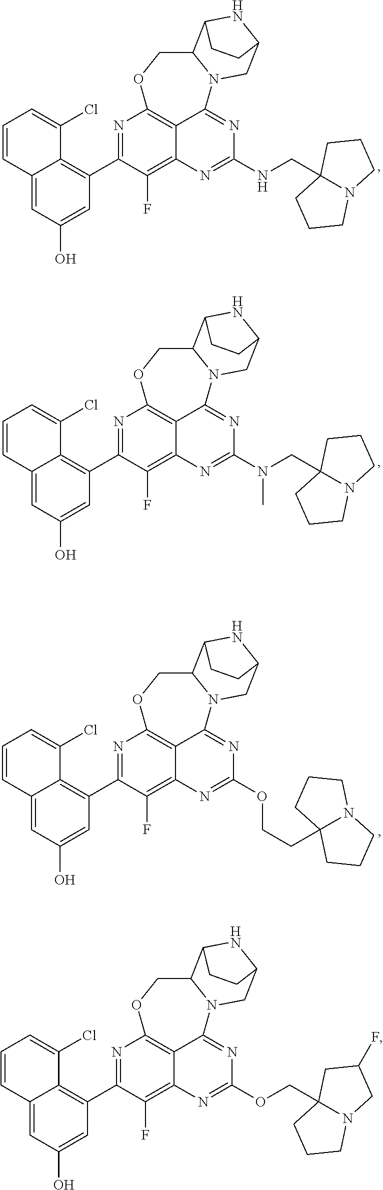

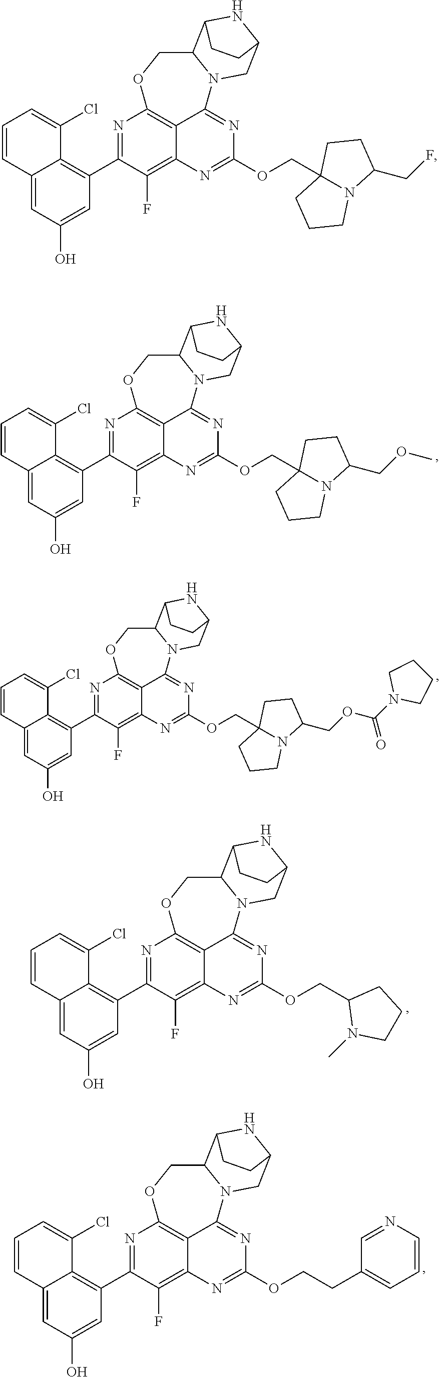

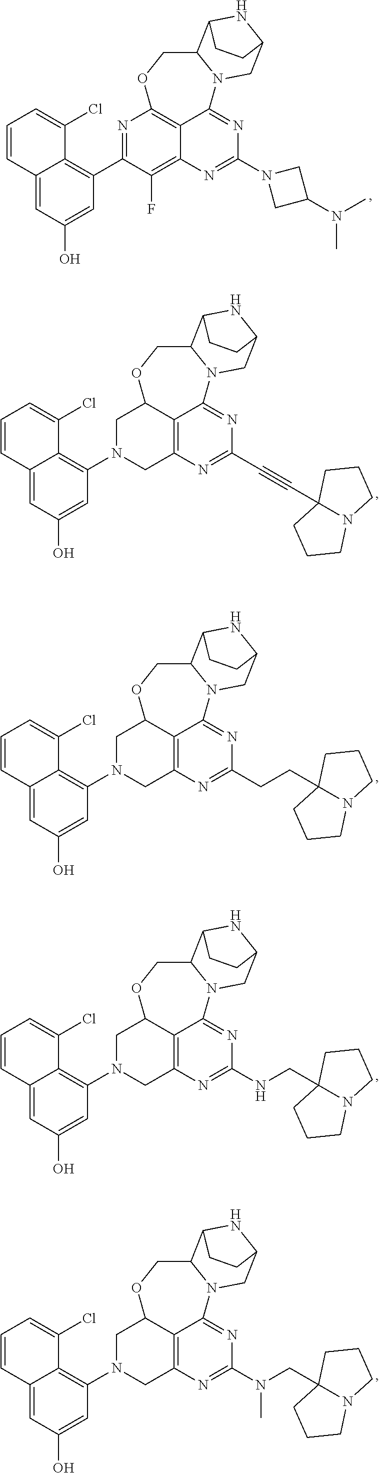

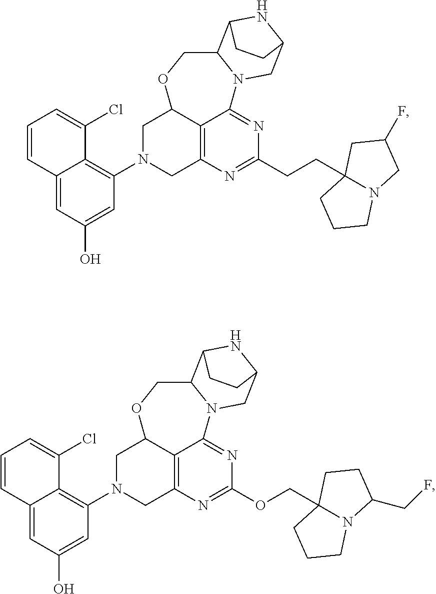 [SMILES: Oc1cc(N2Cc3nc(CCC45CCCN4CC(F)C5)nc4c3C(C2)OCC2C3CCC(CN42)N3)c2c(Cl)cccc2c1 ; Oc1cc(N2Cc3nc(OCC45CCCN4C(CF)CC5)nc4c3C(C2)OCC2C3CCC(CN42)N3)c2c(Cl)cccc2c1]

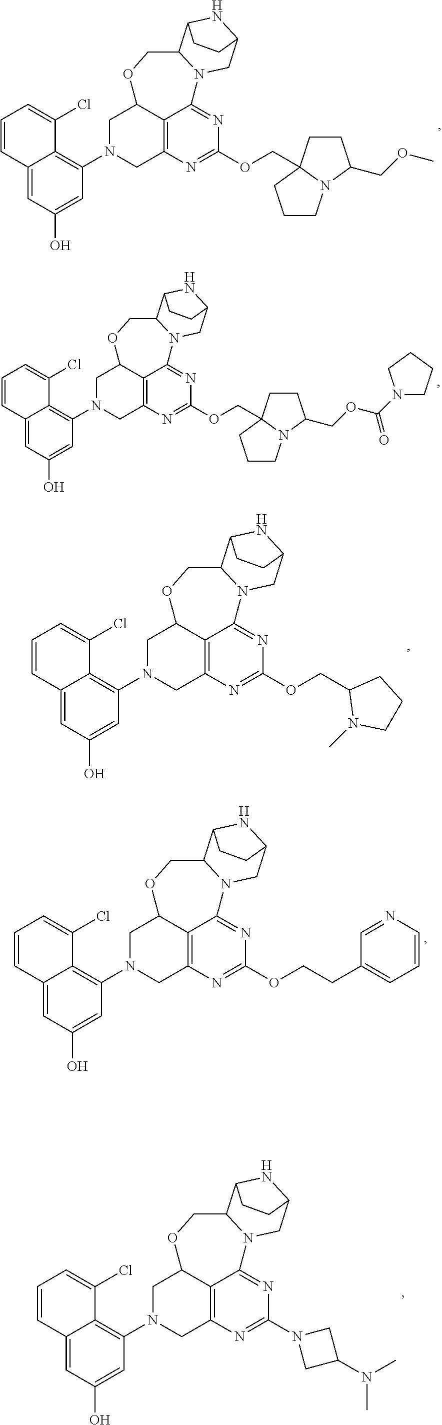 [SMILES: COCC1CCC2(COc3nc4c5c(n3)N3CC6CCC(N6)C3COC5CN(c3cc(O)cc5cccc(Cl)c35)C4)CCCN12 ; O=C(OCC1CCC2(COc3nc4c5c(n3)N3CC6CCC(N6)C3COC5CN(c3cc(O)cc5cccc(Cl)c35)C4)CCCN12)N1CCCC1 ; CN1CCCC1COc1nc2c3c(n1)N1CC4CCC(N4)C1COC3CN(c1cc(O)cc3cccc(Cl)c13)C2 ; Oc1cc(N2Cc3nc(OCCc4cccnc4)nc4c3C(C2)OCC2C3CCC(CN42)N3)c2c(Cl)cccc2c1 ; CN(C)C1CN(c2nc3c4c(n2)N2CC5CCC(N5)C2COC4CN(c2cc(O)cc4cccc(Cl)c24)C3)C1]

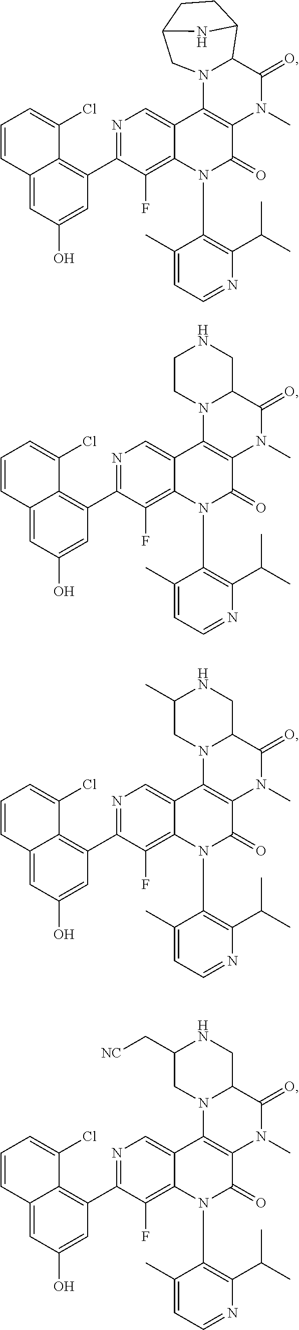

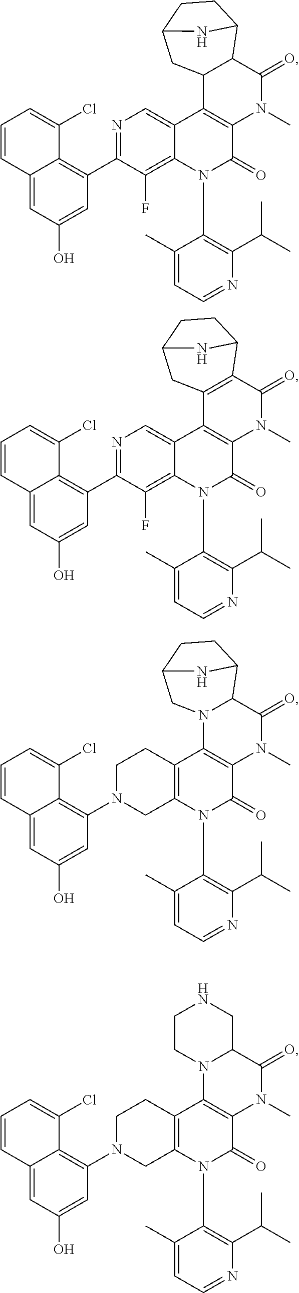

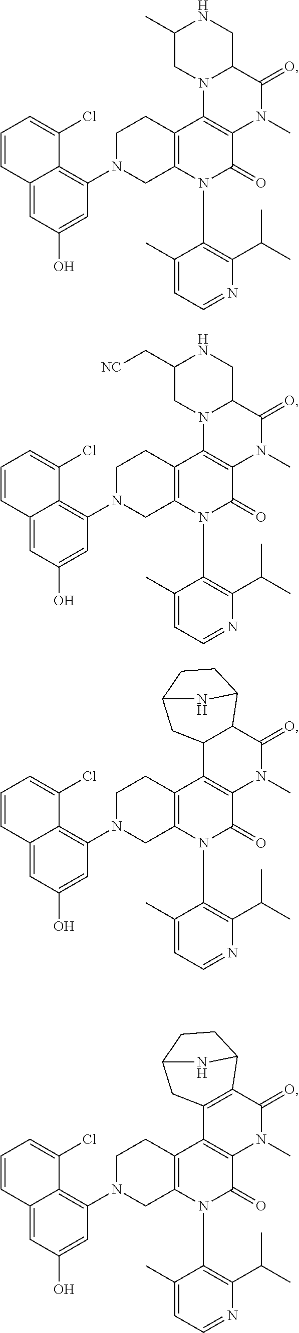

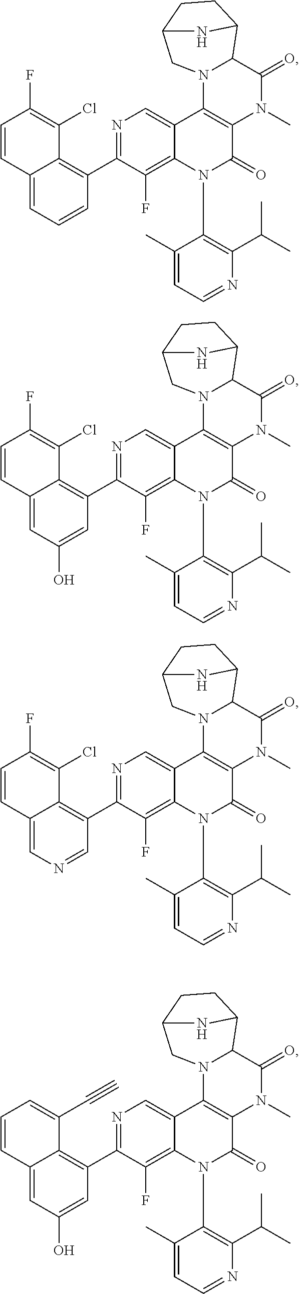

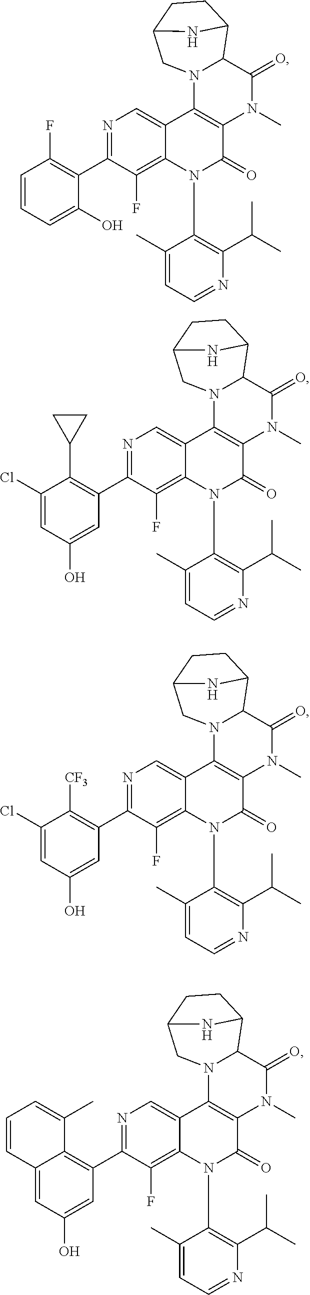

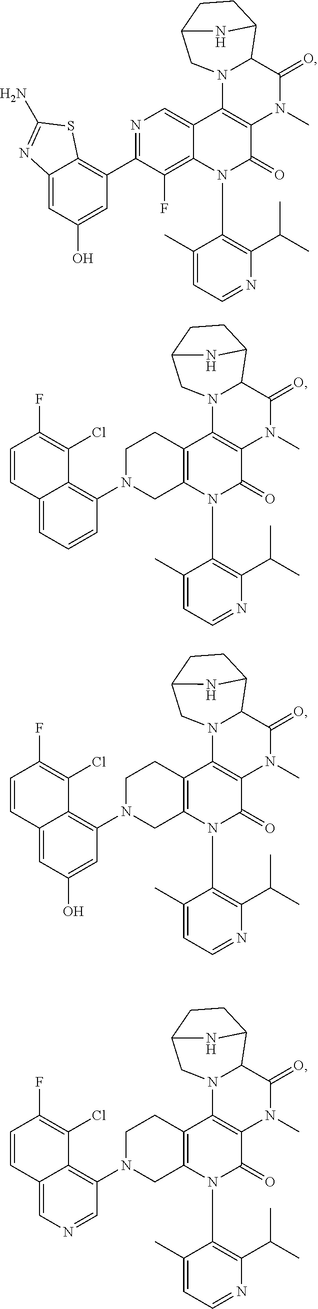

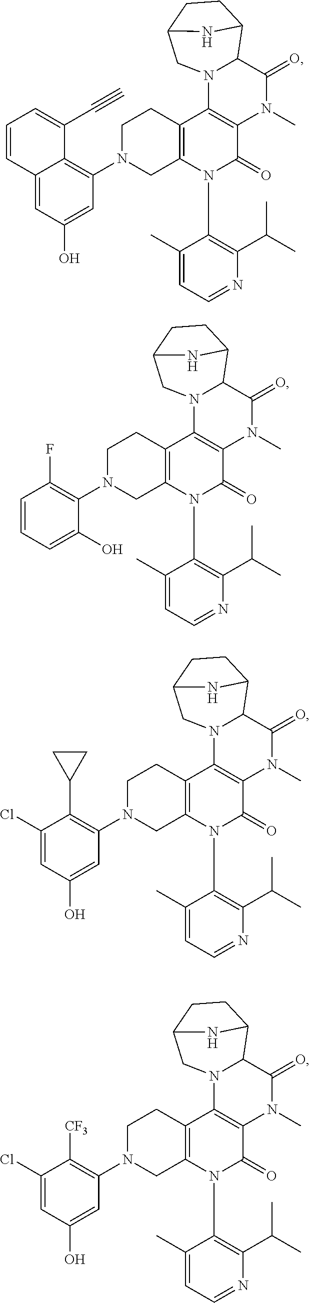

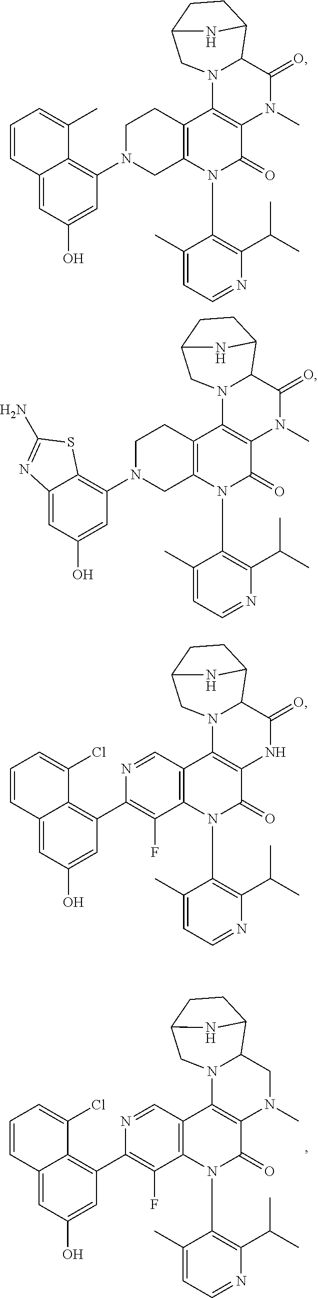

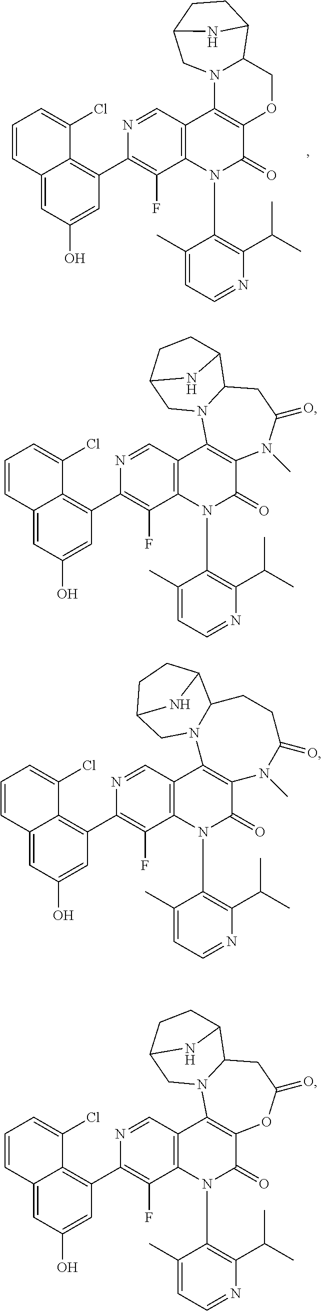

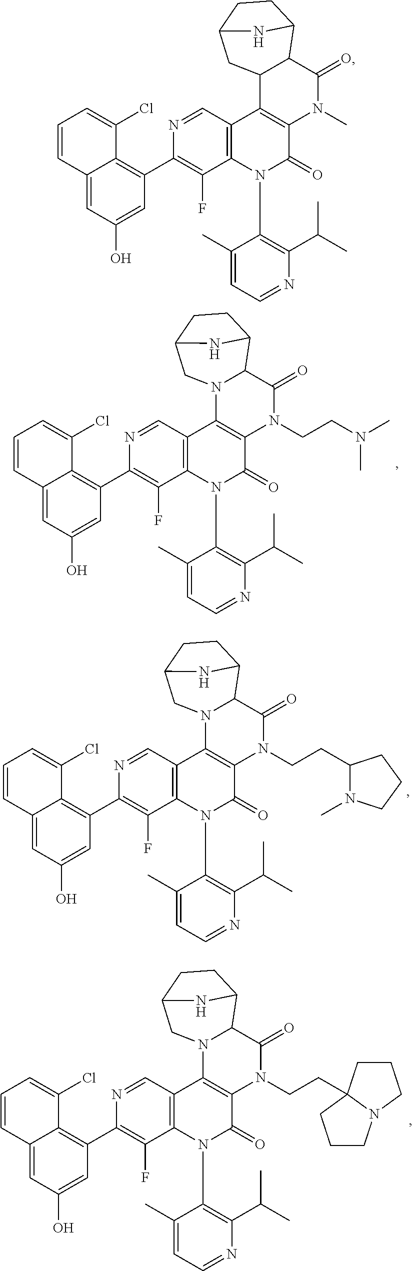

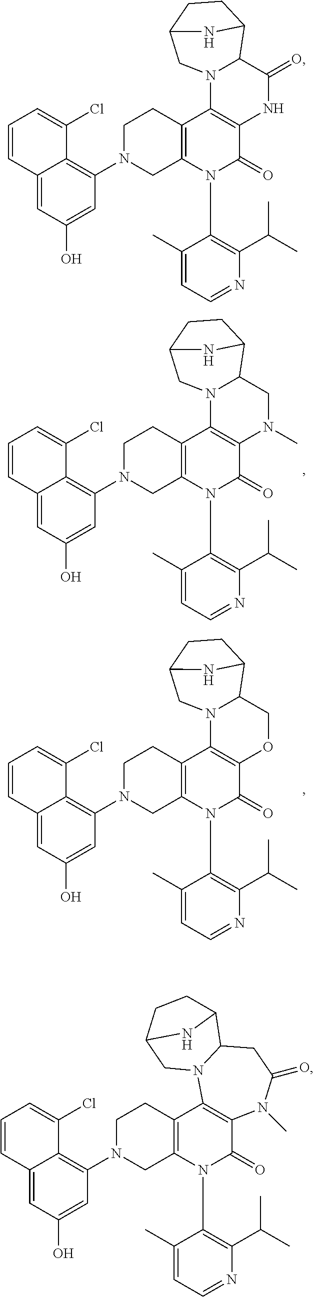

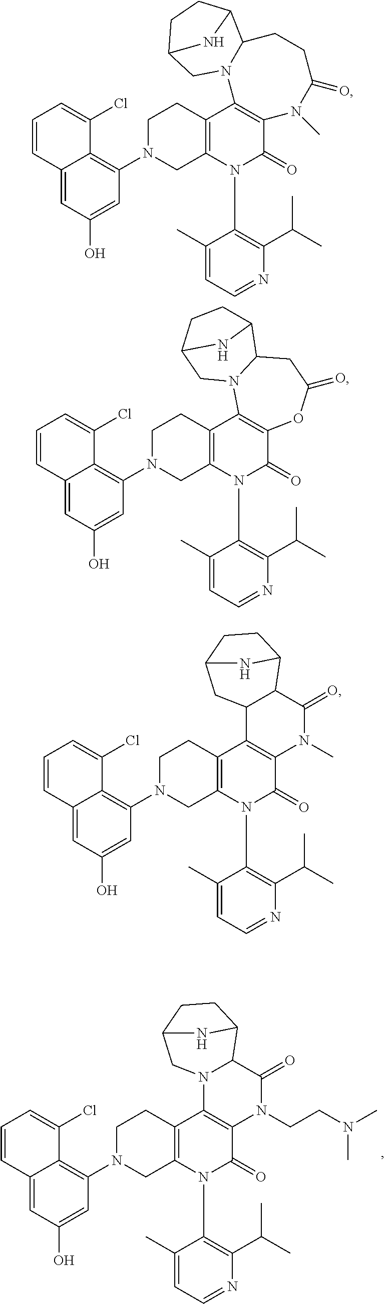

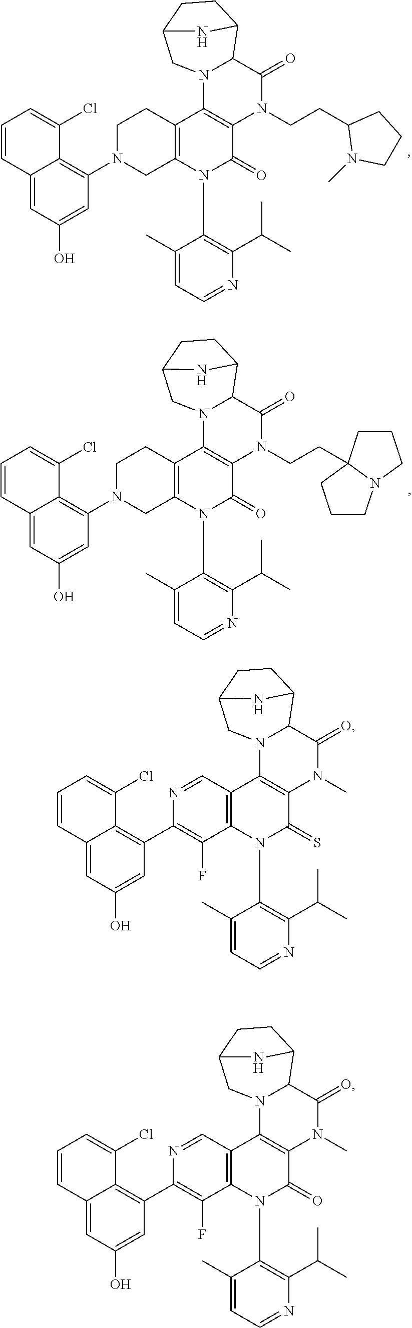

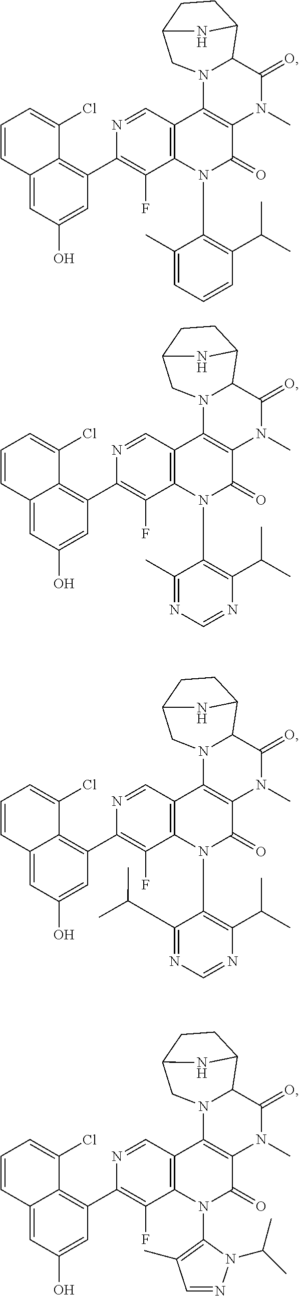

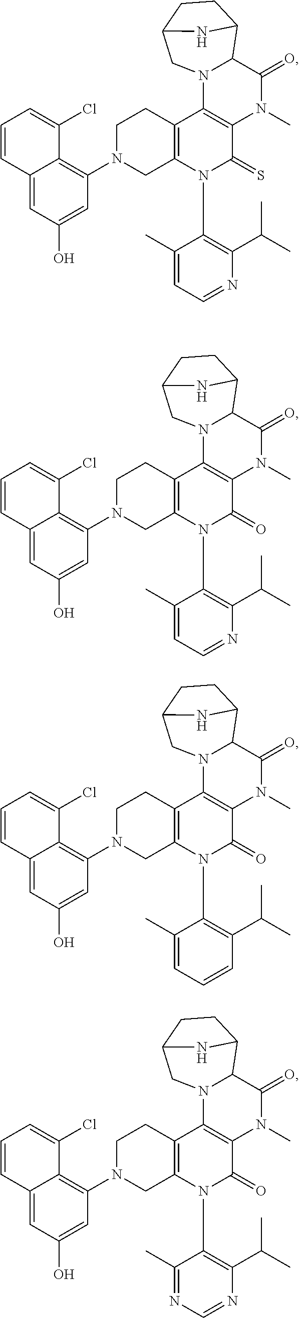

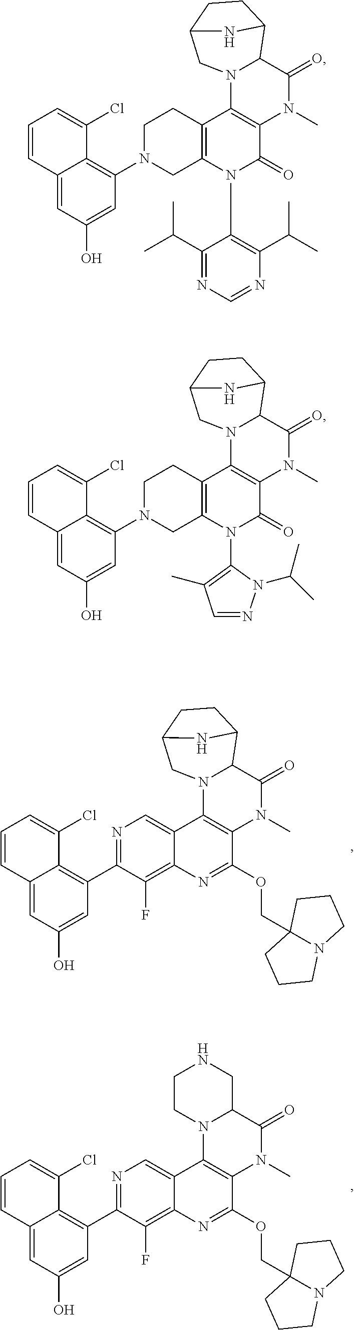 [SMILES: CC(C)c1ncnc(C(C)C)c1-n1c2c(c3c(c1=O)N(C)C(=O)C1C4CCC(CN31)N4)CCN(c1cc(O)cc3cccc(Cl)c13)C2 ; Cc1cnn(C(C)C)c1-n1c2c(c3c(c1=O)N(C)C(=O)C1C4CCC(CN31)N4)CCN(c1cc(O)cc3cccc(Cl)c13)C2 ; CN1C(=O)C2C3CCC(CN2c2c1c(OCC14CCCN1CCC4)nc1c(F)c(-c4cc(O)cc5cccc(Cl)c45)ncc21)N3 ; CN1C(=O)C2CNCCN2c2c1c(OCC13CCCN1CCC3)nc1c(F)c(-c3cc(O)cc4cccc(Cl)c34)ncc21]

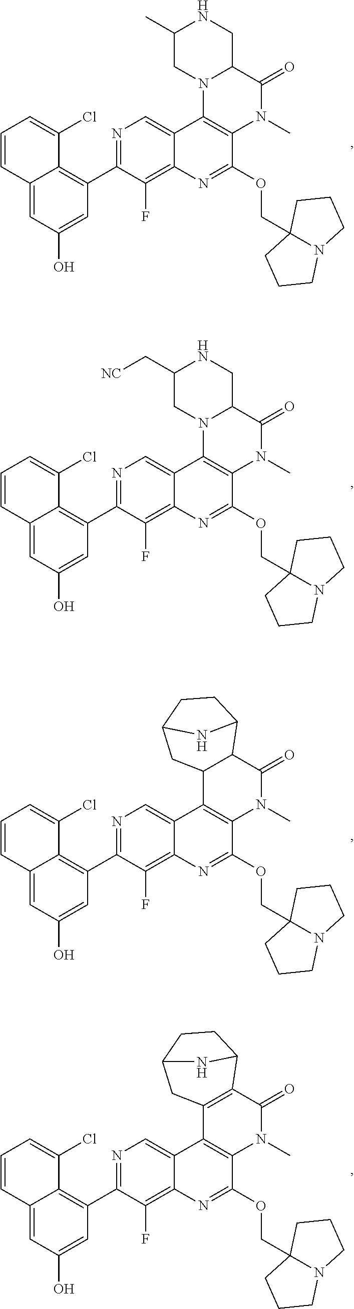 [SMILES: CC1CN2c3c(c(OCC45CCCN4CCC5)nc4c(F)c(-c5cc(O)cc6cccc(Cl)c56)ncc34)N(C)C(=O)C2CN1 ; CN1C(=O)C2CNC(CC#N)CN2c2c1c(OCC13CCCN1CCC3)nc1c(F)c(-c3cc(O)cc4cccc(Cl)c34)ncc21 ; CN1C(=O)C2C3CCC(CC2c2c1c(OCC14CCCN1CCC4)nc1c(F)c(-c4cc(O)cc5cccc(Cl)c45)ncc21)N3 ; Cn1c(=O)c2c(c3c4cnc(-c5cc(O)cc6cccc(Cl)c56)c(F)c4nc(OCC45CCCN4CCC5)c31)CC1CCC2N1]

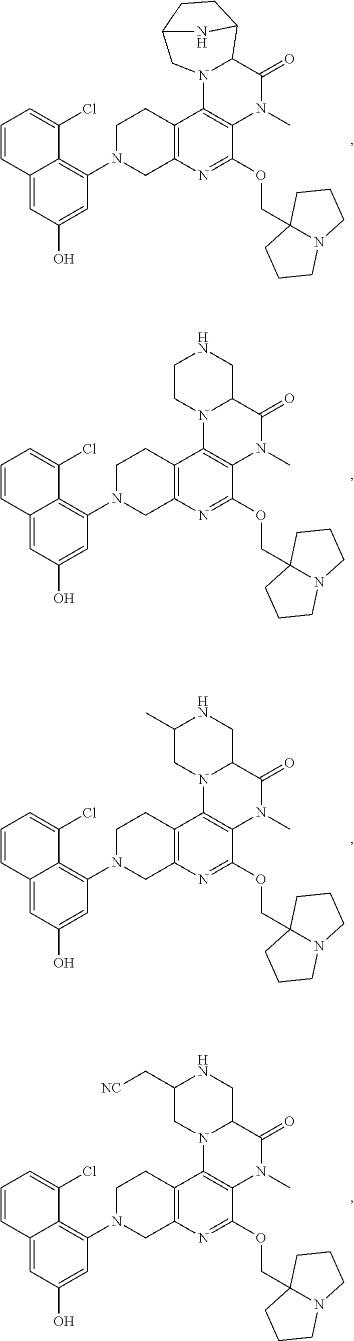 [SMILES: CN1C(=O)C2C3CCC(CN2c2c4c(nc(OCC56CCCN5CCC6)c21)CN(c1cc(O)cc2cccc(Cl)c12)CC4)N3 ; CN1C(=O)C2CNCCN2c2c3c(nc(OCC45CCCN4CCC5)c21)CN(c1cc(O)cc2cccc(Cl)c12)CC3 ; CC1CN2c3c4c(nc(OCC56CCCN5CCC6)c3N(C)C(=O)C2CN1)CN(c1cc(O)cc2cccc(Cl)c12)CC4 ; CN1C(=O)C2CNC(CC#N)CN2c2c3c(nc(OCC45CCCN4CCC5)c21)CN(c1cc(O)cc2cccc(Cl)c12)CC3]

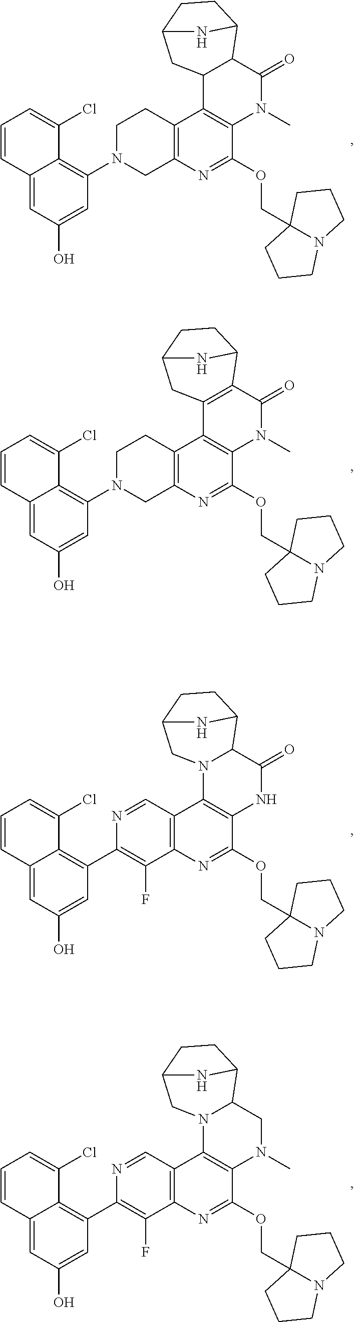 [SMILES: CN1C(=O)C2C3CCC(CC2c2c4c(nc(OCC56CCCN5CCC6)c21)CN(c1cc(O)cc2cccc(Cl)c12)CC4)N3 ; Cn1c(=O)c2c(c3c4c(nc(OCC56CCCN5CCC6)c31)CN(c1cc(O)cc3cccc(Cl)c13)CC4)CC1CCC2N1 ; O=C1Nc2c(OCC34CCCN3CCC4)nc3c(F)c(-c4cc(O)cc5cccc(Cl)c45)ncc3c2N2CC3CCC(N3)C12 ; CN1CC2C3CCC(CN2c2c1c(OCC14CCCN1CCC4)nc1c(F)c(-c4cc(O)cc5cccc(Cl)c45)ncc21)N3]

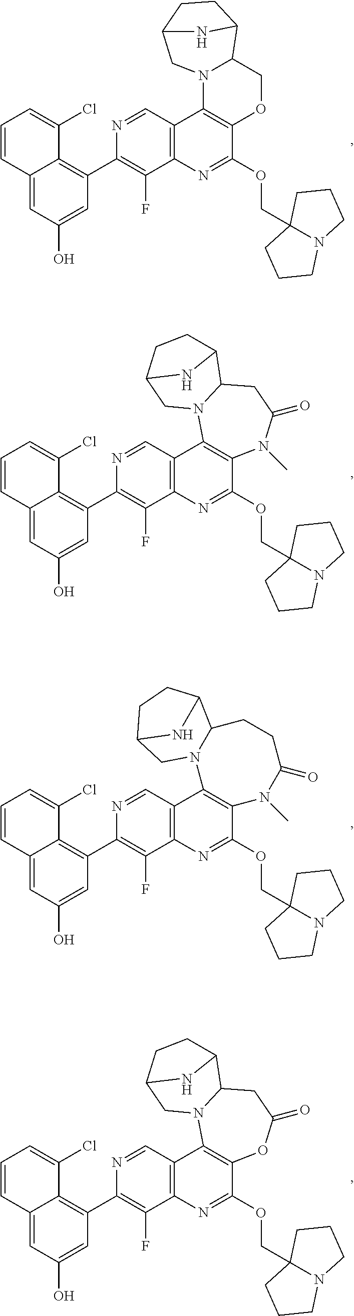 [SMILES: Oc1cc(-c2ncc3c4c(c(OCC56CCCN5CCC6)nc3c2F)OCC2C3CCC(CN42)N3)c2c(Cl)cccc2c1 ; CN1C(=O)CC2C3CCC(CN2c2c1c(OCC14CCCN1CCC4)nc1c(F)c(-c4cc(O)cc5cccc(Cl)c45)ncc21)N3 ; CN1C(=O)CCC2C3CCC(CN2c2c1c(OCC14CCCN1CCC4)nc1c(F)c(-c4cc(O)cc5cccc(Cl)c45)ncc21)N3 ; O=C1CC2C3CCC(CN2c2c(c(OCC45CCCN4CCC5)nc4c(F)c(-c5cc(O)cc6cccc(Cl)c56)ncc24)O1)N3]

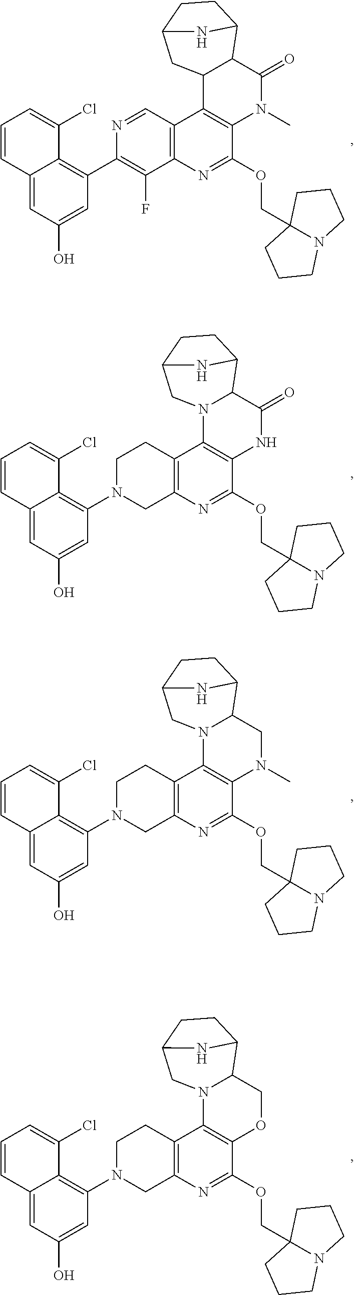 [SMILES: CN1C(=O)C2C3CCC(CC2c2c1c(OCC14CCCN1CCC4)nc1c(F)c(-c4cc(O)cc5cccc(Cl)c45)ncc21)N3 ; O=C1Nc2c(OCC34CCCN3CCC4)nc3c(c2N2CC4CCC(N4)C12)CCN(c1cc(O)cc2cccc(Cl)c12)C3 ; CN1CC2C3CCC(CN2c2c4c(nc(OCC56CCCN5CCC6)c21)CN(c1cc(O)cc2cccc(Cl)c12)CC4)N3 ; Oc1cc(N2CCc3c(nc(OCC45CCCN4CCC5)c4c3N3CC5CCC(N5)C3CO4)C2)c2c(Cl)cccc2c1]

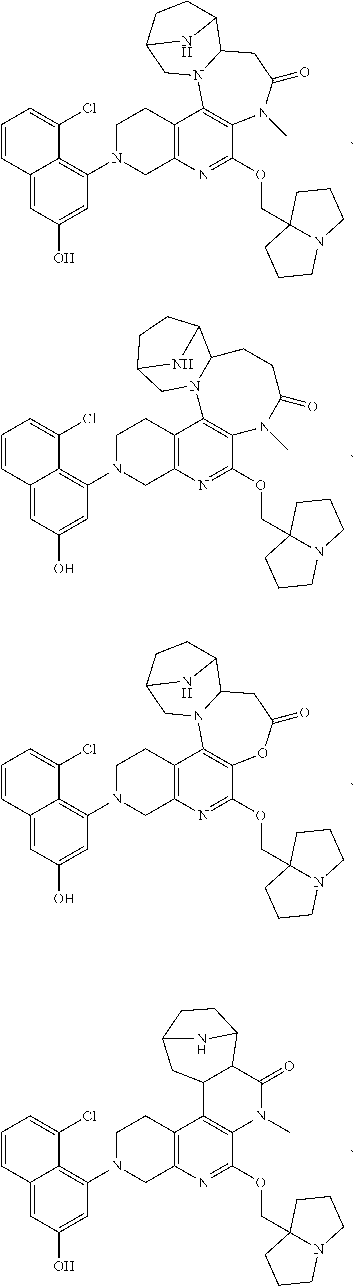 [SMILES: CN1C(=O)CC2C3CCC(CN2c2c4c(nc(OCC56CCCN5CCC6)c21)CN(c1cc(O)cc2cccc(Cl)c12)CC4)N3 ; CN1C(=O)CCC2C3CCC(CN2c2c4c(nc(OCC56CCCN5CCC6)c21)CN(c1cc(O)cc2cccc(Cl)c12)CC4)N3 ; O=C1CC2C3CCC(CN2c2c4c(nc(OCC56CCCN5CCC6)c2O1)CN(c1cc(O)cc2cccc(Cl)c12)CC4)N3 ; CN1C(=O)C2C3CCC(CC2c2c4c(nc(OCC56CCCN5CCC6)c21)CN(c1cc(O)cc2cccc(Cl)c12)CC4)N3]

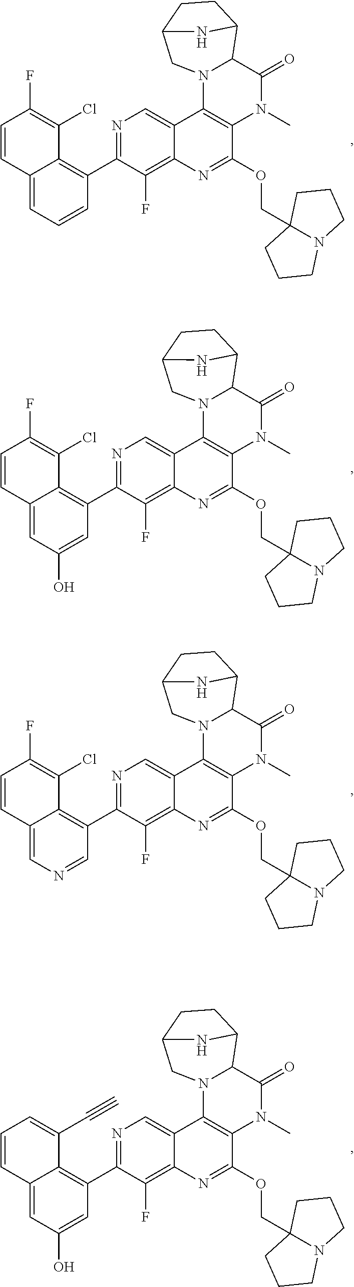 [SMILES: CN1C(=O)C2C3CCC(CN2c2c1c(OCC14CCCN1CCC4)nc1c(F)c(-c4cccc5ccc(F)c(Cl)c45)ncc21)N3 ; CN1C(=O)C2C3CCC(CN2c2c1c(OCC14CCCN1CCC4)nc1c(F)c(-c4cc(O)cc5ccc(F)c(Cl)c45)ncc21)N3 ; CN1C(=O)C2C3CCC(CN2c2c1c(OCC14CCCN1CCC4)nc1c(F)c(-c4cncc5ccc(F)c(Cl)c45)ncc21)N3 ; C#Cc1cccc2cc(O)cc(-c3ncc4c5c(c(OCC67CCCN6CCC7)nc4c3F)N(C)C(=O)C3C4CCC(CN53)N4)c12]

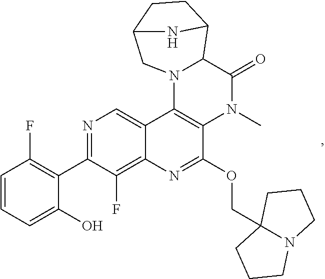 [SMILES: CN1C(=O)C2C3CCC(CN2c2c1c(OCC14CCCN1CCC4)nc1c(F)c(-c4c(O)cccc4F)ncc21)N3]

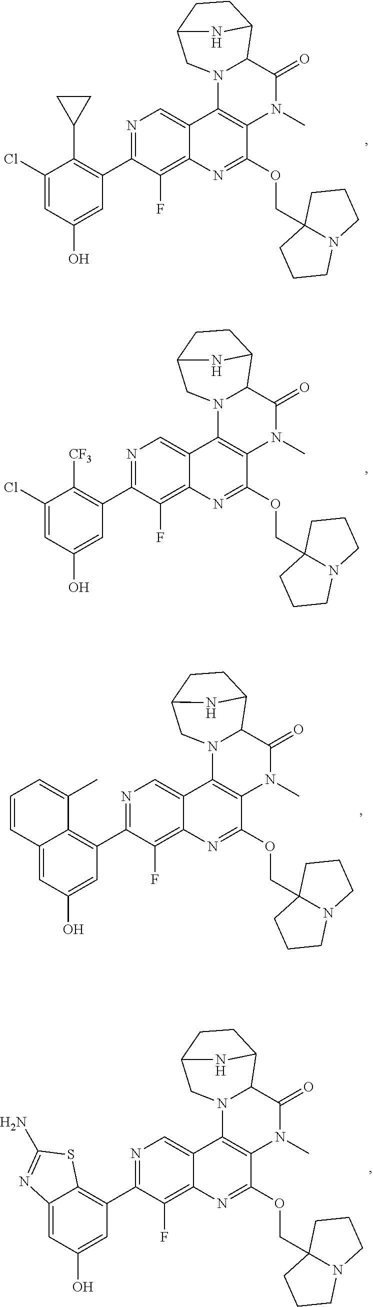 [SMILES: CN1C(=O)C2C3CCC(CN2c2c1c(OCC14CCCN1CCC4)nc1c(F)c(-c4cc(O)cc(Cl)c4C4CC4)ncc21)N3 ; CN1C(=O)C2C3CCC(CN2c2c1c(OCC14CCCN1CCC4)nc1c(F)c(-c4cc(O)cc(Cl)c4C(F)(F)F)ncc21)N3 ; Cc1cccc2cc(O)cc(-c3ncc4c5c(c(OCC67CCCN6CCC7)nc4c3F)N(C)C(=O)C3C4CCC(CN53)N4)c12 ; CN1C(=O)C2C3CCC(CN2c2c1c(OCC14CCCN1CCC4)nc1c(F)c(-c4cc(O)cc5nc(N)sc45)ncc21)N3]

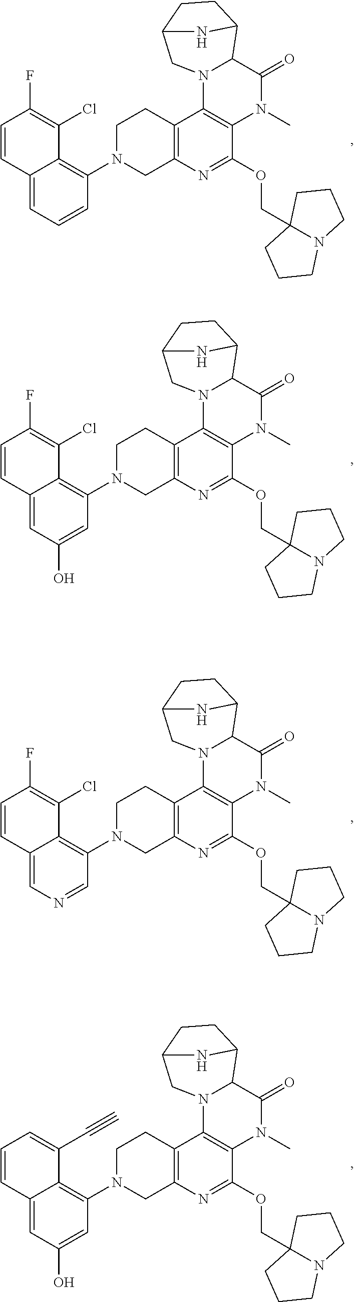 [SMILES: CN1C(=O)C2C3CCC(CN2c2c4c(nc(OCC56CCCN5CCC6)c21)CN(c1cccc2ccc(F)c(Cl)c12)CC4)N3 ; CN1C(=O)C2C3CCC(CN2c2c4c(nc(OCC56CCCN5CCC6)c21)CN(c1cc(O)cc2ccc(F)c(Cl)c12)CC4)N3 ; CN1C(=O)C2C3CCC(CN2c2c4c(nc(OCC56CCCN5CCC6)c21)CN(c1cncc2ccc(F)c(Cl)c12)CC4)N3 ; C#Cc1cccc2cc(O)cc(N3CCc4c(nc(OCC56CCCN5CCC6)c5c4N4CC6CCC(N6)C4C(=O)N5C)C3)c12]

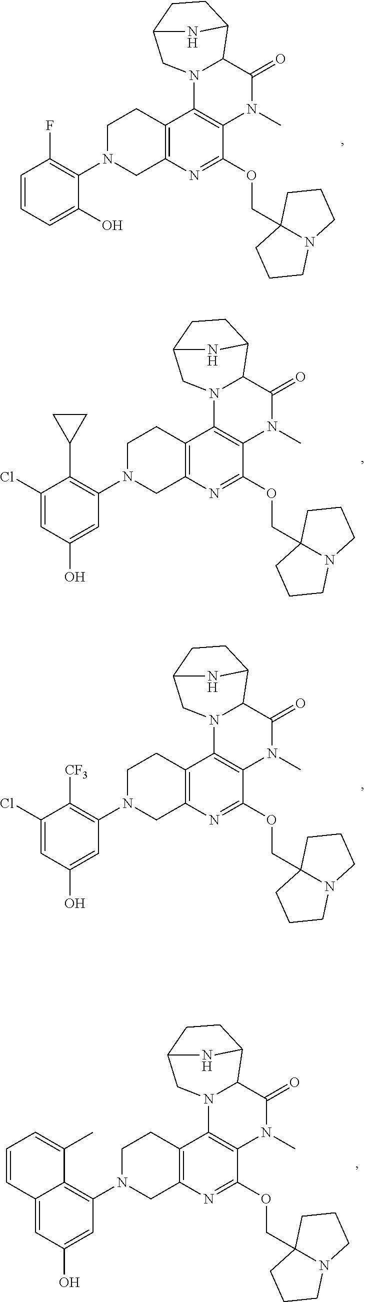 [SMILES: CN1C(=O)C2C3CCC(CN2c2c4c(nc(OCC56CCCN5CCC6)c21)CN(c1c(O)cccc1F)CC4)N3 ; CN1C(=O)C2C3CCC(CN2c2c4c(nc(OCC56CCCN5CCC6)c21)CN(c1cc(O)cc(Cl)c1C1CC1)CC4)N3 ; CN1C(=O)C2C3CCC(CN2c2c4c(nc(OCC56CCCN5CCC6)c21)CN(c1cc(O)cc(Cl)c1C(F)(F)F)CC4)N3 ; Cc1cccc2cc(O)cc(N3CCc4c(nc(OCC56CCCN5CCC6)c5c4N4CC6CCC(N6)C4C(=O)N5C)C3)c12]

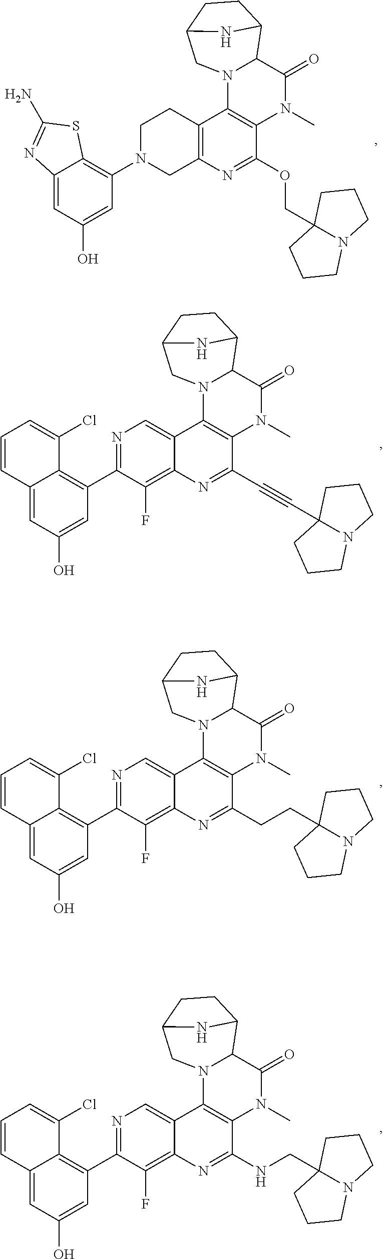 [SMILES: CN1C(=O)C2C3CCC(CN2c2c4c(nc(OCC56CCCN5CCC6)c21)CN(c1cc(O)cc2nc(N)sc12)CC4)N3 ; CN1C(=O)C2C3CCC(CN2c2c1c(C#CC14CCCN1CCC4)nc1c(F)c(-c4cc(O)cc5cccc(Cl)c45)ncc21)N3 ; CN1C(=O)C2C3CCC(CN2c2c1c(CCC14CCCN1CCC4)nc1c(F)c(-c4cc(O)cc5cccc(Cl)c45)ncc21)N3 ; CN1C(=O)C2C3CCC(CN2c2c1c(NCC14CCCN1CCC4)nc1c(F)c(-c4cc(O)cc5cccc(Cl)c45)ncc21)N3]

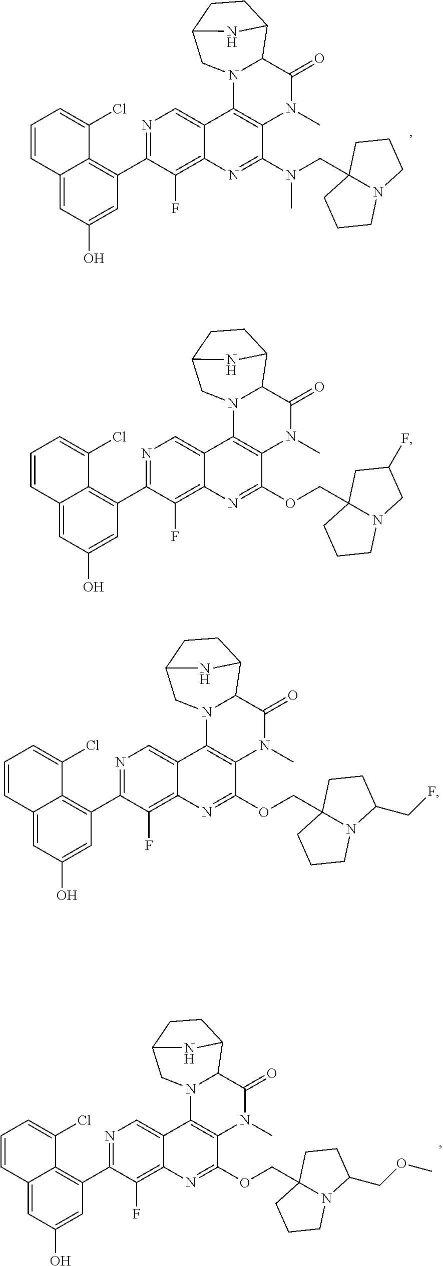 [SMILES: CN(CC12CCCN1CCC2)c1nc2c(F)c(-c3cc(O)cc4cccc(Cl)c34)ncc2c2c1N(C)C(=O)C1C3CCC(CN21)N3 ; CN1C(=O)C2C3CCC(CN2c2c1c(OCC14CCCN1CC(F)C4)nc1c(F)c(-c4cc(O)cc5cccc(Cl)c45)ncc21)N3 ; CN1C(=O)C2C3CCC(CN2c2c1c(OCC14CCCN1C(CF)CC4)nc1c(F)c(-c4cc(O)cc5cccc(Cl)c45)ncc21)N3 ; COCC1CCC2(COc3nc4c(F)c(-c5cc(O)cc6cccc(Cl)c56)ncc4c4c3N(C)C(=O)C3C5CCC(CN43)N5)CCCN12]

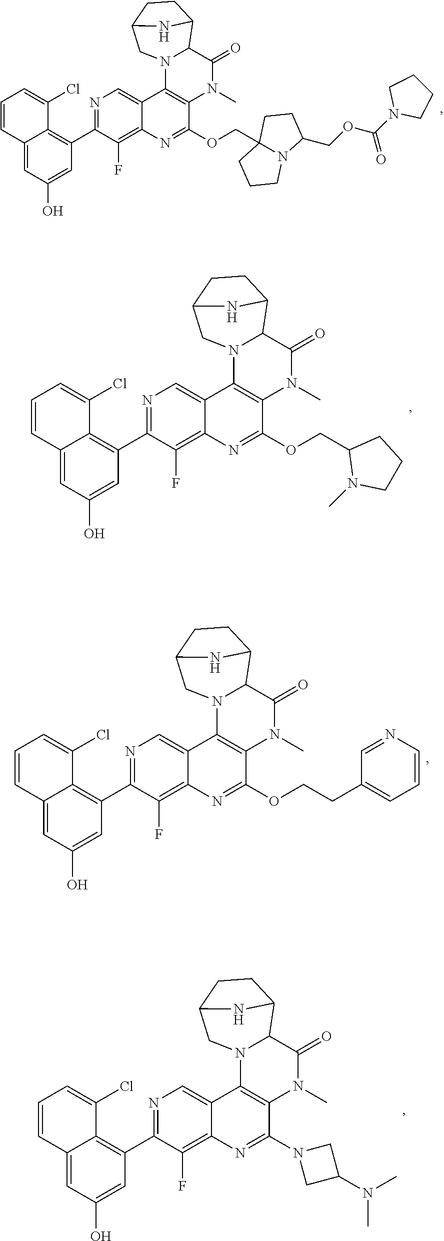 [SMILES: CN1C(=O)C2C3CCC(CN2c2c1c(OCC14CCCN1C(COC(=O)N1CCCC1)CC4)nc1c(F)c(-c4cc(O)cc5cccc(Cl)c45)ncc21)N3 ; CN1C(=O)C2C3CCC(CN2c2c1c(OCC1CCCN1C)nc1c(F)c(-c4cc(O)cc5cccc(Cl)c45)ncc21)N3 ; CN1C(=O)C2C3CCC(CN2c2c1c(OCCc1cccnc1)nc1c(F)c(-c4cc(O)cc5cccc(Cl)c45)ncc21)N3 ; CN1C(=O)C2C3CCC(CN2c2c1c(N1CC(N(C)C)C1)nc1c(F)c(-c4cc(O)cc5cccc(Cl)c45)ncc21)N3]

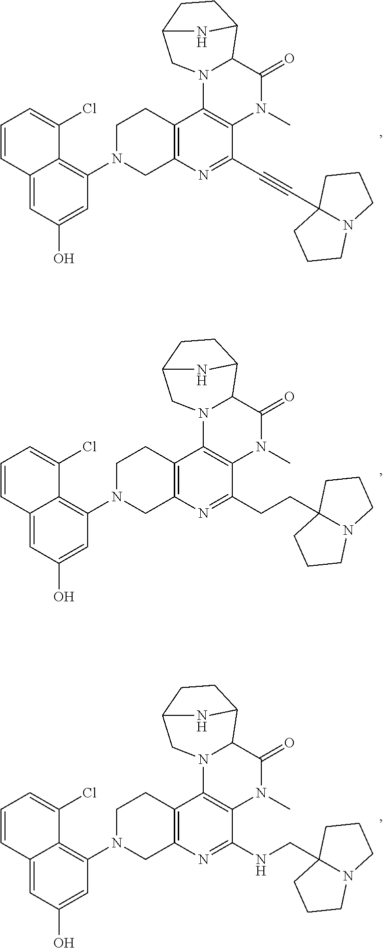 [SMILES: CN1C(=O)C2C3CCC(CN2c2c4c(nc(C#CC56CCCN5CCC6)c21)CN(c1cc(O)cc2cccc(Cl)c12)CC4)N3 ; CN1C(=O)C2C3CCC(CN2c2c4c(nc(CCC56CCCN5CCC6)c21)CN(c1cc(O)cc2cccc(Cl)c12)CC4)N3 ; CN1C(=O)C2C3CCC(CN2c2c4c(nc(NCC56CCCN5CCC6)c21)CN(c1cc(O)cc2cccc(Cl)c12)CC4)N3]

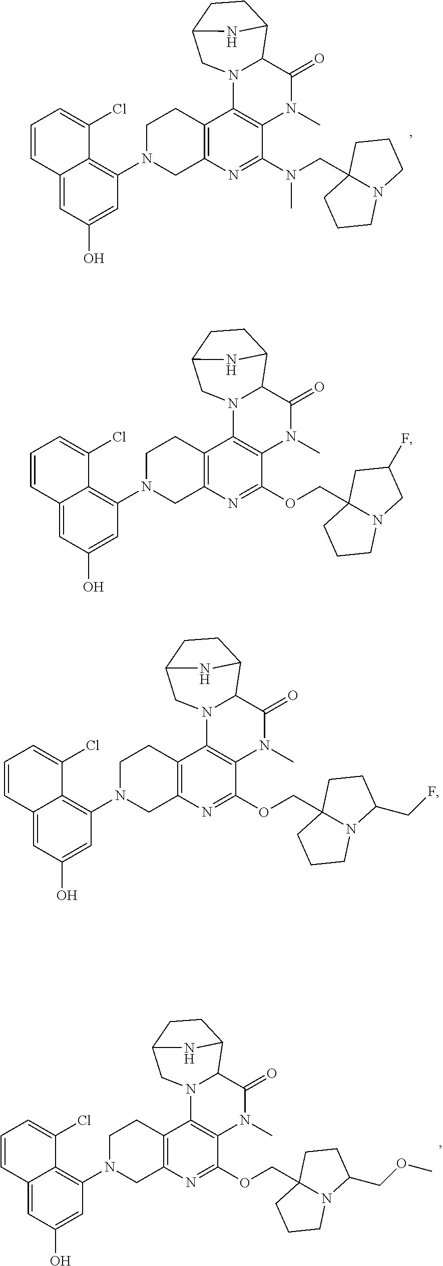 [SMILES: CN(CC12CCCN1CCC2)c1nc2c(c3c1N(C)C(=O)C1C4CCC(CN31)N4)CCN(c1cc(O)cc3cccc(Cl)c13)C2 ; CN1C(=O)C2C3CCC(CN2c2c4c(nc(OCC56CCCN5CC(F)C6)c21)CN(c1cc(O)cc2cccc(Cl)c12)CC4)N3 ; CN1C(=O)C2C3CCC(CN2c2c4c(nc(OCC56CCCN5C(CF)CC6)c21)CN(c1cc(O)cc2cccc(Cl)c12)CC4)N3 ; COCC1CCC2(COc3nc4c(c5c3N(C)C(=O)C3C6CCC(CN53)N6)CCN(c3cc(O)cc5cccc(Cl)c35)C4)CCCN12]

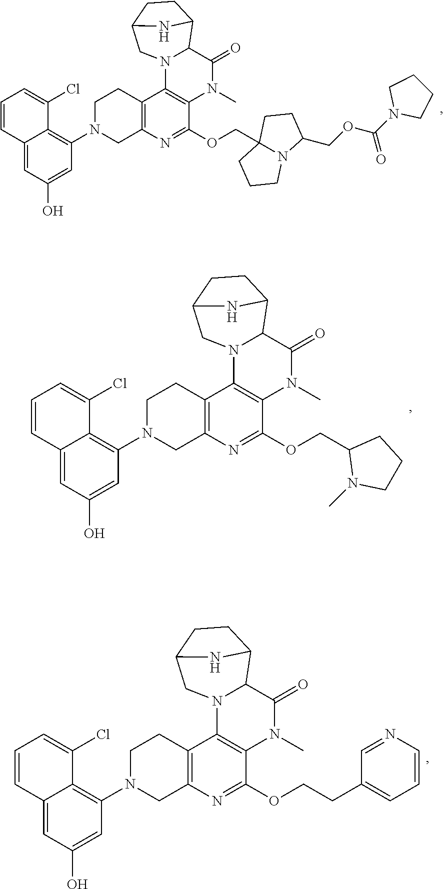 [SMILES: CN1C(=O)C2C3CCC(CN2c2c4c(nc(OCC56CCCN5C(COC(=O)N5CCCC5)CC6)c21)CN(c1cc(O)cc2cccc(Cl)c12)CC4)N3 ; CN1C(=O)C2C3CCC(CN2c2c4c(nc(OCC5CCCN5C)c21)CN(c1cc(O)cc2cccc(Cl)c12)CC4)N3 ; CN1C(=O)C2C3CCC(CN2c2c4c(nc(OCCc5cccnc5)c21)CN(c1cc(O)cc2cccc(Cl)c12)CC4)N3]

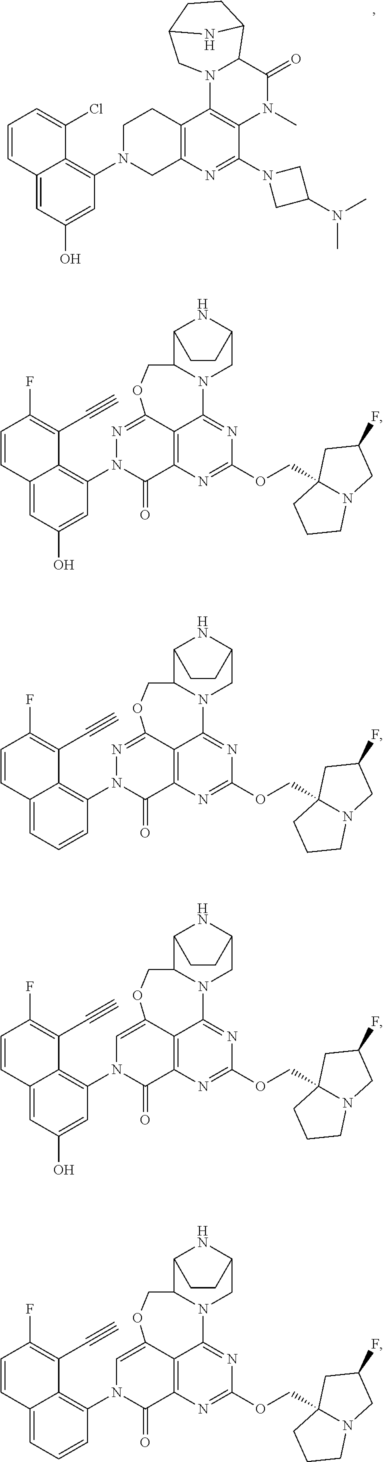 [SMILES: CN1C(=O)C2C3CCC(CN2c2c4c(nc(N5CC(N(C)C)C5)c21)CN(c1cc(O)cc2cccc(Cl)c12)CC4)N3 ; C#Cc1c(F)ccc2cc(O)cc(-n3nc4c5c(nc(OC[C@@]67CCCN6C[C@H](F)C7)nc5c3=O)N3CC5CCC(N5)C3CO4)c12 ; C#Cc1c(F)ccc2cccc(-n3nc4c5c(nc(OC[C@@]67CCCN6C[C@H](F)C7)nc5c3=O)N3CC5CCC(N5)C3CO4)c12 ; C#Cc1c(F)ccc2cc(O)cc(-n3cc4c5c(nc(OC[C@@]67CCCN6C[C@H](F)C7)nc5c3=O)N3CC5CCC(N5)C3CO4)c12 ; C#Cc1c(F)ccc2cccc(-n3cc4c5c(nc(OC[C@@]67CCCN6C[C@H](F)C7)nc5c3=O)N3CC5CCC(N5)C3CO4)c12]

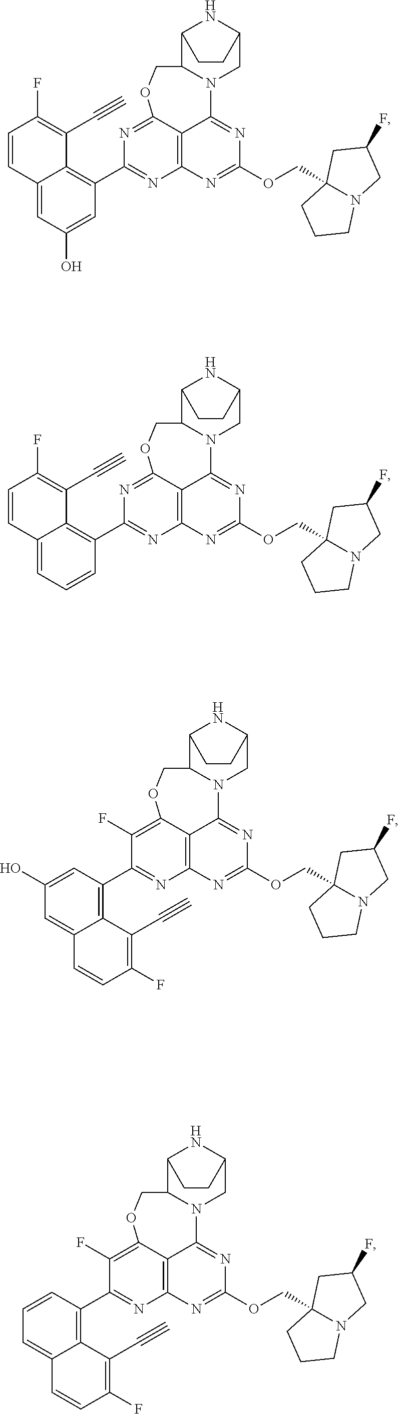 [SMILES: C#Cc1c(F)ccc2cc(O)cc(-c3nc4c5c(nc(OC[C@@]67CCCN6C[C@H](F)C7)nc5n3)N3CC5CCC(N5)C3CO4)c12 ; C#Cc1c(F)ccc2cccc(-c3nc4c5c(nc(OC[C@@]67CCCN6C[C@H](F)C7)nc5n3)N3CC5CCC(N5)C3CO4)c12 ; C#Cc1c(F)ccc2cc(O)cc(-c3nc4nc(OC[C@@]56CCCN5C[C@H](F)C6)nc5c4c(c3F)OCC3C4CCC(CN53)N4)c12 ; C#Cc1c(F)ccc2cccc(-c3nc4nc(OC[C@@]56CCCN5C[C@H](F)C6)nc5c4c(c3F)OCC3C4CCC(CN53)N4)c12]

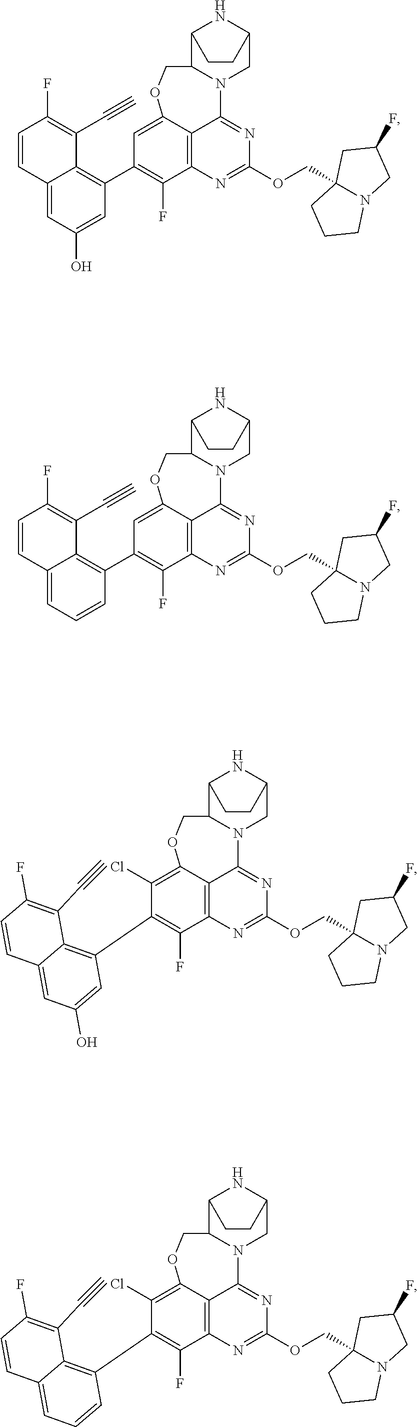 [SMILES: C#Cc1c(F)ccc2cc(O)cc(-c3cc4c5c(nc(OC[C@@]67CCCN6C[C@H](F)C7)nc5c3F)N3CC5CCC(N5)C3CO4)c12 ; C#Cc1c(F)ccc2cccc(-c3cc4c5c(nc(OC[C@@]67CCCN6C[C@H](F)C7)nc5c3F)N3CC5CCC(N5)C3CO4)c12 ; C#Cc1c(F)ccc2cc(O)cc(-c3c(Cl)c4c5c(nc(OC[C@@]67CCCN6C[C@H](F)C7)nc5c3F)N3CC5CCC(N5)C3CO4)c12 ; C#Cc1c(F)ccc2cccc(-c3c(Cl)c4c5c(nc(OC[C@@]67CCCN6C[C@H](F)C7)nc5c3F)N3CC5CCC(N5)C3CO4)c12]

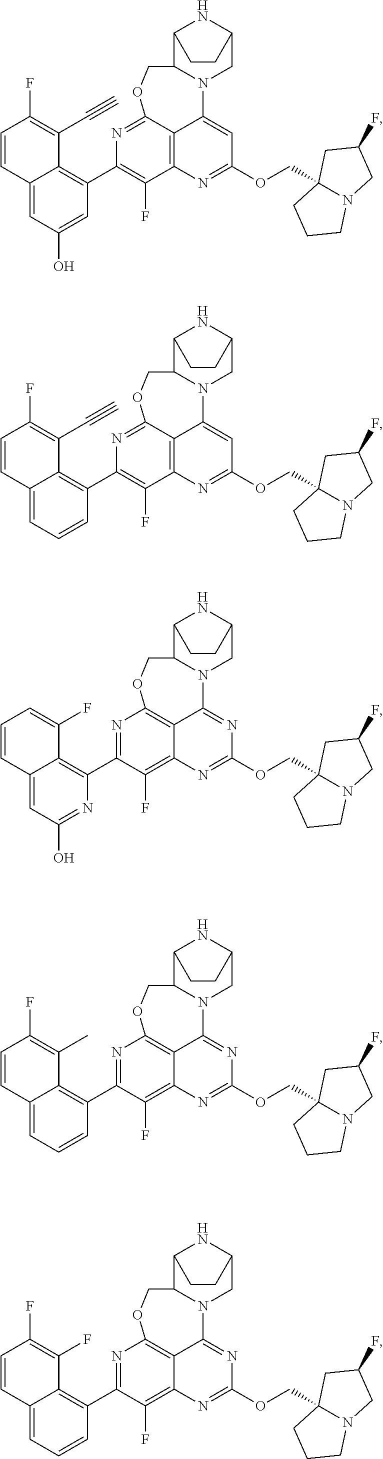 [SMILES: C#Cc1c(F)ccc2cc(O)cc(-c3nc4c5c(cc(OC[C@@]67CCCN6C[C@H](F)C7)nc5c3F)N3CC5CCC(N5)C3CO4)c12 ; C#Cc1c(F)ccc2cccc(-c3nc4c5c(cc(OC[C@@]67CCCN6C[C@H](F)C7)nc5c3F)N3CC5CCC(N5)C3CO4)c12 ; Oc1cc2cccc(F)c2c(-c2nc3c4c(nc(OC[C@@]56CCCN5C[C@H](F)C6)nc4c2F)N2CC4CCC(N4)C2CO3)n1 ; Cc1c(F)ccc2cccc(-c3nc4c5c(nc(OC[C@@]67CCCN6C[C@H](F)C7)nc5c3F)N3CC5CCC(N5)C3CO4)c12 ; Fc1ccc2cccc(-c3nc4c5c(nc(OC[C@@]67CCCN6C[C@H](F)C7)nc5c3F)N3CC5CCC(N5)C3CO4)c2c1F]

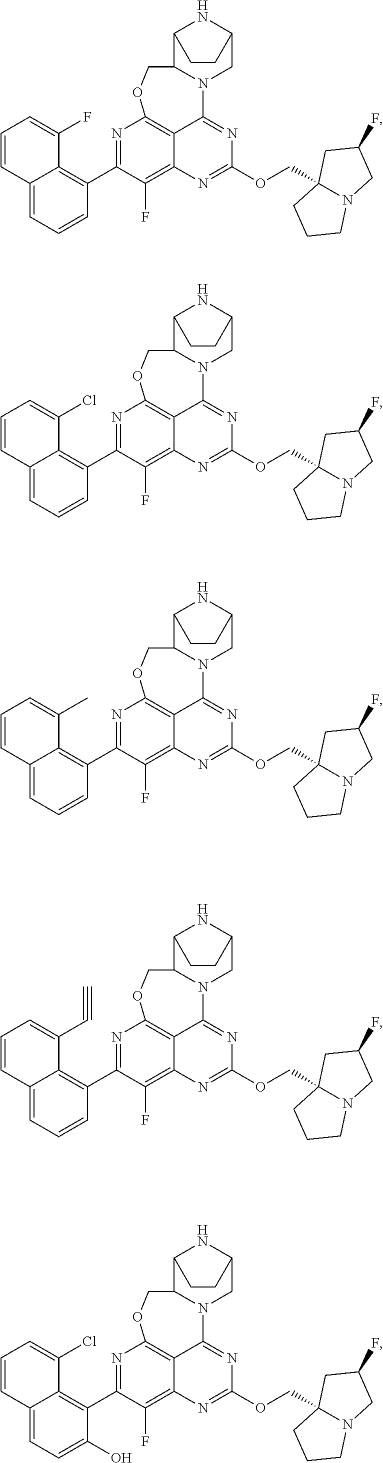 [SMILES: Fc1c(-c2cccc3cccc(F)c23)nc2c3c(nc(OC[C@@]45CCCN4C[C@H](F)C5)nc13)N1CC3CCC(N3)C1CO2 ; Fc1c(-c2cccc3cccc(Cl)c23)nc2c3c(nc(OC[C@@]45CCCN4C[C@H](F)C5)nc13)N1CC3CCC(N3)C1CO2 ; Cc1cccc2cccc(-c3nc4c5c(nc(OC[C@@]67CCCN6C[C@H](F)C7)nc5c3F)N3CC5CCC(N5)C3CO4)c12 ; C#Cc1cccc2cccc(-c3nc4c5c(nc(OC[C@@]67CCCN6C[C@H](F)C7)nc5c3F)N3CC5CCC(N5)C3CO4)c12 ; Oc1ccc2cccc(Cl)c2c1-c1nc2c3c(nc(OC[C@@]45CCCN4C[C@H](F)C5)nc3c1F)N1CC3CCC(N3)C1CO2]

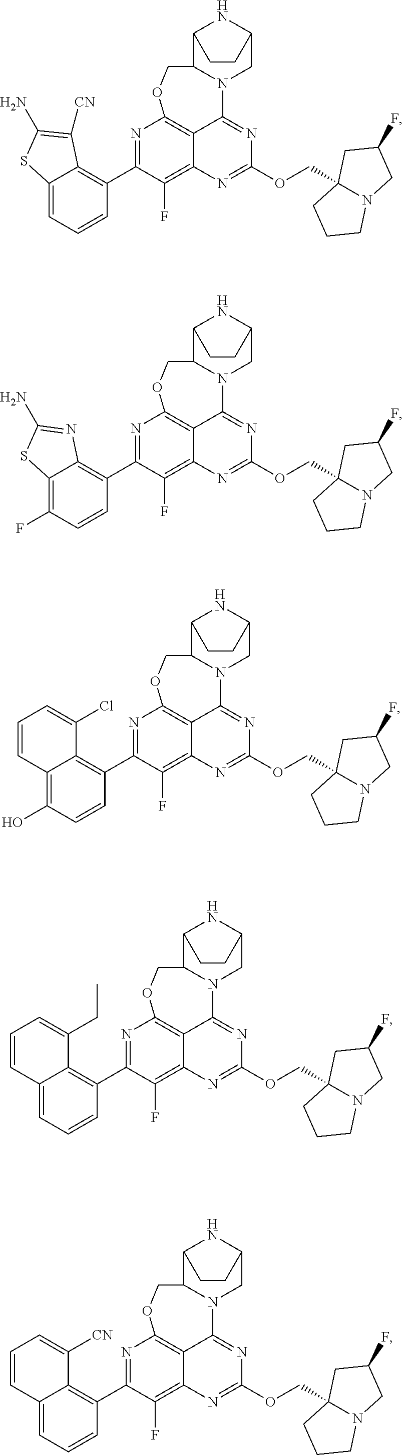 [SMILES: N#Cc1c(N)sc2cccc(-c3nc4c5c(nc(OC[C@@]67CCCN6C[C@H](F)C7)nc5c3F)N3CC5CCC(N5)C3CO4)c12 ; Nc1nc2c(-c3nc4c5c(nc(OC[C@@]67CCCN6C[C@H](F)C7)nc5c3F)N3CC5CCC(N5)C3CO4)ccc(F)c2s1 ; Oc1ccc(-c2nc3c4c(nc(OC[C@@]56CCCN5C[C@H](F)C6)nc4c2F)N2CC4CCC(N4)C2CO3)c2c(Cl)cccc12 ; CCc1cccc2cccc(-c3nc4c5c(nc(OC[C@@]67CCCN6C[C@H](F)C7)nc5c3F)N3CC5CCC(N5)C3CO4)c12 ; N#Cc1cccc2cccc(-c3nc4c5c(nc(OC[C@@]67CCCN6C[C@H](F)C7)nc5c3F)N3CC5CCC(N5)C3CO4)c12]

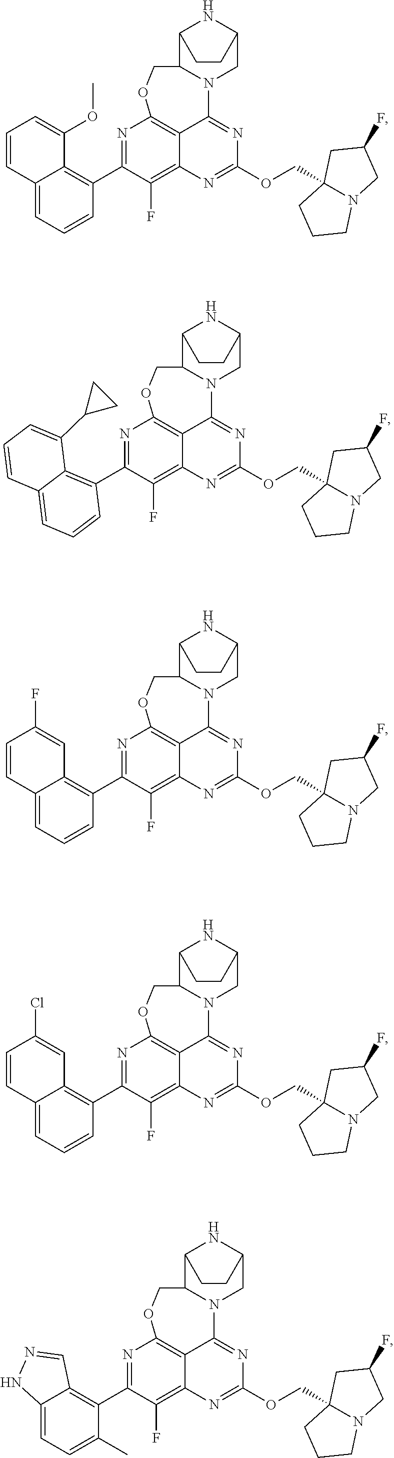 [SMILES: COc1cccc2cccc(-c3nc4c5c(nc(OC[C@@]67CCCN6C[C@H](F)C7)nc5c3F)N3CC5CCC(N5)C3CO4)c12 ; Fc1c(-c2cccc3cccc(C4CC4)c23)nc2c3c(nc(OC[C@@]45CCCN4C[C@H](F)C5)nc13)N1CC3CCC(N3)C1CO2 ; Fc1ccc2cccc(-c3nc4c5c(nc(OC[C@@]67CCCN6C[C@H](F)C7)nc5c3F)N3CC5CCC(N5)C3CO4)c2c1 ; Fc1c(-c2cccc3ccc(Cl)cc23)nc2c3c(nc(OC[C@@]45CCCN4C[C@H](F)C5)nc13)N1CC3CCC(N3)C1CO2 ; Cc1ccc2[nH]ncc2c1-c1nc2c3c(nc(OC[C@@]45CCCN4C[C@H](F)C5)nc3c1F)N1CC3CCC(N3)C1CO2]

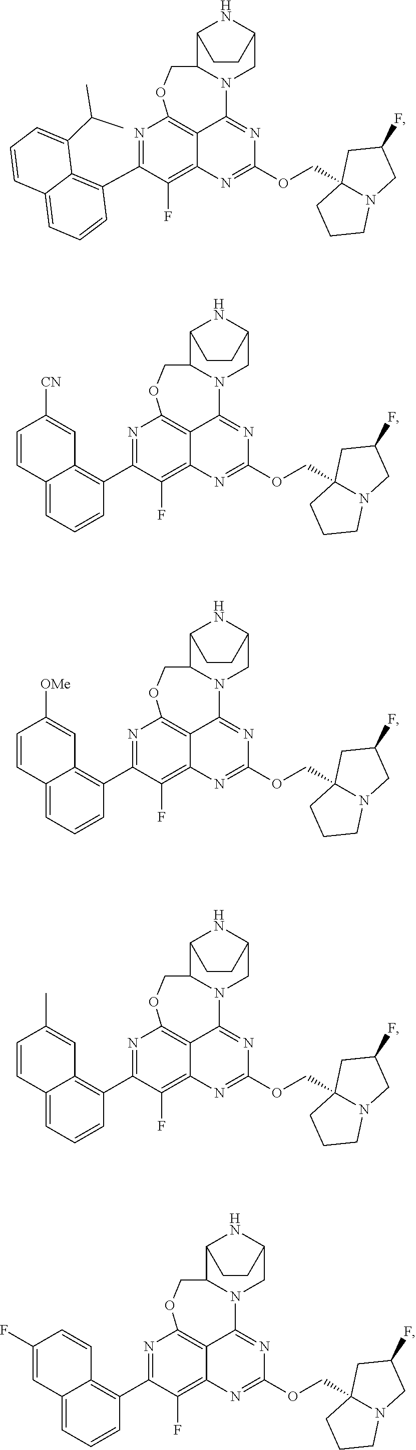 [SMILES: CC(C)c1cccc2cccc(-c3nc4c5c(nc(OC[C@@]67CCCN6C[C@H](F)C7)nc5c3F)N3CC5CCC(N5)C3CO4)c12 ; N#Cc1ccc2cccc(-c3nc4c5c(nc(OC[C@@]67CCCN6C[C@H](F)C7)nc5c3F)N3CC5CCC(N5)C3CO4)c2c1 ; COc1ccc2cccc(-c3nc4c5c(nc(OC[C@@]67CCCN6C[C@H](F)C7)nc5c3F)N3CC5CCC(N5)C3CO4)c2c1 ; Cc1ccc2cccc(-c3nc4c5c(nc(OC[C@@]67CCCN6C[C@H](F)C7)nc5c3F)N3CC5CCC(N5)C3CO4)c2c1 ; Fc1ccc2c(-c3nc4c5c(nc(OC[C@@]67CCCN6C[C@H](F)C7)nc5c3F)N3CC5CCC(N5)C3CO4)cccc2c1]

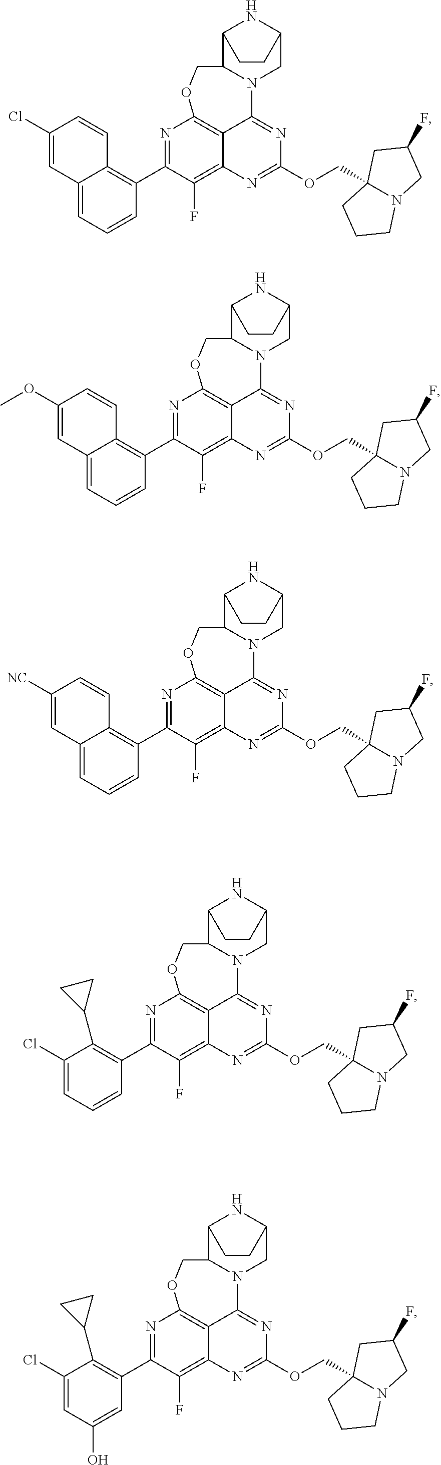 [SMILES: Fc1c(-c2cccc3cc(Cl)ccc23)nc2c3c(nc(OC[C@@]45CCCN4C[C@H](F)C5)nc13)N1CC3CCC(N3)C1CO2 ; COc1ccc2c(-c3nc4c5c(nc(OC[C@@]67CCCN6C[C@H](F)C7)nc5c3F)N3CC5CCC(N5)C3CO4)cccc2c1 ; N#Cc1ccc2c(-c3nc4c5c(nc(OC[C@@]67CCCN6C[C@H](F)C7)nc5c3F)N3CC5CCC(N5)C3CO4)cccc2c1 ; Fc1c(-c2cccc(Cl)c2C2CC2)nc2c3c(nc(OC[C@@]45CCCN4C[C@H](F)C5)nc13)N1CC3CCC(N3)C1CO2 ; Oc1cc(Cl)c(C2CC2)c(-c2nc3c4c(nc(OC[C@@]56CCCN5C[C@H](F)C6)nc4c2F)N2CC4CCC(N4)C2CO3)c1]

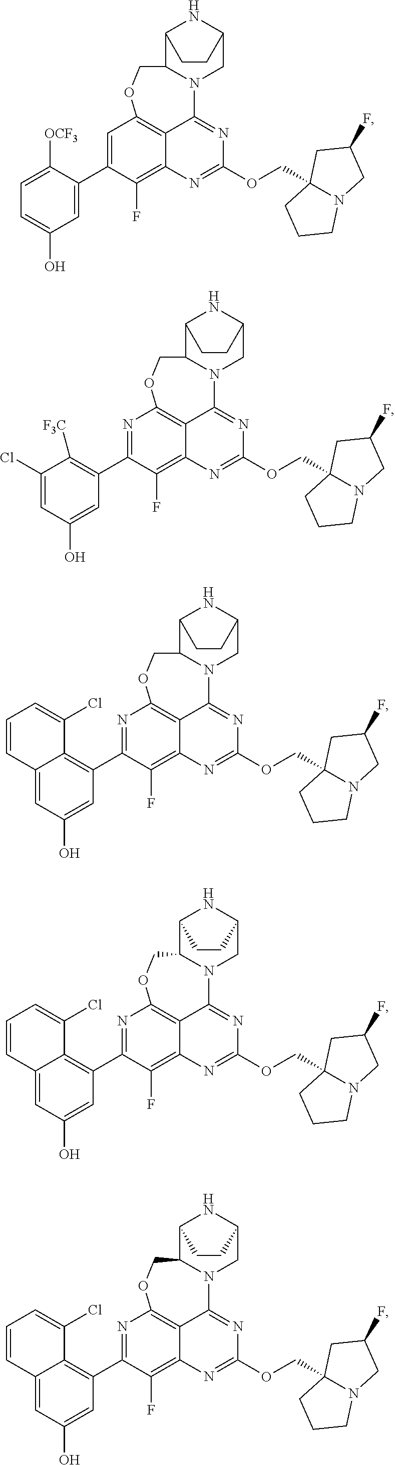 [SMILES: Oc1ccc(OC(F)(F)F)c(-c2cc3c4c(nc(OC[C@@]56CCCN5C[C@H](F)C6)nc4c2F)N2CC4CCC(N4)C2CO3)c1 ; Oc1cc(Cl)c(C(F)(F)F)c(-c2nc3c4c(nc(OC[C@@]56CCCN5C[C@H](F)C6)nc4c2F)N2CC4CCC(N4)C2CO3)c1 ; Oc1cc(-c2nc3c4c(nc(OC[C@@]56CCCN5C[C@H](F)C6)nc4c2F)N2CC4CCC(N4)C2CO3)c2c(Cl)cccc2c1 ; Oc1cc(-c2nc3c4c(nc(OC[C@@]56CCCN5C[C@H](F)C6)nc4c2F)N2C[C@H]4CC[C@H](N4)[C@H]2CO3)c2c(Cl)cccc2c1 ; Oc1cc(-c2nc3c4c(nc(OC[C@@]56CCCN5C[C@H](F)C6)nc4c2F)N2C[C@H]4CC[C@H](N4)[C@@H]2CO3)c2c(Cl)cccc2c1]

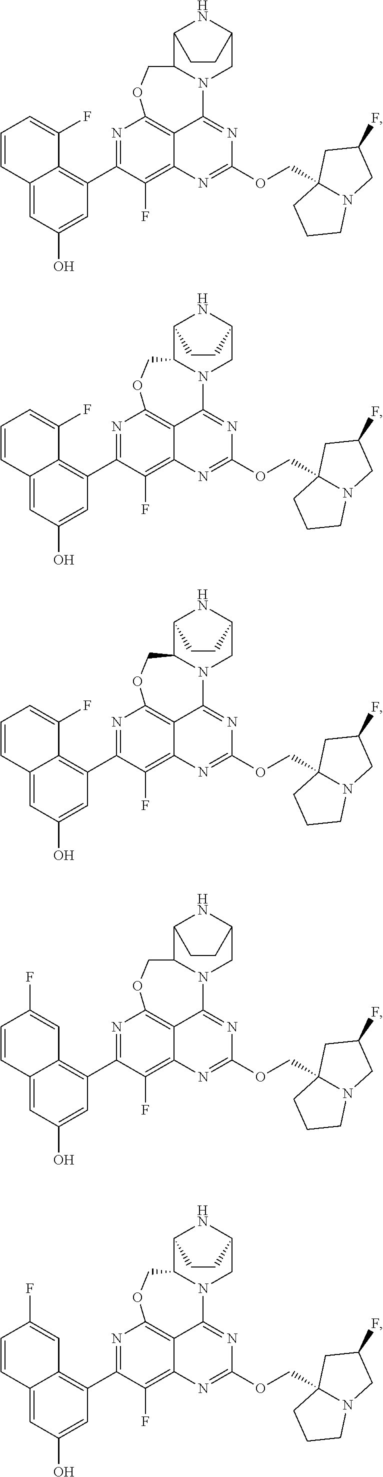 [SMILES: Oc1cc(-c2nc3c4c(nc(OC[C@@]56CCCN5C[C@H](F)C6)nc4c2F)N2CC4CCC(N4)C2CO3)c2c(F)cccc2c1 ; Oc1cc(-c2nc3c4c(nc(OC[C@@]56CCCN5C[C@H](F)C6)nc4c2F)N2C[C@H]4CC[C@H](N4)[C@H]2CO3)c2c(F)cccc2c1 ; Oc1cc(-c2nc3c4c(nc(OC[C@@]56CCCN5C[C@H](F)C6)nc4c2F)N2C[C@H]4CC[C@H](N4)[C@@H]2CO3)c2c(F)cccc2c1 ; Oc1cc(-c2nc3c4c(nc(OC[C@@]56CCCN5C[C@H](F)C6)nc4c2F)N2CC4CCC(N4)C2CO3)c2cc(F)ccc2c1 ; Oc1cc(-c2nc3c4c(nc(OC[C@@]56CCCN5C[C@H](F)C6)nc4c2F)N2C[C@H]4CC[C@H](N4)[C@H]2CO3)c2cc(F)ccc2c1]

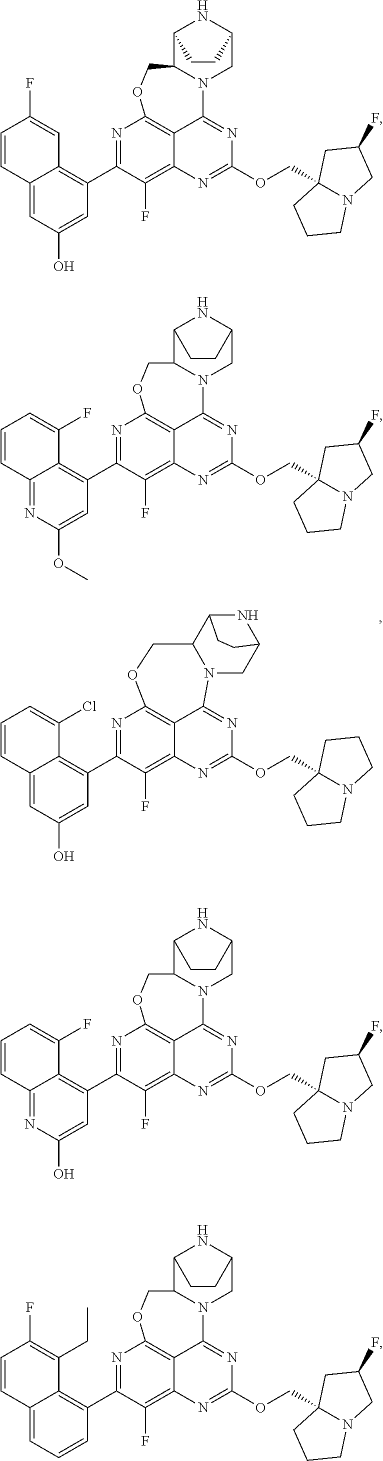

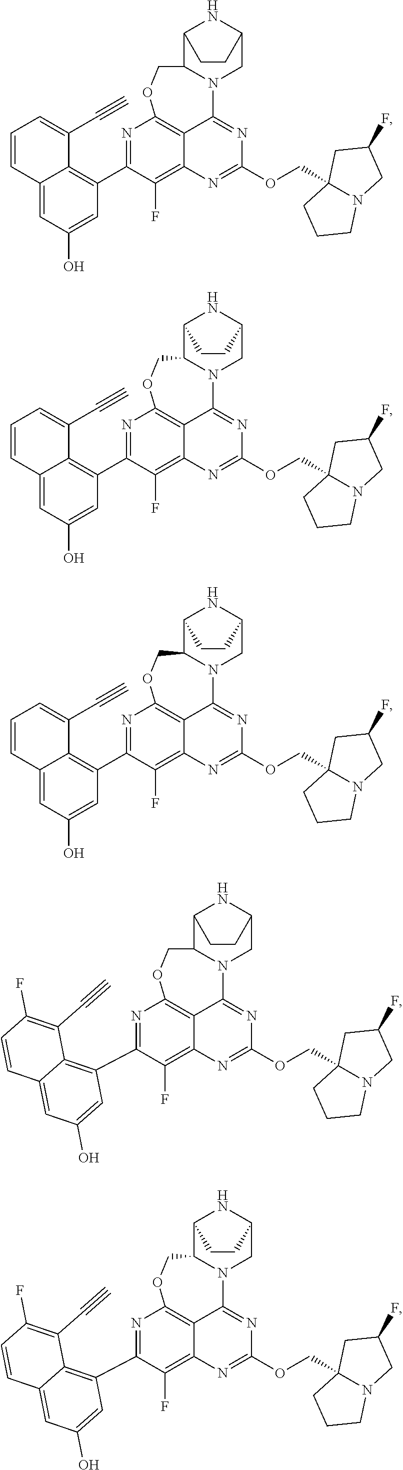 [SMILES: C#Cc1cccc2cc(O)cc(-c3nc4c5c(nc(OC[C@@]67CCCN6C[C@H](F)C7)nc5c3F)N3CC5CCC(N5)C3CO4)c12 ; C#Cc1cccc2cc(O)cc(-c3nc4c5c(nc(OC[C@@]67CCCN6C[C@H](F)C7)nc5c3F)N3C[C@H]5CC[C@H](N5)[C@H]3CO4)c12 ; C#Cc1cccc2cc(O)cc(-c3nc4c5c(nc(OC[C@@]67CCCN6C[C@H](F)C7)nc5c3F)N3C[C@H]5CC[C@H](N5)[C@@H]3CO4)c12 ; C#Cc1c(F)ccc2cc(O)cc(-c3nc4c5c(nc(OC[C@@]67CCCN6C[C@H](F)C7)nc5c3F)N3CC5CCC(N5)C3CO4)c12 ; C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

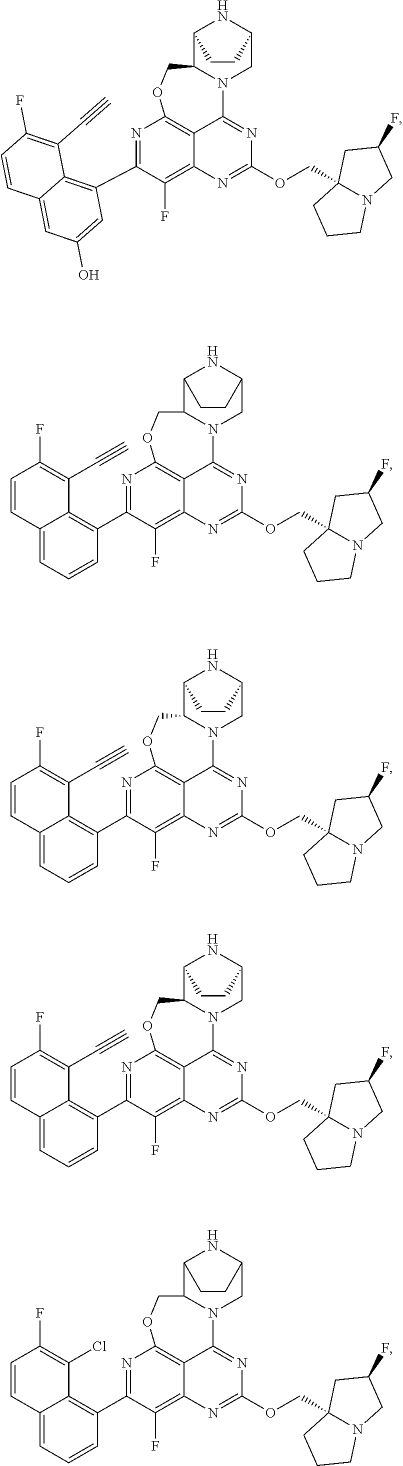 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@@H]3CO4)c12 ; C#Cc1c(F)ccc2cccc(-c3nc4c5c(nc(OC[C@@]67CCCN6C[C@H](F)C7)nc5c3F)N3CC5CCC(N5)C3CO4)c12 ; C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12 ; C#Cc1c(F)ccc2cccc(-c3nc4c5c(nc(OC[C@@]67CCCN6C[C@H](F)C7)nc5c3F)N3C[C@H]5CC[C@H](N5)[C@@H]3CO4)c12 ; Fc1ccc2cccc(-c3nc4c5c(nc(OC[C@@]67CCCN6C[C@H](F)C7)nc5c3F)N3CC5CCC(N5)C3CO4)c2c1Cl]

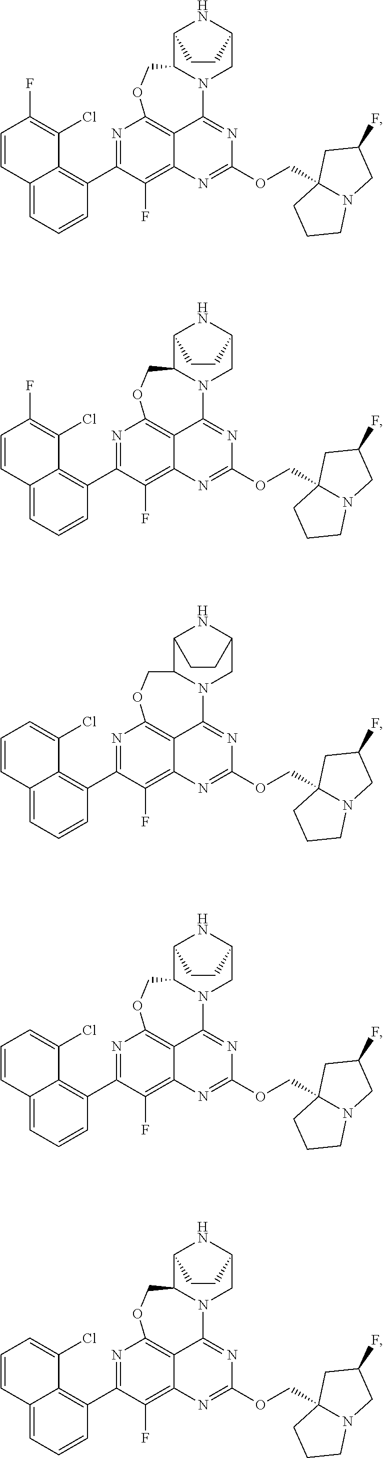 [SMILES: Fc1ccc2cccc(-c3nc4c5c(nc(OC[C@@]67CCCN6C[C@H](F)C7)nc5c3F)N3C[C@H]5CC[C@H](N5)[C@H]3CO4)c2c1Cl ; Fc1ccc2cccc(-c3nc4c5c(nc(OC[C@@]67CCCN6C[C@H](F)C7)nc5c3F)N3C[C@H]5CC[C@H](N5)[C@@H]3CO4)c2c1Cl ; Fc1c(-c2cccc3cccc(Cl)c23)nc2c3c(nc(OC[C@@]45CCCN4C[C@H](F)C5)nc13)N1CC3CCC(N3)C1CO2 ; Fc1c(-c2cccc3cccc(Cl)c23)nc2c3c(nc(OC[C@@]45CCCN4C[C@H](F)C5)nc13)N1C[C@H]3CC[C@H](N3)[C@H]1CO2 ; Fc1c(-c2cccc3cccc(Cl)c23)nc2c3c(nc(OC[C@@]45CCCN4C[C@H](F)C5)nc13)N1C[C@H]3CC[C@H](N3)[C@@H]1CO2]

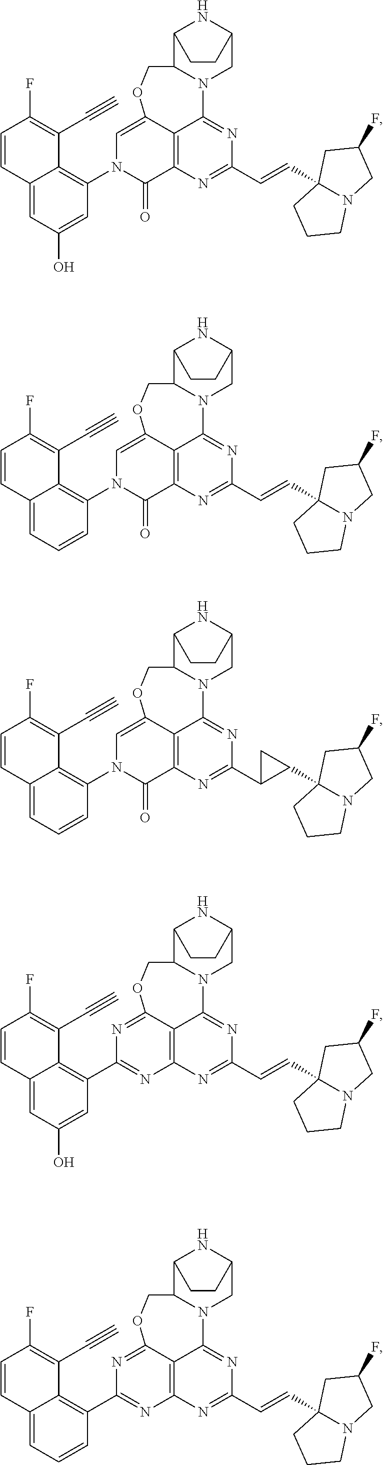 [SMILES: C#Cc1c(F)ccc2cc(O)cc(-n3cc4c5c(nc(/C=C/[C@@]67CCCN6C[C@H](F)C7)nc5c3=O)N3CC5CCC(N5)C3CO4)c12 ; C#Cc1c(F)ccc2cccc(-n3cc4c5c(nc(/C=C/[C@@]67CCCN6C[C@H](F)C7)nc5c3=O)N3CC5CCC(N5)C3CO4)c12 ; C#Cc1c(F)ccc2cccc(-n3cc4c5c(nc(C6CC6[C@@]67CCCN6C[C@H](F)C7)nc5c3=O)N3CC5CCC(N5)C3CO4)c12 ; C#Cc1c(F)ccc2cc(O)cc(-c3nc4c5c(nc(/C=C/[C@@]67CCCN6C[C@H](F)C7)nc5n3)N3CC5CCC(N5)C3CO4)c12 ; C#Cc1c(F)ccc2cccc(-c3nc4c5c(nc(/C=C/[C@@]67CCCN6C[C@H](F)C7)nc5n3)N3CC5CCC(N5)C3CO4)c12]

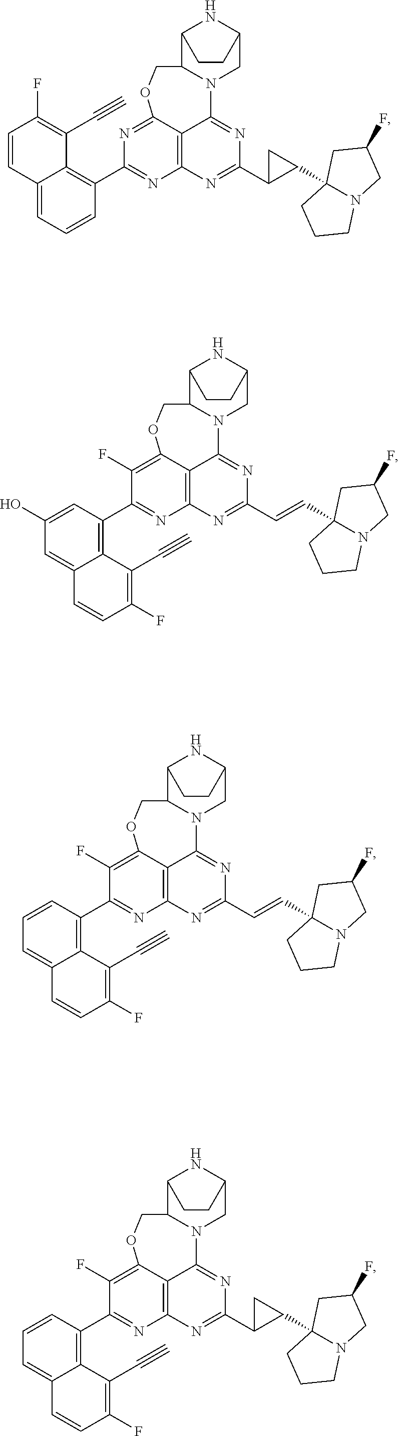 [SMILES: C#Cc1c(F)ccc2cccc(-c3nc4c5c(nc(C6CC6[C@@]67CCCN6C[C@H](F)C7)nc5n3)N3CC5CCC(N5)C3CO4)c12 ; C#Cc1c(F)ccc2cc(O)cc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(C5CC5[C@@]56CCCN5C[C@H](F)C6)nc5c4c(c3F)OCC3C4CCC(CN53)N4)c12]

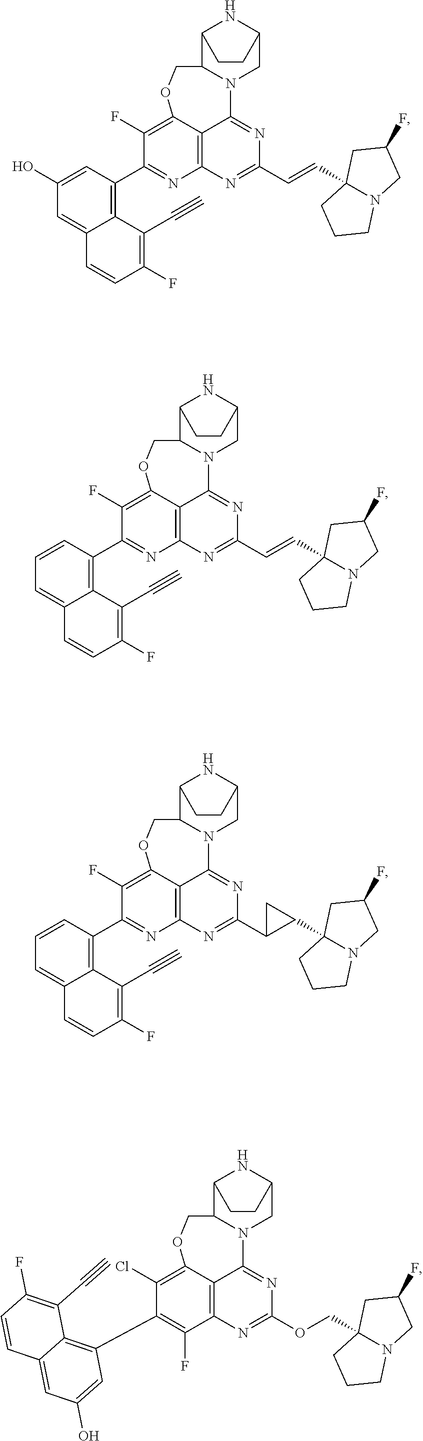 [SMILES: C#Cc1c(F)ccc2cc(O)cc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(C5CC5[C@@]56CCCN5C[C@H](F)C6)nc5c4c(c3F)OCC3C4CCC(CN53)N4)c12 ; C#Cc1c(F)ccc2cc(O)cc(-c3c(Cl)c4c5c(nc(OC[C@@]67CCCN6C[C@H](F)C7)nc5c3F)N3CC5CCC(N5)C3CO4)c12]

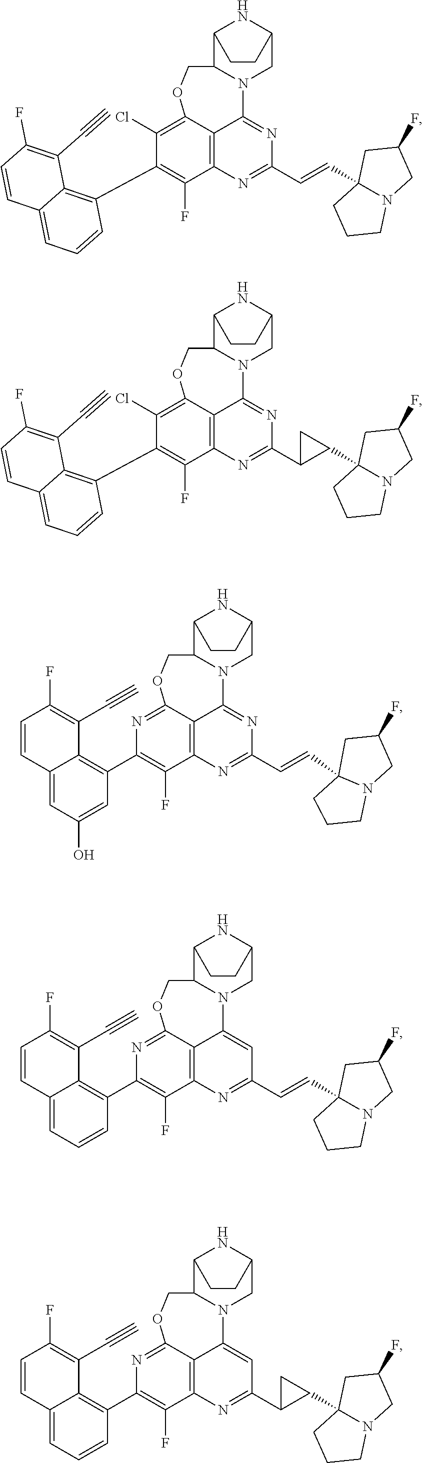 [SMILES: C#Cc1c(F)ccc2cccc(-c3c(Cl)c4c5c(nc(/C=C/[C@@]67CCCN6C[C@H](F)C7)nc5c3F)N3CC5CCC(N5)C3CO4)c12 ; C#Cc1c(F)ccc2cccc(-c3c(Cl)c4c5c(nc(C6CC6[C@@]67CCCN6C[C@H](F)C7)nc5c3F)N3CC5CCC(N5)C3CO4)c12 ; C#Cc1c(F)ccc2cc(O)cc(-c3nc4c5c(nc(/C=C/[C@@]67CCCN6C[C@H](F)C7)nc5c3F)N3CC5CCC(N5)C3CO4)c12 ; C#Cc1c(F)ccc2cccc(-c3nc4c5c(cc(/C=C/[C@@]67CCCN6C[C@H](F)C7)nc5c3F)N3CC5CCC(N5)C3CO4)c12 ; C#Cc1c(F)ccc2cccc(-c3nc4c5c(cc(C6CC6[C@@]67CCCN6C[C@H](F)C7)nc5c3F)N3CC5CCC(N5)C3CO4)c12]

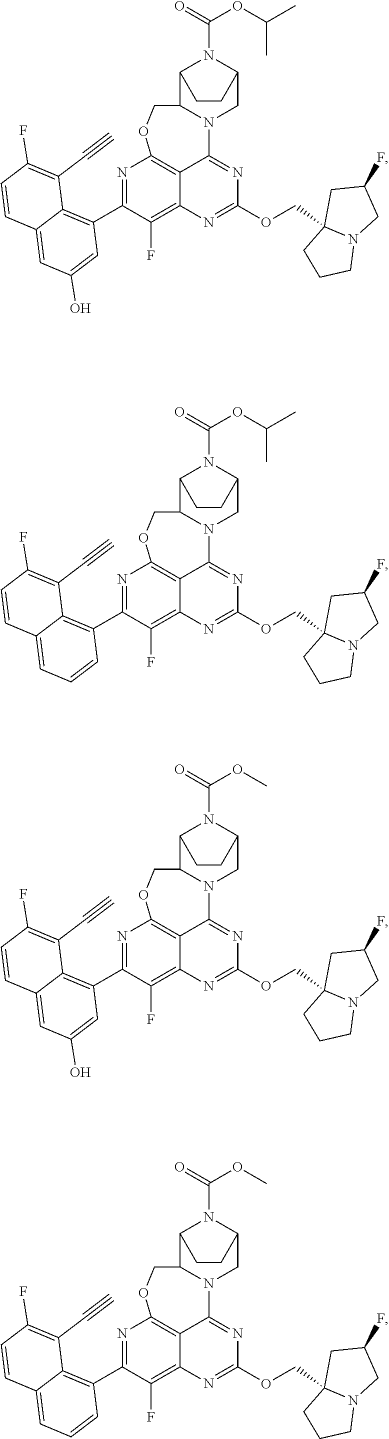 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)C)c12 ; C#Cc1c(F)ccc2cccc(-c3nc4c5c(nc(OC[C@@]67CCCN6C[C@H](F)C7)nc5c3F)N3CC5CCC(C3CO4)N5C(=O)OC(C)C)c12 ; C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC)c12 ; C#Cc1c(F)ccc2cccc(-c3nc4c5c(nc(OC[C@@]67CCCN6C[C@H](F)C7)nc5c3F)N3CC5CCC(C3CO4)N5C(=O)OC)c12]

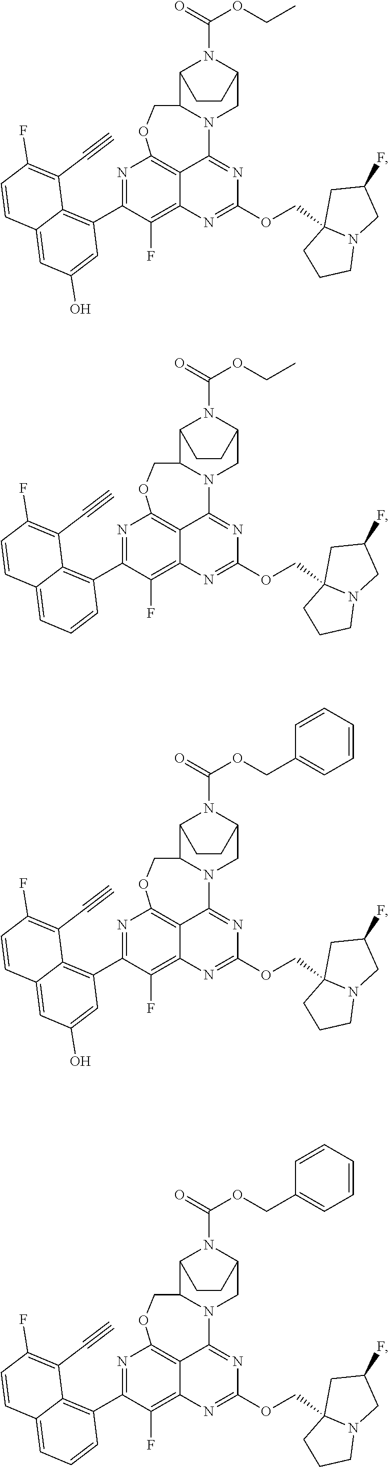 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OCC)c12 ; C#Cc1c(F)ccc2cccc(-c3nc4c5c(nc(OC[C@@]67CCCN6C[C@H](F)C7)nc5c3F)N3CC5CCC(C3CO4)N5C(=O)OCC)c12 ; C#Cc1c(F)ccc2cc(O)cc(-c3nc4c5c(nc(OC[C@@]67CCCN6C[C@H](F)C7)nc5c3F)N3CC5CCC(C3CO4)N5C(=O)OCc3ccccc3)c12 ; C#Cc1c(F)ccc2cccc(-c3nc4c5c(nc(OC[C@@]67CCCN6C[C@H](F)C7)nc5c3F)N3CC5CCC(C3CO4)N5C(=O)OCc3ccccc3)c12]

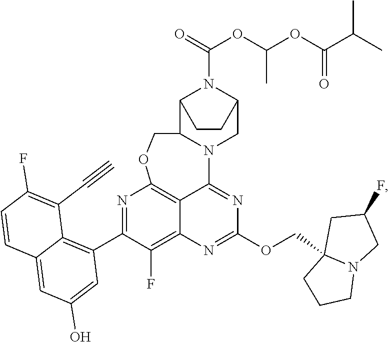 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)OC(=O)C(C)C)c12]

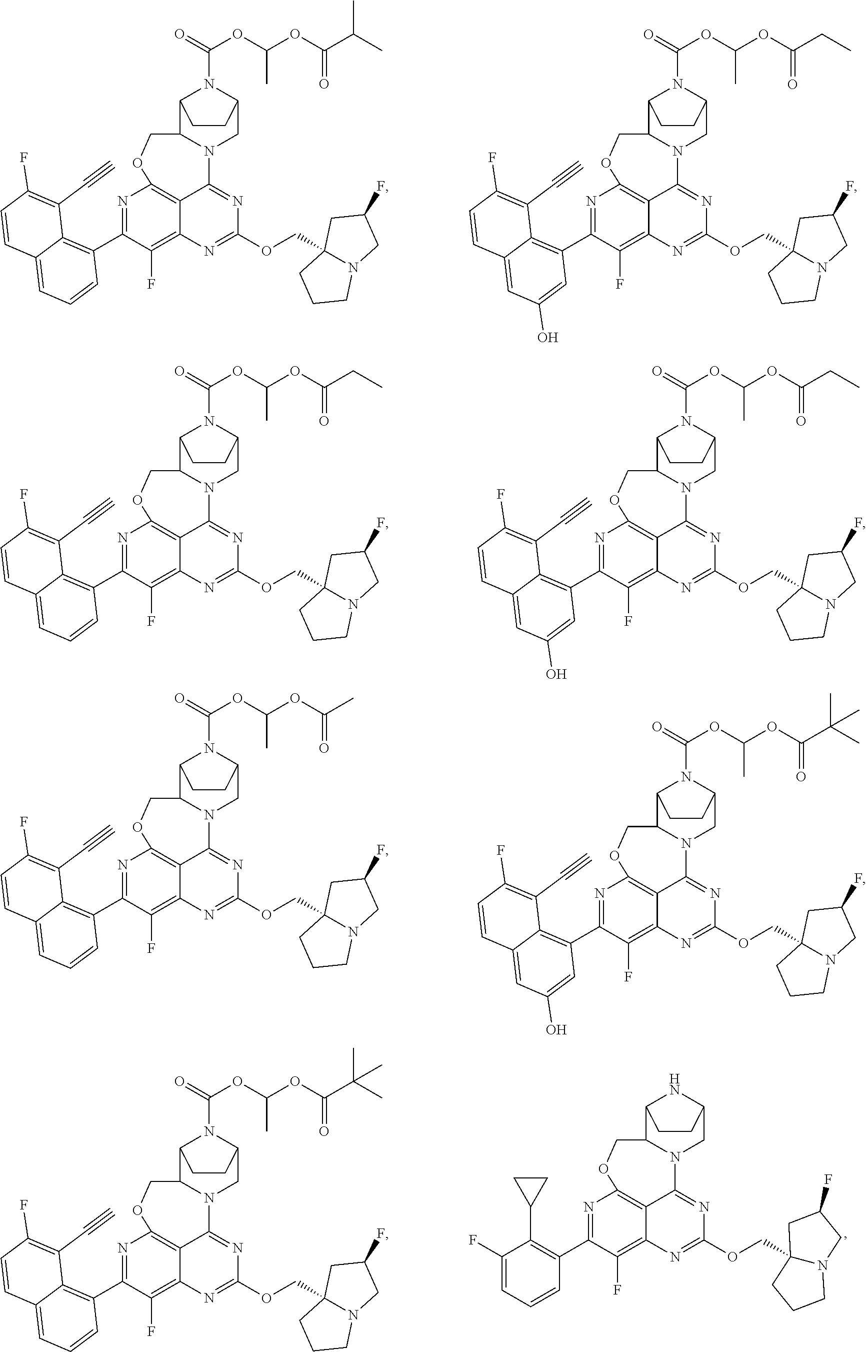

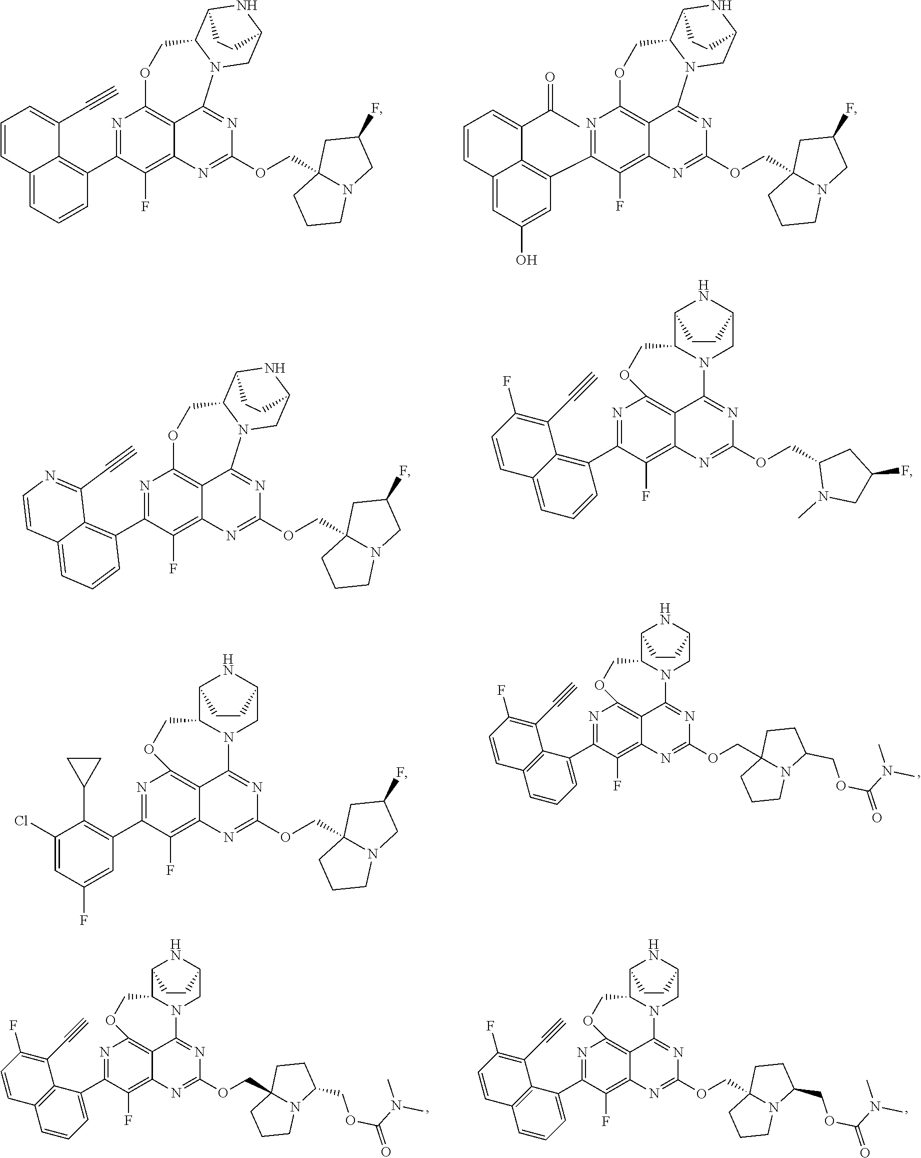

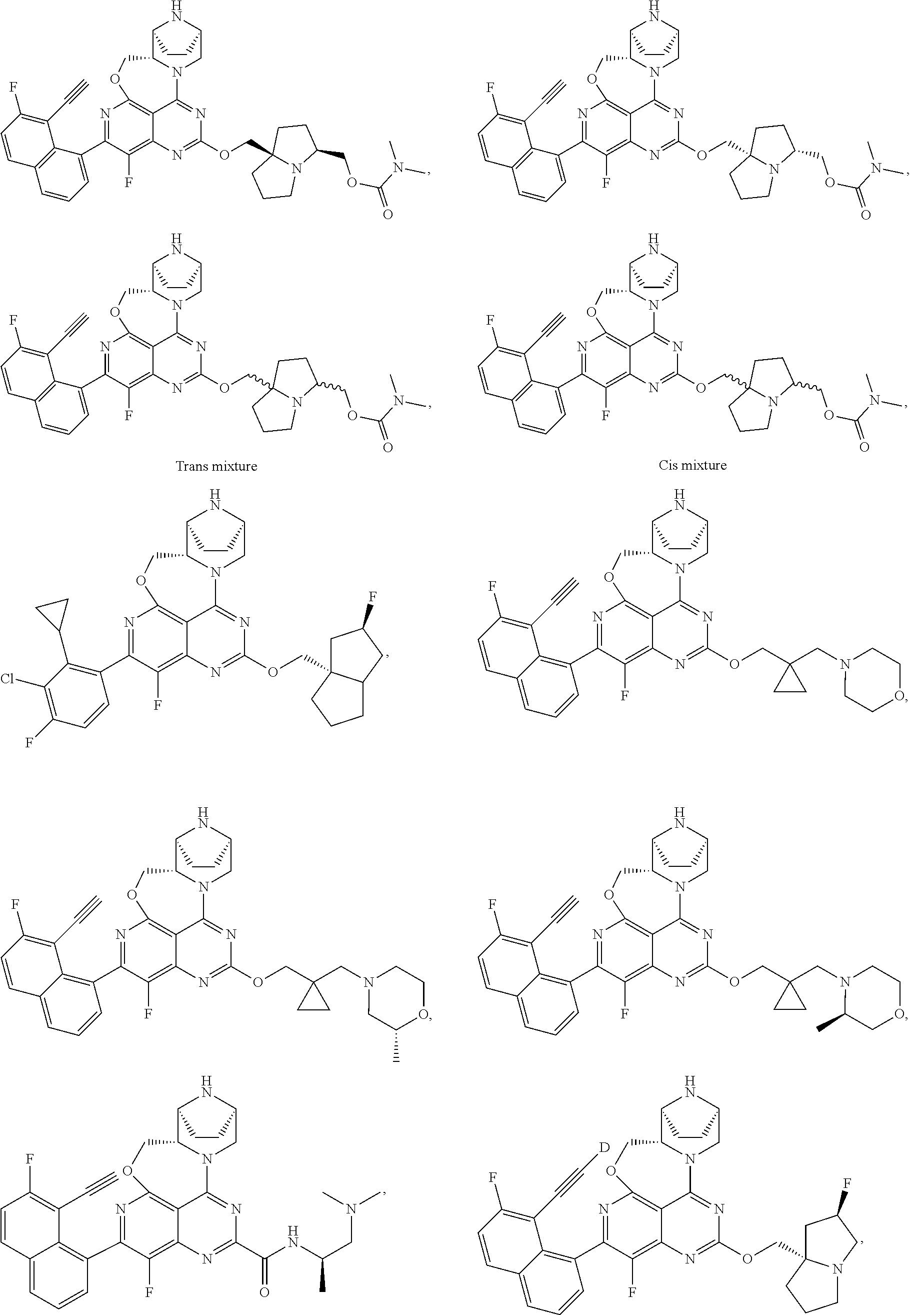

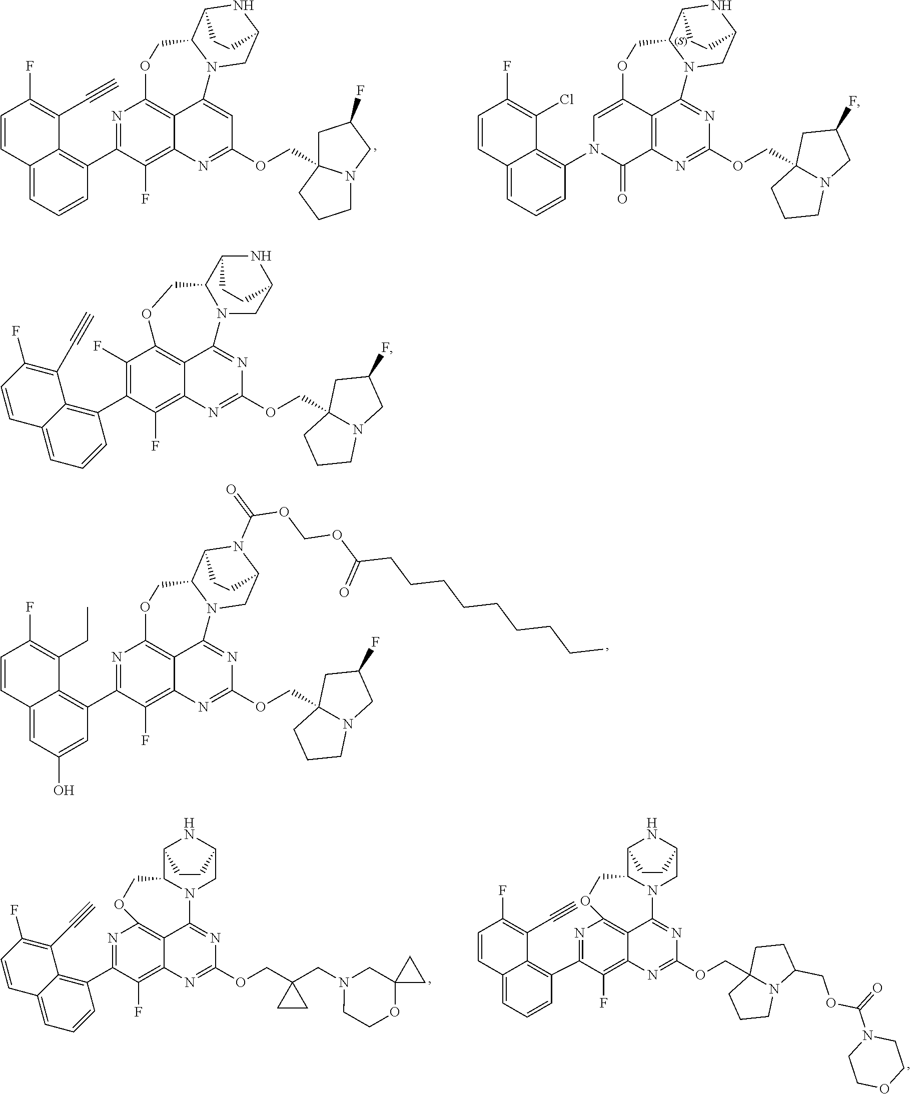

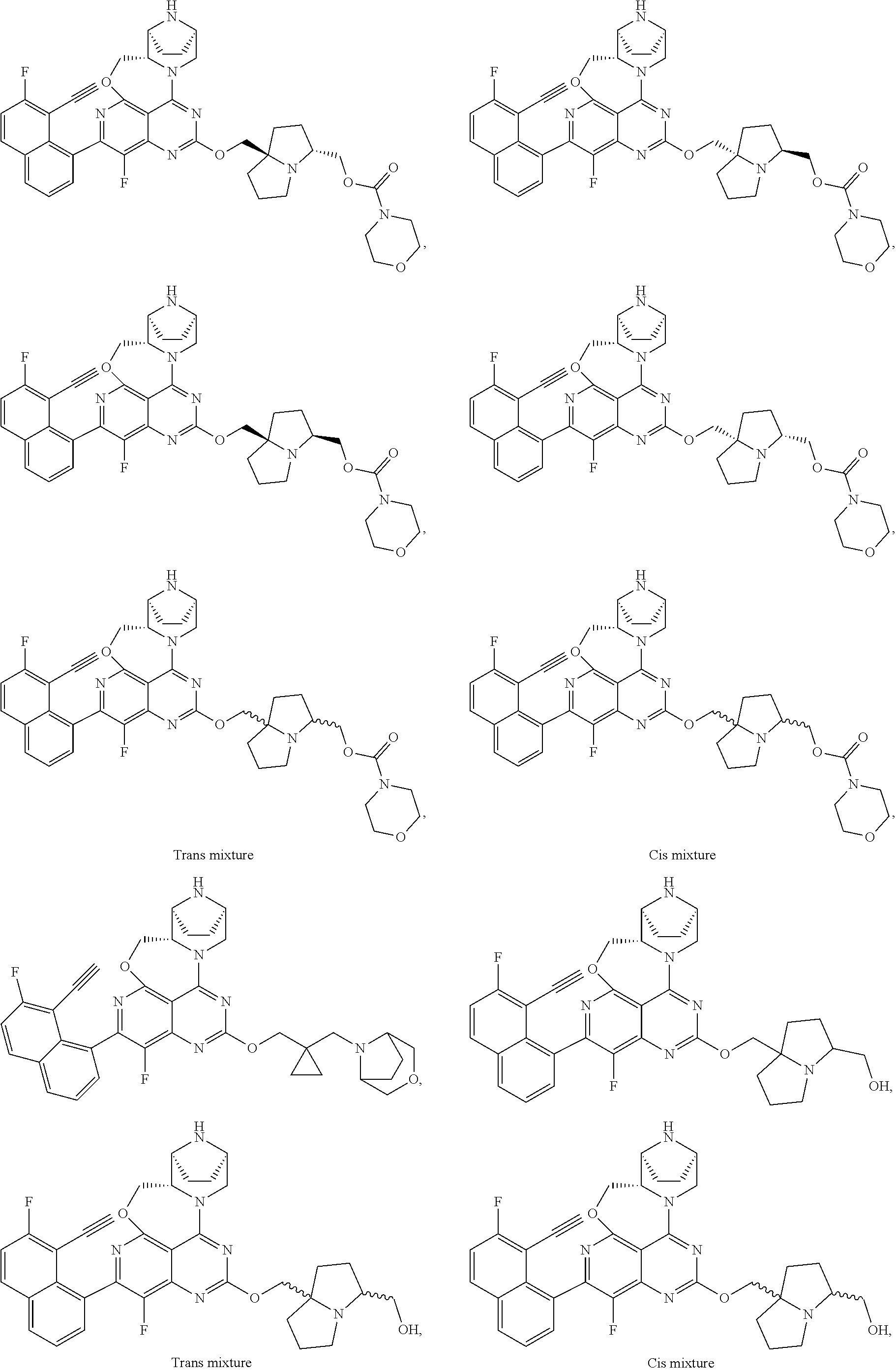

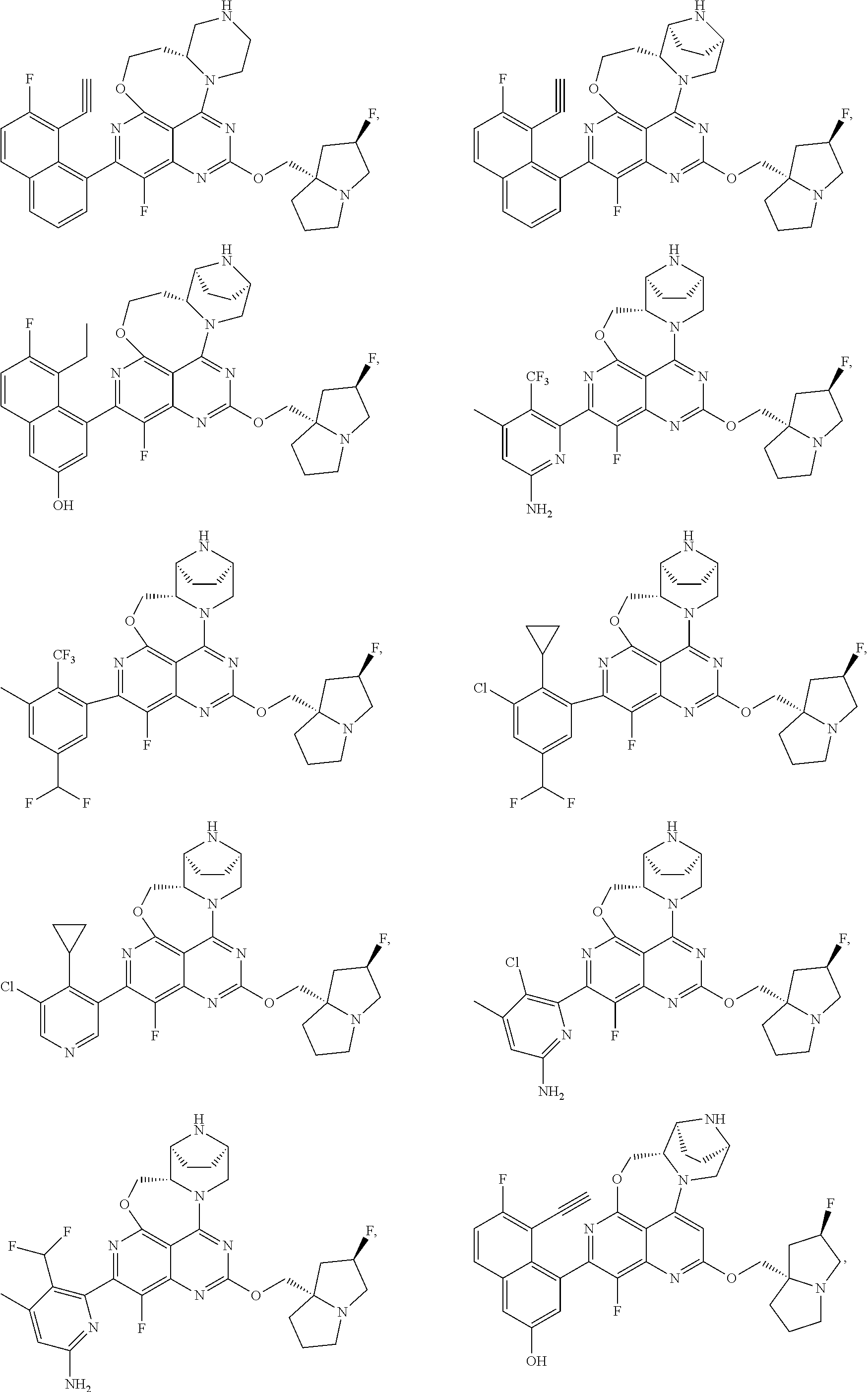

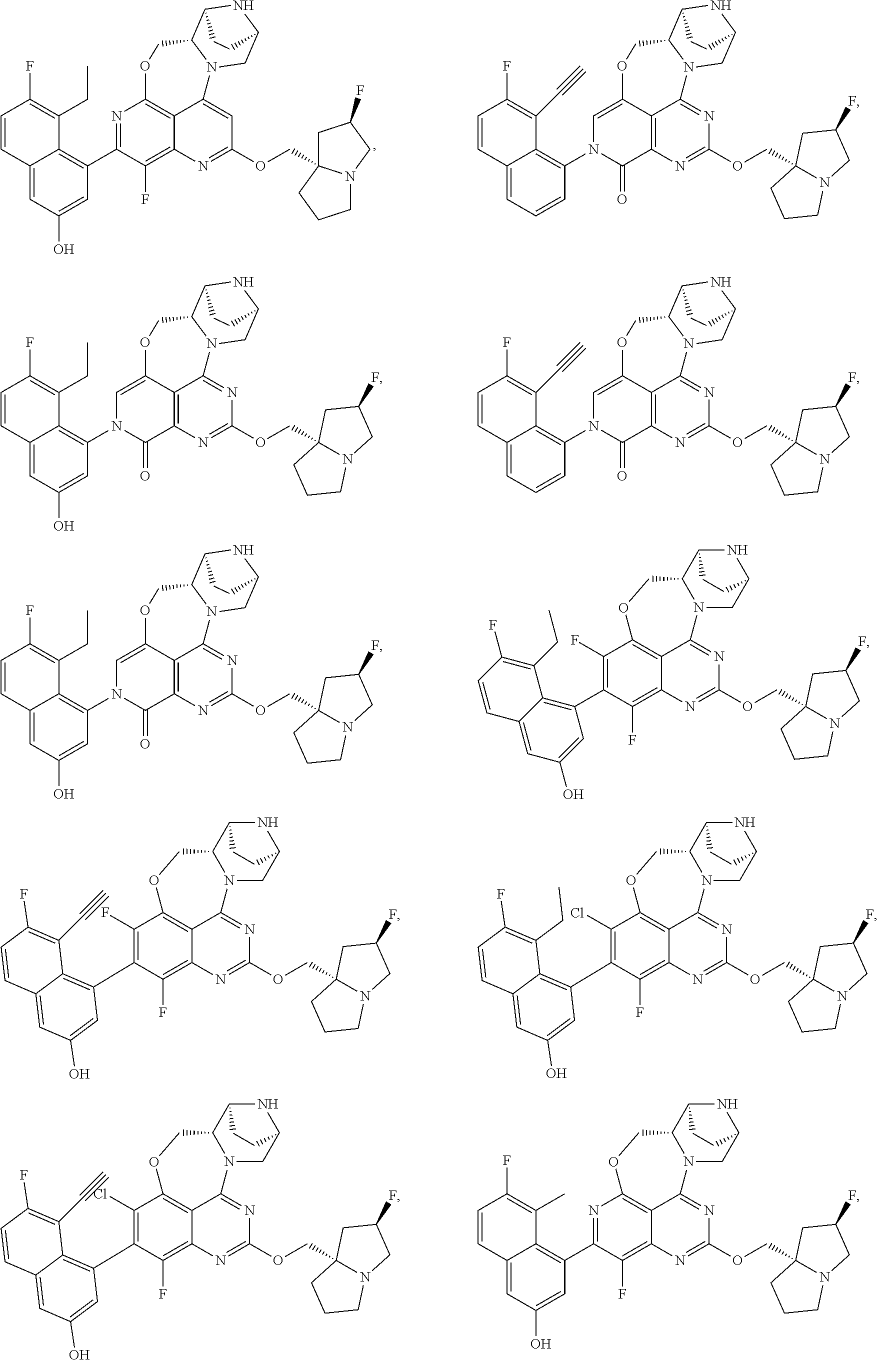

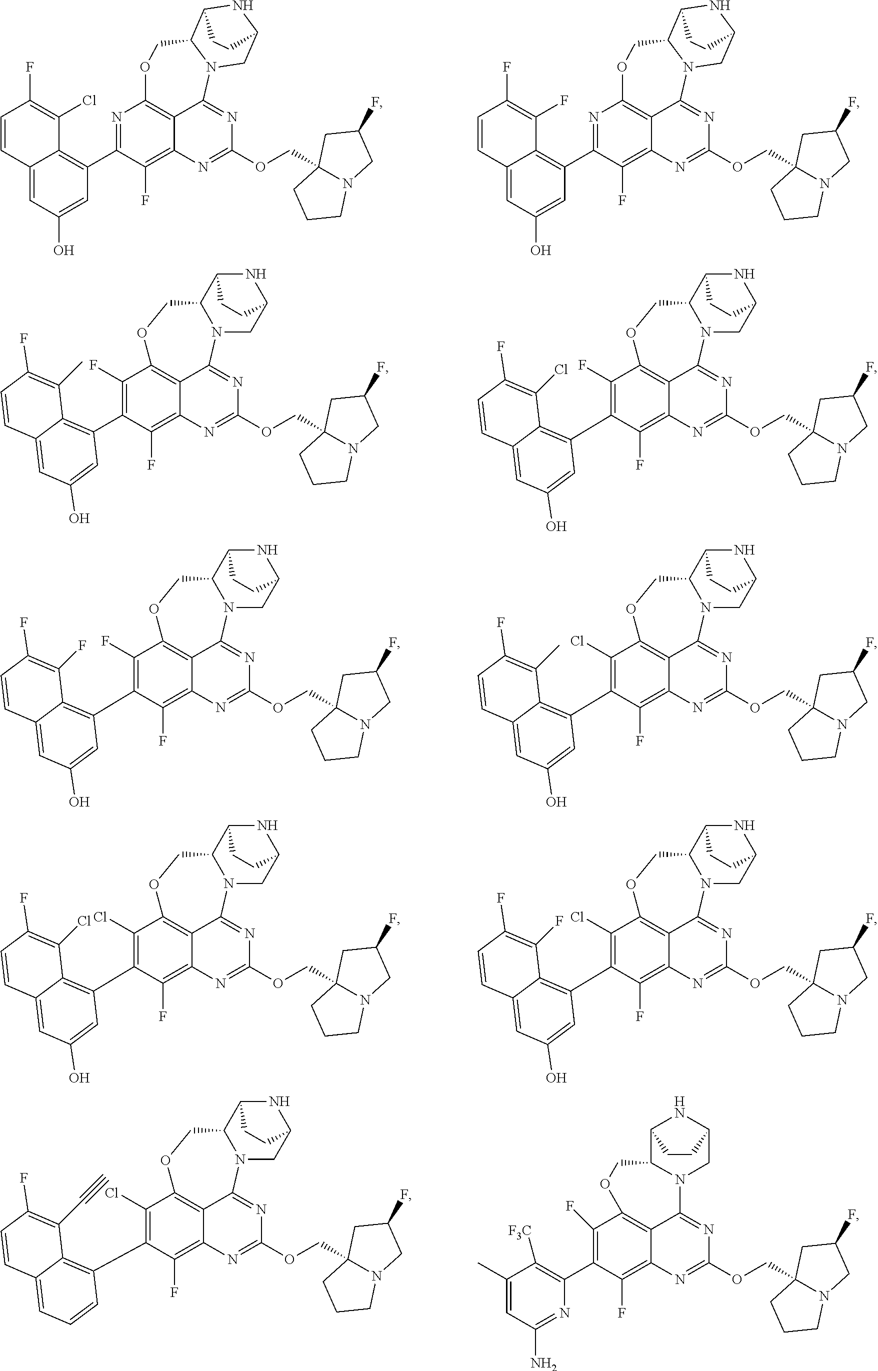

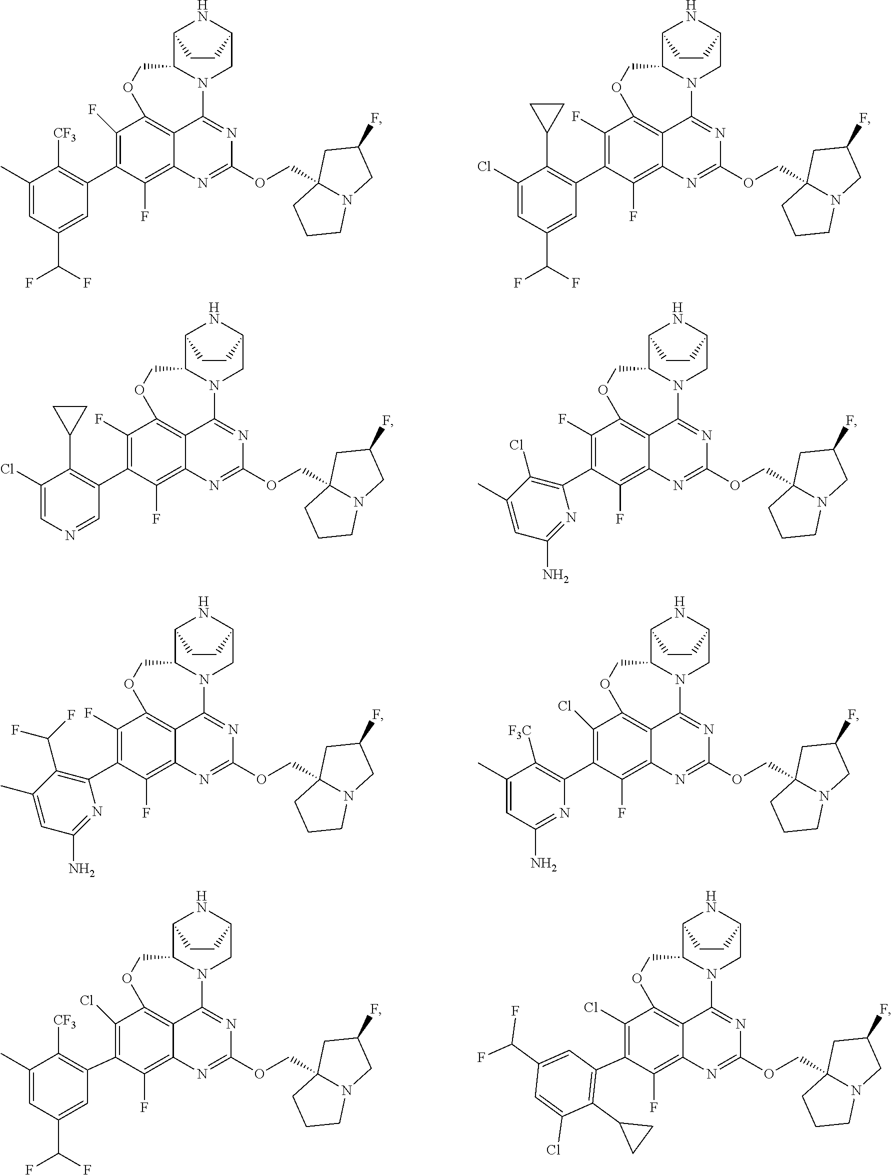

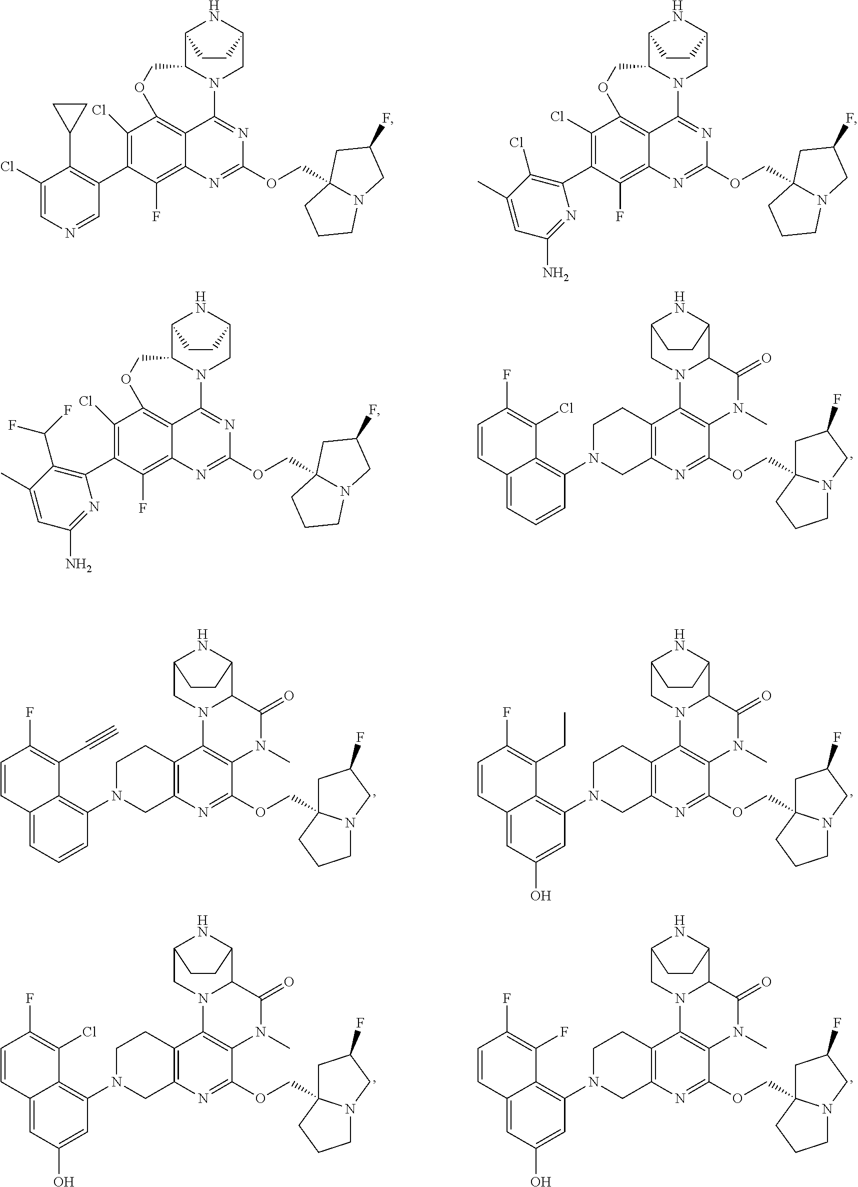

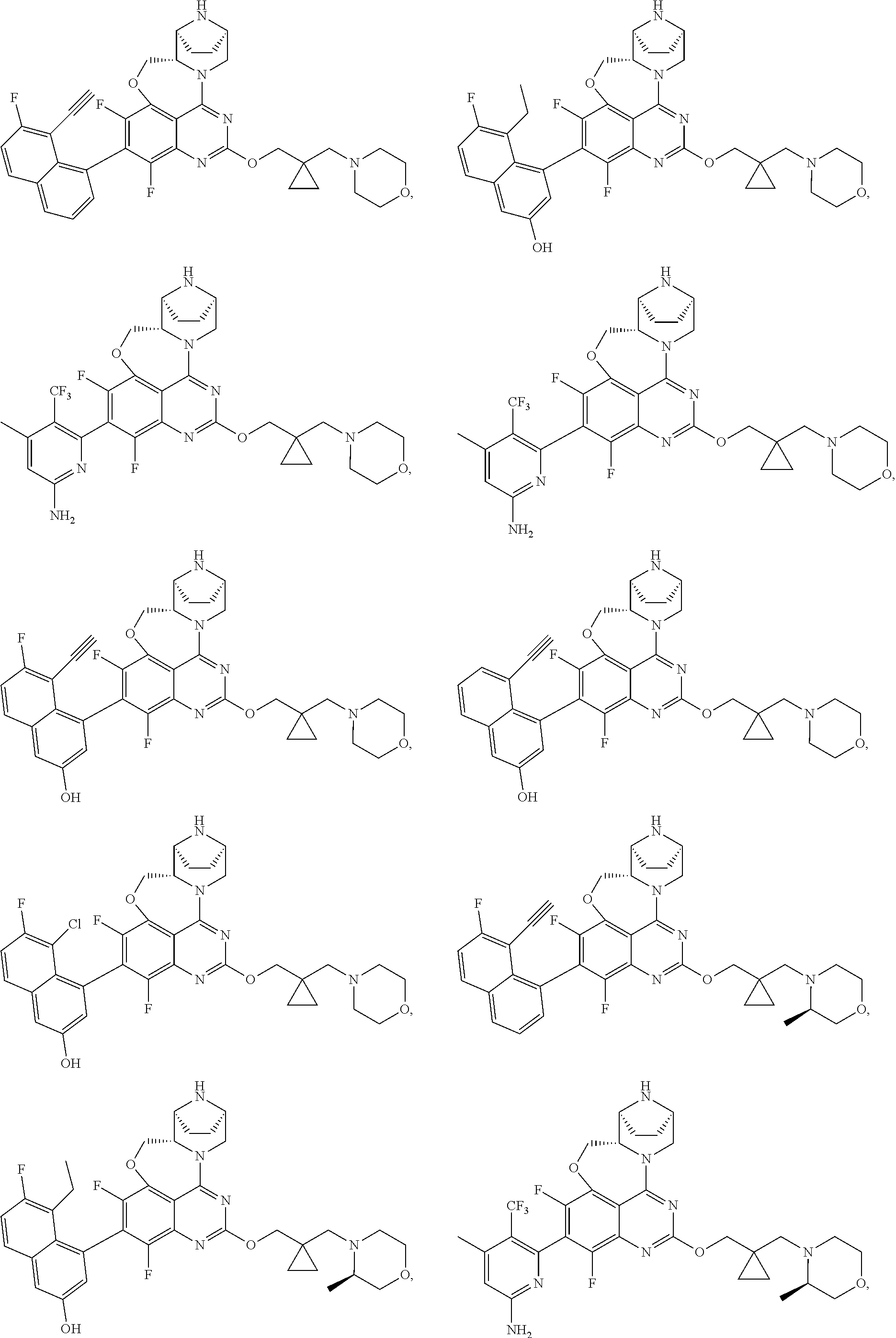

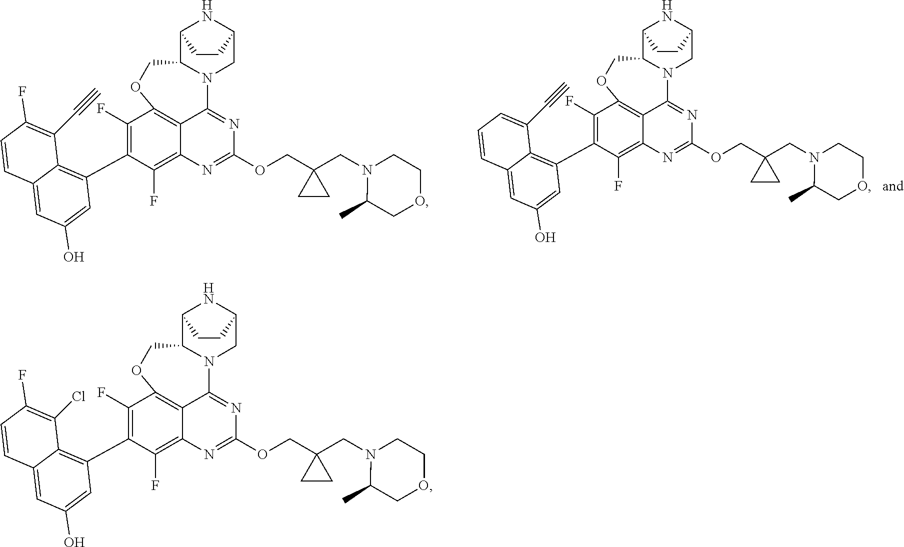

or a pharmaceutically acceptable salt thereof.

Compounds provided herein are described with reference to both generic formulae and specific compounds. In addition, the compounds of the present disclosure may exist in a number of different forms or derivatives, including but not limited to prodrugs, soft drugs, active metabolic derivatives (active metabolites), and their pharmaceutically acceptable salts, all within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

Those of skill in the art will appreciate that compounds of the present disclosure may exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

### SYNTHESIS OF COMPOUNDS

The compounds provided herein can be prepared using any known organic synthesis techniques and can be synthesized according to any of numerous possible synthetic routes

Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G.M. Wuts, Greene's Protective Groups in Organic Synthesis, 5th Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6 (6), 874-883, which is incorporated herein by reference in its entirety), and normal phase silica chromatography.

### USE OF COMPOUNDS

In an aspect, the present disclosure provides compounds capable of inhibiting KRAS protein, in particular KRAS G12D protein.

As used herein, the term “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Therapy” can also mean prolonging survival as compared to expected survival if not receiving it. Those in need of therapy include those already with the condition or disorder as well as those prone to have the condition or disorder or those in which the condition or disorder is to be prevented. The term “therapy” also encompasses prophylaxis unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In a further aspect, the present disclosure provides use of the compound of the present disclosure or a pharmaceutically acceptable salt thereof or the pharmaceutical composition of the present disclosure for use in therapy, for example, for use in therapy associated with KRAS protein, in particular, in therapy associated with KRAS G12D protein.

In a further aspect, the present disclosure provides use of the compound of the present disclosure or a pharmaceutically acceptable salt thereof or the pharmaceutical composition of the present disclosure, in the manufacture of a medicament for treating cancer.

In some embodiments, the cancer is mediated by KRAS protein. In some embodiments, the cancer is mediated by KRAS G12D protein.

### PHARMACEUTICAL COMPOSITIONS

In a further aspect, there is provided pharmaceutical compositions comprising one or more compounds of the present disclosure, or a pharmaceutically acceptable salt thereof.

In another aspect, there is provided pharmaceutical composition comprising one or more compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

In some embodiments, the second active ingredient has complementary activities to the compound provided herein such that they do not adversely affect each other. Such ingredients are suitably present in combination in amounts that are effective for the purpose intended.

### Method of Treatment of Disease

In a further aspect, the present disclosure provides a method for treating cancer, comprising administering an effective amount of the compound or a pharmaceutically acceptable salt thereof or the pharmaceutical composition provided herein to a subject in need thereof.

In some embodiments, the compounds or pharmaceutically acceptable salts thereof and the compositions provided herein may be used for the treatment of a KRAS G12D-associated cancer in a subject in need thereof, comprising administering to said subject a therapeutically effective amount of a compound provided herein, a pharmaceutically acceptable salt thereof, or a pharmaceutical composition comprising the compound or pharmaceutically acceptable salt thereof.

In some embodiments, the compounds or pharmaceutically acceptable salts thereof and the compositions provided herein may be used for the treatment of a wide variety of cancers including tumors such as lung, prostate, breast, brain, skin, cervical carcinomas, testicular carcinomas, etc. More particularly, cancers that may be treated by the compounds or pharmaceutically acceptable salts thereof and the compositions provided herein include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, the compounds or pharmaceutically acceptable salts thereof and the compositions provided herein can be used to treat:
(i)Cardiac: sarcoma (angiosarcoma, fibrosarcoma, rhabdomyosarcoma, liposarcoma), myxoma, rhabdomyoma, fibroma, lipoma and teratoma;
(ii) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
(iii) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
(iv) Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
(v) Liver: hepatoma (hepatocellular carcinoma), cholangiocarcinoma, hepatoblastoma, angiosarcoma, hepatocellular adenoma, hemangioma;
(vi) Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
(vii) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);
(viii) Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
(ix) Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
(x) Skin: malignant melanoma, basal cell carcinoma, squamous cell carcinoma, Kaposi's sarcoma, moles dysplastic nevi, lipoma, angioma, dermatofibroma, keloids, psoriasis; and
(xi) Adrenal glands: neuroblastoma.

In certain embodiments, the cancer that can be treated with the compounds or pharmaceutically acceptable salts thereof and the compositions provided herein is non-small cell lung cancer, small cell lung cancer, colorectal cancer, rectal cancer or pancreatic cancer.

The concentration and route of administration to the subject will vary depending on the cancer to be treated. In certain embodiments, the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

In some embodiments, the compounds, pharmaceutically acceptable salts thereof and pharmaceutical compositions comprising such compounds and salts can be administered simultaneously, separately or sequentially with one or more additional therapeutic agents. In certain embodiments, the additional therapeutic agent is selected from an anti-PD-1 antagonist, an MEK inhibitor, a SHP2 inhibitor, a platinum agent or pemetrexed. In certain embodiments, the anti-PD-1 antagonist is selected from nivolumab, pembrolizumab, or AMB 404. In certain embodiments, the MEK inhibitor is trametinib. In certain embodiments, the SHP2 inhibitor is RMC-4630.

In another aspect, the present disclosure also provides a method for treating cancer in a subject in need thereof, the method comprising:
(a) acquiring the knowledge that the cancer is associated with KRAS G12D mutation; and
(b) administering to the subject an effective amount of a compound or a pharmaceutically acceptable salt thereof or the pharmaceutical composition of the present disclosure.

In another aspect, the present disclosure provides a method for inhibiting KRAS G12D activity in a subject in need thereof, comprising administering the compound or a pharmaceutically acceptable salt thereof or the pharmaceutical composition of the present disclosure to the subject.

### EXAMPLES

For the purpose of illustration, the following examples are included. However, it is to be understood that these examples do not limit the present disclosure and are only meant to suggest a method of practicing the present disclosure.

### General Synthetic Route

In some embodiments, compounds of Formula (Ib) provided herein may be prepared by the synthetic route as shown in Scheme 1:

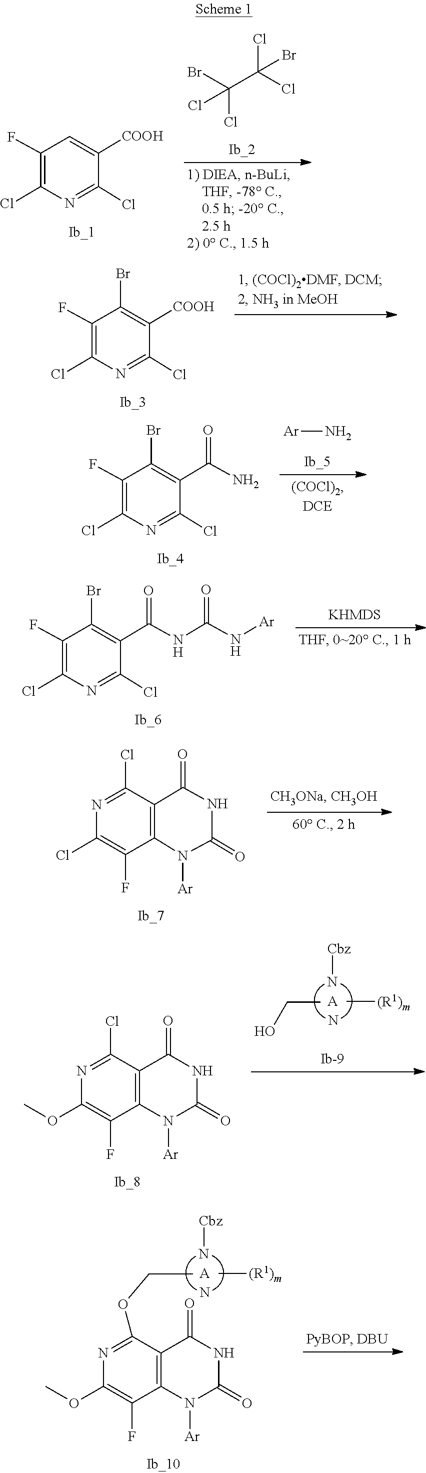

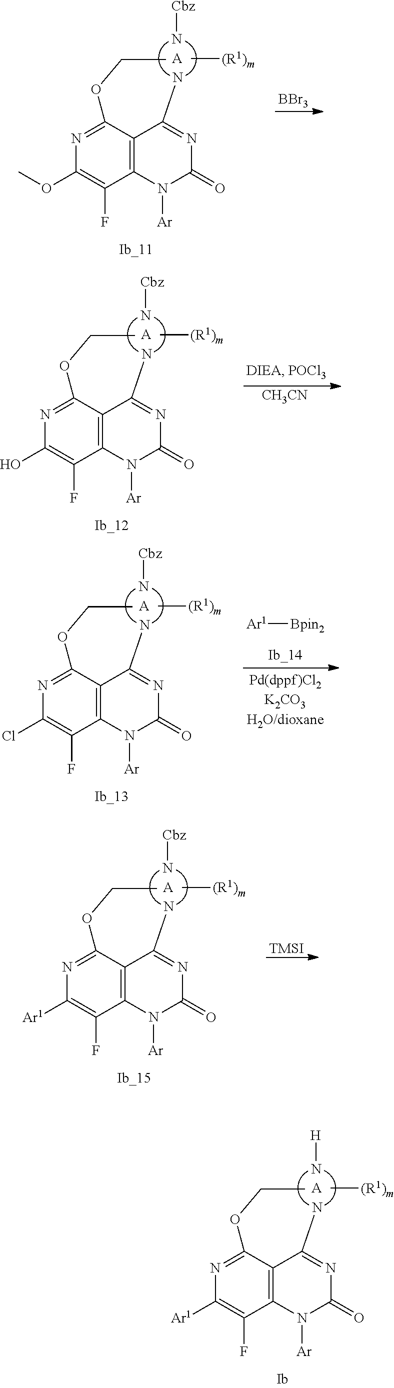

### Step 1:

The starting material of Formula (Ib_1) is commercially available. Compound of Formular (Ib_3) may be prepared by bromination reaction of a compound of Formula (IIb_1) with a compound of Formula (Ib_2) in the presence of organolithium reagent (e.g, n-BuLi) under standard conditions.

### Step 2:

Compound of Formula (Ib_4) may be prepared by the amidation reaction of a compound of Formula (Ib_3) with ammonia under standard conditions.

### Step 3:

Compound of Formula (Ib_6) may be prepared by arene formylation of a compound of Formula (Ib_4) with a compound of Formula (Ib_5) in the presence of oxalyl chloride under standard conditions.

### Step 4:

Compound of Formula (Ib_7) may be prepared by the intramolecular cyclization reaction of a compound of Formula (Ib_6) with base (e.g, KHMDS) under standard conditions.

### Step 5:

Compound of Formula (Ia_8) may be prepared by the methylation of a compound of Formula (Ia_7) with methylation reagent (e.g, MeONa).

### Step 6:

Compound of Formula (Ib_10) may be prepared by substitution reaction of a compound of Formula (Ib_8) and a compound of Formula (Ib_9) in the presence of base (e.g, DIPEA) under standard conditions.

### Step 7:

Compound of Formula (Ib_11) may be prepared by intramolecular coupling reaction of a compound of Formula (Ib_10) in the presence of phosphonium salts (e.g, PyBOP) and base (e.g, DBU) under standard conditions.

### Step 8:

Compound of Formula (Ib_12) may be prepared by demethylation reaction of a compound of Formula (Ib_11) with dealkylating reagent (e.g, BBr₃) under standard conditions.

### Step 9:

Compound of Formula (Ib_13) may be prepared the chlorination reaction of a compound of Formula (Ib_12) with chloride reagents (e.g, POCl₃) in the presence of base (e.g, DIPEA) under standard conditions.

### Step 10:

Compound of Formula (Ib_15) may be prepared by the Suzuki coupling reaction of a compound of Formular (Ib_13) with a compound of Formula (Ib_16) in the presence of Palladium catalyst (e.g, PddppfCl₂) and base (e.g, Na₂CO₃) under standard conditions.

### Step 11:

Compound of Formula (Ib) may be prepared by the removing the Cbz protective group of a compound of Formula (Ib_15) with TMSI under standard condition.

In some embodiments, compounds of Formula (IIb) provided herein may be prepared by the synthetic route as shown in Scheme 2:

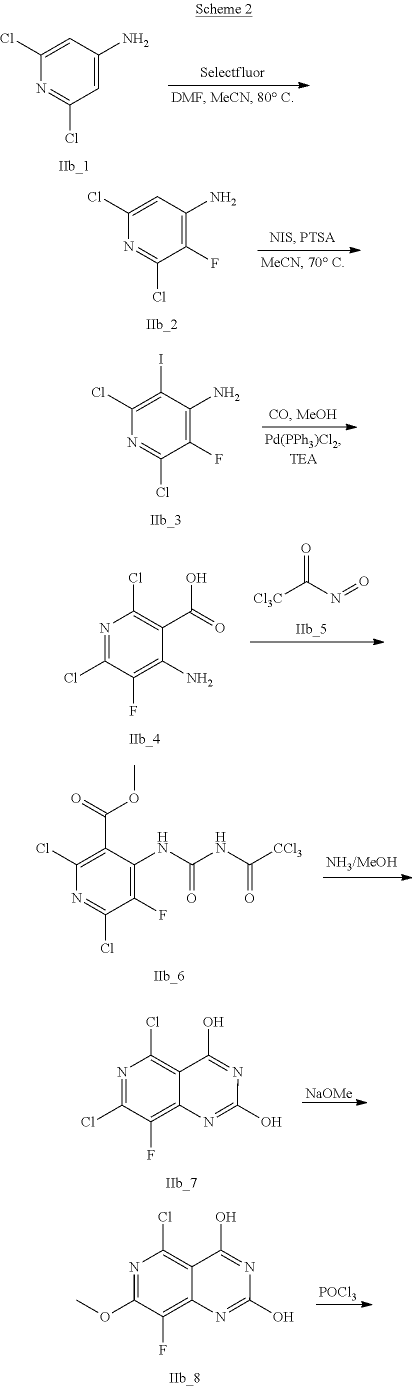

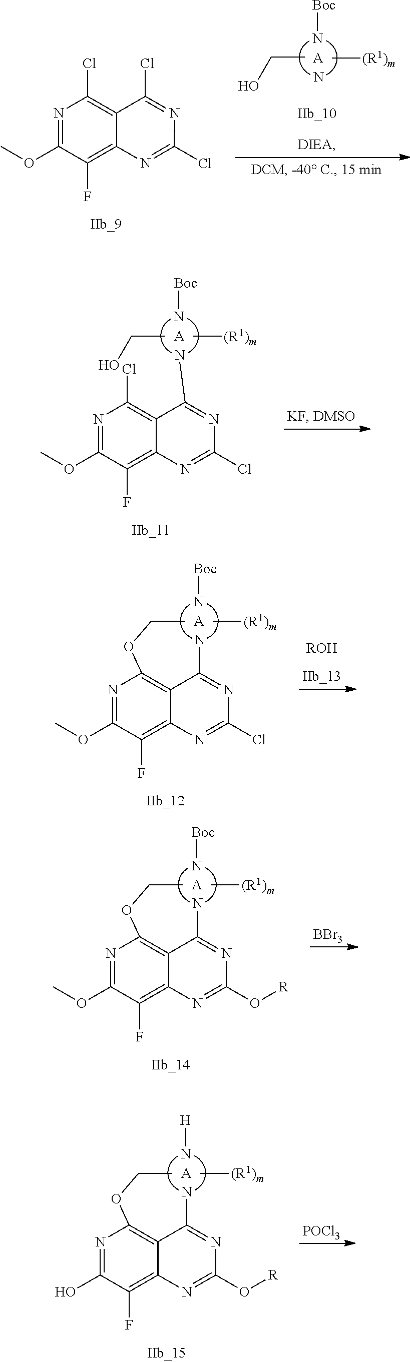

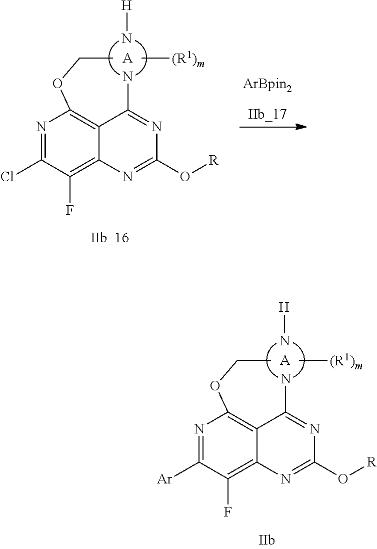

### Step 1:

The starting material of Formula (IIb_1) is commercially available. Compound of Formular (IIb_2) may be prepared by electrophilic fluorination of a compound of Formula (IIb_1) with fluorine donor (e.g, Selectfluor) under standard conditions.

### Step 2:

Compound of Formula (IIb_3) may be prepared by the iodization of a compound of Formula (IIb_2) with N-iodosuccinimide under standard condition.

### Step 3:

Compound of Formula (IIb_4) may be prepared by the carbonylation reaction of a compound of Formula (IIIb_3) with carbon monoxide in the presence of Palladium catalyst (e.g, Pd(Ph₃P)₄) and base (e.g, triethylamine) under standard conditions.

### Step 4:

Compound of Formula (IIb_6) may be prepared by the trichloroacetyl isocyanate reaction of a compound of Formula (IIb_4) with a compound of Formula (IIb_5) under standard conditions.

### Step 5:

Compound of Formula (IIa_7) may be prepared by the pyrimidinedione cyclization reaction of a compound of Formula (IIa_6) with ammonia under standard conditions.

### Step 6:

Compound of Formula (IIa_8) may be prepared by the methylation of a compound of Formula (IIa_7) with methylation reagent (e.g, MeONa).

### Step 7:

Compound of Formula (IIb_9) may be prepared by the chlorination reaction of a compound of Formula (IIb_8) with chloride reagents (e.g, POCl₃) in the presence of base (e.g, DIPEA) under standard conditions.

### Step 8:

Compound of Formula (IIb_11) may be prepared by substitution reaction with a compound of Formula (IIb_9) and a compound of Formula (IIb_10) in the presence of base (e.g, DIPEA) under standard conditions.

### Step 9:

Compound of Formula (IIb_12) may be prepared by intramolecular cyclization reaction of a compound of Formula (IIb_11) with base (e.g, KF) under standard conditions.

### Step 10:

Compound of Formula (IIb_14) may be prepared by nucleophilic substitution reaction of a compound of Formula (IIb_12) with a compound of Formula (IIb_13) in the presence of base (e.g, DIPEA) under standard conditions.

### Step 11:

Compound of Formula (IIb_15) may be prepared by demethylation reaction of a compound of Formula (IIb_14) with dealkylating reagent (e.g, BBr₃) under standard conditions.

### Step 12:

Compound of Formula (IIb_16) may be prepared by the chlorination reaction of a compound of Formula (IIb_15) with chloride reagents (e.g, POCl₃) in the presence of base (e.g, DIPEA) under standard conditions.

### Step 13:

Compound of Formula (IIb) may be prepared by the Suzuki coupling reaction of a compound of Formular (IIb_16) with a compound of Formula (IIb_17) in the presence of Palladium catalyst (e.g, PddppfCl₂) and base (e.g, Na₂CO₃) under standard conditions.

In some embodiments, compounds of Formula (IIIb) provided herein may be prepared by the synthetic route as shown in Scheme 3:

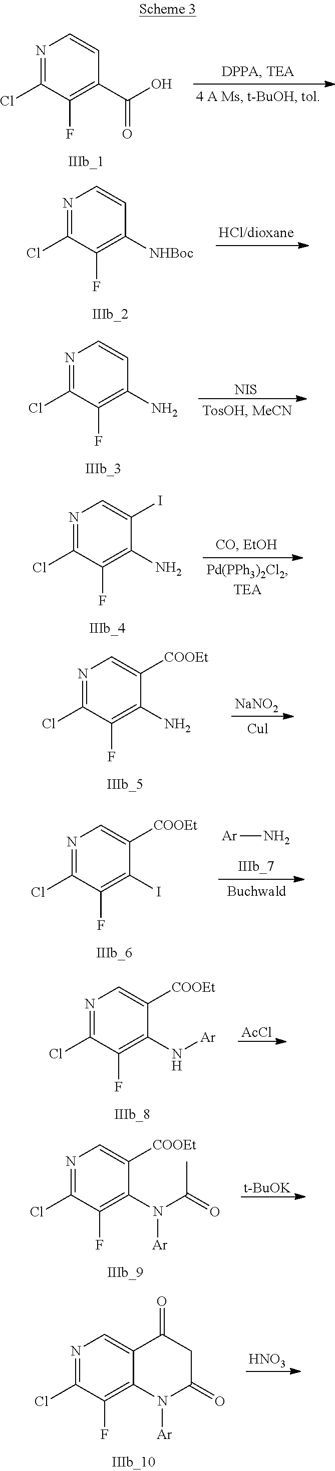

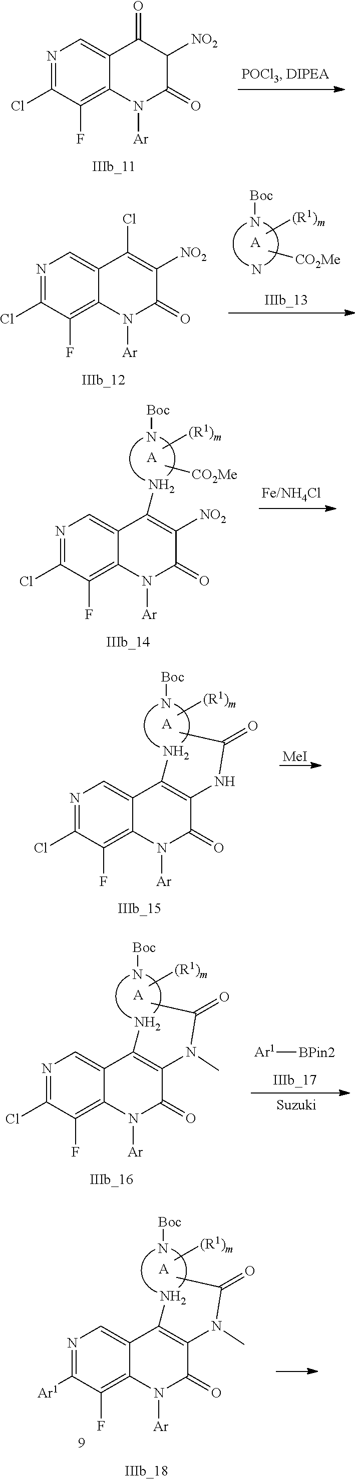

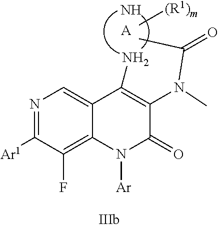

### Step 1:

The starting material of Formula (IIIb_1) is commercially available. Compound of Formular (IIIb_2) may be prepared by the Curtius rearrangement reaction with a compound of Formula (IIIb_1) in the presence of diphenyl phosphorazidate (DPPA) under standard conditions.

### Step 2:

Compound of Formula (IIIb_3) may be prepared by the removing the Boc protective group with acid (e.g, TFA) under standard condition.

### Step 3:

Compound of Formula (IIIb_4) may be prepared by the iodination reaction of a compound of Formula (IIIb_3) with N-Iodosuccinimide (NIS) under standard conditions.

### Step 4:

Compound of Formula (IIIb_5) may be prepared by the carbonylation reaction of a compound of Formula (IIIb_4) with carbon monoxide in the presence of Palladium catalyst (e.g, Pd(Ph₃P)₄) and base (e.g, triethylamine) under standard conditions.

### Step 5:

Compound of Formula (IIIb_6) may be prepared by the diazotization reaction of a compound of Formula (IIIb_5) with diazotization reagent (e.g, NaNO₂) and Iodination reagent (e.g, CuI) under standard conditions.

### Step 6:

Compound of Formula (IIIb_8) may be prepared by the Buchwald reaction of a compound of Formula (IIIb_6) and a compound of Formula (IIIb_7) in the presence of Palladium catalyst (e.g, Pd(OAc)₂), ligand (e.g, BINAP) and base (e.g, Cs₂CO₃) under standard conditions.

### Step 7:

Compound of Formula (IIIb_9) may be prepared by the acylation of a compound of Formula (IIIb_8) with acetylchloride under standard conditions.

### Step 8:

Compound of Formula (IIIb_10) may be prepared by the intramolecular cyclization of a compound of Formula (IIIb_9) in the presence of base (e.g, t-BuOK) under standard conditions.

### Step 9:

Compound of Formula (IIIb_11) may be prepared by the nitration reaction of a compound of Formula (IIIb_10) with nitric acid under standard conditions.

### Step 10:

Compound of Formula (IIIb_12) may be prepared by the chlorination reaction of a compound of Formula (III_11) with chloride reagents (e.g, POCl₃) in the presence of base (e.g, DIPEA) under standard conditions.

### Step 11:

Compound of Formula (IIIb_14) may be prepared by substitution reaction with a compound of Formula (III_12) and a compound of Formula (IIIb_13) in the presence of base (e.g, DIPEA, NaHCO₃) under standard conditions.

### Step 12:

Compound of Formula (IIIb_15) may be prepared by the reduction of a compound of Formula (IIIb_14) with standard reduction conditions (e.g, Fe/NH₄Cl).

### Step 13:

Compound of Formula (IIIb_16) may be prepared by the methylation reaction of a compound of Formula (IIIb_15) with methylation reagents (e.g, Mel) under standard conditions.

### Step 14:

Compound of Formula (IIIb_18) may be prepared by the Suzuki coupling reaction of a compound of Formular (IIIb_16) with a compound of Formula (IIIb_17) in the presence of Palladium catalyst (e.g, PddppfCl₂) and base (e.g, Na₂CO₃) under standard conditions.

### Step 15:

Compound of Formula (IIIb) may be prepared by the removing the Boc protective group with acid (e.g, TFA) under standard condition.

In some embodiments, compounds of Formula (IVa) provided herein may be prepared by the synthetic route as shown in Scheme 4:

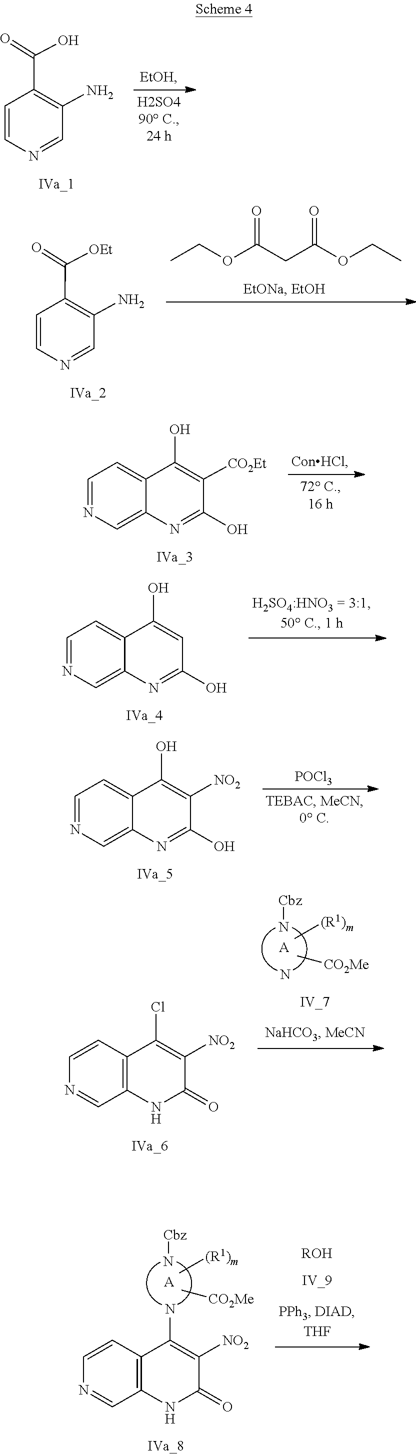

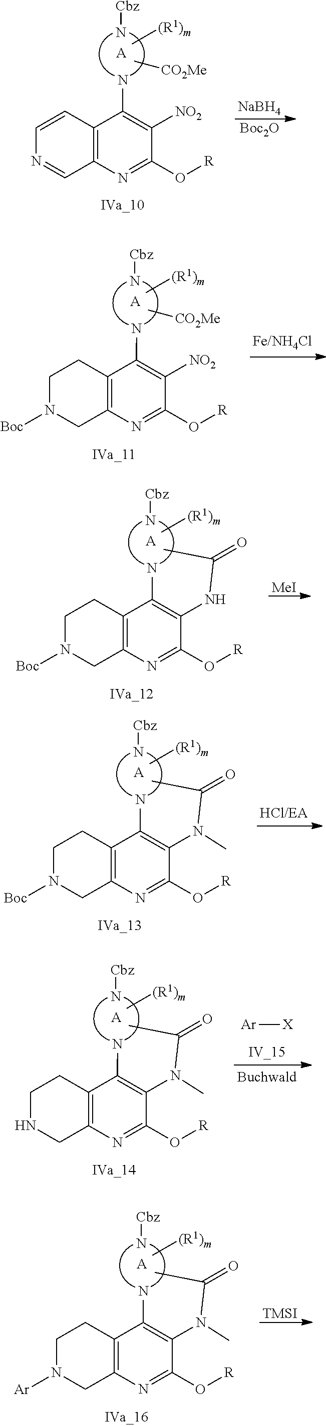

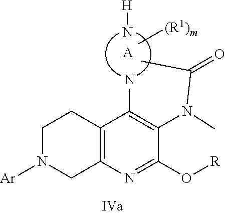

### Step 1:

The starting material of Formula (IVa_1) is commercially available. Compound of Formula (IVa_2) may be prepared by the esterification of a compound of Formula (IVa_1) with ethanol in the presence of acid (e.g, H₂SO₄) under standard condition.

### Step 2:

Compound of Formula (IVa_3) may be prepared by the pyridone cyclization reaction of a compound of Formula (IVa_2) with diethyl malonate in the presence of base (e.g, EtONa) under standard conditions.

### Step 3:

Compound of Formula (IVa_4) may be prepared by the decarboxylation reaction of a compound of Formula (IVa_3) with concentrated HCl under standard conditions.

### Step 4:

Compound of Formula (IVa_5) may be prepared by the nitration reaction of a compound of Formula (IVa_4) with nitric acid under standard conditions.

### Step 5:

Compound of Formula (IVa_6) may be prepared by the chlorination reaction of a compound of Formula (IVa_5) with chloride reagents (e.g, POCl₃) in the presence of base (e.g, TEBAC) under standard conditions.

### Step 6:

Compound of Formula (IVa_8) may be prepared by substitution reaction with a compound of Formula (IVa_6) and a compound of Formula (IVa_7) in the presence of base (e.g, DIPEA, NaHCO₃) under standard conditions.

### Step 7:

Compound of Formula (IVa_10) may be prepared by the Mitsunobu reaction with a compound of Formula (IVa_8) and a compound of Formula (IVa_9) in the presence of triphenylphosphine and azodicarboxylate (e.g, DIAD) under standard conditions.

### Step 8:

Compound of Formula (IVa_11) may be prepared by the reduction reaction with a compound of Formula (IVa_10) with reduction reagent (e.g, NaBH₄) under standard conditions.

### Step 9:

Compound of Formula (IVa_12) may be prepared by the reduction of a compound of Formula (IVa_11) with standard reduction conditions (e.g, Fe/NH₄Cl).

### Step 10:

Compound of Formula (IVa_13) may be prepared by the methylation reaction of a compound of Formula (IVa_12) with methylation reagents (e.g, Mel) under standard conditions.

### Step 11:

Compound of Formula (IVa_14) may be prepared by the removing the Boc protective group of a compound of Formula (IVa_13) with acid (e.g, HCl) under standard condition.

### Step 12:

Compound of Formula (IVa_16) may be prepared by the Buchwald reaction of a compound of Formula (IVa_14) and a compound of Formula (IVa_15) in the presence of Palladium catalyst (e.g, Pd(OAc)₂), ligand (e.g, BINAP) and base (e.g, Cs₂CO₃) under standard conditions.

### Step 13:

Compound of Formula (IVa) may be prepared by the removing the Cbz protective group of a compound of Formula (IVa_16) with TMSI under standard condition.

In some embodiments, an intermediate INT1 useful in the present disclosure may be prepared by the synthetic route as shown in Scheme 5:

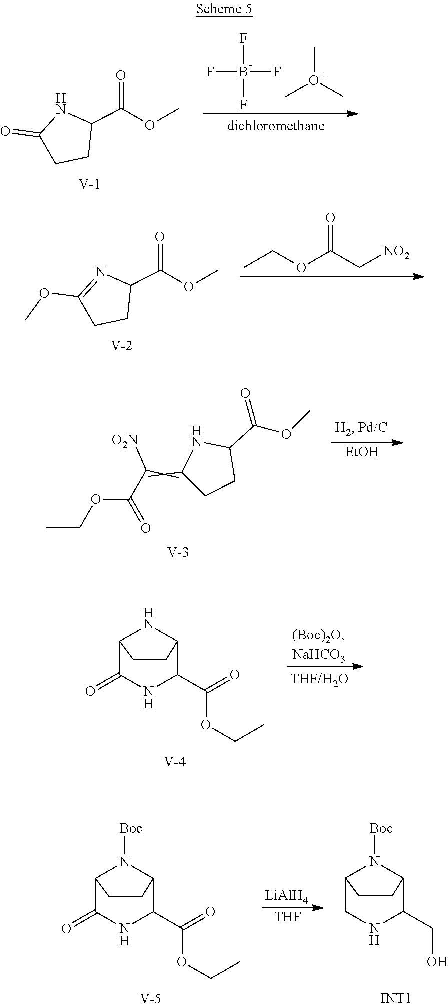

### Step 1:

The starting material racemic mixture (V-1) is commercially available. Compound of Formula (V-2) may be prepared by the methylation of a compound of Formula (V-1) in the presence of trimethyloxonium tetrafluoroborate under standard condition.

### Step 2:

Formula (V-3) as a E/Z mixture could be prepared via condensation reaction between V-2 and ethyl 2-nitroacetate under standard condition.

### Step 3:

Formula (V-4) as a mixture of 4 diastereomers can be prepared via Pd/C catalyzed hydrogenation/condensation reaction under standard condition. A pair of two minor diastereomers was removed via column chromatography and the two major diastereomers are carried over for the next step.

### Step 4:

Formula (V-5) as a 1:1 diastereomer mixture can be prepared via Boc protection of Formula (V-4) under standard condition.

### Step 5:

INT1 as a 1:1 diastereomer mixture can be prepared via reduction of Formula (V-5) with a reducing agent (e.g, LiAlH₄) under standard condition.

In some embodiments, compounds of Formula (IIb) provided herein may be prepared by the synthetic route as shown in Scheme 6:

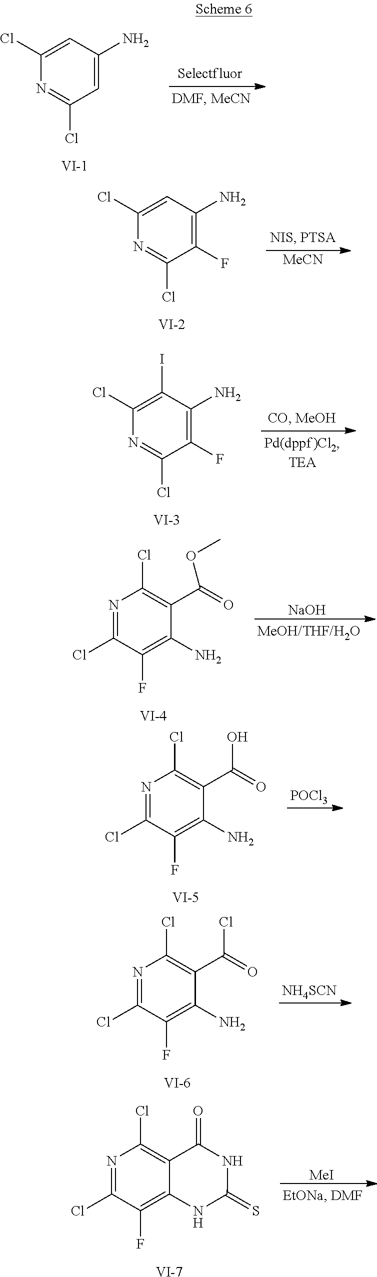

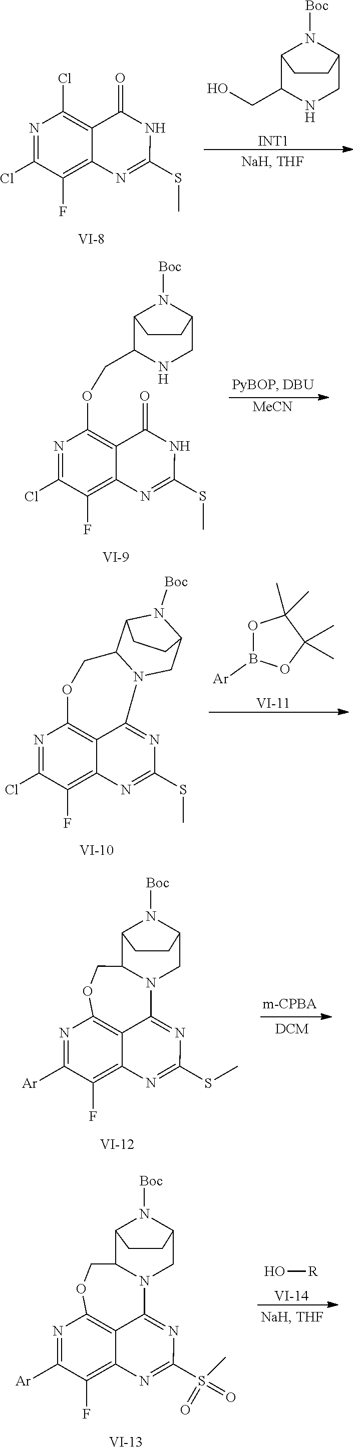

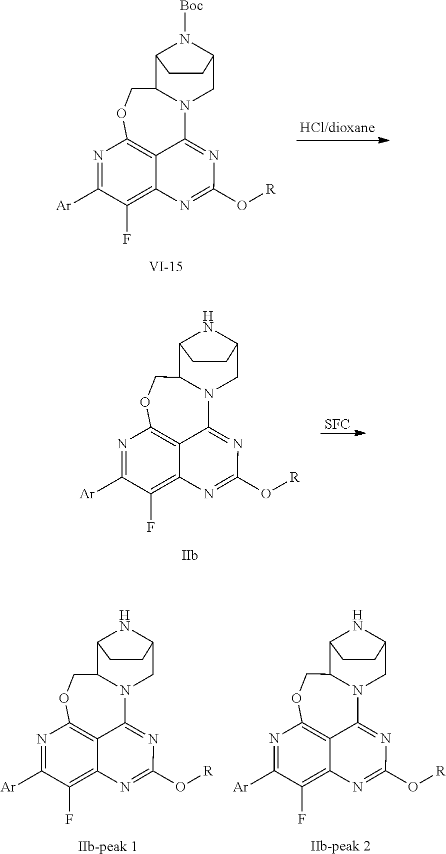

### Step 1:

Formula (VI-2) can be prepared via fluorination of commercially available Formula (VI-1) with a fluorinating reagent (e.g, Selectfluor) under standard condition.

### Step 2:

Formula (VI-3) can be prepared via iodination of Formula (VI-2) with an iodinating reagent (e.g, NIS) under standard condition.

### Step 3:

Formula (VI-4) can be prepared via palladium-catalyzed alkoxycarbonylation of aryl iodide Formula (VI-3) with carbon monoxide, a base (e.g, TEA) and a palladium catalyst (e.g, Pd(dppf)Cl₂) in methanol under standard conditions.

### Step 4:

Formula (VI-5) can be prepared via hydrolysis reaction of methyl ester Formula (VI-4) under standard conditions.

### Step 5:

Formula (VI-6) can be prepared via chlorination of carboxylic acid Formula (VI-5) using a chlorinating reagent (e.g, POCl₃) under standard conditions.

### Step 6:

Formula (VI-7) can be prepared via condensation cyclization between ammonium thiocyanate and Formula (VI-6) under standard conditions.

### Step 7:

Formula (VI-8) can be prepared via methylation of Formula (VI-7) with methyl iodide under standard conditions.

### Step 8:

Formula (VI-9) as a 1:1 diastereomer mixture can be prepared via SNAr reaction between Formula (VI-8) and INT1 in presence of a base (e.g, NaH) under standard conditions.

### Step 9:

Formula (VI-10) as a 1:1 diastereomer mixture can be prepared via intramolecular cyclization of Formula (VI-9) in presence of a peptide coupling reagent (e.g, PyBOP) and a base (e.g, DBU) under standard conditions.

### Step 10:

Formula (VI-12) as a 1:1 diastereomer mixture can be prepared via Suzuki coupling reaction between an aryl boronic ester Formula (VI-11) and Formula (VI-10) in presence of a palladium catalyst (e.g, XPhos-Pd-G₂) and a base (e.g, K₂CO₃) under standard conditions.

### Step 11:

Formula (VI-13) as a 1:1 diastereomer mixture can be prepared via sulfur oxidation of Formula (VI-12) with an oxidant (e.g, mCPBA) under standard conditions.

### Step 12:

Formula (VI-15) as a 1:1 diastereomer mixture can be prepared via SNAr reaction between Formula (VI-13) and an alcohol Formula (VI-14) in presence of a base (e.g, NaH) under standard conditions.

### Step 13:

Formula (IIb) as a 1:1 diastereomer mixture can be prepared by deprotection of Formula (VI-15) with an acid (e.g, HCl) under standard conditions.

### Step 14:

Formula (IIb-peak1) and Formula (Ilb-peak2) as single diastereomer can be prepared by SFC separation of diastereomer mixture (IIb) with a proper column under standard conditions.

In some embodiments, intermediate of Formula (VI-8) in Scheme 6 may also be prepared by the synthetic route as shown in Scheme 7:

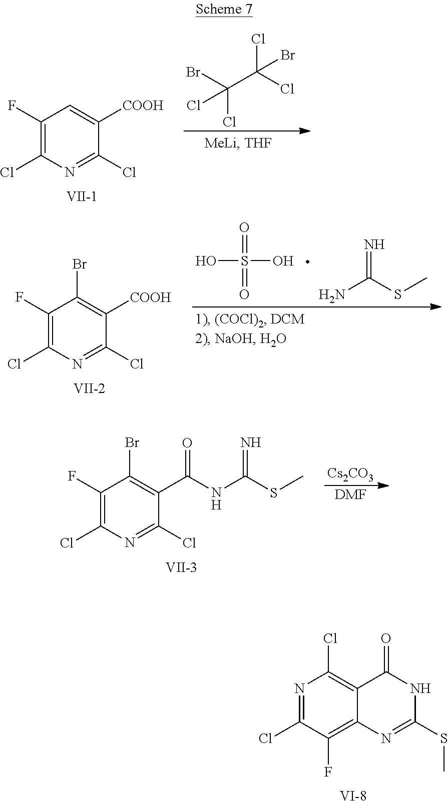

### Step 1:

Formula (VII-2) can be prepared via bromination of commercially available Formula (VI-1) with 1,2-dibromo-1,1,2,2-tetrachloroethane in presence of methyl lithium under standard condition.

### Step 2:

Formula (VII-3) can be prepared via condensation reaction between acid chloride of Formula (VII-2) and 2-methyl-2-thiopseudourea sulfate in presence of a base (e.g, NaOH) under standard condition.

Step 3: Formula (VI-8) can be prepared via intramolecular SNAr cyclization of Formula (VII-3) in presence of a base (e.g, Cs₂CO₃) under standard condition.

In some embodiments, intermediate INT2 provided herein may be prepared by the synthetic route as shown in Scheme 8:

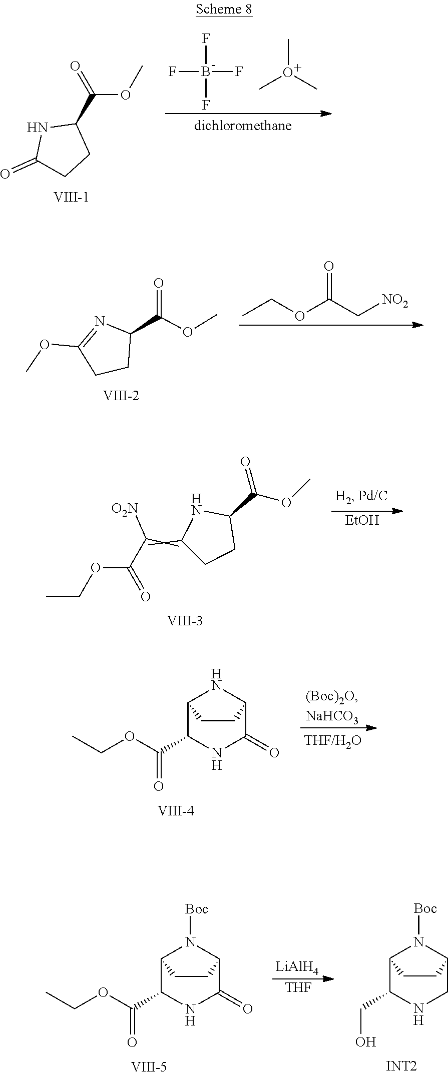

### Step 1:

The starting material methyl (R)-5-oxopyrrolidine-2-carboxylate (VIII-1) is commercially available. Compound of Formula (VIII-2) may be prepared by the methylation of a compound of Formula (VIII-1) in the presence of trimethyloxonium tetrafluoroborate under standard condition.

### Step 2:

Formula (VIII-3) as a E/Z mixture could be prepared via condensation reaction between VIII-2 and ethyl 2-nitroacetate under standard condition.

### Step 3:

Formula (VIII-4) as a mixture of two diastereomers can be prepared via Pd/C catalyzed hydrogenation/condensation reaction under standard condition. The minor diastereomer is removed via column chromatography and the major diastereomer is carried over for the next step.

### Step 4:

Formula (VIII-5) as can be prepared via Boc protection of Formula (VIII-4) under standard condition.

### Step 5:

INT2 as a single diastereomer can be prepared via reduction of Formula (VIII-5) with a reducing agent (e.g, LiAlH₄) under standard condition.

In some embodiments, compounds of Formula (IX-7) provided herein may be prepared by the synthetic route as shown in Scheme 9:

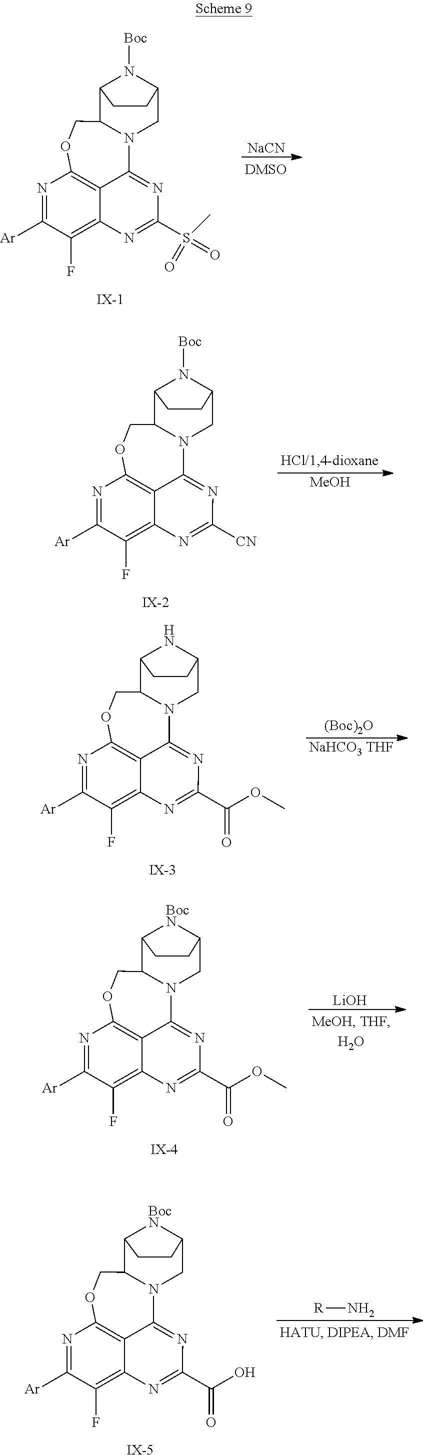

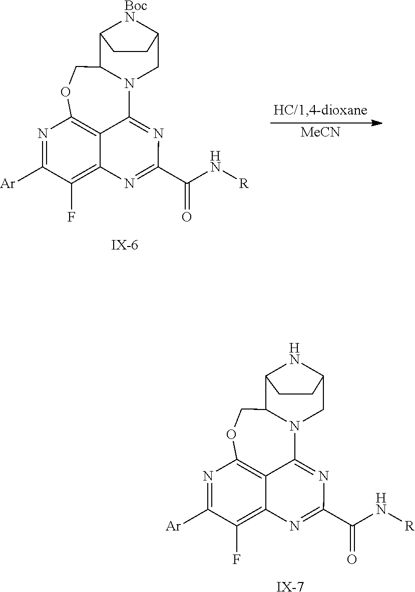

### Step 1:

Compound of Formula (IX-2) may be prepared by the substitution of sulfone group on Formula (IX-1) with sodium cyanate under standard condition.

### Step 2:

Compound of Formula (IX-3) may be prepared by hydrolysis of cyanide of Formula (IX-2) to acid and in situ formation of methyl ester in presence of methanol under acidic condition.

### Step 3:

Compound of Formula (IX-4) may be prepared by Boc protection of Formula (IX-3) under standard condition.

### Step 4:

Compound of Formula (IX-5) may be prepared by hydrolysis of methyl ester of Formula (IX-4) under basic condition.

### Step 5:

Compound of Formula (IX-6) may be prepared by HATU coupling between Formula (IX-5) and an appropriate primary amine under standard condition.

### Step 6:

Compound of Formula (IX-7) may be prepared by Boc deprotection of Formula (IX-6) under acidic condition.

In some embodiments, compounds of Formula (X-3) provided herein may be prepared by the synthetic route as shown in Scheme 10:

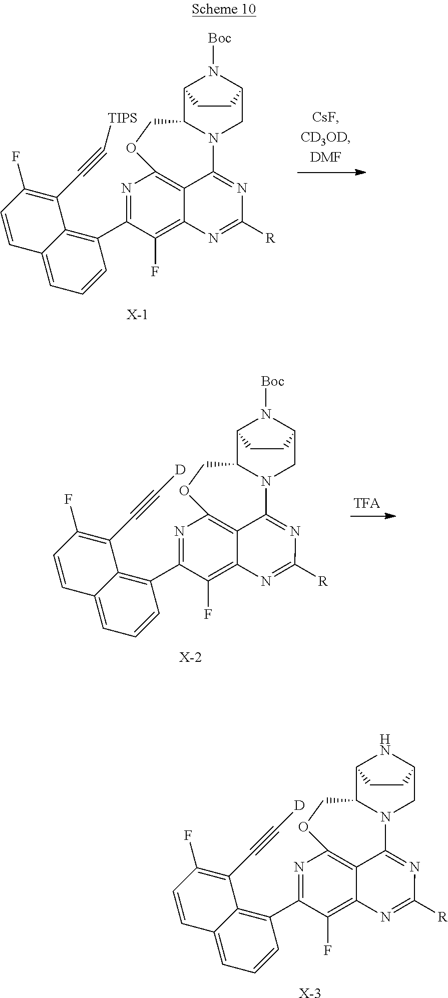

### Step 1:

Compound of Formula (X-2) may be prepared by the treatment of Formula (X-1) with CSF in presence of CD₃OD in DMF.

### Step 2:

Compound of Formula (X-3) may be prepared by Boc deprotection of Formula (X-2) under acidic condition.

In some embodiments, compounds of Formula (II′a) provided herein may be prepared by the synthetic route as shown in Scheme 11:

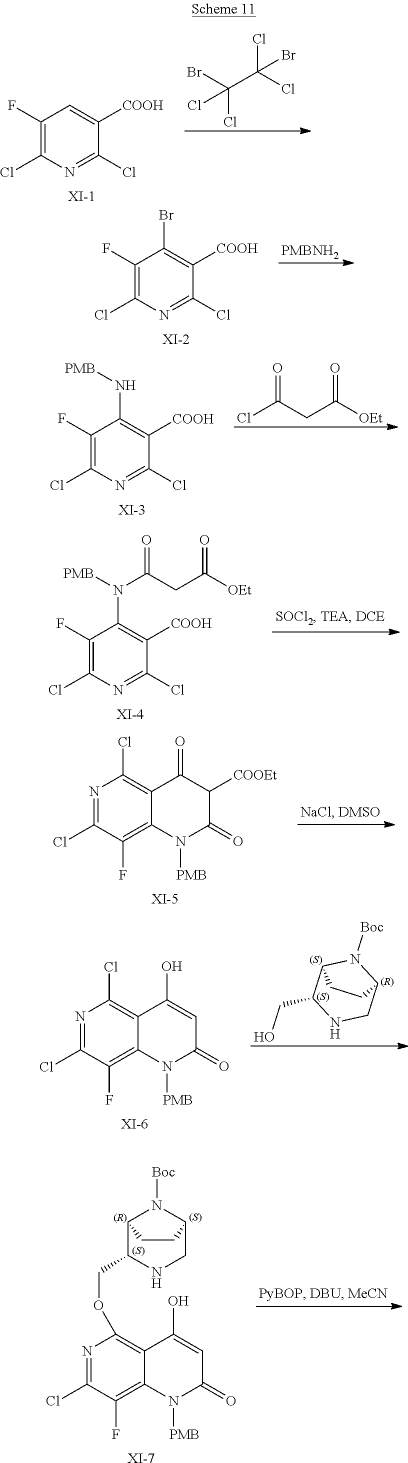

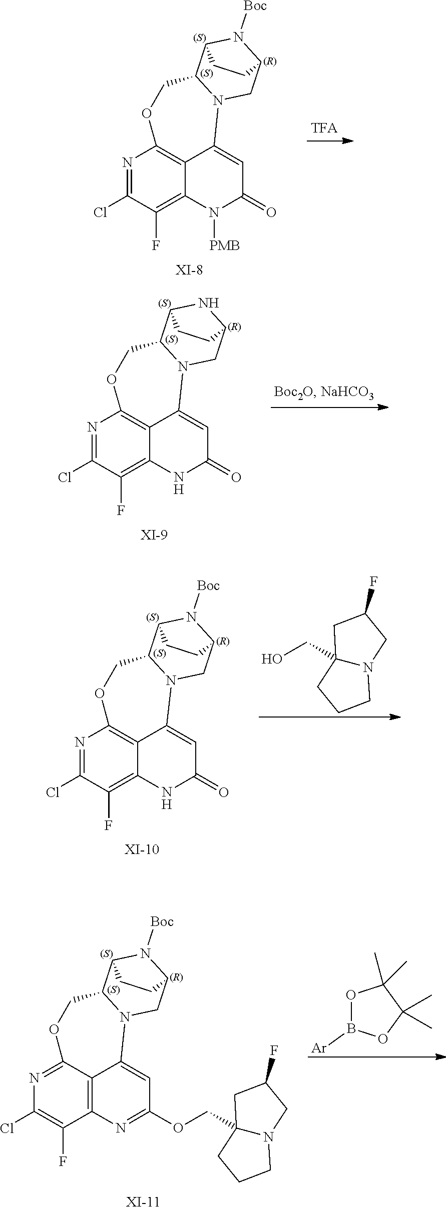

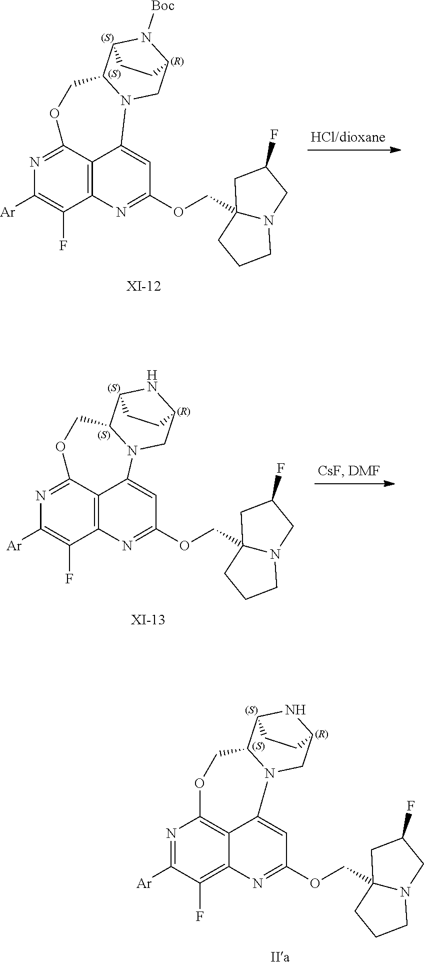

### Step 1:

Compound of Formula (XI-2) may be prepared by the treatment of Formula (XI-1) with bromination reagent such as 1,2-Dibromotetrachloroethane under standard condition.

### Step 2:

Formula (XI-3) may be prepared by substitution of bromine of Formula (XI-2) with PMBNH₂ under standard condition.

### Step 3:

Formula (XI-4) may be prepared by amide coupling between acid chloride and amine of Formula (XI-3) under standard condition.

### Step 4;

Formula (XI-5) may be prepared by intermolecular cyclization of Formula (XI-4) in presence of thionyl chloride and a base such as TEA.

### Step 5:

Formula (XI-6) may be prepared by decarboxylation of Formula (XI-5) in presence of sodium chloride under microwave heating condition.

### Step 6:

Formula (XI-7) may be prepared by substitution of chlorine of Formula (XI-6) with an alcohol under basic condition.

### Step 7:

Formula (XI-8) may be prepared by intermolecular cyclization of Formula (XI-7) under Mistunobu reaction condition.

### Step 8:

Formula (XI-9) may be prepared by PMB deprotection of Formula (XI-8) under acidic condition.

### Step 9:

Formula (XI-10) may be prepared by Boc protection of secondary amine of Formula (XI-9) under standard condition.

### Step 10:

Formula (XI-11) may be prepared by Mistunobu coupling between an alcohol and Formula (XI-10).

### Step 11:

Formula (XI-12) may be prepared by Suzuki coupling between Formula (XI-11) and boronic ester under standard conditions.

### Step 12:

Formula (XI-13) may be prepared by Boc deprotection of Formula (XI-12) under acidic condition.

### Step 13:

Formula (II′a) may be prepared by TIPS deprotection of Formula (XI-13) in presence of CsF under standard condition.

In some embodiments, compounds of Formula (XII-13) provided herein may be prepared by the synthetic route as shown in Scheme 12:

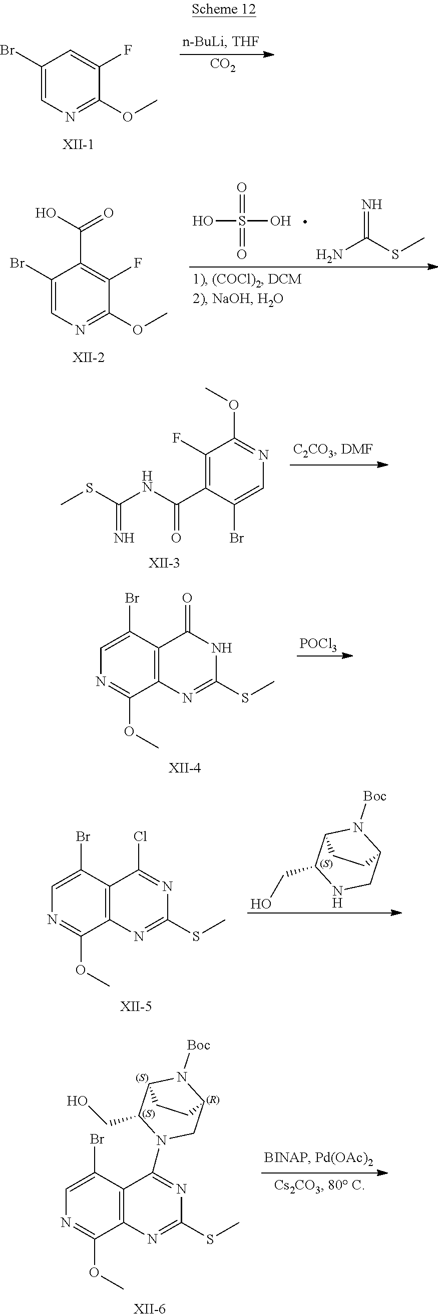

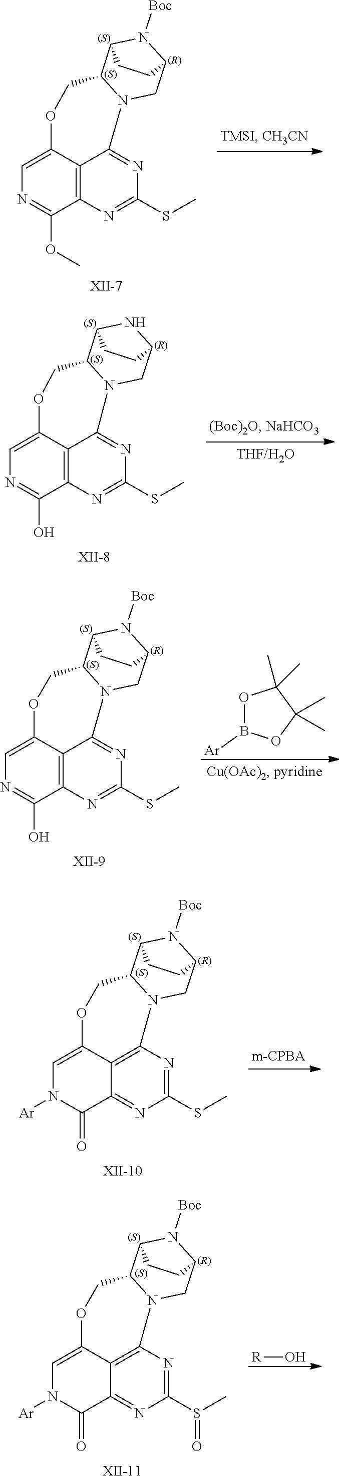

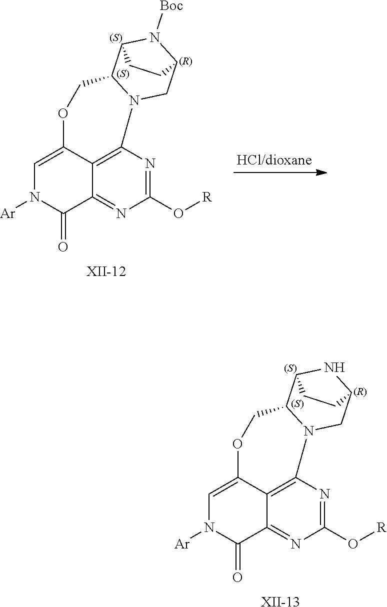

### Step 1:

Compound of Formula (XII-2) may be prepared by deprotonation of Formula (XII-1) with a base such as n-BuLi, followed by the addition of CO₂ under standard condition.

### Step 2:

Compound of Formula (XII-3) may be prepared by coupling between acid chloride of Formula (XII-2) with 2-methyl-2-thio-pseudourehydrogensulfate under basic condition.

### Step 3:

Compound of Formula (XII-4) may be prepared by intramolecular cyclization of Formula (XII-3) under basic condition.

### Step 4:

Compound of Formula (XII-5) may be prepared by treatment of Formula (XII-4) with POCl₃ under standard conditions.

### Step 5:

Compound of Formula (XII-6) may be prepared by SNAr reaction of Formula (XII-5) with tert-butyl (1S,2S,5R)-2-(hydroxymethyl)-3,8-diazabicyclo[3.2.1]octane-8-carboxylate under basic condition.

### Step 6:

Compound of Formula (XII-7) may be prepared by intramolecular coupling of Formula (XII-6) in presence of a palladium (II) catalyst, i.e. Pd(OAc)₂ and a ligand i.e. BINAP, under standard condition.

### Step 7:

Compound of Formula (XII-8) may be prepared by demethylation of Formula (XII-7) with TMSI under standard conditions.

### Step 8:

Compound of Formula (XII-9) may be prepared by Boc protection of Formula (XII-8) under standard conditions.

### Step 9:

Compound of Formula (XII-10) may be prepared by Chan-Lam coupling between Formula (XII-9) and an aromatic boronic ester in presence of a copper (II) catalyst, i.e. Cu(OAc)₂ with pyridine as solvent.

### Step 10:

Compound of Formula (XII-11) may be prepared by sulfur oxidation of Formula (XII-10) with m-CPBA under standard conditions.

### Step 11:

Compound of Formula (XII-12) may be prepared by SNAr reaction of Formula (XII-11) with an alcohol under basic condition.

### Step 12:

Compound of Formula (XII-13) may be prepared by Boc deprotection of Formula (XII-12) under standard conditions.

In some embodiments, compounds of Formula (XIII-11) provided herein may be prepared by the synthetic route as shown in Scheme 13:

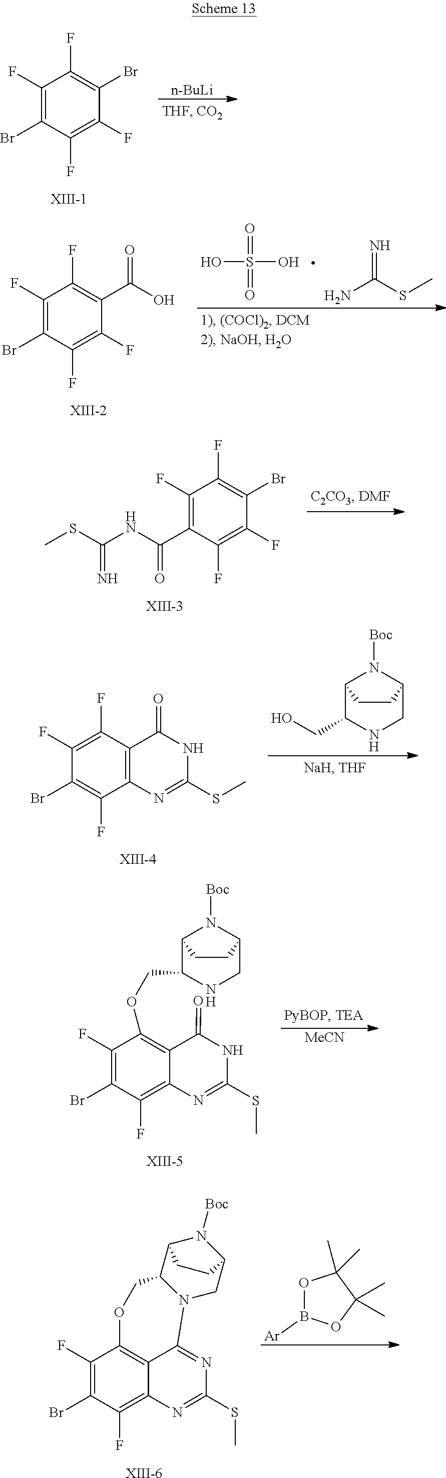

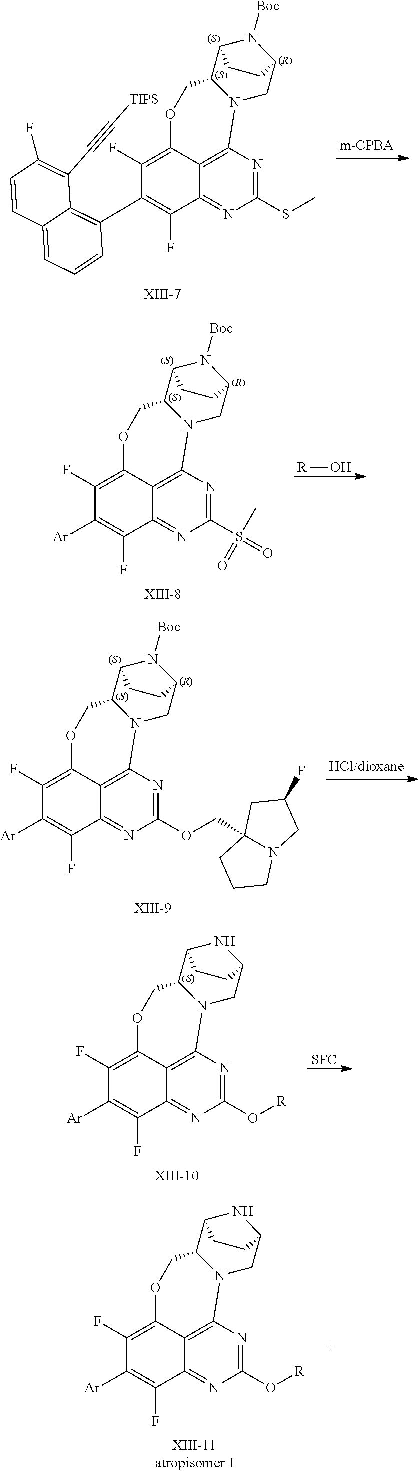

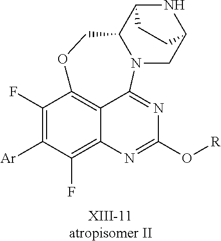

### Step 1:

Compound of Formula (XIII-2) may be prepared by deprotonation of Formula (XII-1) with a base such as n-BuLi, followed by the addition of CO₂ under standard condition.

### Step 2:

Compound of Formula (XIII-3) may be prepared by coupling between acid chloride of Formula (XIII-2) with 2-methyl-2-thio-pseudourehydrogensulfate under basic condition.

### Step 3:

Compound of Formula (XIII-4) may be prepared by intramolecular cyclization of Formula (XIII-3) under basic condition.

### Step 4:

Compound of Formula (XIII-5) may be prepared by SNAr reaction of Formula (XIII-4) with tert-butyl (1S,2S,5R)-2-(hydroxymethyl)-3,8-diazabicyclo[3.2.1]octane-8-carboxylate under basic condition.

### Step 5:

Compound of Formula (XIII-6) may be prepared by intramolecular cyclization of Formula (XIII-5) in presence of PyBOP under basic condition.

### Step 6:

Compound of Formula (XIII-7) may be prepared by Suzuki coupling between a boronic ester and Formula (XIII-6) under standard condition.

### Step 7:

Compound of Formula (XIII-8) may be prepared by sulfur oxidation of Formula (XIII-7) with m-CPBA under standard conditions.

### Step 8:

Compound of Formula (XIII-9) may be prepared by SNAr reaction of Formula (XIII-8) with an alcohol under basic condition.

### Step 9:

Compound of Formula (XIII-10) may be prepared by Boc deprotection of Formula (XIII-9) under standard conditions.

### Step 10:

Atropisomers I and II of Formula (XIII-11) may be prepared by SFC separation of Formula (XIII-10) under standard chiral SFC separation conditions.

In some embodiments, compounds of Formula (XIV-2) provided herein may be prepared by the synthetic route as shown in Scheme 14:

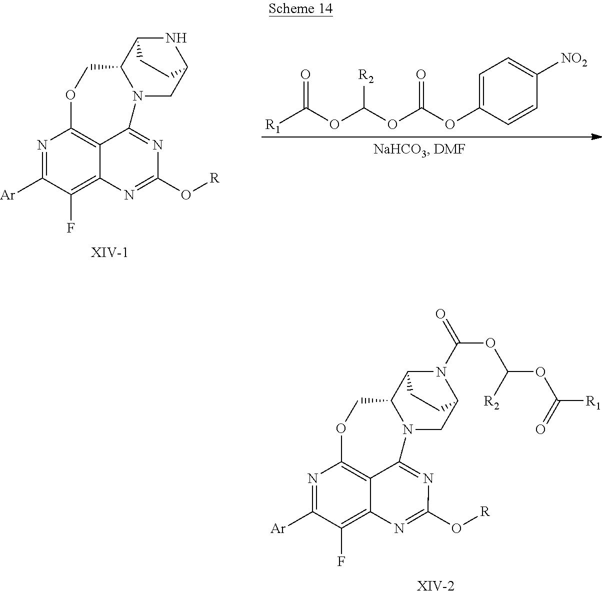

Compound of Formula (XIV-2) may be prepared by reacting Formula (XIV-1) with a carbonate reagent in presence of a base under standard condition.

### Example 1

### Preparation of intermediate tert-butyl (1S,2S,5R)-2-(hydroxymethyl)-3,8-diazabicyclo[3.2.1]octane-8-carboxylate (INT1)

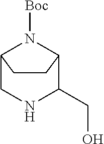 [SMILES: CC(C)(C)OC(=O)N1C2CCC1C(CO)NC2]

Intermediate INT1 was prepared following the synthetic route as shown in Scheme 5.

### Methyl 5-methoxy-3,4-dihydro-2H-pyrrole-2-carboxylate

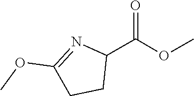

To a solution of methyl 5-oxopyrrolidine-2-carboxylate (42 mL, 349 mmol) in DCM (300 mL) was added trimethyloxonium tetrafluoroborate (57 g, 384 mmol), and the reaction was stirred at room temperature for 18 hours. The reaction was quenched with saturated NaHCO₃ solution at 0° C. The organic layer was separated, washed with saturated NaHCO₃ solution, and concentrated in vacuo. The residue was purified using silica gel column chromatography to afford the title compound methyl 5-methoxy-3,4-dihydro-2H-pyrrole-2-carboxylate (21 g, 38%) as a yellow oil.

LC/MS ESI (m/z): 158 [M+H]⁺

¹H NMR (400 MHZ, CDCl₃) δ 4.55 (dd, J=7.7, 6.5 Hz, 1H), 4.55 (dd, J=7.7, 6.5 Hz, 1H), 3.87 (s, 3H), 3.77 (d, J=11.8 Hz, 3H), 2.65-2.47 (m, 2H), 2.39-2.29 (m, 1H), 2.24-2.15 (m, 1H).

### Methyl (Z)-5-(2-ethoxy-1-nitro-2-oxoethylidene) pyrrolidine-2-carboxylate and methyl (E)-5-(2-ethoxy-1-nitro-2-oxoethylidene) pyrrolidine-2-carboxylate

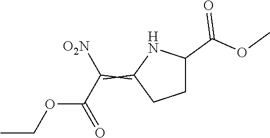 [SMILES: CCOC(=O)C(=C1CCC(C(=O)OC)N1)[N+](=O)[O-]]

To a flask containing methyl 5-methoxy-3,4-dihydro-2H-pyrrole-2-carboxylate (27.5 g, 175 mmol) was added ethyl 2-nitroacetate (39 mL, 349.9 mmol) at room temperature. The mixture was stirred at 60° C. for 18 hours. The resulting mixture was concentrated in vacuo. The residue was purified using silica gel column chromatography to provide the title compounds (Z)-5-(2-ethoxy-1-nitro-2-oxoethylidene) pyrrolidine-2-carboxylate and methyl (E)-5-(2-ethoxy-1-nitro-2-oxoethylidene) pyrrolidine-2-carboxylate (16 g, 62 mmol, 35.4%) as a yellow gum.

LC/MS ESI (m/z): 259 [M+H]⁺.

¹H NMR (400 MHZ, DMSO-d₆) δ 10.19 (s, 1H), 4.63 (dd, J=9.3, 4.4 Hz, 1H), 4.19 (q, J=7.1 Hz, 2H), 3.72 (d, J=14.4 Hz, 3H), 3.06 (s, 2H), 2.44-2.34 (m, 1H), 2.05 (dt, J=18.6, 6.5 Hz, 1H), 1.28-1.20 (m, 3H).

### Ethyl-4-oxo-3,8-diazabicyclo[3.2.1]octane-2-carboxylate

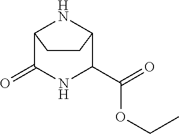

To a solution of (Z)-5-(2-ethoxy-1-nitro-2-oxoethylidene) pyrrolidine-2-carboxylate and methyl (E)-5-(2-ethoxy-1-nitro-2-oxoethylidene) pyrrolidine-2-carboxylate in EtOH (300 mL) was added Pd/C (131 mmol). The reaction was stirred at room temperature for 3 days under H₂ atmosphere (20 atm). The reaction was filtered and concentrated in vacuo. The residue was purified using silica gel column chromatography eluting with methanol in chloroform (0˜10%) to afford the title compound (1:1 major diastereomeric mixture)ethyl-4-oxo-3,8-diazabicyclo[3.2.1]octane-2-carboxylate (4 g, 20 mmol, 37%) as a yellow solid and minor diastereomeric mixture ethyl-4-oxo-3,8-diazabicyclo[3.2.1]octane-2-carboxylate (170.0 mg, 1.6%) as a white solid (discarded).

Major diastereomeric mixture: LC/MS ESI (m/z): 199 [M+H]⁺.

¹H NMR (400 MHZ, DMSO-d₆) δ 7.25 (s, 1H), 4.25 (d, J=4.4 Hz, 1H), 4.19-4.08 (m, 2H), 3.74 (t, J=5.1 Hz, 1H), 3.38 (d, J=6.6 Hz, 1H), 1.86-1.65 (m, 3H), 1.51-1.39 (m, 1H), 1.20 (t, J=7.1 Hz, 3H).

### 8-(Tert-butyl)₂-ethyl-4-oxo-3,8-diazabicyclo[3.2.1]octane-2,8-dicarboxylate

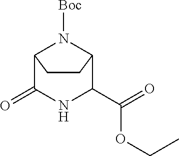 [SMILES: CCOC(=O)C1NC(=O)C2CCC1N2C(=O)OC(C)(C)C]

To a solution of ethyl-4-oxo-3,8-diazabicyclo[3.2.1]octane-2-carboxylate (2 g, 10 mmol) in THF (20 mL) and H₂O (5 mL) was added NaHCO₃ (1.7 g, 20 mmol) and (Boc)₂O (2.2 mL, 10 mmol), and the reaction was stirred at room temperature for 24 hours. The reaction was diluted with ethyl acetate and water. The organic layer was separated, washed with saturated NaCl solution, and concentrated in vacuo. The residue was purified using silica gel column chromatography to afford the title compound (1:1 major diastereomeric mixture) 8-tert-butyl 2-ethyl 4-oxo-3,8-diazabicyclo[3.2.1]octane-2,8-dicarboxylate (2.45 g, 81%) as a white solid.

LC/MS ESI (m/z): 299 [M+H]⁺.

¹H NMR (400 MHz, DMSO-d₆) δ 7.64 (s, 1H), 4.43 (s, 2H), 4.22-4.13 (m, 2H), 4.10 (d, J=6.6 Hz, 1H), 2.12-1.97 (m, 2H), 1.81 (t, J=9.2 Hz, 1H), 1.57 (t, J=8.6 Hz, 1H), 1.41 (s, 9H), 1.22 (t, J=7.1 Hz, 3H).

### Tert-butyl-2-(hydroxymethyl)-3,8-diazabicyclo[3.2.1]octane-8-carboxylate

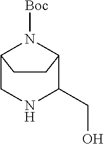 [SMILES: CC(C)(C)OC(=O)N1C2CCC1C(CO)NC2]

To a suspension of LiAlH₄ (1.22 g, 32 mmol) in THF (20 mL) was added 8-tert-butyl 2-ethyl 4-oxo-3,8-diazabicyclo[3.2.1]octane-2,8-dicarboxylate (1.2 g, 4.0 mmol) in THF (20 mL) dropwise at 0° C. The reaction was stirred at 0° C. for 5 hours under N₂. The reaction was quenched with saturated aqueous Na₂SO₄ solution. The mixture was filtered and the resulting filtrate was washed with DCM/MeOH (10/1). The combined organic phase was concentrated in vacuo to afford the title compound (1:1 major diastereomeric mixture) tert-butyl 2-(hydroxymethyl)-3,8-diazabicyclo[3.2.1]octane-8-carboxylate (0.8 g, 82%) as a yellow oil which was used directly without further purification.

LC/MS ESI (m/z): 243 [M+H]⁺.

¹H NMR (400 MHZ, DMSO-d₆) δ 4.53 (d, J=60.7 Hz, 1H), 3.95 (s, 2H), 3.35 (s, 1H), 3.19 (d, J=6.4 Hz, 2H), 2.73 (d, J=11.4 Hz, 2H), 2.56 (d, J=11.5 Hz, 1H), 1.82-1.62 (m, 3H), 1.56 (s, 1H), 1.40 (s, 9H).

### Example 2

### Preparation of intermediate 4-Bromo-2,6-dichloro-5-fluoronicotinic acid (VI-8)

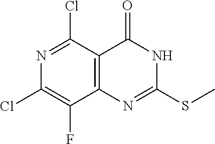 [SMILES: CSc1nc2c(F)c(Cl)nc(Cl)c2c(=O)[nH]1]

Intermediate of Formula (VI-8) was prepared following the synthetic route as shown in Scheme 7.

### 4-bromo-2,6-dichloro-5-fluoronicotinic Acid

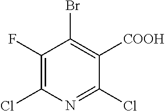 [SMILES: O=C(O)c1c(Cl)nc(Cl)c(F)c1Br]

To a cooled solution of 2,6-dichloro-5-fluoronicotinic acid (6.0 mL, 47.6 mmol) in anhydrous THF (100 mL), was added MeLi (76.0 mL, 98.8 mmol, 1.3 M in THF) at −78° C. over 30 min. The reaction was warmed to −20˜−30° C. for 2 hours. The reaction mixture was cooled to −78° C., followed by addition of 1,2-dibromo-1,1,2,2-tetrachloroethane (6.30 mL, 52 mmol) in anhydrous THF (30 mL). The reaction mixture was stirred at 0° C. for 1.5 hours. The reaction solution was diluted with ice water (150 mL) followed by extraction with chloroform (30 mL). The aqueous layer was separated and adjusted to pH=2 by addition of 1N hydrochloric acid. The aqueous layer was then extracted by ethyl acetate (3×50 mL). The organic layer was combined and dried over Na₂SO₄, filtered, and concentrated to afford titled compound 4-bromo-2,6-dichloro-5-fluoronicotinic acid (12 g, 87%) as a white solid which was in next step without further purification.

LC/MS ESI (m/z): 288 [M+H]⁺.

### Methyl (4-bromo-2,6-dichloro-5-fluoronicotinoyl) carbamimidothioate

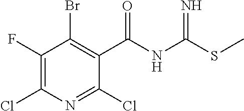 [SMILES: CSC(=N)NC(=O)c1c(Cl)nc(Cl)c(F)c1Br]

To a solution of 4-bromo-2,6-dichloro-5-fluoronicotinic acid (6.65 g, 23 mmol) in DCM (60 mL) were added oxalyl dichloride (4.38 g, 34.5 mmol) under N₂ atmosphere at 0° C., and the reaction was stirred at room temperature for 3 hours. The reaction concentrated in vacuo to give crude 4-bromo-2,6-dichloro-5-fluoronicotinoyl chloride as reddish brown oil. To a solution of NaOH (4.26 g, 106.6 mmol) in H₂O (80 mL) were added 2-methyl-2-thiopseudourea sulfate (8.0 g, 42.6 mmol) in small batches at 0° C., and the resulting mixture was stirred at 0° C. for 30 minutes. The above mixture was added to the solution of 4-bromo-2,6-dichloro-5-fluoronicotinoyl chloride in DCM (20 mL) at 0° C., and the reaction was stirred at room temperature for 1 hour. The organic layer was separated, and the aqueous layer was extracted with EA (2×40 mL). Combined the organic layer was dried with anhydrous sodium sulfate, filtered and concentrated in vacuo. The residue was purified using silica gel column chromatography to afford the title compound methyl (4-bromo-2,6-dichloro-5-fluoronicotinoyl) carbamimidothioate (2.6 g, 31.3%) as a white solid.

LCMS (ESI) m/z: 361 [M+H]⁺.

### 5,7-dichloro-8-fluoro-2-(methylthio)pyrido[4,3-d]pyrimidin-4 (3H)-one

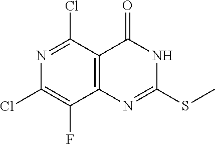 [SMILES: CSc1nc2c(F)c(Cl)nc(Cl)c2c(=O)[nH]1]

To a solution of methyl (4-bromo-2,6-dichloro-5-fluoronicotinoyl) carbamimidothioate (2.6 g, 7.2 mmol) in DMF (30 mL) were added Cs₂CO₃ (3.52 g, 10.8 mmol), and the reaction stirred at 90° C. for 1 hour. The reaction was cooled to room temperature and pulled into ice water (20 mL). The pH of aqueous phase was adjusted to 2˜3, solid precipitated. The solid was collected by filtration, and washed with water. The residue was dried in vacuum dryer to afford the 5,7-dichloro-8-fluoro-2-(methylthio)pyrido[4,3-d]pyrimidin-4 (3H)-one (1.8 g, 89%) as a white solid.

LC/MS ESI (m/z): 280 [M+H]⁺.

The following examples can be prepared following the synthetic route as shown in Scheme 6.

### Example 3

### Preparation of 5-Chloro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol (Compound 1)

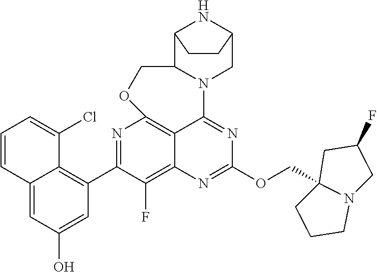 [SMILES: Oc1cc(-c2nc3c4c(nc(OC[C@@]56CCCN5C[C@H](F)C6)nc4c2F)N2CC4CCC(N4)C2CO3)c2c(Cl)cccc2c1]

### 2,6-Dichloro-3-fluoropyridin-4-amine

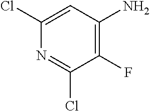

To a solution of 2,6-dichloropyridin-4-amine (6 g, 36.8 mmol) in DMF (20 mL) and MeCN (20 ml), Selectfluor (15.6 g, 44.2 mmol) was added in one portion. The mixture was stirred at 80° C. for 4 hours. The crude reaction mixture was filtered, and the filtrate was concentrated to give the crude product which was further purified by silica gel column chromatography to afford 2,6-dichloro-3-fluoropyridin-4-amine (3.2 g, 48% yield) as a white solid.

LC/MS (ESI) m/z: 181 [M+H]⁺.

### 2,6-Dichloro-3-fluoro-5-iodopyridin-4-amine

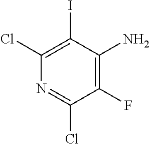 [SMILES: Nc1c(F)c(Cl)nc(Cl)c1I]

To a mixture of 2,6-dichloro-3-fluoropyridin-4-amine (3.2 g, 17.68 mmol) in MeCN (20 mL) was added N-iodosuccinimide (3.67 g, 21.21 mmol), p-toluenesulfonic acid (0.17 g, 0.88 mmol). The mixture was stirred at 70° C. for 16 hours. The reaction mixture was filtered and the filtrate was concentrated to get crude product which was further purified by silica gel column chromatography to afford 2,6-dichloro-3-fluoro-5-iodopyridin-4-amine as a white solid.

LC/MS (ESI) m/z: 307 [M+H]⁺.

### Methyl 4-amino-2,6-dichloro-5-fluoronicotinate

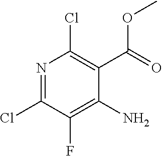

A mixture of 2,6-dichloro-3-fluoro-5-iodopyridin-4-amine (4.6 g, 15 mmol), Pd(dppf)Cl₂ (2.19 g, 3 mmol) and TEA (12.7 mL, 90 mmol) in MeOH (50 mL) was stirred at 65° C. under carbon monoxide atmosphere (15 psi) for 12 hours. The mixture was filtered through Celite, and the filtrate was concentrated to give the crude product. The crude product was purified by silica gel column chromatography to afford methyl 4-amino-2,6-dichloro-5-fluoropyridine-3-carboxylate (2.5 g, 10.46 mmol, 70%) as a pink powder.

LC/MS (ESI) m/z: 239 [M+H]⁺.

¹H NMR (400 MHZ, DMSO-d₆) δ 7.31 (s, 2H), 3.87 (s, 3H).

### 4-Amino-2,6-dichloro-5-fluoronicotinic Acid

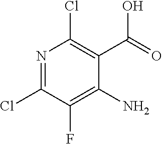 [SMILES: Nc1c(F)c(Cl)nc(Cl)c1C(=O)O]

To a solution of methyl 4-amino-2,6-dichloro-5-fluoropyridine-3-carboxylate (1.5 g, 6.28 mmol) in MeOH (30 mL) were added THF (10 mL), followed by a solution of NaOH (0.75 g, 18.83 mmol) in H₂O (10 mL). The resulting solution was stirred at room temperature for 16 hours. The reaction mixture was treated with ethyl acetate and water. The aqueous layer was separated and washed with petroleum ether before being acidified to pH=5 with aq. HCl (3 N). Then, the resulting aqueous layer was concentrated to dryness and co-evaporated with EtOH twice to give crude 4-amino-2,6-dichloro-5-fluoropyridine-3-carboxylic acid (1.9 g, crude) containing NaCl as a white solid which was used in next without further purification.

LC/MS ESI (m/z): 225 [M+H]⁺

### 4-Amino-2,6-dichloro-5-fluoronicotinoyl chloride

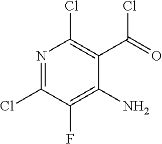 [SMILES: Nc1c(F)c(Cl)nc(Cl)c1C(=O)Cl]

A mixture of crude 4-amino-2,6-dichloro-5-fluoropyridine-3-carboxylic acid (1.9 g, 6.3 mmol) containing NaCl in POCl₃ (20 mL, 214.6 mmol) was stirred at 90° C. for 3 hours. After cooling to rt, the mixture was filtered. The resulting filtrate was concentrated in vacuo by oil pump to afford crude 4-amino-2,6-dichloro-5-fluoronicotinoyl chloride (2.1 g, crude) as a yellow oil which was directly used in the following step.

LC/MS ESI (m/z): 243 [M+H]⁺

### 5,7-Dichloro-8-fluoro-2-thioxo-2,3-dihydropyrido[4,3-d]pyrimidin-4 (1H)-one

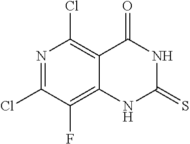 [SMILES: O=c1[nH]c(=S)[nH]c2c(F)c(Cl)nc(Cl)c12]

The crude 4-amino-2,6-dichloro-5-fluoronicotinoyl chloride (2.1 g, 6.3 mmol) was dissolved in dry THF (20 mL) followed by addition of a solution of NH₄SCN (1.44 g, 18.9 mmol) in THF (30 mL) at 0° C. over 10 minutes. The resulting mixture was warmed slowly to room temperature and stirred overnight. The reaction mixture was treated with ethyl acetate and water. The organic layer was separated, washed with brine, dried over Na₂SO₄ and concentrated in vacuo to give crude 5,7-dichloro-8-fluoro-2-sulfanylidene-1H,2H,3H,4H-pyrido[4,3-d]pyrimidin-4-one (2.1 g, 100.10%) as a yellow solid without further purification for the next step.

LC/MS ESI (m/z): 264 [M−H]⁻

### 5,7-dichloro-8-fluoro-2-(methylthio)pyrido[4,3-d]pyrimidin-4 (3H)-one

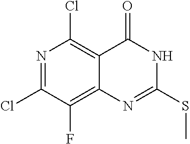 [SMILES: CSc1nc2c(F)c(Cl)nc(Cl)c2c(=O)[nH]1]

To a solution of crude 5,7-dichloro-8-fluoro-2-sulfanylidene-1H,2H,3H,4H-pyrido[4,3-d]pyrimidin-4-one (2.1 g, 6.3 mmol) in dry DMF (30 mL) was added EtONa (0.43 g, 6.3 mmol) at 0° C. The resulting mixture was stirred at room temperature for 10 minutes before iodomethane (0.47 mL, 7.6 mmol) was added dropwise at 0° C. Then, the reaction mixture was stirred at room temperature for 1 hour. The reaction mixture was treated with ethyl acetate and ice water. The organic layer was separated, washed with brine, dried over Na₂SO₄ and concentrated in vacuo. The residue was purified by silica gel column chromatography, followed by trituration with small amount of EtOH to give pure 5,7-dichloro-8-fluoro-2-(methylsulfanyl)-3H,4H-pyrido[4,3-d]pyrimidin-4-one (400 mg, 23%) as a yellow solid.

LC/MS ESI (m/z): 280 [M+H]⁺

### Tert-butyl 2-(((7-chloro-8-fluoro-2-(methylthio)-4-oxo-3,4-dihydropyrido[4,3-d]pyrimidin-5-yl)oxy)methyl)-3,8-diazabicyclo[3.2.1]octane-8-carboxylate

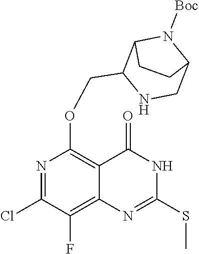 [SMILES: CSc1nc2c(F)c(Cl)nc(OCC3NCC4CCC3N4C(=O)OC(C)(C)C)c2c(=O)[nH]1]

To a solution of INT1 tert-butyl 2-(hydroxymethyl)-3,8-diazabicyclo[3.2.1]octane-8-carboxylate (207.6 mg, 0.86 mmol) (1:1 major diastereomeric mixture) in dry THF (20 mL) was added NaH (68 mg, 1.71 mmol, 60% in mineral oil) at 0° C. The resulting mixture was stirred at room temperature for 0.5 h before 5,7-dichloro-8-fluoro-2-(methylsulfanyl)-3H,4H-pyrido[4,3-d]pyrimidin-4-one (200 mg, 0.71 mmol) was added in one portion at 0° C. Then, the reaction mixture was allowed to stir at room temperature for 2 hours. LCMS showed the reaction was completed. The reaction mixture was quenched with cold sat. NH₄Cl and then extracted with DCM twice. The combined extracts were concentrated and purified by flash column chromatography on silica gel to give 2-(((7-chloro-8-fluoro-2-(methylthio)-4-oxo-3,4-dihydropyrido[4,3-d]pyrimidin-5-yl)oxy)methyl)-3,8-diazabicyclo[3.2.1]octane-8-carboxylate (225 mg, 64.8%) as a white solid (1:1 major diastereomeric mixture).

LC/MS ESI (m/z): 486 [M+H]⁺

### Tert-butyl 2-chloro-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate

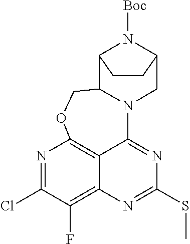 [SMILES: CSc1nc2c3c(nc(Cl)c(F)c3n1)OCC1C3CCC(CN21)N3C(=O)OC(C)(C)C]

To a solution of 2-(((7-chloro-8-fluoro-2-(methylthio)-4-oxo-3,4-dihydropyrido[4,3-d]pyrimidin-5-yl)oxy)methyl)-3,8-diazabicyclo[3.2.1]octane-8-carboxylate (220 mg, 0.45 mmol) in dry MeCN (20 mL) was added PyBOP (471 mg, 0.91 mmol). After cooling to 0° C., DBU (0.27 mL, 1.81 mmol) was added dropwise, and the resulting mixture was stirred at room temperature for 16 hours. LCMS showed the reaction was complete. The reaction mixture was poured into aq. sat. NaHCO₃ and then extracted with ethyl acetate twice. The combined extracts were concentrated in vacuo and the residue was purified by flash column chromatography on silica gel to give tert-butyl 2-chloro-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate (130 mg, 61.4%) (1:1 major diastereomeric mixture) as a colorless gum.

LC/MS ESI (m/z): 468 [M+H]⁺

### Tert-butyl (6R,9S)-2-(8-chloro-3-(methoxymethoxy)naphthalen-1-yl)-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate

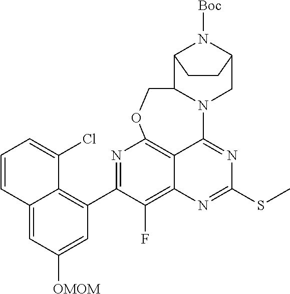

A mixture of tert-butyl 2-chloro-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate (100 mg, 0.2 mmol) and 2-[8-chloro-3-(methoxymethoxy)naphthalen-1-yl]-4,4,5,5-tetramethyl-1,3,2-dioxaborolane (97 mg, 0.28 mmol), K₂CO₃ (59 mg, 0.43 mmol) in THF (1 mL) and water (0.3 mL) was degassed with N₂ three times. Then XPhos-Pd-G2 (16.8 mg, 0.02 mmol). The reaction tube was degassed with N₂ for 10 min and then the mixture was stirred at 60° C. for 2.5 hours under N₂. After completion, the mixture was diluted with ethyl acetate (5 mL) and water (2 mL). The aqueous phase was extracted with ethyl acetate (2×2 mL). The combined organic layer was washed with saturated brine (5 mL), dried over Na₂SO₄, filtered, and concentrated under vacuum. The residue was purified by column chromatography to give the title compound (70 mg, 50%) (1:1 major diastereomeric mixture) as a brown solid.

LC/MS ESI (m/z): 654 [M+H]⁺.

### Tert-butyl (6R,9S)-2-(8-chloro-3-(methoxymethoxy)naphthalen-1-yl)-1-fluoro-12-(methylsulfonyl)-5a,6,7,8,9,10-hexahydro-5H-4-oxa-3,10a,11,13,14-pentaaza-6,9-methanonaphtho[1,8-ab]heptalene-14-carboxylate

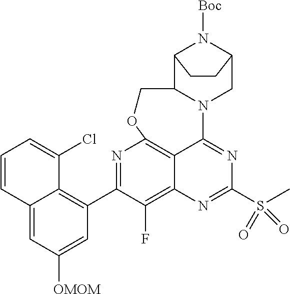

To a flask containing tert-butyl (6R,9S)-2-(8-chloro-3-(methoxymethoxy)naphthalen-1-yl)-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate (45 mg, 0.07 mmol) was added DCM (3 mL) followed by the addition of m-CPBA (23 mg, 0.14 mmol) at 0° C. The mixture was stirred at 0° C. for 5 min. The result mixture was quenched by NaHCO₃(aq). The mixture was extracted with DCM (3×3 mL). Combined the CH₂Cl₂ layer was dried by Na₂SO₄, filtered and concentrated. The crude material was purified by prep-TLC plate to provide the title compound tert-butyl (6R,9S)-2-(8-chloro-3-(methoxymethoxy)naphthalen-1-yl)-1-fluoro-12-(methylsulfonyl)-5a,6,7,8,9,10-hexahydro-5H-4-oxa-3,10a,11,13,14-pentaaza-6,9-methanonaphtho[1,8-ab]heptalene-14-carboxylate (42 mg, 89%) (1:1 major diastereomeric mixture).

LCMS (ESI) m/z: 686 [M+H]⁺.

### Tert-butyl (6R,9S)-2-(8-chloro-3-(methoxymethoxy)naphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

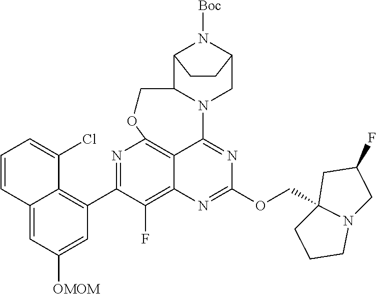

To a solution of ((2R,7aS)-2-fluorotetrahydro-1H-pyrrolizin-7a(5H)-yl) methanol (8 mg, 0.05 mmol) in dry THF (3 mL) was added NaH (4 mg, 0.1 mmol, 60% in mineral oil) at 0° C. The resulting mixture was stirred at room temperature for 0.5 hour before a solution of tert-butyl (6R,9S)-2-(8-chloro-3-(methoxymethoxy)naphthalen-1-yl)-1-fluoro-12-(methylsulfonyl)-5a,6,7,8,9,10-hexahydro-5H-4-oxa-3,10a,11,13,14-pentaaza-6,9-methanonaphtho[1,8-ab]heptalene-14-carboxylate (35 mg, 0.05 mmol) in dry THF (1 mL) was added dropwise at 0° C. LCMS showed the starting material was fully consumed. The reaction mixture was poured into sat. NH₄Cl and then extracted with DCM twice. The combined extracts were concentrated in vacuo and the residue was purified by prep-TLC to give tert-butyl (6R,9S)-2-(8-chloro-3-(methoxymethoxy)naphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (20 mg, 51%) as a white solid (1:1 major diastereomeric mixture).

LC/MS ESI (m/z): 765 [M+H]⁺

### 5-Chloro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

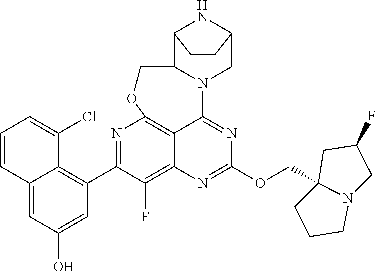 [SMILES: Oc1cc(-c2nc3c4c(nc(OC[C@@]56CCCN5C[C@H](F)C6)nc4c2F)N2CC4CCC(N4)C2CO3)c2c(Cl)cccc2c1]

To a flask containing tert-butyl (6R,9S)-2-(8-chloro-3-(methoxymethoxy)naphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (20 mg, 0.03 mmol) was added DCM (3 mL) followed by the addition of HCl/dioxane (4M, 1 mL). The mixture was stirred at room temperature for 30 min. The result mixture was concentrated and the residue was purified by prep-HPLC with to afford the title compound 5-chloro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol (6 mg, 37%) (1:1 major diastereomeric mixture).

LC/MS ESI (m/z): 621 [M+H]⁺.

¹HNMR (400 MHZ, Methanol-d₄) δ 8.45 (s, 1H), 7.78-7.70 (m, 1H), 7.40-7.29 (m, 3H), 7.16 (dd, J=52.4, 2.0 Hz, 1H), 5.45 (d, J=52.5 Hz, 1H), 5.09 (t, J=15.3 Hz, 1H), 4.67-4.44 (m, 4H), 4.21 (d, J=8.3 Hz, 1H), 3.88-3.55 (m, 5H), 3.27 (s, 2H), 2.62-2.38 (m, 2H), 2.31 (s, 1H), 2.25-2.15 (m, 2H), 2.11-1.78 (m, 5H).

The following compounds can be prepared in a similar way to Compound 1, except for using other appropriate aryl boronic esters and alcohols.

### Compound 2: 5-chloro-4-((6R,9S)-1-fluoro-12-((tetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

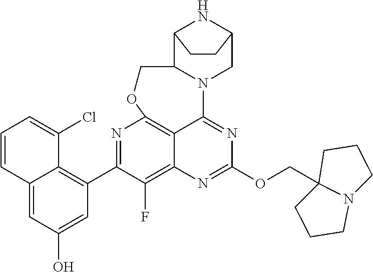 [SMILES: Oc1cc(-c2nc3c4c(nc(OCC56CCCN5CCC6)nc4c2F)N2CC4CCC(N4)C2CO3)c2c(Cl)cccc2c1]

LC/MS (ESI) m/z: 603 [M+H]⁺.

¹H NMR (400 MHZ, Methanol-d₄) δ 8.41 (s, 2H), 7.77-7.71 (m, 1H), 7.37-7.31 (m, 3H), 7.17 (dd, J=52.5, 2.5 Hz, 1H), 5.08 (d, J=17.5 Hz, 1H), 4.64 (d, J=3.7 Hz, 2H), 4.56-4.50 (m, 1H), 4.23 (d, J=8.4 Hz, 1H), 3.84 (d, J=23.3 Hz, 2H), 3.71-3.62 (m, 2H), 3.30-3.24 (m, 4H), 2.31 (dd, J=12.1, 6.8 Hz, 2H), 2.24-2.07 (m, 6H), 2.00-1.84 (m, 4H). (1:1 major diastereomeric mixture)

### Compound 3: 6-fluoro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

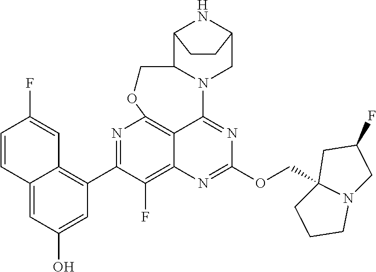 [SMILES: Oc1cc(-c2nc3c4c(nc(OC[C@@]56CCCN5C[C@H](F)C6)nc4c2F)N2CC4CCC(N4)C2CO3)c2cc(F)ccc2c1]

LC/MS (ESI) m/z: 605 [M+H]⁺

¹H NMR (400 MHZ, Methanol-d₄) δ 7.80 (dd, J=8.9, 5.7 Hz, 1H), 7.35-7.20 (m, 4H), 5.50 (d, J=52.4 Hz, 1H), 5.13 (d, J=13.7 Hz, 1H), 4.67 (d, J=11.7 Hz, 1H), 4.62 (d, J=11.9 Hz, 1H), 4.58-4.51 (m, 2H), 4.25 (d, J=6.5 Hz, 1H), 3.95-3.67 (m, 5H), 3.34 (m, 2H), 2.68-2.45 (m, 2H), 2.36 (m, 1H), 2.27 (d, J=5.5 Hz, 2H), 2.14-1.88 (m, 5H). (1:1 major diastereomeric mixture)

### Compound 4: (6R,9S)-2-(8-ethyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

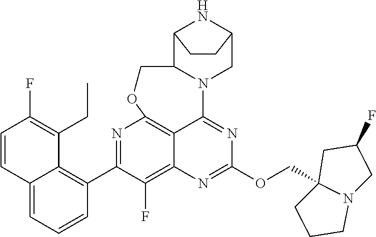 [SMILES: CCc1c(F)ccc2cccc(-c3nc4c5c(nc(OC[C@@]67CCCN6C[C@H](F)C7)nc5c3F)N3CC5CCC(N5)C3CO4)c12]

LC/MS (ESI) m/z: 617 [M+H]⁺.

¹H NMR (400 MHZ, Methanol-d₄) δ 8.04 (d, J=8.1 Hz, 1H), 7.91 (dd, J=8.8, 6.1 Hz, 1H), 7.55-7.33 (m, 3H), 5.50 (d, J=52 Hz, 1H), 5.16 (m, 1H), 4.68-4.51 (m, 4H), 4.29 (m, 1H), 3.98 (m, 2H), 3.80-3.62 (m, 3H), 3.43-3.32 (m, 2H), 2.60 (d, J=9.7 Hz, 1H), 2.55-2.47 (m, 2H), 2.38-2.20 (m, 4H), 2.14-1.91 (m, 5H), 0.88 (dt, J=32.8, 7.3 Hz, 3H). (1:1 major diastereomeric mixture)

### Compound 5: 5-ethynyl-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

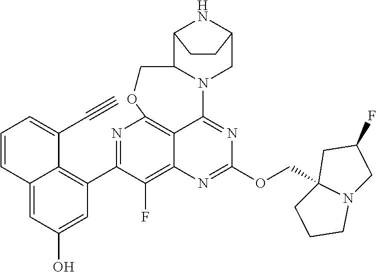 [SMILES: C#Cc1cccc2cc(O)cc(-c3nc4c5c(nc(OC[C@@]67CCCN6C[C@H](F)C7)nc5c3F)N3CC5CCC(N5)C3CO4)c12]

LC/MS (ESI) m/z: 611 [M+H]⁺.

¹H NMR (400 MHZ, Methanol-d₄) δ 8.09-7.47 (m, 3H), 7.44-7.07 (m, 2H), 5.46 (d, J=53.0 Hz, 1H), 5.08 (d, J=13.7 Hz, 1H), 4.68-4.38 (m, 4H), 4.19 (m, 1H), 3.81 (d, J=18.7 Hz, 2H), 3.62 (m, 3H), 3.33 (m, 1H), 3.22 (d, J=2.5 Hz, 2H), 2.56 (m, 1H), 2.45 (m, 1H), 2.29 (m, 1H), 2.20 (d, J=6.0 Hz, 2H), 2.08-1.85 (m, 5H). (1:1 major diastereomeric mixture)

### Compound 6: 5-fluoro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

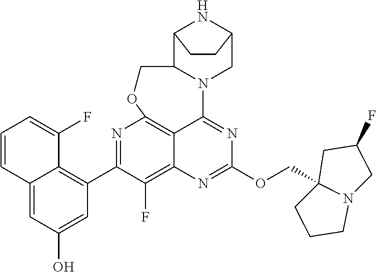 [SMILES: Oc1cc(-c2nc3c4c(nc(OC[C@@]56CCCN5C[C@H](F)C6)nc4c2F)N2CC4CCC(N4)C2CO3)c2c(F)cccc2c1]

LC/MS (ESI) m/z: 605 [M+H]⁺.

¹H NMR (400 MHZ, Methanol-d₄) δ 7.57 (d, J=8.0 Hz, 1H), 7.38 (s, 1H), 7.31 (s, 1H), 7.20-7.06 (m, 1H), 6.92 (d, J=7.5 Hz, 1H), 5.47 (d, J=52.1 Hz, 1H), 5.07 (m, 1H), 4.63 (m, 1H), 4.58 (m, 3H), 4.48 (m, 2H), 4.19 (m, 1H), 3.82 (m, 1H), 3.76 (m, 1H), 3.65 (m, 3H), 2.50 (m, 2H), 2.32 (m, 1H), 2.21 (m, 2H), 2.03 (m, 1H), 1.89 (m, 4H). (1:1 major diastereomeric mixture)

### Compound 7: 5-ethynyl-6-fluoro-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

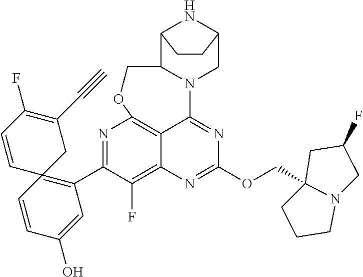

LC/MS ESI (m/z): 629 [M+H]⁺.

¹HNMR (400 MHZ, Methanol-d₄) δ 7.88-7.80 (m, 1H), 7.36-7.28 (m, 2H), 7.28-7.11 (m, 1H), 5.47 (d, J=52.9 Hz, 1H), 5.10 (d, J=13.2 Hz, 1H), 4.67-4.46 (m, 4H), 4.21 (m, 1H), 3.90-3.54 (m, 6H), 3.47 (d, J=4.3 Hz, 1H), 2.63-2.42 (m, 2H), 2.33 (m, 1H), 2.22 (m, 2H), 2.09-1.85 (m, 5H). (1:1 major diastereomeric mixture)

### Compound 8: (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

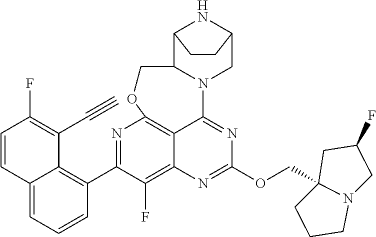 [SMILES: C#Cc1c(F)ccc2cccc(-c3nc4c5c(nc(OC[C@@]67CCCN6C[C@H](F)C7)nc5c3F)N3CC5CCC(N5)C3CO4)c12]

LC/MS (ESI) m/z: 613 [M+H]⁺.

¹H NMR (400 MHZ, Methanol-d₄) δ 8.10 (td, J=7.5, 6.7, 2.4 Hz, 2H), 7.71-7.57 (m, 2H), 7.44 (td, J=8.9, 4.4 Hz, 1H), 5.50 (d, J=52.5 Hz, 1H), 5.16 (d, J=13.7 Hz, 1H), 4.72-4.51 (m, 4H), 4.30 (s, 1H), 4.08-3.92 (m, 2H), 3.92-3.68 (m, 3H), 3.66-3.55 (m, 1H), 3.46-3.32 (m, 2H), 2.70-2.45 (m, 2H), 2.36 (s, 1H), 2.25 (d, J=9.6 Hz, 2H), 2.16-1.90 (m, 5H). (1:1 major diastereomeric mixture)

The 1:1 diastereomeric mixture of (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene (60 mg) was further separated on ChiralPak IB, 250×21.2 mm (I.D., 5 μm) with mobile phase A for CO₂ and B for EtOH+0.1% NH₃H₂O to give faster eluting diastereomer (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene (peak 1, Compound 9, 26 mg, 86%) and slower eluting diastereomer (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene (peak 2, Compound 10, 25 mg, 83%).

### Compound 9: (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

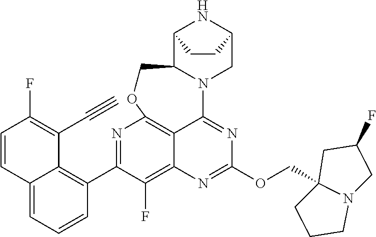 [SMILES: C#Cc1c(F)ccc2cccc(-c3nc4c5c(nc(OC[C@@]67CCCN6C[C@H](F)C7)nc5c3F)N3C[C@H]5CC[C@H](N5)[C@@H]3CO4)c12]

LC/MS (ESI) m/z: 613.5 [M+H]⁺.

¹H NMR (400 MHZ, Methanol-d₄) δ 8.08 (tt, J=6.0, 3.1 Hz, 2H), 7.68-7.55 (m, 2H), 7.42 (td, J=8.9, 5.3 Hz, 1H), 5.30 (d, J=54.9 Hz, 1H), 5.05 (ddd, J=13.5, 7.8, 2.2 Hz, 1H), 4.63-4.56 (m, 1H), 4.49-4.40 (m, 1H), 4.29 (dd, J=10.5, 3.7 Hz, 1H), 4.20 (dd, J=10.5, 7.6 Hz, 1H), 4.15-4.10 (m, 1H), 3.72 (m, 1H), 3.63 (d, J=2.9 Hz, 2H), 3.29-3.18 (m, 4H), 3.07-2.97 (m, 1H), 2.39-2.18 (m, 2H), 2.12 (d, J=9.6 Hz, 1H), 2.03-1.80 (m, 7H).

### Compound 10: (6R,9S)-2-(8-ethynyl-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

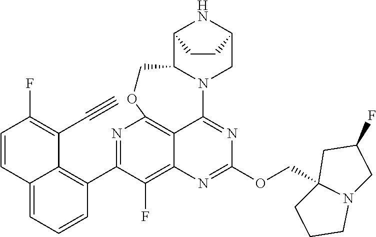 [SMILES: C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 613.5 [M+H]⁺.

¹H NMR (400 MHZ, Methanol-d₄) δ 8.08 (tt, J=6.0, 2.9 Hz, 2H), 7.66-7.56 (m, 2H), 7.43 (td, J=8.9, 5.3 Hz, 1H), 5.32 (d, J=53.8 Hz, 1H), 5.06 (ddd, J=13.5, 6.6, 2.3 Hz, 1H), 4.61 (ddd, J=13.2, 6.8, 2.0 Hz, 1H), 4.45 (dt, J=13.5, 6.7 Hz, 1H), 4.35-4.22 (m, 2H), 4.14 (d, J=5.8 Hz, 1H), 3.73 (d, J=2.9 Hz, 1H), 3.68-3.61 (m, 2H), 3.42-3.32 (m, 1H), 3.24 (d, J=21.8 Hz, 3H), 3.10-3.01 (m, 1H), 2.41-2.23 (m, 2H), 2.21-2.14 (m, 1H), 2.06-1.99 (m, 2H), 1.97-1.80 (m, 5H).

### Compound 11: (6R,9S)-2-(8-chloro-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

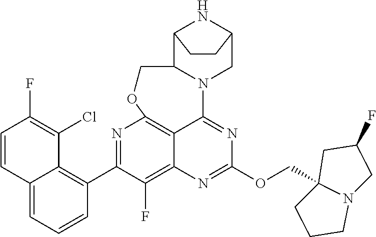 [SMILES: Fc1ccc2cccc(-c3nc4c5c(nc(OC[C@@]67CCCN6C[C@H](F)C7)nc5c3F)N3CC5CCC(N5)C3CO4)c2c1Cl]

LC/MS (ESI) m/z: 623.0 [M+H]⁺.

¹H NMR (400 MHZ, Methanol-d₄) δ 8.14 (dd, J=6.6, 3.2 Hz, 1H), 8.10-8.03 (m, 1H), 7.70-7.57 (m, 2H), 7.52 (td, J=8.9, 5.3 Hz, 1H), 5.47 (d, J=52.0 Hz, 1H), 5.11 (t, J=14.1 Hz, 1H), 4.65 (d, J=13.0 Hz, 1H), 4.58-4.45 (m, 3H), 4.22 (dd, J=16.5, 6.5 Hz, 1H), 3.91-3.78 (m, 2H), 3.77-3.46 (m, 4H), 3.27 (d, J=11.3 Hz, 1H), 2.62-2.42 (m, 2H), 2.41-1.85 (m, 10H). (1:1 major diastereomeric mixture)

### Compound 12: (6R,9S)-2-(8-chloronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

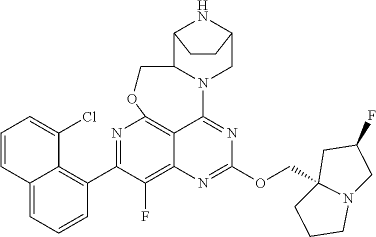 [SMILES: Fc1c(-c2cccc3cccc(Cl)c23)nc2c3c(nc(OC[C@@]45CCCN4C[C@H](F)C5)nc13)N1CC3CCC(N3)C1CO2]

LC/MS (ESI) m/z: 605.1 [M+H]⁺

¹H NMR (400 MHZ, Methanol-d₄) δ 8.15-8.10 (m, 1H), 8.00 (d, J=8.2 Hz, 1H), 7.71-7.60 (m, 2H), 7.60-7.46 (m, 2H), 5.50 (d, J=52.5 Hz, 1H), 5.15 (t, J=15.1 Hz, 1H), 4.59 (tt, J=26.3, 13.3 Hz, 4H), 4.30 (d, J=5.0 Hz, 1H), 4.00-3.89 (m, 2H), 3.87-3.62 (m, 4H), 3.36 (d, J=11.0 Hz, 1H), 2.67-2.46 (m, 2H), 2.35 (s, 1H), 2.30-2.21 (m, 2H), 2.14-1.90 (m, 5H). (1:1 major diastereomeric mixture)

### Compound 13: (6R,9S)-1-fluoro-2-(7-fluoro-8-methylnaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

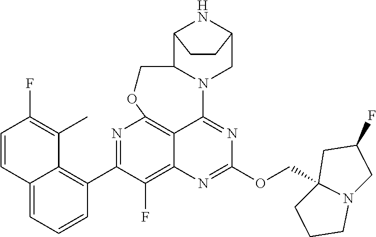 [SMILES: Cc1c(F)ccc2cccc(-c3nc4c5c(nc(OC[C@@]67CCCN6C[C@H](F)C7)nc5c3F)N3CC5CCC(N5)C3CO4)c12]

LC/MS (ESI) m/z: 603.2 [M+H]⁺.

¹H NMR (400 MHZ, Methanol-d₄) δ 8.02 (d, J=7.5 Hz, 1H), 7.93-7.86 (m, 1H), 7.56 (d, J=6.8 Hz, 1H), 7.49 (dd, J=24.8, 7.3 Hz, 1H), 7.35 (dt, J=14.6, 7.3 Hz, 1H), 5.30 (d, J=53.8 Hz, 1H), 5.05 (t, J=14.5 Hz, 1H), 4.60 (d, J=13.2 Hz, 1H), 4.53-4.42 (m, 1H), 4.29 (m, 1H), 4.22 (d, J=10.5 Hz, 1H), 4.14 (d, J=9.0 Hz, 1H), 3.72 (d, J=5.5 Hz, 1H), 3.63 (m, 1H), 3.24 (m, 2H), 3.19 (d, J=7.5 Hz, 2H), 3.01 (d, J=5.4 Hz, 1H), 2.42-2.25 (m, 1H), 2.22 (d, J=10.9 Hz, 1H), 2.19-2.12 (m, 1H), 2.05 (s, 1H), 2.03-1.97 (m, 2H), 1.94 (m, 3H), 1.87 (m, 2H), 1.84 (m, 2H) (1:1 major diastereomeric mixture)

### Compound 14: (6R,9S)-2-(7,8-difluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

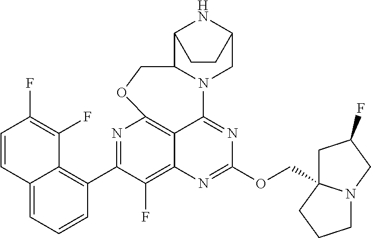 [SMILES: Fc1ccc2cccc(-c3nc4c5c(nc(OC[C@@]67CCCN6C[C@H](F)C7)nc5c3F)N3CC5CCC(N5)C3CO4)c2c1F]

LC/MS (ESI) m/z: 607.1 [M+H]⁺.

¹H NMR (400 MHZ, Methanol-d₄) δ 8.10 (d, J=7.3 Hz, 1H), 7.94-7.81 (m, 1H), 7.72-7.46 (m, 3H), 5.50 (d, J=59.6 Hz, 1H), 5.21-5.02 (m, 1H), 4.69-4.49 (m, 4H), 4.23 (s, 1H), 3.95-3.59 (m, 6H), 2.65-2.31 (m, 3H), 2.26-2.21 (m, 2H), 2.13-1.84 (m, 5H), 1.39-1.23 (m, 1H). (1:1 major diastereomeric mixture)

### Compound 15: (6R,9S)-1-fluoro-2-(8-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

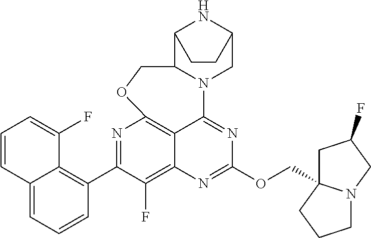 [SMILES: Fc1c(-c2cccc3cccc(F)c23)nc2c3c(nc(OC[C@@]45CCCN4C[C@H](F)C5)nc13)N1CC3CCC(N3)C1CO2]

LC/MS (ESI) m/z: 589.3 [M+H]⁺.

¹H NMR (400 MHZ, Methanol-d₄) δ 8.08 (d, J=8.2 Hz, 1H), 7.82 (dd, J=8.2, 2.1 Hz, 1H), 7.67 (q, J=7.8 Hz, 1H), 7.63-7.52 (m, 1H), 7.51 (dd, J=5.6, 3.0 Hz, 1H), 7.18 (td, J=12.3, 7.2 Hz, 1H), 5.30 (d, J=54.0 Hz, 1H), 5.05 (dd, J=13.6, 2.5 Hz, 1H), 4.59 (d, J=13.2 Hz, 1H), 4.46 (ddd, J=13.3, 9.6, 7.6 Hz, 1H), 4.30 (ddd, J=10.3, 5.2, 2.5 Hz, 1H), 4.22 (dd, J=10.5, 4.1 Hz, 1H), 4.13 (t, J=7.0 Hz, 1H), 3.72 (d, J=5.9 Hz, 1H), 3.63 (d, J=5.4 Hz, 1H), 3.25 (m, 2H), 3.19 (m, 2H), 3.02 (m, 1H), 2.40-2.20 (m, 2H), 2.17-2.12 (m, 1H), 2.01 (dd, J=13.0, 7.0 Hz, 2H), 1.90 (m, 2H), 1.88-1.83 (m, 2H), 1.82 (m, 1H). (1:1 major diastereomeric mixture)

### Compound 16: (6R,9S)-1-fluoro-2-(7-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

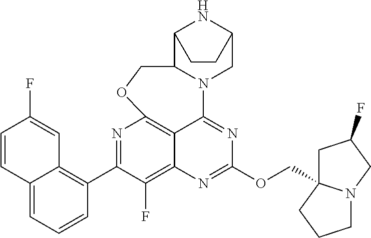 [SMILES: Fc1ccc2cccc(-c3nc4c5c(nc(OC[C@@]67CCCN6C[C@H](F)C7)nc5c3F)N3CC5CCC(N5)C3CO4)c2c1]

LC/MS (ESI) m/z: 589 (M+H)⁺.

¹H NMR (400 MHZ, CDCl₃) δ 8.01-7.81 (m, 2H), 7.70 (d, J=7.1 Hz, 1H), 7.60-7.43 (m, 2H), 7.33-7.27 (m, 1H), 5.45-5.32 (m, 1H), 5.06 (d, J=12.8 Hz, 1H), 4.88-4.74 (m, 4H), 4.57-4.41 (m, 3H), 4.39-4.19 (m, 2H), 3.90 (s, 1H), 3.81-3.58 (m, 3H), 3.44-3.22 (m, 2H), 3.18-3.03 (m, 1H), 2.56-2.28 (m, 3H), 2.17-2.03 (m, 3H). (1:1 major diastereomeric mixture)

### Compound 17: (6R,9S)-1-fluoro-12-(((2R,7As)-2-fluorotetrahydro-1H-pyrrolizin-7a(5H)-yl)methoxy)-2-(7-methoxynaphthalen-1-yl)-5a,6,7,8,9,10-hexahydro-5H-4-oxa-3,10a,11,13,14-pentaaza-6,9-methanonaphtho[1,8-ab]heptalene

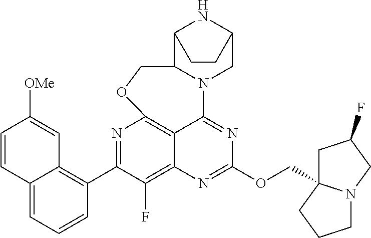 [SMILES: COc1ccc2cccc(-c3nc4c5c(nc(OC[C@@]67CCCN6C[C@H](F)C7)nc5c3F)N3CC5CCC(N5)C3CO4)c2c1]

MS (ESI) m/z: 601 (M+H)⁺.

¹H NMR (400 MHZ, CDCl₃) δ 7.89-7.83 (m, 1H), 7.80 (d, J=9.7 Hz, 1H), 7.63 (d, J=7.1 Hz, 1H), 7.41 (t, J=7.6 Hz, 1H), 7.19-7.14 (m, 2H), 5.34-5.22 (m, 1H), 5.06 (d, J=13.3 Hz, 1H), 4.48 (d, J=13.0 Hz, 1H), 4.31-4.25 (m, 2H), 4.13 (dd, J=19.1, 8.8 Hz, 2H), 3.80 (s, 3H), 3.77 (s, 1H), 3.59 (s, 1H), 3.32-3.23 (m, 2H), 3.20-3.12 (m, 2H), 3.02-2.95 (m, 1H), 2.31-2.17 (m, 3H), 1.98-1.93 (m, 2H), 1.88-1.84 (m, 3H), 1.82-1.76 (m, 3H). (1:1 major diastereomeric mixture)

### Compound 18: (6R,9S)-2-(3,8-difluoroisoquinolin-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

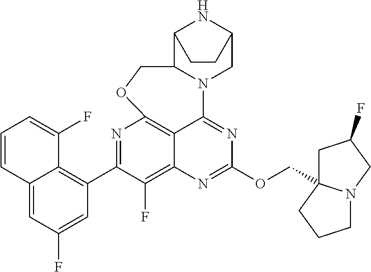

LC/MS (ESI) m/z: 608.6 [M+H]⁺.

¹H NMR (400 MHZ, Methanol-d₄) δ 7.92 (d, J=8.2 Hz, 1H), 7.81 (dt, J=12.9, 6.4 Hz, 1H), 7.71 (s, 1H), 7.35-7.25 (m, 1H), 5.50 (d, J=52.2 Hz, 1H), 5.14 (d, J=13.6 Hz, 1H), 4.69-4.53 (m, 4H), 4.28 (d, J=5.9 Hz, 1H), 3.97-3.87 (m, 2H), 3.82-3.67 (m, 3H), 3.39-3.32 (m, 2H), 2.65-2.43 (m, 2H), 2.40-2.32 (m, 1H), 2.29-2.21 (m, 2H), 2.13-2.06 (m, 1H), 2.03-1.87 (m, 4H). (1:1 major diastereomeric mixture)

### Compound 19: (6R,9S)-2-(8-ethynylnaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

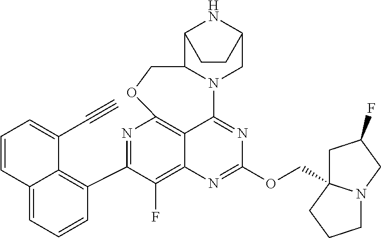 [SMILES: C#Cc1cccc2cccc(-c3nc4c5c(nc(OC[C@@]67CCCN6C[C@H](F)C7)nc5c3F)N3CC5CCC(N5)C3CO4)c12]

LC/MS (ESI) m/z: 595.2 [M+H]⁺.

¹H NMR (400 MHZ, Methanol-d₄) δ 8.07 (dd, J=17.9, 8.1 Hz, 2H), 7.79-7.71 (m, 1H), 7.70-7.46 (m, 3H), 5.49 (d, J=52.0 Hz, 1H), 5.13 (d, J=14.4 Hz, 1H), 4.66 (dd, J=13.3, 6.4 Hz, 1H), 4.59 (d, J=11.6 Hz, 1H), 4.52 (dt, J=13.6, 6.4 Hz, 2H), 4.25 (s, 1H), 3.93 (s, 1H), 3.87 (d, J=6.1 Hz, 1H), 3.83 (s, 1H), 3.72-3.61 (m, 2H), 3.37 (s, 2H), 3.22 (s, 1H), 2.69-2.51 (m, 1H), 2.48 (d, J=5.1 Hz, 1H), 2.35 (s, 1H), 2.24 (s, 2H), 2.03 (d, J=9.4 Hz, 2H), 2.01-1.93 (m, 2H), 1.92 (s, 1H). (1:1 major diastereomeric mixture)

### Compound 20: 8-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-1-naphthonitrile

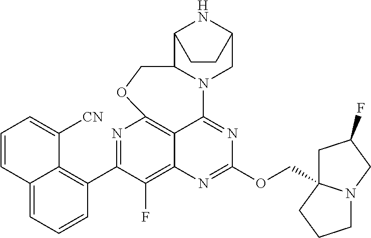 [SMILES: N#Cc1cccc2cccc(-c3nc4c5c(nc(OC[C@@]67CCCN6C[C@H](F)C7)nc5c3F)N3CC5CCC(N5)C3CO4)c12]

LC/MS ESI (m/z): 596.3 [M+1]⁺

¹H NMR (400 MHZ, Methanol-d₄) δ 8.36 (d, J=7.9 Hz, 1H), 8.21 (d, J=9.3 Hz, 1H), 8.10-7.99 (m, 1H), 7.82-7.65 (m, 3H), 5.39 (d, J=54.0 Hz, 1H), 5.10 (d, J=13.4 Hz, 1H), 4.60 (s, 1H), 4.47 (dd, J=18.3, 9.1 Hz, 2H), 4.37 (d, J=11.1 Hz, 1H), 4.19 (s, 1H), 3.81-3.67 (m, 2H), 3.47 (s, 3H), 3.21 (d, J=28.1 Hz, 2H), 2.51-2.22 (m, 3H), 2.12 (s, 2H), 2.02-1.81 (m, 5H). (1:1 major diastereomeric mixture)

### Compound 21: (6R,9S)-2-(2-cyclopropyl-3-fluorophenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

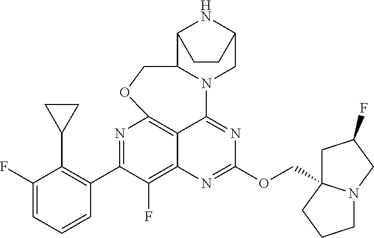 [SMILES: Fc1cccc(-c2nc3c4c(nc(OC[C@@]56CCCN5C[C@H](F)C6)nc4c2F)N2CC4CCC(N4)C2CO3)c1C1CC1]

LCMS ESI (m/z): 579 [M+H]⁺

¹HNMR (400 MHZ, Methanol-d₄) δ 7.39-7.31 (m, 1H), 7.18 (dd, J=17.0, 7.8 Hz, 2H), 5.53 (d, J=52.2 Hz, 1H), 5.18 (d, J=13.4 Hz, 1H), 4.70-4.52 (m, 4H), 4.32 (d, J=6.1 Hz, 1H), 4.03 (dd, J=16.7, 5.1 Hz, 2H), 3.94-3.70 (m, 3H), 3.39 (d, J=14.8 Hz, 2H), 2.70-2.47 (m, 2H), 2.38 (d, J=4.4 Hz, 1H), 2.28 (td, J=12.2, 6.6 Hz, 2H), 2.12 (d, J=10.9 Hz, 1H), 2.01 (td, J=13.9, 6.4 Hz, 4H), 1.84 (t, J=5.4 Hz, 1H), 0.67 (d, J=8.6 Hz, 2H), 0.38 (t, J=4.9 Hz, 2H). (1:1 major diastereomeric mixture)

### Compound 22: 2-amino-4-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)benzo[b]thiophene-3-carbonitrile

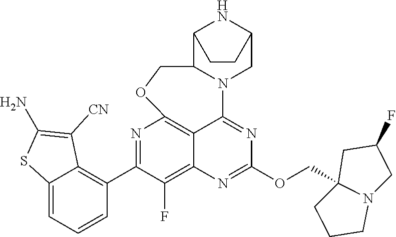 [SMILES: N#Cc1c(N)sc2cccc(-c3nc4c5c(nc(OC[C@@]67CCCN6C[C@H](F)C7)nc5c3F)N3CC5CCC(N5)C3CO4)c12]

MS (ESI) m/z: 617.4 [M+H]⁺.

¹H NMR (400 MHz, Methanol-d₄) δ 7.69 (dd, J=7.9, 1.0 Hz, 1H), 7.36 (d, J=6.7 Hz, 1H), 7.26 (t, J=7.7 Hz, 1H), 5.47 (d, J=51.1 Hz, 1H), 5.11 (d, J=13.7 Hz, 1H), 4.60 (ddd, J=18.6, 12.7, 4.4 Hz, 3H), 4.49 (dd, J=9.2, 4.4 Hz, 2H), 4.20 (d, J=6.7 Hz, 1H), 3.85-3.74 (m, 3H), 3.71-3.65 (m, 2H), 2.59-2.20 (m, 6H), 2.04 (d, J=11.6 Hz, 1H), 1.91 (dt, J=21.0, 6.4 Hz, 4H). (1:1 major diastereomeric mixture)

Compound 23: (6R,9S)-2-(3-chloro-2-cyclopropylphenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

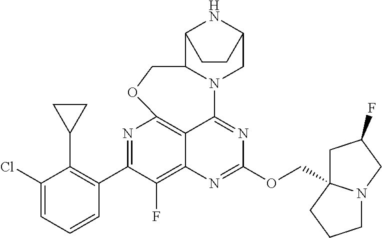 [SMILES: Fc1c(-c2cccc(Cl)c2C2CC2)nc2c3c(nc(OC[C@@]45CCCN4C[C@H](F)C5)nc13)N1CC3CCC(N3)C1CO2]

MS (ESI): m/z=595 [M+1]⁺.

¹H NMR (400 MHZ, Methanol-d₄) δ 7.53 (dd, J=6.2, 3.1 Hz, 1H), 7.38-7.31 (m, 2H), 5.52 (d, J=52.5 Hz, 1H), 5.22-5.08 (m, 1H), 4.64 (dd, J=13.5, 11.5 Hz, 2H), 4.58-4.49 (m, 2H), 4.26 (d, J=6.9 Hz, 1H), 3.99-3.72 (m, 5H), 3.42-3.34 (m, 2H), 2.69-2.49 (m, 2H), 2.37 (s, 1H), 2.27 (dq, J=12.1, 6.3 Hz, 2H), 2.17-1.90 (m, 6H), 0.74 (s, 2H), 0.24-0.10 (m, 2H). (1:1 major diastereomeric mixture)

### Compound 24: 3-chloro-4-cyclopropyl-5-((6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)phenol

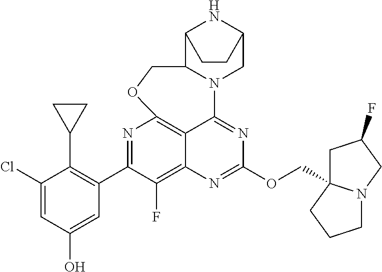 [SMILES: Oc1cc(Cl)c(C2CC2)c(-c2nc3c4c(nc(OC[C@@]56CCCN5C[C@H](F)C6)nc4c2F)N2CC4CCC(N4)C2CO3)c1]

LC/MS (ESI): m/z=611 [M+1]⁺.

¹H NMR (400 MHZ, Methanol-d₄) δ 6.95 (d, J=2.6 Hz, 1H), 6.75 (d, J=2.5 Hz, 1H), 5.53 (d, J=52.6 Hz, 1H), 5.18 (d, J=14.2 Hz, 1H), 4.70-4.59 (m, 3H), 4.58-4.51 (m, 1H), 4.29 (d, J=5.5 Hz, 1H), 4.01 (dd, J=16.0, 6.1 Hz, 2H), 3.96-3.77 (m, 3H), 3.46-3.33 (m, 2H), 2.72-2.52 (m, 2H), 2.38 (s, 1H), 2.30 (dt, J=10.9, 6.2 Hz, 2H), 2.15 (s, 1H), 2.08-1.93 (m, 4H), 1.88-1.79 (m, 1H), 0.64 (s, 2H), 0.11 (d, J=5.1 Hz, 2H). (1:1 major diastereomeric mixture)

### Example 4

### Preparation of Single Diastereomer tert-butyl (1S,2S,5R)-2-(hydroxymethyl)-3,8-diazabicyclo[3.2.1]octane-8-carboxylate (INT2)

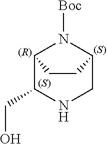

### Methyl (2R)-5-methoxy-3,4-dihydro-2H-pyrrole-2-carboxylate

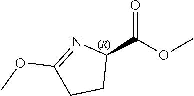

To a solution of methyl (2R)-5-oxopyrrolidine-2-carboxylate (88 g, 0.61 mol) in DCM (1 L) was added trimethyloxonium tetrafluoroborate (100 g, 0.67 mol). The reaction mixture was stirred at room temperature for 18 hrs. The reaction mixture was quenched with saturated aq. NaHCO₃ solution at 0° C. The layers were separated. The organic layer was washed with saturated aq. NaHCO₃ solution and brine, dried over anhydrous Na₂SO₄, filtered and concentrated under reduced pressure to dryness. The residue was purified by flash chromatography (silica gel, 0-30% EtOAc in PE) to afford methyl (2R)-5-methoxy-3,4-dihydro-2H-pyrrole-2-carboxylate (69 g, 71% yield) as a yellow oil. MS ESI (m/z): 158 (M+H)⁺.

### Methyl (R)-5-(2-ethoxy-1-nitro-2-oxoethylidene) pyrrolidine-2-carboxylate

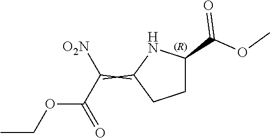

A mixture of methyl (2R)-5-methoxy-3,4-dihydro-2H-pyrrole-2-carboxylate (69 g, 0.44 mol) and ethyl 2-nitroacetate (38.9 mL, 0.35 mol) was stirred at 60° C. for 18 hrs. The mixture was concentrated to dryness and the residue was purified by flash chromatography (silica gel, 0-25% EtOAc in PE) to give methyl (R)-5-(2-ethoxy-1-nitro-2-oxoethylidene) pyrrolidine-2-carboxylate (37 g, 33% yield) as a yellow oil. MS ESI (m/z): 259 (M+H)⁺.

### Ethyl (1S,2S,5R)-4-oxo-3,8-diazabicyclo[3.2.1]octane-2-carboxylate

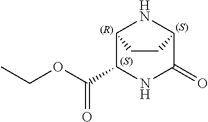

To a solution of methyl (R)-5-(2-ethoxy-1-nitro-2-oxoethylidene) pyrrolidine-2-carboxylate (32 g, 0.12 mol) in EtOH (200 mL) was added Pd/C (10 g, 10% wt). The reaction mixture was degassed under N₂ atmosphere for three times and stirred under a H₂ balloon at 50° C. for 20 hrs then at 80° C. for 48 hrs. The mixture was filtered, and the filtrate was concentrated under reduced pressure to dryness. The residue was purified by flash chromatography (silica gel, 0-10% MeOH in DCM) to give ethyl (1S,2S,5R)-4-oxo-3,8-diazabicyclo[3.2.1]octane-2-carboxylate (10 g, 41% yield) as a white solid. MS ESI (m/z): 199 (M+H)⁺.

### 8-Tert-butyl 2-ethyl (1S,2S,5R)-4-oxo-3,8-diazabicyclo[3.2.1]octane-2,8-dicarboxylate

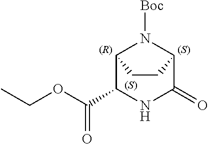

To a solution of ethyl (1S,2S,5R)-4-oxo-3,8-diazabicyclo[3.2.1]octane-2-carboxylate (1.54 g, 7.77 mmol) in THF (20 mL) and water (5 mL) was added NaHCO₃ (2.20 g, 10.1 mmol) and Boc₂O (2.2 g, 10.1 mmol) and the mixture was stirred at room temperature for 16 hrs. The mixture was diluted with EtOAc, washed with water and brine, dried over anhydrous Na₂SO₄, filtered and concentrated to dryness. The residue was purified by flash chromatography (silica gel, 0-10% MeOH in DCM) to give 8-tert-butyl 2-ethyl (1S,2S,5R)-4-oxo-3,8-diazabicyclo[3.2.1]octane-2,8-dicarboxylate (1.9 g, 81% yield) as a white solid. MS ESI (m/z): 299 (M+H)⁺.

### Tert-butyl (1S,2S,5R)-2-(hydroxymethyl)-3,8-diazabicyclo[3.2.1]octane-8-carboxylate

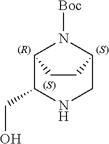

To a suspension of LiAlH₄ (1.91 g, 50.4 mmol) in THF (20 mL) was added a solution of 8-tert-butyl 2-ethyl (1R,2R,5S)-4-oxo-3,8-diazabicyclo[3.2.1]octane-2,8-dicarboxylate (1.88 g, 6.30 mmol) in THF (20 mL) dropwise at 0° C. and the mixture was stirred under N₂ atmosphere at 0° C. for 5 hrs. The reaction was quenched successively with water (1.9 mL), aq NaOH (1.9 mL, 15% w.t.) and water (5.7 mL) at 0° C. The mixture was stirred at 0° C. for 30 mins and filtered. The filter cake was washed with DCM/MeOH (2×50 mL, 10/1). The filtrate was dried over anhydrous Na₂SO₄, filtered and concentrated under reduced pressure to dryness. The residue was purified by flash chromatography (silica gel, 0-15% MeOH in DCM) to give tert-butyl (1S,2S,5R)-2-(hydroxymethyl)-3,8-diazabicyclo[3.2.1]octane-8-carboxylate (900 mg, 59% yield) as a yellow oil.

MS (ESI) m/z: 243 (M+H)⁺.

¹H NMR (400 MHZ, DMSO) δ 4.60 (t, J=5.3 Hz, 1H), 3.95 (s, 2H), 3.21-3.17 (m, 2H), 2.73 (d, J=10.8 Hz, 2H), 2.55 (d, J=11.4 Hz, 1H), 1.82-1.51 (m, 4H), 1.43-1.38 (m, 9H).

The following compounds can be prepared with single diastereomer INT2 and an appropriate boronic ester following the procedure described in Scheme 6.

### Compound 25: (5aS,6S,9R)-2-(8-ethynylnaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

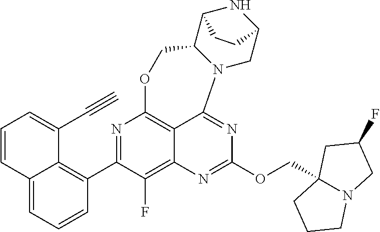 [SMILES: C#Cc1cccc2cccc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 595.5 [M+H]⁺.

¹H NMR (400 MHZ, Methanol-d₄) δ 8.07 (dd, J=17.5, 8.3 Hz, 2H), 7.78-7.71 (m, 1H), 7.66 (q, J=7.6 Hz, 1H), 7.62-7.49 (m, 2H), 5.48 (d, J=52.6 Hz, 1H), 5.12 (dd, J=13.9, 2.3 Hz, 1H), 4.69-4.63 (m, 1H), 4.63-4.56 (m, 1H), 4.50 (dt, J=13.3, 4.9 Hz, 2H), 4.23 (s, 1H), 3.91 (s, 1H), 3.87-3.65 (m, 4H), 3.34 (d, J=3.9 Hz, 2H), 3.21 (s, 1H), 2.64-2.43 (m, 2H), 2.34 (s, 1H), 2.25 (d, J=6.7 Hz, 2H), 1.97 (ddd, J=29.7, 14.4, 4.5 Hz, 5H).

### Compound 26: 1-(8-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-6-hydroxynaphthalen-1-yl) ethan-1-one

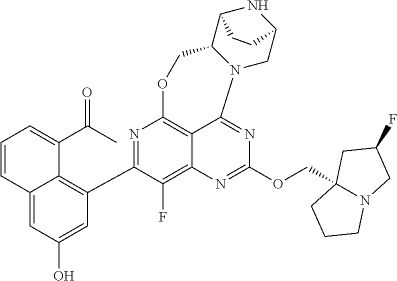 [SMILES: CC(=O)c1cccc2cc(O)cc(-c3nc4c5c(nc(OC[C@@]67CCCN6C[C@H](F)C7)nc5c3F)N3C[C@H]5CC[C@H](N5)[C@H]3CO4)c12]

LC/MS ESI (m/z): 629 [M+H]⁺.

¹H NMR (400 MHZ, Methanol-d₄) δ 8.04 (s, 1H), 7.79 (d, J=8.2 Hz, 1H), 7.66 (d, J=7.2 Hz, 1H), 7.50 (t, J=7.7 Hz, 1H), 7.22 (d, J=1.8 Hz, 1H), 5.44 (d, J=52.8 Hz, 1H), 4.45 (d, J=11.2 Hz, 1H), 4.36 (d, J=11.2 Hz, 1H), 4.08 (s, 1H), 3.80 (s, 1H), 3.74 (d, J=11.5 Hz, 1H), 3.65 (d, J=2.5 Hz, 1H), 3.57 (d, J=15.0 Hz, 3H), 3.34 (s, 2H), 3.30 (s, 4H), 2.54 (d, J=7.3 Hz, 2H), 2.46-2.40 (m, 1H), 2.30-2.23 (m, 1H), 2.16 (dd, J=11.1, 6.2 Hz, 2H), 2.05-1.87 (m, 5H).

### Compound 27: 5-chloro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

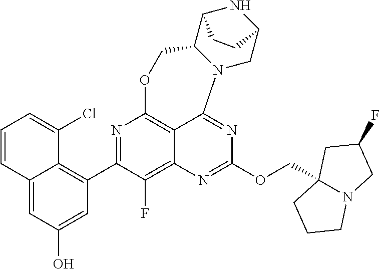 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(Cl)cccc2c1]

LC/MS (ESI) m/z: 621.4 [M+H]⁺.

¹H NMR (400 MHZ, Methanol-d₄) δ 7.80-7.69 (m, 1H), 7.40-7.30 (m, 3H), 7.15 (dd, J=34.3, 2.5 Hz, 1H), 5.50 (d, J=52.1 Hz, 1H), 5.20-5.08 (m, 1H), 4.70-4.44 (m, 5H), 4.30-4.20 (m, 1H), 3.98-3.67 (m, 5H), 3.36 (s, 1H), 2.66-2.22 (m, 5H), 2.12-1.88 (m, 5H).

### Compound 28: 5-ethyl-6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

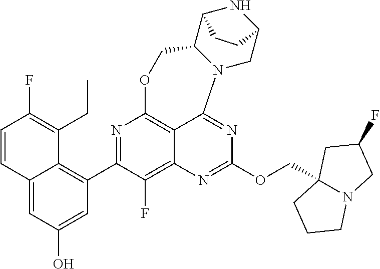 [SMILES: CCc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 633.5 [M+H]⁺.

¹H NMR (400 MHZ, Methanol-d₄) δ 7.69-7.63 (m, 1H), 7.25 (ddd, J=14.6, 7.0, 3.9 Hz, 2H), 7.04 (dd, J=38.2, 2.5 Hz, 1H), 5.45 (d, J=52.6 Hz, 1H), 5.09 (dd, J=18.8, 14.6 Hz, 1H), 4.63 (dd, J=12.3, 6.0 Hz, 1H), 4.56-4.43 (m, 3H), 4.19 (dd, J=15.1, 7.0 Hz, 1H), 3.83 (d, J=5.8 Hz, 1H), 3.79-3.58 (m, 4H), 3.27 (s, 1H), 2.61-1.76 (m, 13H), 0.85 (dt, J=36.9, 7.4 Hz, 3H).

### Compound 29: (5aS,6S,9R)-2-(1-ethynylisoquinolin-8-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

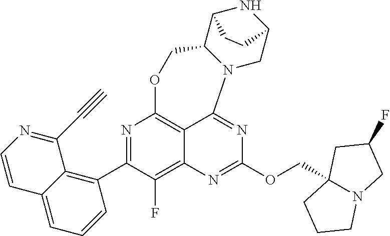 [SMILES: C#Cc1nccc2cccc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 596 [M+H]⁺.

¹H NMR (400 MHZ, Methanol-d₄) δ 8.50 (t, J=5.6 Hz, 1H), 8.17 (d, J=8.3 Hz, 1H), 8.00 (dd, J=5.6, 2.9 Hz, 1H), 7.96-7.90 (m, 1H), 7.77 (dd, J=28.1, 6.6 Hz, 1H), 5.49 (d, J=52.3 Hz, 1H), 5.15 (dd, J=14.0, 2.1 Hz, 1H), 4.70-4.49 (m, 5H), 4.28 (s, 1H), 3.95-3.87 (m, 2H), 3.77-3.67 (m, 3H), 3.36 (d, J=11.8 Hz, 2H), 2.52 (dd, J=30.3, 15.0 Hz, 2H), 2.35 (d, J=5.7 Hz, 1H), 2.25 (d, J=10.7 Hz, 2H), 2.04-1.91 (m, 5H).

### Compound 30: 6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

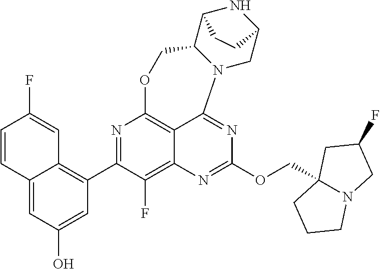 [SMILES: Oc1cc(-c2nc3c4c(nc(OC[C@@]56CCCN5C[C@H](F)C6)nc4c2F)N2C[C@H]4CC[C@H](N4)[C@H]2CO3)c2cc(F)ccc2c1]

LC/MS (ESI) m/z: 605.4 [M+H]⁺.

¹H NMR (400 MHZ, Methanol-d₄) δ 7.78 (dd, J=9.6, 5.7 Hz, 1H), 7.29 (d, J=2.4 Hz, 1H), 7.28-7.25 (m, 2H), 7.23 (s, 1H), 5.33 (d, J=54.0 Hz, 1H), 5.06 (d, J=11.4 Hz, 1H), 4.60 (s, 2H), 4.48 (dd, J=13.3, 7.4 Hz, 1H), 4.30 (dd, J=28.0, 10.6 Hz, 2H), 4.15 (d, J=7.5 Hz, 1H), 3.76-3.72 (m, 2H), 3.68-3.64 (m, 3H), 3.23 (d, J=19.1 Hz, 2H), 3.06 (d, J=5.6 Hz, 1H), 2.28-2.17 (m, 2H), 2.07-1.79 (m, 8H).

### Compound 31: 5-ethynyl-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

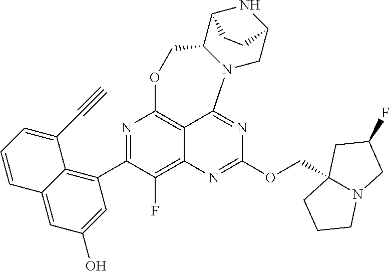 [SMILES: C#Cc1cccc2cc(O)cc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 611.5 [M+H]⁺.

¹H NMR (400 MHZ, Methanol-d₄) δ 8.39 (s, 1H), 7.80 (d, J=8.1 Hz, 1H), 7.54-7.47 (m, 1H), 7.42-7.35 (m, 1H), 7.31 (s, 1H), 7.14 (dd, J=31.9, 2.5 Hz, 1H), 5.47 (d, J=52.3 Hz, 1H), 5.09 (d, J=12.2 Hz, 1H), 4.64-4.45 (m, 5H), 4.20 (s, 1H), 3.74 (dd, J=61.3, 20.6 Hz, 5H), 3.48 (s, 1H), 3.18 (d, J=37.4 Hz, 1H), 2.63-2.42 (m, 2H), 2.33 (s, 1H), 2.22 (s, 2H), 1.94 (dd, J=34.4, 22.0 Hz, 5H).

### Compound 32: (5aS,6R,9S)-2-(8-ethynyl-7-fluoronaphthalen-1-yl)-1-fluoro-12-(((2S,4R)-4-fluoro-1-methylpyrrolidin-2-yl)methoxy)-5a,6,7,8,9,10-hexahydro-5H-4-oxa-3,10a,11,13,14-pentaaza-6,9-methanonaphtho[1,8-ab]heptalene

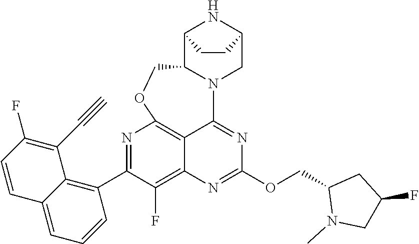 [SMILES: C#Cc1c(F)ccc2cccc(-c3nc4c5c(nc(OC[C@@H]6C[C@@H](F)CN6C)nc5c3F)N3C[C@H]5CC[C@H](N5)[C@H]3CO4)c12]

LC/MS (ESI) m/z: 587.4 [M+H]⁺.

¹H NMR (400 MHZ, Methanol-d₄) δ 8.08 (tt, J=6.0, 2.9 Hz, 2H), 7.65-7.55 (m, 2H), 7.42 (td, J=9.0, 5.1 Hz, 1H), 5.18 (d, J=55.5 Hz, 1H), 5.09-5.01 (m, 1H), 4.60 (ddd, J=13.3, 7.0, 2.1 Hz, 1H), 4.50 (dd, J=5.2, 2.4 Hz, 2H), 4.48-4.41 (m, 1H), 4.13 (d, J=7.3 Hz, 1H), 3.72 (s, 1H), 3.66-3.61 (m, 1H), 3.57-3.43 (m, 2H), 3.24 (t, J=12.1 Hz, 1H), 3.16-3.11 (m, 1H), 2.72-2.60 (m, 1H), 2.55 (s, 3H), 2.31 (d, J=18.4 Hz, 1H), 2.09-2.01 (m, 1H), 1.96-1.81 (m, 4H).

### Compound 36: (5aS,6R,9S)-2-(3-chloro-2-cyclopropyl-5-fluorophenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

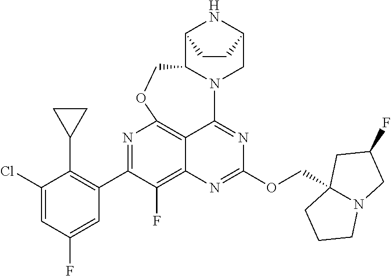 [SMILES: Fc1cc(Cl)c(C2CC2)c(-c2nc3c4c(nc(OC[C@@]56CCCN5C[C@H](F)C6)nc4c2F)N2C[C@H]4CC[C@H](N4)[C@H]2CO3)c1]

LC/MS (ESI) m/z: 613 (M+H)⁺.

¹H NMR (400 MHZ, CDCl₃) δ 7.05-7.01 (m, 1H), 6.98-6.96 (m, 1H), 5.22-5.08 (m, 2H), 4.88 (dd, J=13.3, 2.1 Hz, 1H), 4.36-4.30 (m, 1H), 4.17-4.01 (m, 4H), 3.95 (d, J=7.6 Hz, 1H), 3.62 (s, 1H), 3.45 (s, 1H), 3.24-2.99 (m, 5H), 2.87-2.86 (m, 1H), 2.22-2.01 (m, 5H), 1.17-1.10 (m, 7H).

### Compound 37: (7a-((((5aS,6R,9S)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalen-12-yl)oxy)methyl)hexahydro-1H-pyrrolizin-3-yl)methyl dimethylcarbamate (Trans Mixture)

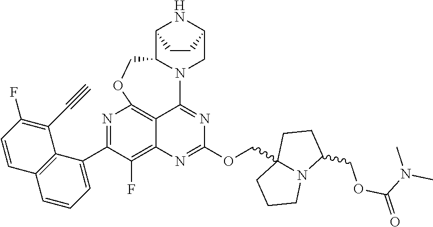 [SMILES: C#Cc1c(F)ccc2cccc(-c3nc4c5c(nc(OCC67CCCN6C(COC(=O)N(C)C)CC7)nc5c3F)N3C[C@H]5CC[C@H](N5)[C@H]3CO4)c12]

LC/MS (ESI) m/z: 696 (M+H)⁺.

¹H NMR (400 MHZ, CDCl₃) δ 7.93-7.88 (m, 2H), 7.56-7.53 (m, 2H), 7.33-7.29 (m, 1H), 5.08-4.98 (m, 1H), 4.47-4.44 (m, 1H), 4.35-4.24 (m, 5H), 4.13-4.05 (m, 1H), 3.78 (s, 1H), 3.56-3.53 (m, 2H), 3.20 (t, J=12.5 Hz, 1H), 3.07-2.73 (m, 9H), 2.28-2.23 (m, 2H), 1.85-1.79 (m, 10H).

### Compound 38: (7a-((((5aS,6R,9S)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalen-12-yl)oxy)methyl)hexahydro-1H-pyrrolizin-3-yl)methyl dimethylcarbamate (Cis Mixture)

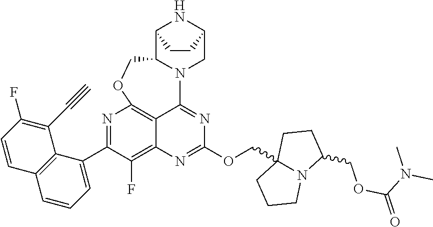 [SMILES: C#Cc1c(F)ccc2cccc(-c3nc4c5c(nc(OCC67CCCN6C(COC(=O)N(C)C)CC7)nc5c3F)N3C[C@H]5CC[C@H](N5)[C@H]3CO4)c12]

LC/MS (ESI) m/z: 696 (M+H)⁺.

¹H NMR (400 MHZ, CDCl₃) δ 7.94-7.88 (m, 2H), 7.62-7.55 (m, 2H), 7.33-7.28 (m, 1H), 5.17-5.08 (m, 1H), 4.63-4.34 (m, 5H), 4.18-4.01 (m, 4H), 3.64-3.40 (m, 1H), 3.32-3.03 (m, 4H), 2.90 (s, 6H), 2.42-2.13 (m, 5H), 1.95-1.54 (m, 7H).

### Compound 39: (5aS,6R,9S)-2-(3-chloro-2-cyclopropyl-4-fluorophenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

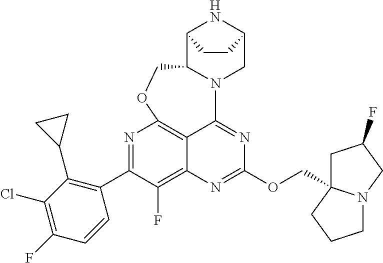 [SMILES: Fc1ccc(-c2nc3c4c(nc(OC[C@@]56CCCN5C[C@H](F)C6)nc4c2F)N2C[C@H]4CC[C@H](N4)[C@H]2CO3)c(C2CC2)c1Cl]

LC/MS (ESI) m/z: 613 (M+H)⁺.

¹H NMR (400 MHZ, CDCl₃) δ 7.42 (t, J=7.5 Hz, 1H), 6.79 (d, J=8.3 Hz, 1H), 5.28 (d, J=53.0 Hz, 1H), 5.00 (d, J=12.2 Hz, 1H), 4.46 (d, J=12.4 Hz, 1H), 4.28-4.15 (m, 3H), 4.06 (d, J=7.1 Hz, 1H), 3.74-3.58 (m, 2H), 3.32-3.10 (m, 4H), 3.02-2.96 (m, 1H), 2.31-2.19 (m, 5H), 1.92-1.81 (m, 7H), 1.09-1.06 (m, 2H), 0.76-0.74 (m, 2H).

### Compound 40: (5aS)-2-(8-ethynyl-7-fluoronaphthalen-1-yl)-1-fluoro-12-((1-(morpholinomethyl)cyclopropyl)methoxy)-5a,6,7,8,9,10-hexahydro-5H-4-oxa-3,10a,11,13,14-pentaaza-6,9-methanonaphtho[1,8-ab]heptalene

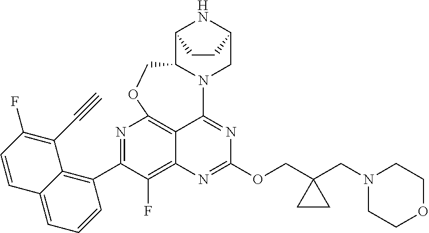 [SMILES: C#Cc1c(F)ccc2cccc(-c3nc4c5c(nc(OCC6(CN7CCOCC7)CC6)nc5c3F)N3C[C@H]5CC[C@H](N5)[C@H]3CO4)c12]

LC/MS (ESI) (m/z): 625.8 (M+H)⁺.

¹H NMR (400 MHZ, CDCl₃) δ 7.97-7.95 (m, 2H), 7.59-7.54 (m, 2H), 7.34-7.28 (m, 1H), 5.08-5.02 (m, 1H), 4.53-4.48 (m, 3H), 4.31-4.29 (m, 1H), 4.17-4.12 (m, 1H), 3.82 (s, 1H), 3.71-3.68 (m, 5H), 3.28-3.26 (m, 1H), 2.98 (d, J=19.8 Hz, 1H), 2.55-2.48 (m, 6H), 1.96-1.83 (m, 4H), 0.73 (s, 2H), 0.51 (s, 2H).

### Compound 41: (5aS)-2-(8-ethynyl-7-fluoronaphthalen-1-yl)-1-fluoro-12-((1-(((R)-2-methylmorpholino)methyl)cyclopropyl)methoxy)-5a,6,7,8,9,10-hexahydro-5H-4-oxa-3,10a,11,13,14-pentaaza-6,9-methanonaphtho[1,8-ab]heptalene

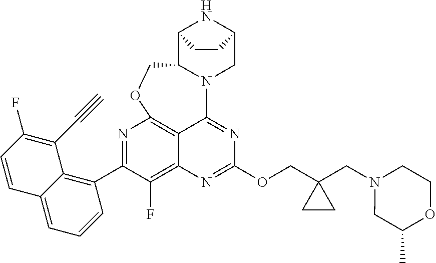 [SMILES: C#Cc1c(F)ccc2cccc(-c3nc4c5c(nc(OCC6(CN7CCO[C@H](C)C7)CC6)nc5c3F)N3C[C@H]5CC[C@H](N5)[C@H]3CO4)c12]

LC/MS (ESI) m/z: 639 (M+H)⁺.

¹H NMR (400 MHZ, CDCl₃) δ 7.95-7.92 (m, 2H), 7.66-7.51 (m, 2H), 7.35-7.29 (m, 1H), 5.04-4.99 (m, 1H), 4.52-4.35 (m, 3H), 4.32-4.22 (m, 1H), 4.13-4.04 (m, 1H), 3.81-3.78 (m, 2H), 3.67-3.60 (m, 3H), 3.24-3.18 (m, 1H), 2.98-2.84 (m, 3H), 2.42-2.31 (m, 2H), 1.93-1.72 (m, 6H), 1.14-1.09 (m, 3H), 0.73-0.70 (s, 2H), 0.48-0.45 (s, 2H).

### Compound 42: (5aS)-2-(8-ethynyl-7-fluoronaphthalen-1-yl)-1-fluoro-12-((1-(((R)-3-methylmorpholino)methyl)cyclopropyl)methoxy)-5a,6,7,8,9,10-hexahydro-5H-4-oxa-3,10a,11,13,14-pentaaza-6,9-methanonaphtho[1,8-ab]heptalene

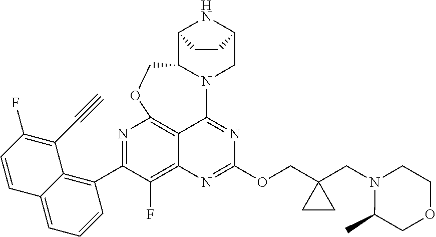 [SMILES: C#Cc1c(F)ccc2cccc(-c3nc4c5c(nc(OCC6(CN7CCOC[C@H]7C)CC6)nc5c3F)N3C[C@H]5CC[C@H](N5)[C@H]3CO4)c12]

LC/MS (ESI) m/z: 639 (M+H)⁺.

¹H NMR (400 MHZ, CDCl₃) δ 7.94-7.89 (m, 2H), 7.67-7.55 (m, 2H), 7.34-7.29 (m, 1H), 5.07-4.99 (m, 1H), 4.73-4.45 (m, 3H), 4.26-4.06 (m, 3H), 3.76-3.74 (m, 2H), 3.63-3.60 (m, 3H), 3.35 (d, J=12.7 Hz, 1H), 3.23-3.19 (m, 2H), 3.04-2.90 (m, 2H), 2.40 (s, 1H), 2.26-2.18 (m, 1H), 1.90-1.78 (m, 4H), 0.97-0.90 (m, 3H), 0.75-0.61 (m, 2H), 0.56-0.36 (m, 2H).

### Compound 46: (5aS,6R,9S)-12-((1-((4-oxa-7-azaspiro[2.5]octan-7-yl)methyl)cyclopropyl)methoxy)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalene

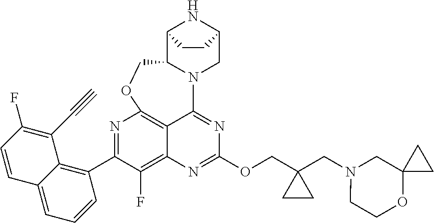 [SMILES: C#Cc1c(F)ccc2cccc(-c3nc4c5c(nc(OCC6(CN7CCOC8(CC8)C7)CC6)nc5c3F)N3C[C@H]5CC[C@H](N5)[C@H]3CO4)c12]

LC/MS (ESI) m/z: 651 (M+H)⁺.

¹H NMR (400 MHZ, CDCl₃) δ 7.88-7.82 (m, 2H), 7.59-7.45 (m, 2H), 7.27-7.22 (m, 1H), 4.93 (t, J=12.2 Hz, 1H), 4.45-4.35 (m, 2H), 4.32-4.30 (m, 1H), 4.29-4.26 (m, 1H), 4.10-3.99 (m, 1H), 3.75-3.68 (m, 3H), 3.58 (s, 1H), 3.17 (t, J=13.5 Hz, 1H), 2.95-2.91 (m, 1H), 2.60-2.45 (m, 7H), 1.86-1.82 (m, 3H), 0.69-0.66 (m, 4H), 0.56-0.54 (m, 2H), 0.45-0.43 (m, 2H).

### Compound 47: (7a-((((5aS,6R,9S)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalen-12-yl)oxy)methyl)hexahydro-1H-pyrrolizin-3-yl)methyl morpholine-4-carboxylate

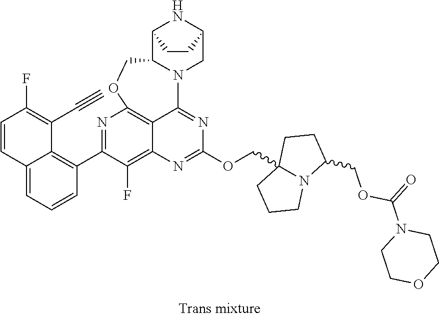 [SMILES: C#Cc1c(F)ccc2cccc(-c3nc4c5c(nc(OCC67CCCN6C(COC(=O)N6CCOCC6)CC7)nc5c3F)N3C[C@H]5CC[C@H](N5)[C@H]3CO4)c12]

LC/MS (ESI) m/z: 738 (M+H)⁺.

¹H NMR (400 MHZ, CDCl₃) δ 7.97-7.87 (m, 2H), 7.66-7.50 (m, 2H), 7.34-7.27 (m, 1H), 5.03 (dd, J=11.9, 7.9 Hz, 1H), 4.52-4.35 (m, 2H), 4.31-4.14 (m, 4H), 4.08 (dd, J=18.1, 7.3 Hz, 1H), 3.75-3.59 (m, 6H), 3.49-3.47 (m, 5H), 3.17 (dd, J=20.1, 9.6 Hz, 1H), 2.96-2.91 (m, 2H), 2.74 (dd, J=16.2, 8.3 Hz, 1H), 2.26-2.24 (m, 1H), 2.02-1.75 (m, 10H), 1.58-1.51 (m, 1H).

### Compound 48: (7a-((((5aS,6R,9S)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalen-12-yl)oxy)methyl)hexahydro-1H-pyrrolizin-3-yl) methanol

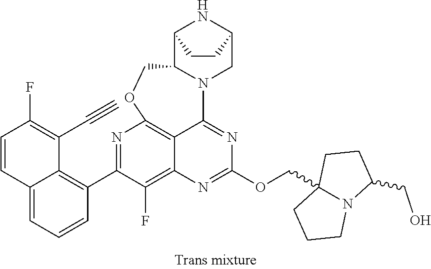 [SMILES: C#Cc1c(F)ccc2cccc(-c3nc4c5c(nc(OCC67CCCN6C(CO)CC7)nc5c3F)N3C[C@H]5CC[C@H](N5)[C@H]3CO4)c12]

LC/MS (ESI) m/z: 625 (M+H)⁺.

¹H NMR (400 MHZ, CDCl₃) δ 7.97-7.89 (m, 2H), 7.57-7.52 (m, 2H), 7.33-7.26 (m, 1H), 4.98-4.95 (m, 1H), 4.57-4.49 (m, 3H), 4.30-4.25 (m, 3H), 4.02-4.01 (m, 1H), 3.93-3.90 (m, 2H), 3.73-3.70 (m, 2H), 3.41-3.32 (m, 1H), 3.13-3.07 (m, 2H), 2.49-2.43 (m, 2H), 1.92-1.78 (m, 10H).

### Compound 49: (5aS,6R,9S)-12-((1-((3-oxa-8-azabicyclo[3.2.1]octan-8-yl)methyl)cyclopropyl)methoxy)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalene

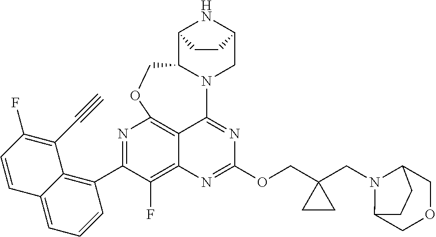 [SMILES: C#Cc1c(F)ccc2cccc(-c3nc4c5c(nc(OCC6(CN7C8CCC7COC8)CC6)nc5c3F)N3C[C@H]5CC[C@H](N5)[C@H]3CO4)c12]

LC/MS (ESI) m/z: 651 (M+H)⁺.

¹H NMR (400 MHZ, CD₃OD) δ 8.12-8.09 (m, 2H), 7.66-7.59 (m, 2H), 7.47-7.41 (m, 1H), 5.26-5.21 (m, 1H), 4.87-4.49 (m, 6H), 4.35-4.22 (m, 5H), 3.86-3.83 (m, 2H), 3.58-3.50 (m, 2H), 3.31-3.25 (m, 2H), 2.31-2.03 (m, 8H), 0.96-0.94 (m, 2H), 0.93-0.90 (m, 2H).

### Compound 50: (R)-2-(8-ethynyl-7-fluoronaphthalen-1-yl)-1-fluoro-12-(((2R,7aS)-2-fluorotetrahydro-1H-pyrrolizin-7a(5H)-yl)methoxy)-6,6a,7,8,9,10-hexahydro-5H-4-oxa-3,8,10a,11,13-pentaazabenzo[4,5]cycloocta[1,2,3-de]naphthalen

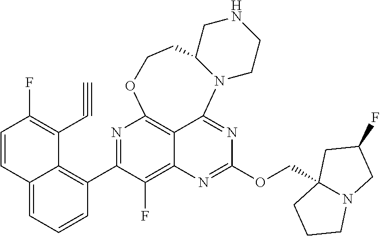 [SMILES: C#Cc1c(F)ccc2cccc(-c3nc4c5c(nc(OC[C@@]67CCCN6C[C@H](F)C7)nc5c3F)N3CCNC[C@H]3CCO4)c12]

MS (ESI) m/z: 601 [M+H]⁺.

¹H NMR (400 MHZ, MeOD) δ 8.14-8.01 (m, 2H), 7.72-7.52 (m, 2H), 7.42 (dd, J=16.2, 8.9 Hz, 1H), 6.31 (d, J=4.7 Hz, 1H), 5.35-5.21 (m, 1H), 4.56 (d, J=14.5 Hz, 4H), 4.45-4.24 (m, 3H), 3.67 (s, 1H), 3.58 (s, 2H), 3.43 (s, 2H), 3.08 (d, J=35.6 Hz, 2H), 2.30-2.10 (m, 4H), 1.98 (dd, J=34.6, 28.6 Hz, 6H).

### Compound 51: (6aR,7S,10R)-2-(8-ethynyl-7-fluoronaphthalen-1-yl)-1-fluoro-13-(((2R,7aS)-2-fluorotetrahydro-1H-pyrrolizin-7a(5H)-yl)methoxy)-5,6,6a,7,8,9,10,11-octahydro-4-oxa-3,11a,12,14,15-pentaaza-7,10-methanocyclohepta[4,5]cycloocta[1,2,3-de]naphthalene

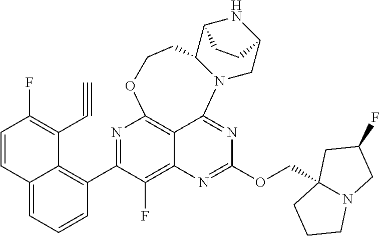 [SMILES: C#Cc1c(F)ccc2cccc(-c3nc4c5c(nc(OC[C@@]67CCCN6C[C@H](F)C7)nc5c3F)N3C[C@H]5CC[C@H](N5)[C@H]3CCO4)c12]

MS (ESI) m/z: 627 [M+H]⁺.

### Compound 52: 5-ethyl-6-fluoro-4-((6aR,7S,10R)-1-fluoro-13-(((2R,7aS)-2-fluorotetrahydro-1H-pyrrolizin-7a(5H)-yl)methoxy)-5,6,6a,7,8,9,10,11-octahydro-4-oxa-3,11a,12,14,15-pentaaza-7,10-methanocyclohepta[4,5]cycloocta[1,2,3-de]naphthalen-2-yl)naphthalen-2-ol

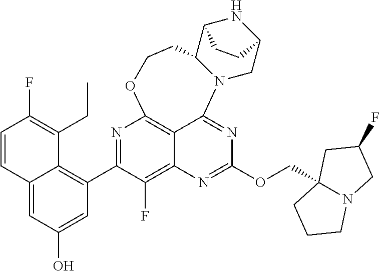 [SMILES: CCc1c(F)ccc2cc(O)cc(-c3nc4c5c(nc(OC[C@@]67CCCN6C[C@H](F)C7)nc5c3F)N3C[C@H]5CC[C@H](N5)[C@H]3CCO4)c12]

MS (ESI) m/z: 647 [M+H]⁺.

### Compound 53: 6-((5aS,6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-4-methyl-5-(trifluoromethyl) pyridin-2-amine

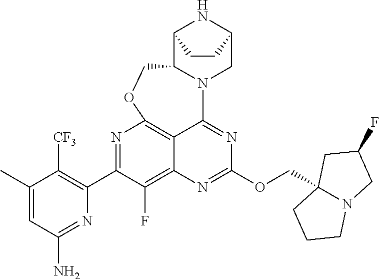 [SMILES: Cc1cc(N)nc(-c2nc3c4c(nc(OC[C@@]56CCCN5C[C@H](F)C6)nc4c2F)N2C[C@H]4CC[C@H](N4)[C@H]2CO3)c1C(F)(F)F]

MS (ESI) m/z: 619 [M+H]⁺.

¹H NMR (400 MHZ, CDCl₃) δ 6.43 (s, 1H), 5.52-5.39 (m, 1H), 5.01-4.92 (m, 3H), 4.68 (d, J=11.2 Hz, 1H), 4.53-4.48 (m, 2H), 4.29-4.24 (m, 2H), 4.01-3.81 (m, 4H), 3.55-3.42 (m, 1H), 3.34 (d, J=13.8 Hz, 1H), 3.22-3.20 (m, 1H), 2.69-2.45 (m, 5H), 2.43 (s, 3H), 2.00-1.90 (m, 5H).

### Compound 54: (5aS,6R,9S)-2-(5-(difluoromethyl)-3-methyl-2-(trifluoromethyl)phenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

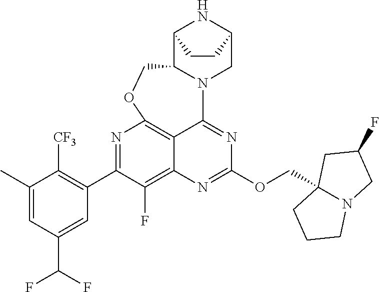 [SMILES: Cc1cc(C(F)F)cc(-c2nc3c4c(nc(OC[C@@]56CCCN5C[C@H](F)C6)nc4c2F)N2C[C@H]4CC[C@H](N4)[C@H]2CO3)c1C(F)(F)F]

MS (ESI) m/z: 653 [M+H]⁺.

### Compound 55: (5aS,6R,9S)-2-(3-chloro-2-cyclopropyl-5-(difluoromethyl)phenyl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

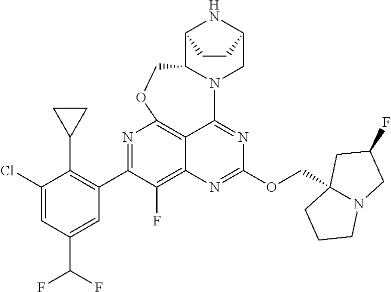 [SMILES: Fc1c(-c2cc(C(F)F)cc(Cl)c2C2CC2)nc2c3c(nc(OC[C@@]45CCCN4C[C@H](F)C5)nc13)N1C[C@H]3CC[C@H](N3)[C@H]1CO2]

MS (ESI) m/z: 645 [M+H]⁺.

### Compound 56: (5aS,6R,9S)-2-(5-chloro-4-cyclopropylpyridin-3-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

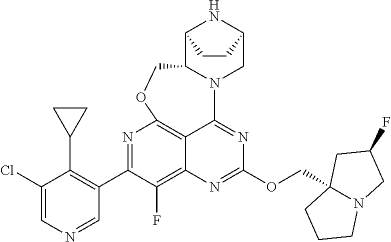 [SMILES: Fc1c(-c2cncc(Cl)c2C2CC2)nc2c3c(nc(OC[C@@]45CCCN4C[C@H](F)C5)nc13)N1C[C@H]3CC[C@H](N3)[C@H]1CO2]

MS (ESI) m/z: 596 [M+H]⁺.

### Compound 57: 5-chloro-6-((5aS,6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-4-methylpyridin-2-amine

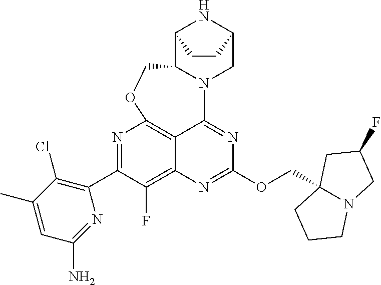 [SMILES: Cc1cc(N)nc(-c2nc3c4c(nc(OC[C@@]56CCCN5C[C@H](F)C6)nc4c2F)N2C[C@H]4CC[C@H](N4)[C@H]2CO3)c1Cl]

MS (ESI) m/z: 585 [M+H]⁺.

¹H NMR (400 MHZ, CDCl₃) δ 6.48 (s, 1H), 5.44-5.28 (m, 1H), 4.98 (d, J=12.6 Hz, 1H), 4.62-4.42 (m, 3H), 4.36 (s, 2H), 4.27-4.22 (m, 1H), 4.13-4.10 (m, 1H), 3.80 (s, 1H), 3.65 (s, 1H), 3.54-3.44 (m, 2H), 3.36-3.28 (m, 1H), 3.20 (d, J=13.1 Hz, 1H), 3.11-3.05 (m, 1H), 2.46-2.36 (m, 2H), 2.33 (s, 3H), 2.28-2.22 (m, 1H), 2.06-2.04 (m, 3H), 1.90-1.82 (m, 4H).

### Compound 58: 5-(difluoromethyl)-6-((5aS,6R,9S)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-4-methylpyridin-2-amine

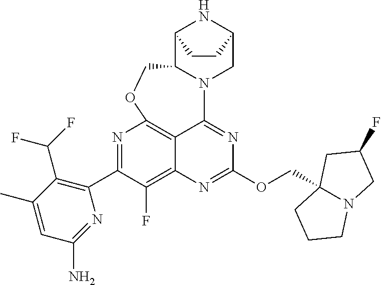 [SMILES: Cc1cc(N)nc(-c2nc3c4c(nc(OC[C@@]56CCCN5C[C@H](F)C6)nc4c2F)N2C[C@H]4CC[C@H](N4)[C@H]2CO3)c1C(F)F]

MS (ESI) m/z: 601 [M+H]⁺.

### Compound 71: 6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)-5-methylnaphthalen-2-ol

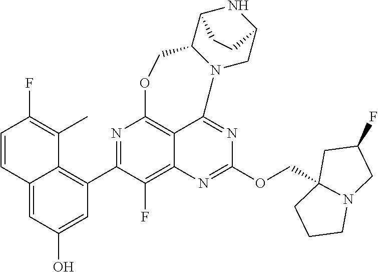 [SMILES: Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

MS (ESI) m/z: 619 [M+H]⁺.

### Compound 72:5-chloro-6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

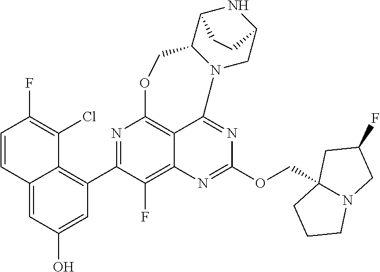 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(Cl)c(F)ccc2c1]

MS (ESI) m/z: 639 [M+H]⁺.

### Compound 73: 5,6-difluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

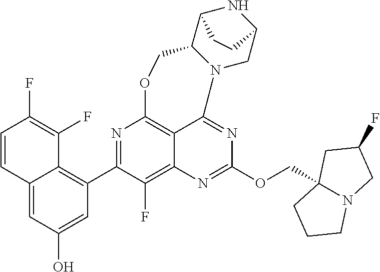 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(F)c(F)ccc2c1]

MS (ESI) m/z: 623 [M+H]⁺.

### Example 5

### Preparation of (5aS,6R,9S)—N—((R)-1-(dimethylamino) propan-2-yl)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalene-12-carboxamide (Compound 33)

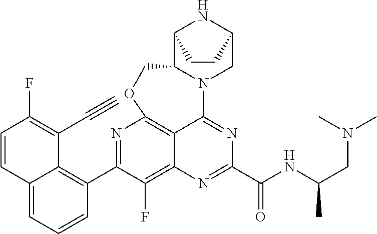 [SMILES: C#Cc1c(F)ccc2cccc(-c3nc4c5c(nc(C(=O)N[C@H](C)CN(C)C)nc5c3F)N3C[C@H]5CC[C@H](N5)[C@H]3CO4)c12]

### Tert-butyl (5aS,6R,9S)-12-cyano-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate

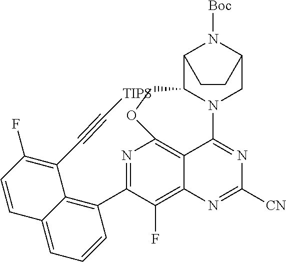

To a mixture of tert-butyl (5aS,6R,9S)-1-fluoro-2-(7-fluoro-8-((triisopropylsilyl)ethynyl)naphthalen-1-yl)-12-(methylsulfonyl)-5a,6,7,8,9,10-hexahydro-5H-4-oxa-3,10a,11,13,14-pentaaza-6,9-methanonaphtho[1,8-ab]heptalene-14-carboxylate (100 mg, 0.13 mmol) in DMSO (2 mL) was added NaCN (12.4 mg, 0.25 mmol) and the mixture was stirred at 85° C. for 2 hrs. The reaction mixture was diluted with EtOAc, washed with water and brine, dried over anhydrous Na₂SO₄ and filtered. The filtrate was concentrated under reduced pressure to dryness. The residue was purified by flash chromatography (silica gel, 0-10% EtOAc in PE) to give tert-butyl (5aS,6R,9S)-12-cyano-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate (40 mg, 43% yield) as a red solid. LCMS (ESI) m/z: 737 (M+H)⁺.

### Methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylate

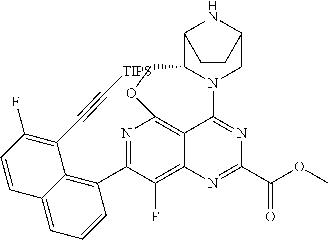

To a solution of tert-butyl (5aS,6R,9S)-12-cyano-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate (30 mg, 0.04 mmol) in MeOH (1 mL) was added HCl/1,4-dioxane (1 mL, 4M) and the mixture was stirred at 70° C. for 2 hrs. The mixture was concentrated under reduced pressure to dryness to give methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylate (26 mg, 95% yield) as a red solid. LCMS (ESI) m/z: 670 (M+H)⁺.

### 14-(Tert-butyl) 12-methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12,14-dicarboxylate

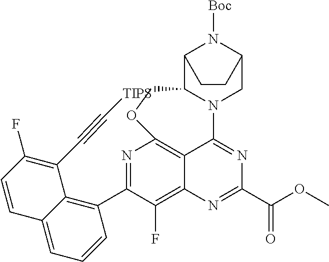

To a mixture of methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylate (26 mg, 0.04 mmol) and saturated aq. NaHCO₃ solution (1 mL) in THF (1 mL) was added Boc₂O (13 mg, 0.06 mmol) and the mixture was stirred at 25° C. for 2 hrs. The reaction mixture partitioned with EtOAc and water. The separated organic layer was dried over anhydrous Na₂SO₄, filtered and concentrated under reduced pressure to dryness to give crude 14-(tert-butyl) 12-methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12,14-dicarboxylate (39 mg, 100% yield) as a yellow solid. LCMS (ESI) m/z: 770 (M+H)⁺.

### (5aS,6R,9S)-14-(tert-butoxycarbonyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylic Acid

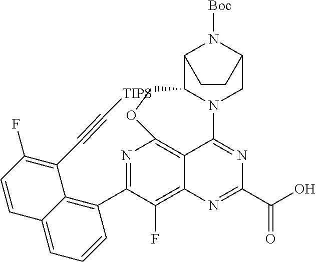

To a mixture of 14-(tert-butyl) 12-methyl (5aS,6R,9S)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12,14-dicarboxylate (39 mg, 0.04 mmol) in THF (0.5 mL), MeOH (0.5 mL) and water (0.5 mL) was added LiOH·H₂O (7 mg, 0.15 mmol) and the mixture was stirred at r.t. for 2 hrs. The mixture was concentrated under reduced pressure to dryness to give crude (5aS,6R,9S)-14-(tert-butoxycarbonyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylic acid (40 mg, 100% yield) as a yellow solid. LCMS (ESI) m/z: 756 (M+H)⁺.

### Tert-butyl (6R,9S)-12-(((R)-1-(dimethylamino) propan-2-yl) carbamoyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate

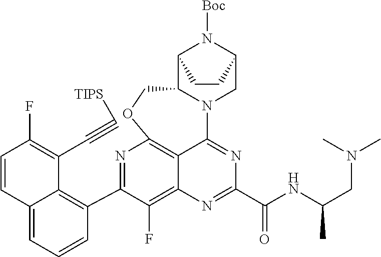

A mixture of crude (5aS,6R,9S)-14-(tert-butoxycarbonyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxylic acid (39 mg, 0.045 mmol), HATU (19.8 mg, 0.05 mmol), DIPEA (0.04 mL, 0.2 mmol) and [(2R)-2-aminopropyl]dimethylamine hydrochloride (7 mg, 0.05 mmol) in DMF (0.5 mL) was stirred at r.t. for 2 hrs. The reaction mixture was diluted with EtOAc (5 mL), washed with water and brine, dried over anhydrous Na₂SO₄, filtered and concentrated to give crude tert-butyl (5aS,6R,9S)-12-(((R)-1-(dimethylamino) propan-2-yl) carbamoyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate (20 mg, 53.2% yield) as a yellow solid. MS (ESI) m/z: 840 (M+H)⁺.

### (5aS,6R,9S)—N—((R)-1-(dimethylamino) propan-2-yl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxamide

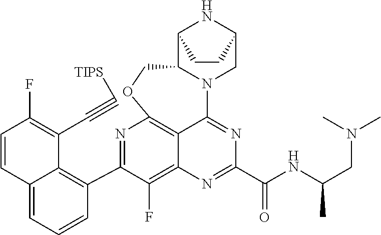

To a mixture of tert-butyl (5aS,6R,9S)-12-(((R)-1-(dimethylamino) propan-2-yl) carbamoyl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-14-carboxylate (20 mg, 0.02 mmol) in DCM (0.3 mL) was added HCl/1,4-dioxane (0.3 mL, 4M) and the mixture was stirred at 25° C. for 2 hrs. The reaction mixture was concentrated under reduced pressure to dryness to give (5aS,6R,9S)—N—((R)-1-(dimethylamino) propan-2-yl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxamide (15 mg, 85% yield) as a yellow solid. MS (ESI) m/z: 740 (M+H)⁺.

### (5aS,6R,9S)—N—((R)-1-(dimethylamino) propan-2-yl)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalene-12-carboxamide

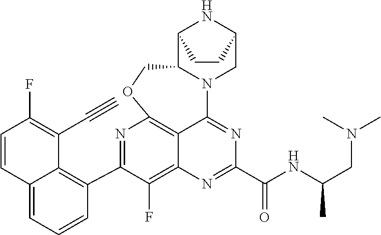 [SMILES: C#Cc1c(F)ccc2cccc(-c3nc4c5c(nc(C(=O)N[C@H](C)CN(C)C)nc5c3F)N3C[C@H]5CC[C@H](N5)[C@H]3CO4)c12]

To a solution of (5aS,6R,9S)—N—((R)-1-(dimethylamino) propan-2-yl)-1-fluoro-2-(7-fluoro-8-((triisopropylsilyl)ethynyl)naphthalen-1-yl)-5a,6,7,8,9,10-hexahydro-5H-4-oxa-3,10a,11,13,14-pentaaza-6,9-methanonaphtho[1,8-ab]heptalene-12-carboxamide (15 mg, 0.02 mmol) in DMF (0.5 mL) was added CsF (15 mg, 0.1 mmol) and the mixture was stirred at 25° C. for 1.5 hrs. The mixture was filtered and the filtrate was concentrated under reduced pressure to dryness. The residue was purified by prep-HPLC to give (5aS,6R,9S)—N—((R)-1-(dimethylamino) propan-2-yl)-2-(8-ethynyl-7-fluoronaphthalen-1-yl)-1-fluoro-5a,6,7,8,9,10-hexahydro-5H-4-oxa-3,10a,11,13,14-pentaaza-6,9-methanonaphtho[1,8-ab]heptalene-12-carboxamide (1.78 mg, 15% yield) as a red solid.

LC/MS (ESI) m/z: 584 (M+H)⁺.

¹H NMR (400 MHZ, CDCl₃) δ 8.41 (d, J=7.8 Hz, 1H), 7.97-7.91 (m, 2H), 7.68-7.54 (m, 2H), 7.35-7.30 (m, 1H), 5.30-5.25 (m, 1H), 4.55-4.27 (m, 4H), 4.19-4.13 (m, 1H), 3.83 (s, 1H), 3.67 (s, 1H), 3.29 (t, J=12.0 Hz, 1H), 2.98-2.93 (m, 2H), 2.47 (s, 6H), 2.02-1.81 (m, 5H), 1.36 (d, J=6.4 Hz, 3H).

### Example 6

### Preparation of (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene (Compound 34)

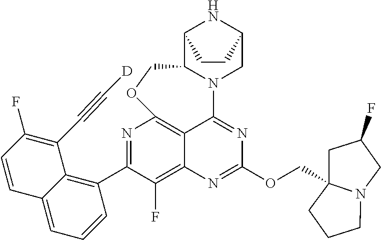 [SMILES: [2H]C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12]

### Tert-butyl (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

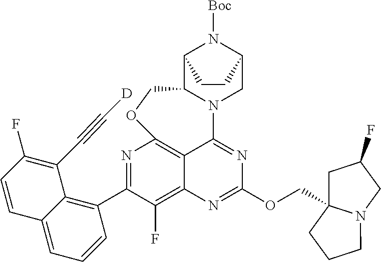 [SMILES: [2H]C#Cc1c(F)ccc2cccc(-c3nc4c5c(nc(OC[C@@]67CCCN6C[C@H](F)C7)nc5c3F)N3C[C@H]5CC[C@@H]([C@H]3CO4)N5C(=O)OC(C)(C)C)c12]

To a solution of tert-butyl (5aS,6R,9S)-1-fluoro-2-(7-fluoro-8-((triisopropylsilyl)ethynyl)naphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (50 mg, 0.06 mmol) in DMF (2 mL) were added CsF (535 mg, 3.5 mmol), CD₃OD (4 mL), and the reaction was stirred at room temperature for 6 hours. LCMS monitored the reaction. The crude product was purified by flash column chromatography on silica gel (DCM/MeOH=10/1) to give the tert-butyl (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (30 mg, 73%) as a pink solid.

LC/MS (ESI) m/z: 714 [M+H]⁺.

### (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene

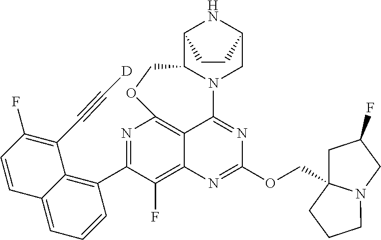 [SMILES: [2H]C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12]

To a solution of tert-butyl (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (30 mg, 0.04 mmol) in DCM (5 mL) were added HCl/Dioxane (3 mL), and the reaction was stirred at room temperature for 1 hour. LCMS monitored the reaction. Filtered and concentrated to get crude product was purified by prep-HPLC to give the (5aS,6R,9S)-2-(8-(ethynyl-d)-7-fluoronaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene (4 mg, 16%) as a white solid.

LC/MS (ESI) m/z: 614 [M+H]⁺.

¹H NMR (400 MHZ, MeOD) δ 8.08 (dq, J=9.2, 3.1 Hz, 2H), 7.66-7.56 (m, 2H), 7.42 (td, J=8.9, 5.5 Hz, 1H), 5.37-5.21 (m, 1H), 5.05 (ddd, J=13.5, 7.3, 2.2 Hz, 1H), 4.62-4.57 (m, 1H), 4.48-4.40 (m, 1H), 4.27 (dd, J=10.4, 7.5 Hz, 1H), 4.20 (dd, J=10.4, 4.5 Hz, 1H), 4.13 (d, J=7.0 Hz, 1H), 3.72 (d, J=5.6 Hz, 1H), 3.65-3.61 (m, 1H), 3.20 (d, J=21.4 Hz, 4H), 3.06-2.95 (m, 1H), 2.32-2.11 (m, 3H), 2.02-1.81 (m, 7H).

### Example 7

### Preparation of (5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de][1,6]naphthyridine (Compound 35)

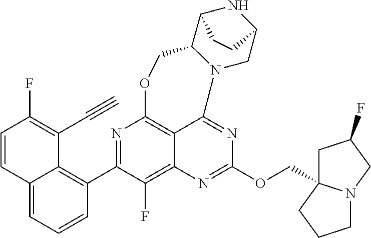 [SMILES: C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12]

### 4-Bromo-2,6-dichloro-5-fluoronicotinic Acid

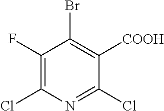 [SMILES: O=C(O)c1c(Cl)nc(Cl)c(F)c1Br]

To a cold solution of 2,6-dichloro-5-fluoropyridine-3-carboxylic acid (10.0 g, 47.6 mmol) in anhydrous THF (100 mL), was added MeLi (1.3 mol/L, 76 mL) at −78° C. over 30 min, And then the reaction was warmed to −20˜−30° C. stirred for 2 hours. The reaction mixture was cooled to −78° C. Followed by adding 1,2-dibromo-1,1,2,2-tetrachloroethane (17.0 g, 52 mmol) in anhydrous THF (100 mL). Then the reaction mixture was stirred at 0° C. for 1.5 hours. LCMS showed the reaction worked well. The reaction solution was diluted with ice water (150 mL) and the reaction was cleaned with chloroform, adjusted PH-2 by adding 1M hydrochloric acid in water and extracted by Ethyl acetate (50 mL×3), the organic layer was combined and dried with Na₂SO₄, then filtered and concentrated to give 4-bromo-2,6-dichloro-5-fluoropyridine-3-carboxylic acid (11.5 g, 83.5%) as a white solid without further purification for the next step. MS (ESI) m/z: 288 [M+H]⁺.

### 2,6-Dichloro-5-fluoro-4-((4-methoxybenzyl)amino)nicotinic Acid

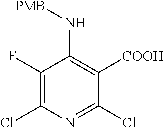

To a flask containing 4-bromo-2,6-dichloro-5-fluoronicotinic acid (5.0 g, 17.3 mmol) was added DMF (70 mL) followed by the addition of PMBNH₂ (2.8 g, 20.7 mmol), and DIEA (8.5 mL, 51.9 mmol). The mixture was stirred at 50° C. for overnight. The result mixture was concentrated in vacuo. The residue was prep-HPLC with (MeCN, H₂O/FA) to afford the title compound 2,6-dichloro-5-fluoro-4-((4-methoxybenzyl)amino)nicotinic acid (1.1 g, 18.4%). MS (ESI) m/z: 345 [M+H]⁺.

### 2,6-Dichloro-4-(3-ethoxy-N-(4-methoxybenzyl)-3-oxopropanamido)-5-fluoronicotinic Acid

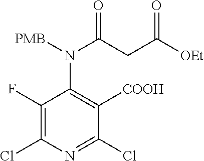

To a solution of 2,6-dichloro-5-fluoro-4-((4-methoxy benzyl)amino)nicotinic acid (1.2 g, 3.5 mmol) in DCM (20 mL) were added TEA (0.9 mL, 6.9 mmol) and ethyl 3-chloro-3-oxopropanoate (0.6 mL, 5.2 mmol), and the reaction was stirred at room temperature for 1.5 hours. The reaction was concentrated in vacuo. The residue was washed with further HCl solution (1M). The aqueous layer was back extracted with EA (3×15 mL). Combined the organic layer and dried by Na₂SO₄, filtered and concentrated to afford the crude compound 2,6-dichloro-4-(3-ethoxy-N-(4-methoxybenzyl)-3-oxopropanamido)-5-fluoronicotinic acid (1.4 g, 87.6%). MS (ESI) m/z: 459 [M+H]⁺.

### Ethyl 5,7-dichloro-8-fluoro-1-(4-methoxybenzyl)-2,4-dioxo-1,2,3,4-tetrahydro-1,6-naphthyridine-3-carboxylate

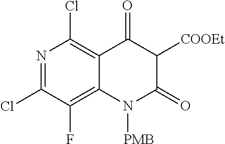

To a solution of 2,6-dichloro-4-(3-ethoxy-N-(4-methoxybenzyl)-3-oxopropanamido)-5-fluoronicotinic acid (1.4 g, 3.0 mmol) in DCE (20 mL) were added TEA (0.8 mL, 6.1 mmol) and SOCl₂ (0.3 mL, 4.6 mmol) at 0° C., and the reaction was stirred at room temperature for 3 hours. The reaction was diluted with DCM and saturated NaHCO₃ solution. The organic layer was separated and concentrated in vacuo. The crude material was loaded on a silica gel plate. The plate was developed using DCM:MeOH=10:1 to afford the title compound ethyl 5,7-dichloro-8-fluoro-1-(4-methoxybenzyl)-2,4-dioxo-1,2,3,4-tetrahydro-1,6-naphthyridine-3-carboxylate (1.0 g, 74.3%). MS (ESI) m/z: 441 [M+H]⁺.

### 5,7-Dichloro-8-fluoro-4-hydroxy-1-(4-methoxybenzyl)-1,6-naphthyridin-2 (1H)-one

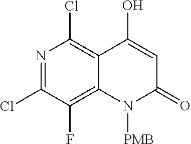

To a solution of ethyl 5,7-dichloro-8-fluoro-1-(4-methoxybenzyl)-2,4-dioxo-1,2,3,4-tetrahydro-1,6-naphthyridine-3-carboxylate (200 mg, 0.45 mmol) in DMSO (5 mL) were added water (0.5 mL, 27.0 mmol) and sodium chloride (316 mg, 5.45 mmol). After being heated at 200° C. under microwave irradiation for 40 min, the mixture was transferred to a separatory funnel, diluted with EA and washed with water. The organic layer was separated, dried over Na₂SO₄, filtered and concentrated. The residue was purified by flash silica gel column chromatography (EA/PE 1:4) to afford 5,7-dichloro-8-fluoro-4-hydroxy-1-(4-methoxybenzyl)-1,6-naphthyridin-2 (1H)-one (50.0 mg, 29.8%) as a yellow solid. MS (ESI) m/z: 367 [M−H]⁻.

### Tert-butyl (1R,2S,5S)-2-(((7-chloro-8-fluoro-4-hydroxy-1-(4-methoxybenzyl)-2-oxo-1,2-dihydro-1,6-naphthyridin-5-yl)oxy)methyl)-3,8-diazabicyclo[3.2.1]octane-8-carboxylate

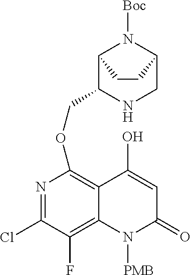

To a flask containing tert-butyl (1S,2S,5R)-2-(hydroxymethyl)-3,8-diazabicyclo[3.2.1]octane-8-carboxylate (102 mg, 0.42 mmol) was added THF (5 mL) followed by the addition of NaH (16.0 mg, 0.40 mmol, 60% in oil) at 0° C. The mixture was stirred at rt for 20 min. The mixture was added 5,7-dichloro-8-fluoro-4-hydroxy-1-(4-methoxybenzyl)-1,6-naphthyridin-2 (1H)-one (130 mg, 0.35 mmol). The mixture was stirred at rt for 1 hour. The reaction was quenched with saturated NH₄Cl solution. The aqueous layer was back extracted with EA (3×5 mL). Combined the EA layer and dried by Na₂SO₄, filtered and concentrated. The crude material was loaded on a silica gel plate. The plate was developed using DCM:MeOH=10:1 to provide the title compound tert-butyl (1R,2S,5S)-2-(((7-chloro-8-fluoro-4-hydroxy-1-(4-methoxybenzyl)-2-oxo-1,2-dihydro-1,6-naphthyridin-5-yl)oxy)methyl)-3,8-diazabicyclo[3.2.1]octane-8-carboxylate (45 mg, 22.2%). MS (ESI) m/z: 575 [M+H]⁺.

### Tert-butyl (5aS,6S,9R)-2-chloro-1-fluoro-14-(4-methoxybenzyl)-13-oxo-5a,6,7,8,9,10,13,14-octahydro-5H-6,9-epiminoazepino[2′,1′:3,4][1,4]oxazepino[5,6,7-de][1,6]naphthyridine-15-carboxylate

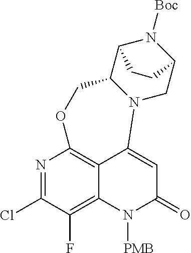

To a solution of tert-butyl (1R,2S,5S)-2-(((7-chloro-8-fluoro-4-hydroxy-1-(4-methoxybenzyl)-2-oxo-1,2-dihydro-1,6-naphthyridin-5-yl)oxy)methyl)-3,8-diazabicyclo[3.2.1]octane-8-carboxylate (45 mg, 0.08 mmol) in MeCN (1 mL) were added PyBOP (81 mg, 0.16 mmol) and DBU (0.06 mL, 0.39 mmol), the reaction was stirred at 60° C. for 2 hours. The reaction was concentrated. The crude material was loaded on a silica gel plate. The plate was developed using DCM:MeOH=20:1 to afford the title compound tert-butyl (5aS,6S,9R)-2-chloro-1-fluoro-14-(4-methoxybenzyl)-13-oxo-5a,6,7,8,9,10,13,14-octahydro-5H-6,9-epiminoazepino[2′,1′:3,4][1,4]oxazepino[5,6,7-de][1,6]naphthyridine-15-carboxylate (20 mg, 45.8%). MS (ESI) m/z: 557 [M+H]⁺.

### (5aS,6S,9R)-2-chloro-1-fluoro-5a,6,7,8,9,10-hexahydro-5H-6,9-epiminoazepino[2′,1′:3,4][1,4]oxazepino[5,6,7-de][1,6]naphthyridin-13 (14H)-one

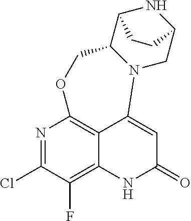 [SMILES: O=c1cc2c3c(nc(Cl)c(F)c3[nH]1)OC[C@@H]1[C@@H]3CC[C@H](CN21)N3]

To a flask containing tert-butyl (5aS,6S,9R)-2-chloro-1-fluoro-14-(4-methoxybenzyl)-13-oxo-5a,6,7,8,9,10,13,14-octahydro-5H-6,9-epiminoazepino[2′,1′:3,4][1,4]oxazepino[5,6,7-de][1,6]naphthyridine-15-carboxylate (10 mg, 0.018 mmol) was added TFA (0.5 mL) followed by the addition of CF₃SO₃H (one drop). The mixture was stirred at room temperature for 2 hours. The mixture was concentrated under vacuum to provide the title compound (5aS,6S,9R)-2-chloro-1-fluoro-5a,6,7,8,9,10-hexahydro-5H-6,9-epiminoazepino[2′,1′:3,4][1,4]oxazepino[5,6,7-de][1,6]naphthyridin-13 (14H)-one (6 mg, 99.9%). MS (ESI) m/z: 337 [M+H]⁺.

### Tert-butyl (5aS,6S,9R)-2-chloro-1-fluoro-13-oxo-5a,6,7,8,9,10,13,14-octahydro-5H-6,9-epiminoazepino[2′,1′:3,4][1,4]oxazepino[5,6,7-de][1,6]naphthyridine-15-carboxylate

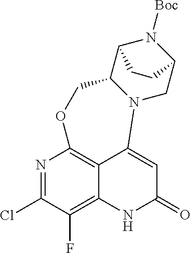 [SMILES: CC(C)(C)OC(=O)N1[C@@H]2CC[C@H]1[C@H]1COc3nc(Cl)c(F)c4[nH]c(=O)cc(c34)N1C2]

To a solution of (5aS,6S,9R)-2-chloro-1-fluoro-5a,6,7,8,9,10-hexahydro-5H-6,9-epiminoazepino[2′,1′:3,4][1,4]oxazepino[5,6,7-de][1,6]naphthyridin-13 (14H)-one (6 mg, 0.018 mmol) in THF (1 mL), H₂O (0.2 mL) were added NaHCO₃ (8 mg, 0.045 mmol) and Boc₂O (5 mg, 0.01 mmol), and the reaction was stirred at room temperature for 1.5 hours. The reaction was diluted with EA and water. The organic layer was separated and concentrated in vacuo to afford the title compound tert-butyl (5aS,6S,9R)-2-chloro-1-fluoro-13-oxo-5a,6,7,8,9,10,13,14-octahydro-5H-6,9-epiminoazepino[2′,1′:3,4][1,4]oxazepino[5,6,7-de][1,6]naphthyridine-15-carboxylate (7 mg, 97.6%). MS (ESI) m/z: 437 [M+H]⁺.

### Tert-butyl (8aS,9S,12R)-5-chloro-4-fluoro-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine-15-carboxylate

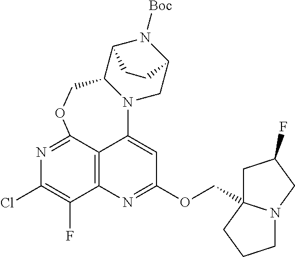

To a solution of tert-butyl (5aS,6S,9R)-2-chloro-1-fluoro-13-oxo-5a,6,7,8,9,10,13,14-octahydro-5H-6,9-epiminoazepino[2′,1′: 3,4][1,4]oxazepino[5,6,7-de][1,6]naphthyridine-15-carboxylate (10 mg, 0.023 mmol) in toluene (1.5 mL) were added ((2R,7aS)-2-fluorotetrahydro-1H-pyrrolizin-7a(5H)-yl) methanol (5 mg, 0.030 mmol) and CMBP (17 mg, 0.069 mmol), and the reaction was stirred at 110° C. for 5 hours. The mixture was concentrated. The crude material was loaded on a silica gel plate. The plate was developed using DCM:MeOH=10:1 to afford the title compound tert-butyl (8aS,9S,12R)-5-chloro-4-fluoro-2-(((2R,7aS)-2-fluorotetrahydro-1H-pyrrolizin-7a(5H)-yl)methoxy)-8a,9,10,11,12,13-hexahydro-8H-9,12-epiminoazepino[2′,11:3,4][1,4]oxazepino[5,6,7-de][1,6]naphthyridine-15-carboxylate (10 mg, 75.5%). MS (ESI) m/z: 578 [M+H]⁺.

### Tert-butyl (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine-15-carboxylate

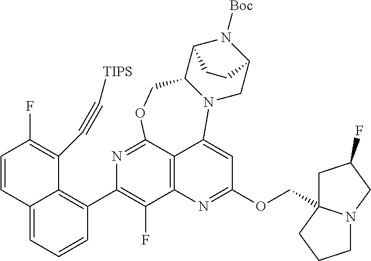

To a solution of tert-butyl (8aS,9S,12R)-5-chloro-4-fluoro-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine-15-carboxylate (10 mg, 0.017 mmol) in THF (1 mL), H₂O (0.2 mL) were added ((2-fluoro-8-(4,4,5,5-tetramethyl-1,3,2-dioxaborolan-2-yl)naphthalen-1-yl)ethynyl)triisopropylsilane (12 mg, 0.026 mmol), X-Phos Pd G2 (2 mg, 0.002 mmol), and K₃PO₄ (11 mg, 0.051 mmol) under N₂, and the reaction was stirred at 60° C. for 1 hour. The reaction was diluted with EA and water. The organic layer was separated and concentrated in vacuo. The residue was purified using silica gel column chromatography eluting with DCM:MeOH=10:1 to afford the title compound tert-butyl (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine-15-carboxylate (7 mg, 46.6%). MS (ESI) m/z: 868 [M+H]⁺.

### (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine

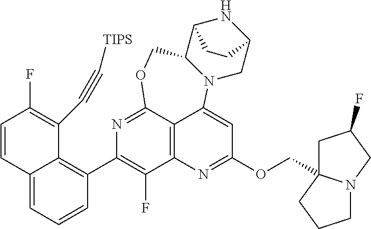

To a flask containing tert-butyl (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′, l′: 3,4][1,4]oxazepino[5,6,7-de][1,6]naphthyridine-15-carboxylate (7 mg, 0.008 mmol) was added DCM (1.5 mL) followed by the addition of 0.5 mL HCl/dioxane (4M, 0.3 mL). The mixture was stirred at rt for 2 hours. The mixture was concentrated to provide the title compound (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine (6 mg, 96.8%). MS (ESI) m/z: 768 [M+H]⁺.

### (5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de][1,6]naphthyridine

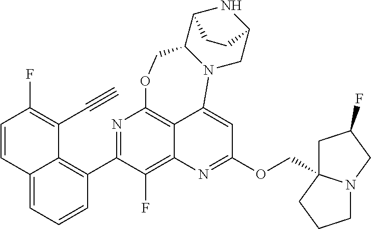 [SMILES: C#Cc1c(F)ccc2cccc(-c3nc4c5c(cc(OC[C@@]67CCCN6C[C@H](F)C7)nc5c3F)N3C[C@H]5CC[C@H](N5)[C@H]3CO4)c12]

To a flask containing (8aS,9S,12R)-4-fluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de][1,6]naphthyridine (6 mg, 0.008 mmol) was added DMF (1 mL) followed by the addition of CsF (24 mg, 0.156 mmol). The mixture was stirred at rt for 2 hours. The mixture was prep-HPLC with (MeCN, H₂O) to provide the title compound (5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de][1,6]naphthyridine (1.3 mg, 27.2%).

MS (ESI) m/z: 612 [M+H]⁺.

¹H NMR (400 MHZ, MeOD) δ 8.14-8.01 (m, 2H), 7.72-7.52 (m, 2H), 7.42 (dd, J=16.2, 8.9 Hz, 1H), 6.31 (d, J=4.7 Hz, 1H), 5.35-5.21 (m, 1H), 4.56 (d, J=14.5 Hz, 4H), 4.45-4.24 (m, 3H), 3.67 (s, 1H), 3.58 (s, 2H), 3.43 (s, 2H), 3.08 (d, J=35.6 Hz, 2H), 2.30-2.10 (m, 4H), 1.98 (dd, J=34.6, 28.6 Hz, 6H).

Compounds 59-60 can be prepared in a similar way to Compound 35, except for using other appropriate aryl boronic esters and alcohols.

### Compound 59: 5-ethynyl-6-fluoro-4-((5aS,6S,9R)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de][1,6]naphthyridin-2-yl)naphthalen-2-ol

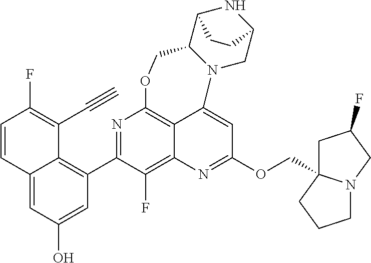 [SMILES: C#Cc1c(F)ccc2cc(O)cc(-c3nc4c5c(cc(OC[C@@]67CCCN6C[C@H](F)C7)nc5c3F)N3C[C@H]5CC[C@H](N5)[C@H]3CO4)c12]

MS (ESI) m/z: 628 [M+H]⁺.

### Compound 60: 5-ethyl-6-fluoro-4-((5aS,6S,9R)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de][1,6]naphthyridin-2-yl)naphthalen-2-ol

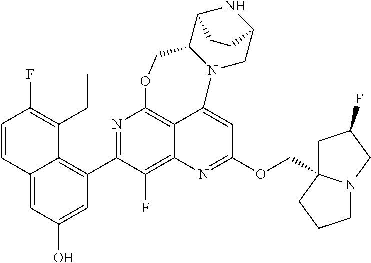 [SMILES: CCc1c(F)ccc2cc(O)cc(-c3nc4c5c(cc(OC[C@@]67CCCN6C[C@H](F)C7)nc5c3F)N3C[C@H]5CC[C@H](N5)[C@H]3CO4)c12]

MS (ESI) m/z: 632 [M+H]⁺.

### Example 8

### Preparation of (5aS,6S,9R)-2-(8-chloro-7-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-2,10a,11,13,14-pentaaza-6,9-methanonaphtho[1,8-ab]heptalen-1 (2H)-one

### Compound 43

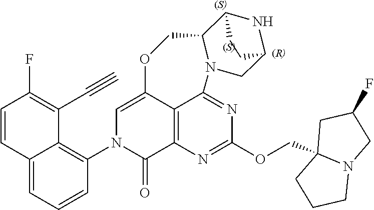

### 5-Bromo-3-fluoro-2-methoxyisonicotinic Acid

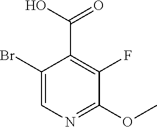

To a solution of 5-bromo-3-fluoro-2-methoxypyridine (20 g, 97 mmol) in dry THF (200 ml), a solution of n-BuLi (2.5 M in hexane, 1.5 eq) was added dropwise over 10 mins at −78° C. and stirred 30 mins at the same temperature. After 30 mins crushed solid dry ice was added portion wise to the above solution at −78° C. Then, the reaction mixture was allowed to warm up to rt over 2 hours. Then the reaction mixture was cooled to 0° C. and neutralized by conc. HCl. Then the reaction mixture was concentrated under reduced pressure to give a crude product. The crude product was dissolved in 5M NaOH solution and washed with ether; the aqueous layer was cooled to 0° C. and acidified to pH 5˜6 by conc. HCl. A precipitate formed. The precipitate was filtered and washed with ether to give 5-bromo-3-fluoro-2-methoxyisonicotinic acid (18 g, 74.16%) as a white solid

MS (ESI) m/z: 250 [M+H]⁺.

### Methyl (5-bromo-3-fluoro-2-methoxyisonicotinoyl) carbamimidothioate

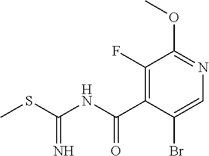 [SMILES: COc1ncc(Br)c(C(=O)NC(=N)SC)c1F]

To a solution of 5-bromo-3-fluoro-2-methoxyisonicotinic acid (18 g, 72 mmol) in DCM (200 mL) were added (COCl)₂ (10 mL) under N₂ atmosphere at 0° C., and the reaction was stirred at room temperature for 3 hours. TLC detected that the reaction is complete. The reaction concentrated in vacuo to give crude product used to next step as reddish brown oil.

To a solution of NaOH (11.5 g, 288 mmol) in H₂O (100 mL) were added 2-methyl-2-thio-pseudourehydrogensulfate (13.1 g, 133.2 mmol) in batches at 0° C., and the reaction was stirred at 0° C. for 30 mins. To this mixture was added the solution of crude acyl chloride product in DCM (50 mL) at 0° C., and the reaction was stirred at room temperature for 1 hour. TLC monitored the reaction and was completed. The organic layer was separated, and the water layer extracted with EA (300 mL×2). Combined the organic layers and dried with anhydrous sodium sulfate, filtered and concentrated in vacuo. The residue was purified using silica gel column chromatography eluting with ethyl acetate in petroleum ether to afford the title methyl (5-bromo-3-fluoro-2-methoxyisonicotinoyl) carbamimidothioate (6.4 g, 27.6%) as a white solid.

MS (ESI) m/z: 322 [M+H]⁺.

¹H NMR (400 MHZ, DMSO-d₆) δ 9.53 (s, 1H), 9.04 (s, 1H), 8.17 (s, 1H), 3.95 (s, 3H), 2.39 (s, 3H).

### 5-Bromo-8-methoxy-2-(methylthio)pyrido[3,4-d]pyrimidin-4 (3H)-one

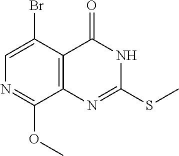 [SMILES: COc1ncc(Br)c2c(=O)[nH]c(SC)nc12]

To a solution of methyl (5-bromo-3-fluoro-2-methoxyisonicotinoyl) carbamimidothioate (6.4 g, 19.8 mmol) and Cs₂CO₃ (9.7 g, 29.8 mmol) in DMF (60 mL). The mixture was stirred at 90° C. for 3 hours. LCMS showed the starting material was consumed completely. The reaction mixture was cooled to room temperature and concentrated in vacuum to remove most of solvent. Water was added in and the aqueous layer was cooled to 0° C. and acidified to pH 3˜4 by conc. HCl. A precipitate was formed. The precipitate was filtered and washed with water to give 5-bromo-8-methoxy-2-(methylthio)pyrido[3,4-d]pyrimidin-4 (3H)-one (3 g, 50%) as a white solid.

MS (ESI) m/z: 302 [M+H]⁺.

¹H NMR (400 MHZ, DMSO-d₆) δ 13.05 (s, 1H), 8.15 (s, 1H), 3.97 (s, 3H), 2.57 (s, 3H).

### 5-Bromo-4-chloro-8-methoxy-2-(methylthio)pyrido[3,4-d]pyrimidine

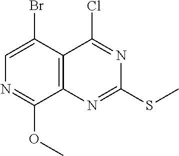 [SMILES: COc1ncc(Br)c2c(Cl)nc(SC)nc12]

To a solution of 5-bromo-8-methoxy-2-(methylthio)pyrido[3,4-d]pyrimidin-4 (3H)-one (500 mg, 1.65 mmol) in DIEA (1.5 mL, 9 mmol) and POCl₃ (30 mL). The reaction mixture waw stirred at 110° C. at for 3 hours. The reaction was monitored by LCMS. The mixture was concentrated afford the crude product. Washed with saturated NaHCO₃ solution. The EA layer was dried by Na₂SO₄, filtered and concentrated. Used for the next reaction step directly.

MS (ESI) m/z: 320 [M+H]⁺.

¹H NMR (400 MHZ, DMSO-d₆) δ 8.56 (s, 1H), 4.13 (s, 3H), 2.65 (s, 3H).

### Tert-butyl (1S,2S,5R)-3-(5-bromo-8-methoxy-2-(methylthio)pyrido[3,4-d]pyrimidin-4-yl)-2-(hydroxymethyl)-3,8-diazabicyclo[3.2.1]octane-8-carboxylate

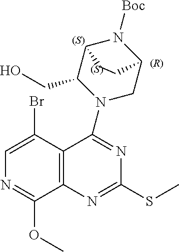

To a solution of 5-bromo-4-chloro-8-methoxy-2-(methylthio)pyrido[3,4-d]pyrimidine (500 mg, 1.56 mmol) and tert-butyl (1S,2S,5R)-2-(hydroxymethyl)-3,8-diazabicyclo[3.2.1]octane-8-carboxylate (378 mg, 1.56 mmol) in MeCN (30 mL), The mixture was 80° C. for 4 hours until the starting material was consumed completely. The reaction mixture was cooled to room temperature and concentrated in vacuum to remove most of solvent. The residue was poured into water and extracted with EtOAc (60 mL). The organic layer was dried over Na₂SO₄, filtered and concentrated. The residue was purified using silica gel column chromatography eluting with PE/EtOAc 1:1 to give tert-butyl (1S,2S,5R)-3-(5-bromo-8-methoxy-2-(methylthio)pyrido[3,4-d]pyrimidin-4-yl)-2-(hydroxymethyl)-3,8-diazabicyclo[3.2.1]octane-8-carboxylate (400 mg, 48.7%) as a solid.

MS (ESI) m/z: 526 [M+H]⁺.

### Tert-butyl (8aS,9S,12R)-4-methoxy-2-(methylthio)-8a,9,10,11,12,13-hexahydro-8H-7-oxa-1,3,5,13a,14-pentaaza-9,12-methanonaphtho[1,8-ab]heptalene-14-carboxylate

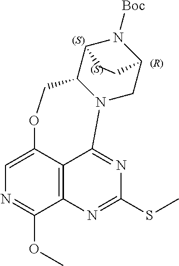

To a solution of tert-butyl (1S,2S,5R)-3-(5-bromo-8-methoxy-2-(methylthio)pyrido[3,4-d]pyrimidin-4-yl)-2-(hydroxymethyl)-3,8-diazabicyclo[3.2.1]octane-8-carboxylate (230 mg, 0.43 mmol) and Pd(OAc)₂ (39 mg, 0.17 mmol), BINAP (108 mg, 0.17 mmol), Cs₂CO₃ (213 mg, 0.65 mmol) in toluene (20 mL), The mixture was heated to 80° C. and stirred for 12 hours until the starting material was consumed completely. The reaction mixture was cooled to room temperature and concentrated in vacuum to remove most of solvent. The residue was poured into water and extracted with EtOAc (60 mL). The organic layer was dried over Na₂SO₄, filtered and concentrated. The residue was purified using silica gel column chromatography eluting with PE/EtOAc 1:1 to give tert-butyl (8aS,9S,12R)-4-methoxy-2-(methylthio)-8a,9, 10, 11, 12,13-hexahydro-8H-7-oxa-1,3,5,13a,14-pentaaza-9,12-methanonaphtho[1,8-ab]heptalene-14-carboxylate (85 mg, 43.67%) as a solid.

MS (ESI) m/z: 446 [M+H]⁺.

¹H NMR (400 MHz, CDCl₃) δ 7.41 (s, 1H), 4.53 (d, J=13.9 Hz, 1H), 4.33 (dd, J=13.7, 4.7 Hz, 2H), 4.10 (d, J=6.9 Hz, 3H), 3.39 (d, J=31.1 Hz, 2H), 2.98 (dd, J=10.9, 2.1 Hz, 1H), 2.65 (s, 3H), 2.49 (t, J=9.0 Hz, 1H), 1.98-1.88 (m, 2H), 1.71 (dd, J=23.7, 16.8 Hz, 2H), 1.50 (d, J=8.0 Hz, 9H).

### (8aS,9S,12R)-2-(methylthio)-8a,9,10,11,12,13-hexahydro-8H-7-oxa-1,3,5,13a,14-pentaaza-9,12-methanonaphtho[1,8-ab]heptalen-4-ol

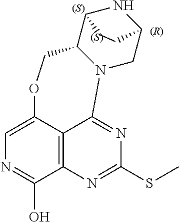

To a solution of tert-butyl (8aS,9S,12R)-4-methoxy-2-(methylthio)-8a,9,10,11,12,13-hexahydro-8H-7-oxa-1,3,5,13a,14-pentaaza-9,12-methanonaphtho[1,8-ab]heptalene-14-carboxylate (85 mg, 0.19 mmol) in MeCN (5 mL) were added TMSI (0.13 mL, 0.57 mmol), and the reaction was stirred at 0° C. for 2 hours. The reaction was monitored by LCMS. Filtered and concentrated to get the (8aS,9S,12R)-2-(methylthio)-8a,9,10,11,12,13-hexahydro-8H-7-oxa-1,3,5,13a,14-pentaaza-9,12-methanonaphtho[1,8-ab]heptalen-4-ol (60 mg, 94.9%) as a white solid.

MS (ESI) m/z: 332 [M+H]⁺.

### Tert-butyl (8aS,9S,12R)-4-hydroxy-2-(methylthio)-8a,9,10,11,12,13-hexahydro-8H-7-oxa-1,3,5,13a,14-pentaaza-9,12-methanonaphtho[1,8-ab]heptalene-14-carboxylate

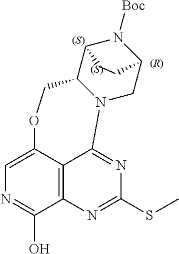

To a solution of (8aS,9S,12R)-2-(methylthio)-8a,9,10,11,12,13-hexahydro-8H-7-oxa-1,3,5,13a,14-pentaaza-9,12-methanonaphtho[1,8-ab]heptalen-4-ol (60 mg, 0.18 mmol) in THF (8 mL) and H₂O (2 mL) were added NaHCO₃ (30.42 mg, 0.36 mmol), (Boc)₂₀ (39.5 mg, 0.18 mmol), and the reaction was stirred at room temperature for 3 hours. LCMS showed the complete consumption of the starting material. The reaction was concentrated in vacuo. The crude product was chromatographed on silica gel (DCM/MeOH 10:1) to give title compound tert-butyl (8aS,9S,12R)-4-hydroxy-2-(methylthio)-8a,9,10,11,12,13-hexahydro-8H-7-oxa-1,3,5,13a,14-pentaaza-9,12-methanonaphtho[1,8-ab]heptalene-14-carboxylate (60 mg, 76.8%) as white solid.

MS (ESI) m/z: 432 [M+H]⁺.

¹H NMR (400 MHZ, CDCl₃) δ 12.55 (s, 1H), 6.68 (s, 1H), 4.50 (d, J=13.7 Hz, 1H), 4.32 (dd, J=13.6, 4.1 Hz, 2H), 3.75-3.70 (m, 1H), 3.67 (dd, J=5.8, 3.5 Hz, 1H), 3.63-3.56 (m, 2H), 3.46 (t, J=6.7 Hz, 1H), 3.20 (d, J=30.2 Hz, 2H), 2.85-2.77 (m, 1H), 2.64 (s, 3H), 2.46 (t, J=9.2 Hz, 1H), 1.49 (s, 9H).

### Tert-butyl (8aS,9S,12R)-5-(7-fluoro-8-((triisopropylsilyl)ethynyl)naphthalen-1-yl)-2-(methylthio)-4-oxo-4,5,8a,9,10,11,12,13-octahydro-8H-7-oxa-1,3,5,13a,14-pentaaza-9,12-methanonaphtho[1,8-ab]heptalene-14-carboxylate

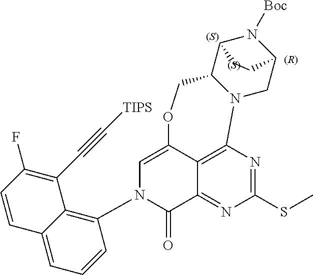

To a solution of tert-butyl (8aS,9S,12R)-4-hydroxy-2-(methylthio)-8a,9,10,11,12,13-hexahydro-8H-7-oxa-1,3,5,13a,14-pentaaza-9,12-methanonaphtho[1,8-ab]heptalene-14-carboxylate (70 mg, 0.16 mmol) in Pyridine (5 mL) were added ((2-fluoro-8-(4,4,5,5-tetramethyl-1,3,2-dioxaborolan-2-yl)naphthalen-1-yl)ethynyl)triisopropylsilane (147 mg, 0.32 mmol), Cu(OAc)₂ (59 mg, 0.32 mmol) and molecular sieve 4 Å (30 mg), and the reaction was stirred at room temperature for 40 hours. LCMS showed the complete consumption of the starting materials. The reaction was concentrated in vacuo. The residue was purified using silica gel column chromatography eluting with DCM/MeOH (10:1) to give the tert-butyl (8aS,9S,12R)-5-(7-fluoro-8-((triisopropylsilyl)ethynyl)naphthalen-1-yl)-2-(methylthio)-4-oxo-4,5,8a,9,10,11,12,13-octahydro-8H-7-oxa-1,3,5,13a,14-pentaaza-9,12-methanonaphtho[1,8-ab]heptalene-14-carboxylate (30 mg, 24.5%) as white solid.

MS (ESI) m/z: 756 [M+H]⁺.

### Tert-butyl (8aS,9S,12R)-5-(7-fluoro-8-((triisopropylsilyl)ethynyl)naphthalen-1-yl)-2-(methylsulfinyl)-4-oxo-4,5,8a,9,10,11,12,13-octahydro-8H-7-oxa-1,3,5,13a,14-pentaaza-9,12-methanonaphtho[1,8-ab]heptalene-14-carboxylate

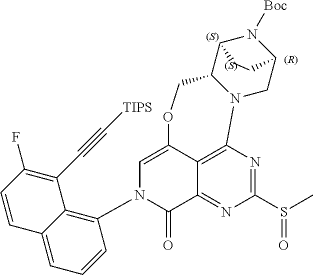

To a flask containing tert-butyl (8aS,9S,12R)-5-(7-fluoro-8-((triisopropylsilyl)ethynyl)naphthalen-1-yl)-2-(methylthio)-4-oxo-4,5,8a,9,10,11,12,13-octahydro-8H-7-oxa-1,3,5,13a,14-pentaaza-9,12-methanonaphtho[1,8-ab]heptalene-14-carboxylate (40 mg, 52.9 μmol) was added DCM (3 mL) followed by the addition of m-CPBA (18 mg, 106 μmol) at 0° C. The mixture was stirred at 0° C. for 10 mins. The mixture was quenched by NaHCO₃(aq). The aqueous layer was extracted with DCM (3×5 mL). Combined the organic layers and dried by Na₂SO₄, filtered and concentrated to afford the crude title compound tert-butyl (8aS,9S,12R)-5-(7-fluoro-8-((triisopropylsilyl)ethynyl)naphthalen-1-yl)-2-(methylsulfinyl)-4-oxo-4,5,8a,9,10, 11, 12, 13-octahydro-8H-7-oxa-1,3,5,13a,14-pentaaza-9,12-methanonaphtho[1,8-ab]heptalene-14-carboxylate (40 mg, 100%) as white foam, which was used for next reaction directly without any further purification.

MS (ESI) m/z: 772 [M+H]⁺.

### Tert-butyl (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-4-oxo-4,5,8a,9,10,11,12,13-octahydro-8H-7-oxa-1,3,5,13a,14-pentaaza-9,12-methanonaphtho[1,8-ab]heptalene-14-carboxylate

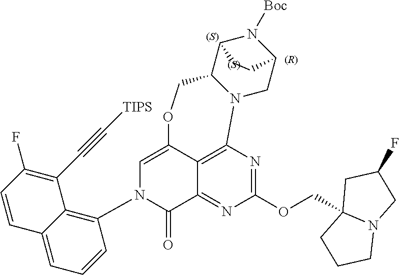

To a oven dried flask ((2R,7aS)-2-fluorotetrahydro-1H-pyrrolizin-7a(5H)-yl) methanol (12.0 mg, 78 μmol), sodium tert-butoxide (5.0 mg, 52 μmol) and Molecular Sieve 4 Å (30 mg) was added, then the flask was heated to 40˜50° C. with a hot air gun while vacuum pump and then degassed with N₂ three times, solid materials in the flask cooled to room temperature followed by the addition of toluene (5 mL) was stirred at rt for 20 mins and continue to cool to 0° C. To the mixture was added tert-butyl (8aS,9S,12R)-5-(7-fluoro-8-((triisopropylsilyl)ethynyl)naphthalen-1-yl)-2-(methylsulfinyl)-4-oxo-4,5,8a,9,10,11,12,13-octahydro-8H-7-oxa-1,3,5,13a,14-pentaaza-9,12-methanonaphtho[1,8-ab]heptalene-14-carboxylate (40 mg, 52 μmol) at 0° C. The mixture warmed to room temperature was stirred at this temperature for 20 mins. The reaction was filtered and concentrated. The crude material was loaded on a silica gel plate. The plate was developed using DCM:MeOH=15:1 to provide the title compound tert-butyl (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-4-oxo-4,5,8a,9,10, 11, 12, 13-octahydro-8H-7-oxa-1,3,5,13a,14-pentaaza-9,12-methanonaphtho[1,8-ab]heptalene-14-carboxylate (20 mg, 45%).

MS (ESI) m/z: 867 [M+H]⁺.

### (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-7-oxa-1,3,5,13a,14-pentaaza-9,12-methanonaphtho[1,8-ab]heptalen-4 (5H)-one

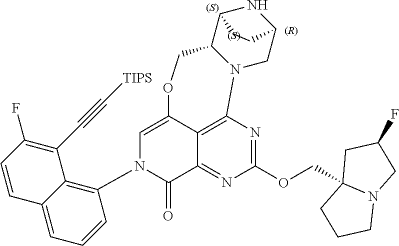

To a flask containing tert-butyl (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-4-oxo-4,5,8a,9,10,11,12,13-octahydro-8H-7-oxa-1,3,5,13a,14-pentaaza-9,12-methanonaphtho[1,8-ab]heptalene-14-carboxylate (20 mg, 23 μmol) was added DCM (1 mL) followed by the addition of HCl/dioxane (4M, 0.5 mL). The mixture was stirred at room temperature for 30 mins. The mixture was concentrated to provide the crude title compound (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-7-oxa-1,3,5,13a,14-pentaaza-9,12-methanonaphtho[1,8-ab]heptalen-4 (5H)-one (˜30 mg, 100%) as a white foam which was used for next reaction step directly.

MS (ESI) m/z: 767 [M+H]⁺.

### (5aS,6S,9R)-2-(8-ethynyl-7-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-2,10a,11,13,14-pentaaza-6,9-methanonaphtho[1,8-ab]heptalen-1 (2H)-one

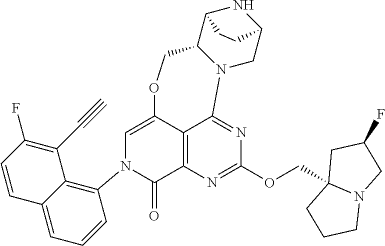 [SMILES: C#Cc1c(F)ccc2cccc(-n3cc4c5c(nc(OC[C@@]67CCCN6C[C@H](F)C7)nc5c3=O)N3C[C@H]5CC[C@H](N5)[C@H]3CO4)c12]

To a flask containing (8aS,9S,12R)-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-7-oxa-1,3,5,13a,14-pentaaza-9,12-methanonaphtho[1,8-ab]heptalen-4 (5H)-one (30 mg, 23 μmol, crude) was added DMF (1 mL) followed by the addition of CsF (7 mg, 46 μmol). The mixture was stirred at rt for 20 mins. The mixture was purified by prep-HPLC with (MeCN, H₂O) to provide the title compound (5aS,6S,9R)-2-(8-ethynyl-7-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-2,10a,11,13,14-pentaaza-6,9-methanonaphtho[1,8-ab]heptalen-1 (2H)-one (1.4 mg, 9.5%).

MS (ESI) m/z: 611 [M+H]⁺.

¹H NMR (400 MHZ, MeOD) δ 8.50 (s, 2H), 8.16 (s, 1H), 7.67 (d, J=25.0 Hz, 1H), 7.49 (s, 1H), 7.34 (d, J=7.8 Hz, 1H), 5.47 (s, 1H), 5.33 (d, J=4.6 Hz, 2H), 4.76 (s, 2H), 4.58 (s, 2H), 3.68 (s, 1H), 3.67 (s, 1H), 3.66 (s, 1H), 3.63 (s, 1H), 3.48 (s, 2H), 3.13 (s, 2H), 2.18 (s, 2H), 2.14 (s, 4H), 2.02 (s, 2H), 1.96 (s, 2H).

Compounds 61-62 can be prepared in a similar way to Compound 43, except for using other appropriate aryl boronic esters and alcohols.

### Compound 61: (5aS,6S,9R)-2-(8-acetyl-7-fluoronaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-2,10a,11,13,14-pentaaza-6,9-methanonaphtho[1,8-ab]heptalen-1 (2H)-one

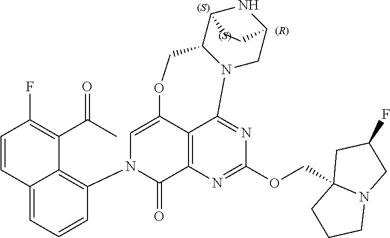

LC/MS (ESI) m/z: 629 [M+H]⁺.

¹H NMR (400 MHZ, MeOD) δ 8.14 (d, J=8.7 Hz, 2H), 7.69 (s, 1H), 7.60 (d, J=7.5 Hz, 1H), 7.47 (s, 1H), 7.07 (d, J=20.6 Hz, 1H), 5.34 (s, 2H), 4.32 (d, J=8.0 Hz, 2H), 3.74 (d, J=11.1 Hz, 2H), 3.65 (d, J=5.3 Hz, 2H), 3.48-3.46 (m, 2H), 3.22 (d, J=8.7 Hz, 2H), 3.15-3.12 (m, 2H), 2.22-2.12 (m, 6H), 2.03 (d, J=5.3 Hz, 7H).

### Compound 62: (5aS,6S,9R)-2-(8-ethyl-7-fluoro-3-hydroxynaphthalen-1-yl)-12-(((2R,7aS)-2-fluorotetrahydro-1H-pyrrolizin-7a(5H)-yl)methoxy)-5a,6,7,8,9,10-hexahydro-5H-4-oxa-2,10a,11,13,14-pentaaza-6,9-methanonaphtho[1,8-ab]heptalen-1 (2H)-one

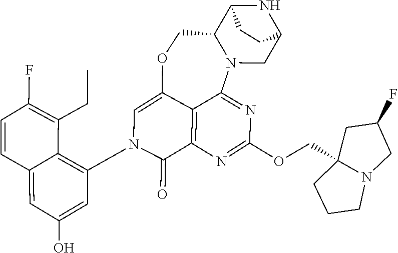

MS (ESI) m/z: 631 [M+H]⁺.

### Example 9

### Preparation of (5aS)-2-(8-ethynyl-7-fluoronaphthalen-1-yl)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline (Compound 44)

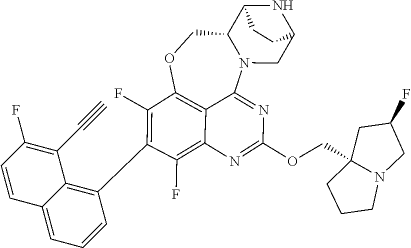

### 4-Bromo-2,3,5,6-tetrafluorobenzoic Acid

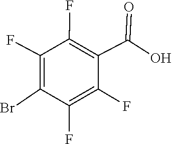 [SMILES: O=C(O)c1c(F)c(F)c(Br)c(F)c1F]

To oven dried one-necked round-bottomed flask with stir bar added 1,4-dibromo-2,3,5,6-tetrafluorobenzene (25 g, 81.2 mmol) under stream of Ar and sealed the flask with rubber septum. To this flask was added dry, freshly distilled THF (500 ml) via cannula. This solution was cooled down to −78° C. by immersing into dry ice-acetone bath for 10-15 mins. To this solution was then slowly added n-butyllithium (1.6 M solutions in hexanes, 53.0 ml, 85.0 mmol) over 20 mins. After 20 minutes, carbon dioxide gas was purged into reaction mixture for 10 mins followed by addition of excess solid dry ice to reaction mixture. The reaction mixture was slowly warmed to 0° C. and quenched with 2M HCl carefully. The solvent was removed on rotary evaporator and product was extracted in methylene chloride. The organic phase was washed with saturated sodium thiosulfate solution, brine solution and dried using anhydrous magnesium sulfate. The solvent was removed on rotary evaporator to yield crude product which was triturated with cold hexane and filtered off to yield pure 4-bromo-2,3,5,6-tetrafluorobenzoic acid (9.8 g, 43% yield).

### Methyl (4-bromo-2,3,5,6-tetrafluorobenzoyl) carbamimidothioate

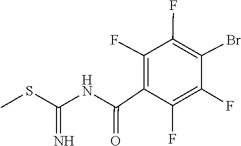 [SMILES: CSC(=N)NC(=O)c1c(F)c(F)c(Br)c(F)c1F]

To a mixture of 4-bromo-2,3,5,6-tetrafluorobenzoic acid (3.4 g, 12.5 mmol) in DCM (50 mL) were added (COCl)₂ (6.35 g, 50 mmol) and DMF (0.1 mL) under N₂ atmosphere at 0° C., and the reaction was stirred at room temperature for 3 hours. TLC indicated the reaction was complete. The reaction was concentrated in vacuo to give 4-bromo-2,3,5,6-tetrafluorobenzoyl chloride (3.5 g, crude) as a yellow oil, which can be used for next step directly.

To a solution of NaOH (2.3 g, 57.5 mmol) in water (100 mL) and THF (50 mL) was added 2-methyl-2-thio-pseudourehydrogensulfate (3.5 g, 12.5 mmol) in batches at 0° C., and the reaction was stirred at 0° C. for 30 min. Then a solution of 4-bromo-2,3,5,6-tetrafluorobenzoyl chloride (3.5 g, crude) in DCM (20 mL) was added at 0° C., and the reaction was stirred at room temperature for 1 hour. TLC (PE:EA=3:1) indicated the reaction was complete. The reaction was extracted with EA (50 mL), dried over anhydrous sodium sulfate and concentrated in vacuo. The residue was purified by silica gel column chromatography (PE:EA=3:1) to give methyl (4-bromo-2,3,5,6-tetrafluorobenzoyl) carbamimidothioate (2.5 g, 58% yield) as a yellow solid.

LC/MS (ESI) (m/z): 345/347 [M+H]⁺.

### 7-Bromo-5,6,8-trifluoro-2-(methylthio) quinazolin-4 (3H)-one

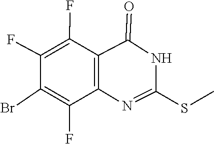 [SMILES: CSc1nc2c(F)c(Br)c(F)c(F)c2c(=O)[nH]1]

A solution of methyl (4-bromo-2,3,5,6-tetrafluorobenzoyl) carbamimidothioate (2.45 g, 7.10 mmol) in DMF (30 mL) was stirred at 120° C. for 3 hours. LCMS showed the reaction was complete. The reaction mixture was poured into water (100 mL) and extracted with EtOAc (30 mL×3). The combined organic phases were washed with brine (30 mL), dried over anhydrous Na₂SO₄ and concentrated to dryness. The residue was purified by column chromatography on silica gel (PE:EtOAc=3:1) to 7-bromo-5,6,8-trifluoro-2-(methylthio) quinazolin-4 (3H)-one (1.66 g, 72% yield) as a yellow solid. LC/MS (ESI) (m/z): 325/327 [M+H]⁺.

### Tert-butyl (1R,5S)-2-(((7-bromo-6,8-difluoro-2-(methylthio)-4-oxo-3,4-dihydroquinazolin-5-yl)oxy)methyl)-3,8-diazabicyclo[3.2.1]octane-8-carboxylate

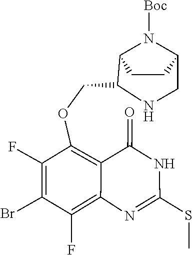

To a solution of tert-butyl (1R,5S)-2-(hydroxymethyl)-3,8-diazabicyclo[3.2.1]octane-8-carboxylate (82 mg, 0.34 mmol) in dry DMF (1 mL) was added NaH (14 mg, 0.34 mmol, 60% in mineral oil) at room temperature. The reaction mixture was stirred at room temperature for 0.5 hour before 7-bromo-5,6,8-trifluoro-2-(methylthio) quinazolin-4 (3H)-one (100 mg, 0.31 mmol) was added at 0° C. The reaction mixture was stirred at 65° C. for 2 hours. LCMS showed the reaction was complete. The reaction mixture was poured into water (10 mL) and extracted with EtOAc (5 mL×3). The combined organic phases were washed with brine (10 mL), dried over anhydrous Na₂SO₄ and concentrated to dryness. The residue was purified by column chromatography on silica gel (EtOAc) to give tert-butyl (1R,5S)-2-(((7-bromo-6,8-difluoro-2-(methylthio)-4-oxo-3,4-dihydroquinazolin-5-yl)oxy)methyl)-3,8-diazabicyclo[3.2.1]octane-8-carboxylate (80 mg, 47.6% yield) as a yellow solid.

LC/MS ESI (m/z): 547/549 [M+H]⁺.

### Tert-butyl 2-bromo-1,3-difluoro-13-(methylthio)-5a,6,7,8,9,10-hexahydro-5H-6,9-epiminoazepino[2′,1′:3,4][1,4]oxazepino[5,6,7-de]quinazoline-15-carboxylate

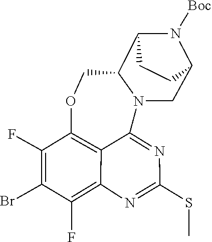 [SMILES: CSc1nc2c3c(c(F)c(Br)c(F)c3n1)OC[C@@H]1[C@@H]3CC[C@H](CN21)N3C(=O)OC(C)(C)C]

To a solution of tert-butyl (1R,5S)-2-(((7-bromo-6,8-difluoro-2-(methylthio)-4-oxo-3,4-dihydroquinazolin-5-yl)oxy)methyl)-3,8-diazabicyclo[3.2.1]octane-8-carboxylate (320 mg, 0.59 mmol) and PyBOP (371 mg, 0.7 mmol) in dry MeCN (10 mL) was added TEA (180 mg, 1.77 mmol) and the resulting mixture was stirred at 80° C. for 2 hours. LCMS showed the reaction was complete. The reaction mixture was diluted with water (10 mL) and extracted with EA (10 mL×2). The combined extracts dried over anhydrous Na₂SO₄ and concentrated to dryness. The residue was purified using silica gel column chromatography eluting with EA/PE=1/1 to give tert-butyl 2-bromo-1,3-difluoro-13-(methylthio)-5a,6,7,8,9,10-hexahydro-5H-6,9-epiminoazepino[2′,1′:3,4][1,4]oxazepino[5,6,7-de]quinazoline-15-carboxylate (200 mg, 71% yield) as a white solid.

LC/MS ESI (m/z): 529/531 [M+H]⁺.

### Tert-butyl 4,6-difluoro-5-(7-fluoro-8-((triisopropylsilyl)ethynyl)naphthalen-1-yl)-2-(methylthio)-8a,9,10,11,12,13-hexahydro-8H-9,12-epiminoazepino[2′,1′:3,4][1,4]oxazepino[5,6,7-de]quinazoline-15-carboxylate

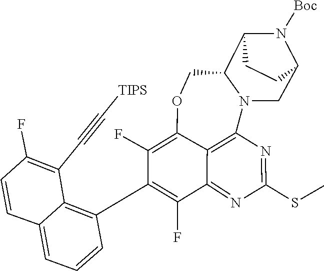

To a solution of tert-butyl 2-bromo-1,3-difluoro-13-(methylthio)-5a,6,7,8,9,10-hexahydro-5H-6,9-epiminoazepino[2′,1′:3,4][1,4]oxazepino[5,6,7-de]quinazoline-15-carboxylate (190 mg, 0.35 mmol) in dioxane (5 mL), H₂O (1 mL) were added ((2-fluoro-8-(4,4,5,5-tetramethyl-1,3,2-dioxaborolan-2-yl)naphthalen-1-yl)ethynyl)triisopropylsilane (240 mg, 0.53 mmol), Pd(PPh₃)₄ (42 mg, 30 μmol) and Na₂CO₃ (115 mg, 1.07 mmol), and the reaction was stirred at 100° C. for 5 hours under N₂. The mixture was extracted with EA (3×5 mL). Combined the EA layer and dried by Na₂SO₄, filtered and concentrated. The crude material was loaded on a silica gel plate. The plate was developed using PE:EA=3:1 to afford the title compound tert-butyl 4,6-difluoro-5-(7-fluoro-8-((triisopropylsilyl)ethynyl)naphthalen-1-yl)-2-(methylthio)-8a,9,10,11,12,13-hexahydro-8H-9,12-epiminoazepino[2′,1′:3,4][1,4]oxazepino[5,6,7-de]quinazoline-15-carboxylate (80 mg, 28.7%).

MS (ESI) m/z: 775 [M+H]⁺.

### Tert-butyl 4,6-difluoro-5-(7-fluoro-8-((triisopropylsilyl)ethynyl)naphthalen-1-yl)-2-(methylsulfonyl)-8a,9,10,11,12,13-hexahydro-8H-9,12-epiminoazepino[2′,1′:3,4][1,4]oxazepino[5,6,7-de]quinazoline-15-carboxylate

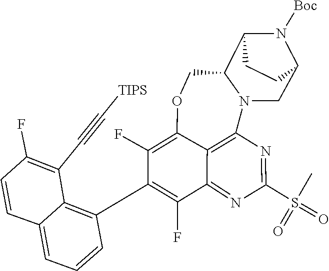

To a flask containing tert-butyl 4,6-difluoro-5-(7-fluoro-8-((triisopropylsilyl)ethynyl)naphthalen-1-yl)-2-(methylthio)-8a,9,10,11,12,13-hexahydro-8H-9,12-epiminoazepino[2′,1′:3,4][1,4]oxazepino[5,6,7-de]quinazoline-15-carboxylate (75 mg, 0.09 mmol) was added DCM (4 mL) followed by the addition of m-CPBA (33 mg, 0.19 mmol) at 0° C. The mixture was stirred at 0° C. for 20 min. The reaction was quenched by NaHCO₃(aq). The mixture was extracted with DCM (3×3 mL) and dried by Na₂SO₄ to afford the title compound tert-butyl 4,6-difluoro-5-(7-fluoro-8-((triisopropylsilyl)ethynyl)naphthalen-1-yl)-2-(methylsulfonyl)-8a,9,10,11,12,13-hexahydro-8H-9,12-epiminoazepino[2′,1′:3,4][1,4]oxazepino[5,6,7-de]quinazoline-15-carboxylate (78 mg, 99.8%).

MS (ESI) m/z: 807 [M+H]⁺.

### Tert-butyl 4,6-difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline-15-carboxylate

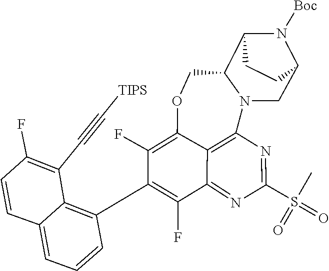

To an oven dried flask containing [(2R,7aS)-2-fluoro-2,3,5,6,7,7a-hexahydro-1H-pyrrolizin-7a-yl]methanol (27 mg, 0.17 mmol) was added toluene (4 mL) followed by the addition of 4 Å MS and t-BuONa (16 mg, 0.17 mmol) at 0° C. The mixture was stirred at rt for 10 min. The mixture was added tert-butyl 4,6-difluoro-5-(7-fluoro-8-((triisopropylsilyl)ethynyl)naphthalen-1-yl)-2-(methylsulfonyl)-8a,9,10,11,12,13-hexahydro-8H-9,12-epiminoazepino[2′, l′: 3,4][1,4]oxazepino[5,6,7-de]quinazoline-15-carboxylate (70 mg, 0.08 mmol). The mixture was stirred at rt for 0.5 hour. The reaction was quenched with saturated NH₄Cl solution and filtered. The aqueous layer was back extracted with EA (3×5 mL). Combined the EA layer and dried by Na₂SO₄, filtered and concentrated. The crude material was loaded on a silica gel plate. The plate was developed using DCM:MeOH=10:1 to provide the title compound tert-butyl 4,6-difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline-15-carboxylate (60 mg, 78.0%).

MS (ESI) m/z: 886 [M+H]⁺.

### 4,6-Difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline

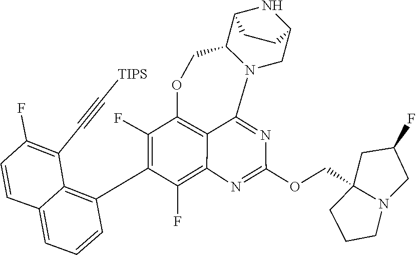

To a flask containing tert-butyl 4,6-difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline-15-carboxylate (55 mg, 0.06 mmol) was added DCM (3 mL) followed by the addition of HCl/dioxane (1 mL, 4M in dioxane). The mixture was stirred at rt for 1 hour. The resulting was concentrated under vacuum to provide the title compound 4,6-difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′, l′: 3,4][1,4]oxazepino[5,6,7-de]quinazoline (48 mg, 98.3%).

MS (ESI) m/z: 786 [M+H]⁺.

### (5aS)-2-(8-ethynyl-7-fluoronaphthalen-1-yl)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

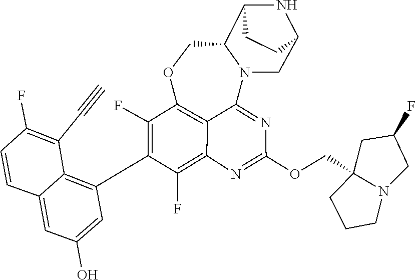 [SMILES: C#Cc1c(F)ccc2cc(O)cc(-c3c(F)c4c5c(nc(OC[C@@]67CCCN6C[C@H](F)C7)nc5c3F)N3C[C@H]5CC[C@H](N5)[C@H]3CO4)c12]

To a flask containing 4,6-difluoro-5-(7-fluoro-8-((triisopropylsilyl)ethynyl)naphthalen-1-yl)-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline (48 mg, 0.06 mmol) was added DMF (3 mL) followed by the addition of CsF (185 mg, 1.22 mmol). The mixture was stirred at rt for 1 hour. The mixture was purified by prep-HPLC (Column: UniHybrid 5-120 C4 150*21.2 mm 5 um; H₂O (0.1% NH₄OH)/CH₃CN) to afford the title compound 5-(8-ethynyl-7-fluoronaphthalen-1-yl)-4,6-difluoro-2-(((2R,7aS)-2-fluorotetrahydro-1H-pyrrolizin-7a(5H)-yl)methoxy)-8a,9,10,11,12,13-hexahydro-8H-9,12-epiminoazepino[2′,1′:3,4][1,4]oxazepino[5,6,7-de]quinazoline (21 mg, 54.6%).

LC-MS (ESI): m/z 630 [M+H]⁺.

¹H NMR (400 MHZ, MeOD) δ 8.12-8.05 (m, 2H), 7.63 (td, J=7.7, 2.6 Hz, 1H), 7.53 (t, J=7.0 Hz, 1H), 7.43 (td, J=8.9, 2.5 Hz, 1H), 5.30 (d, J=54.0 Hz, 1H), 5.07 (ddd, J=29.7, 13.4, 2.4 Hz, 1H), 4.63-4.54 (m, 2H), 4.38-4.23 (m, 2H), 4.20-4.08 (m, 2H), 3.69 (s, 1H), 3.56 (d, J=4.1 Hz, 1H), 3.29-3.11 (m, 4H), 3.01 (td, J=9.6, 5.7 Hz, 1H), 2.34-2.10 (m, 3H), 2.04-1.79 (m, 7H)).

The following compounds can be prepared in a similar way to Compound 44, except for using other appropriate aryl boronic esters and alcohols.

### Compound 63: 4-((5aS,6S,9R)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-ethyl-6-fluoronaphthalen-2-ol

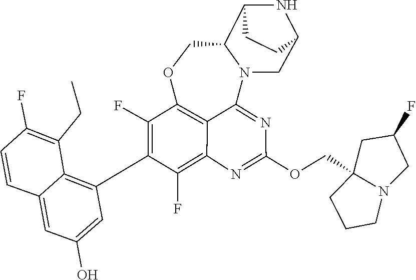 [SMILES: CCc1c(F)ccc2cc(O)cc(-c3c(F)c4c5c(nc(OC[C@@]67CCCN6C[C@H](F)C7)nc5c3F)N3C[C@H]5CC[C@H](N5)[C@H]3CO4)c12]

LC/MS (ESI) m/z: 650 [M+H]⁺.

### Compound 64: 4-((5aS,6S,9R)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-ethynyl-6-fluoronaphthalen-2-ol

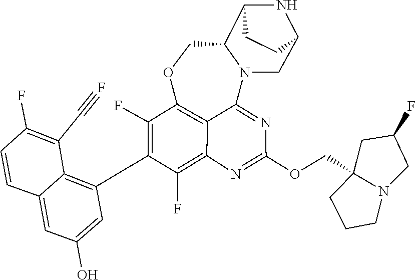

LC/MS (ESI) m/z: 646 [M+H]⁺.

### Compound 65: 4-((5aS,6S,9R)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-ethyl-6-fluoronaphthalen-2-ol

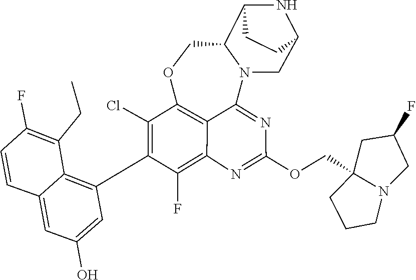 [SMILES: CCc1c(F)ccc2cc(O)cc(-c3c(Cl)c4c5c(nc(OC[C@@]67CCCN6C[C@H](F)C7)nc5c3F)N3C[C@H]5CC[C@H](N5)[C@H]3CO4)c12]

LC/MS (ESI) m/z: 666 [M+H]⁺.

### Compound 66: 4-((5aS,6S,9R)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-ethynyl-6-fluoronaphthalen-2-ol

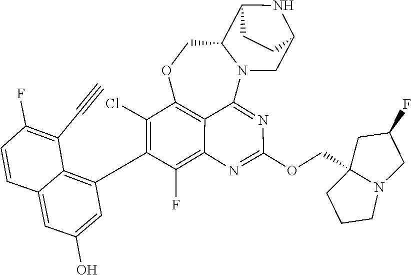 [SMILES: C#Cc1c(F)ccc2cc(O)cc(-c3c(Cl)c4c5c(nc(OC[C@@]67CCCN6C[C@H](F)C7)nc5c3F)N3C[C@H]5CC[C@H](N5)[C@H]3CO4)c12]

LC/MS (ESI) m/z: 662 [M+H]⁺.

### Compound 74: 4-((5aS,6S,9R)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-6-fluoro-5-methylnaphthalen-2-ol

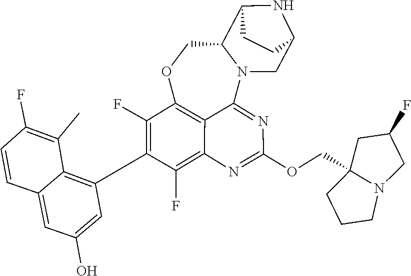 [SMILES: Cc1c(F)ccc2cc(O)cc(-c3c(F)c4c5c(nc(OC[C@@]67CCCN6C[C@H](F)C7)nc5c3F)N3C[C@H]5CC[C@H](N5)[C@H]3CO4)c12]

LC/MS (ESI) m/z: 636 [M+H]⁺.

### Compound 75: 5-chloro-4-((5aS,6S,9R)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-6-fluoronaphthalen-2-ol

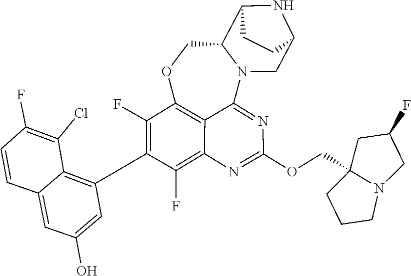 [SMILES: Oc1cc(-c2c(F)c3c4c(nc(OC[C@@]56CCCN5C[C@H](F)C6)nc4c2F)N2C[C@H]4CC[C@H](N4)[C@H]2CO3)c2c(Cl)c(F)ccc2c1]

LC/MS (ESI) m/z: 656 [M+H]⁺.

### Compound 76: 4-((5aS,6S,9R)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5,6-difluoronaphthalen-2-ol

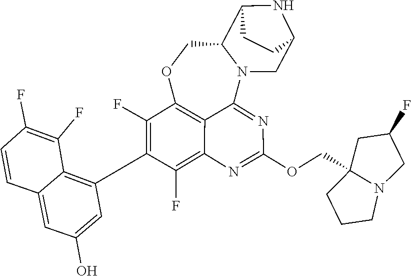 [SMILES: Oc1cc(-c2c(F)c3c4c(nc(OC[C@@]56CCCN5C[C@H](F)C6)nc4c2F)N2C[C@H]4CC[C@H](N4)[C@H]2CO3)c2c(F)c(F)ccc2c1]

LC/MS (ESI) m/z: 640 [M+H]⁺.

### Compound 77: 4-((5aS,6S,9R)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-6-fluoro-5-methylnaphthalen-2-ol

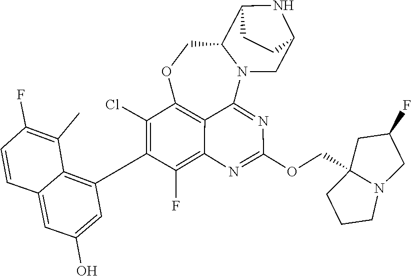 [SMILES: Cc1c(F)ccc2cc(O)cc(-c3c(Cl)c4c5c(nc(OC[C@@]67CCCN6C[C@H](F)C7)nc5c3F)N3C[C@H]5CC[C@H](N5)[C@H]3CO4)c12]

LC/MS (ESI) m/z: 652 [M+H]⁺.

### Compound 78: 5-chloro-4-((5aS,6S,9R)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-6-fluoronaphthalen-2-ol

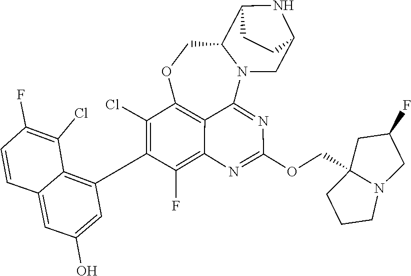 [SMILES: Oc1cc(-c2c(Cl)c3c4c(nc(OC[C@@]56CCCN5C[C@H](F)C6)nc4c2F)N2C[C@H]4CC[C@H](N4)[C@H]2CO3)c2c(Cl)c(F)ccc2c1]

LC/MS (ESI) m/z: 672 [M+H]⁺.

### Compound 79: 4-((5aS,6S,9R)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5,6-difluoronaphthalen-2-ol

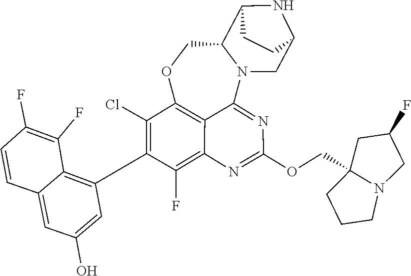 [SMILES: Oc1cc(-c2c(Cl)c3c4c(nc(OC[C@@]56CCCN5C[C@H](F)C6)nc4c2F)N2C[C@H]4CC[C@H](N4)[C@H]2CO3)c2c(F)c(F)ccc2c1]

LC/MS (ESI) m/z: 656 [M+H]⁺.

### Compound 80: (5aS,6S,9R)-3-chloro-2-(8-ethynyl-7-fluoronaphthalen-1-yl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

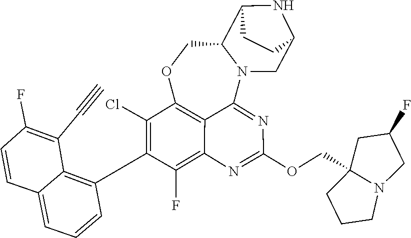 [SMILES: C#Cc1c(F)ccc2cccc(-c3c(Cl)c4c5c(nc(OC[C@@]67CCCN6C[C@H](F)C7)nc5c3F)N3C[C@H]5CC[C@H](N5)[C@H]3CO4)c12]

LC/MS (ESI) m/z: 646 [M+H]⁺.

### Compound 81: 6-((5aS,6R,9S)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl) pyridin-2-amine

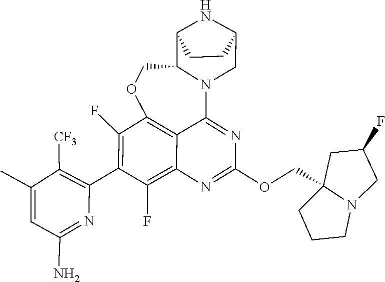 [SMILES: Cc1cc(N)nc(-c2c(F)c3c4c(nc(OC[C@@]56CCCN5C[C@H](F)C6)nc4c2F)N2C[C@H]4CC[C@H](N4)[C@H]2CO3)c1C(F)(F)F]

LC/MS (ESI) m/z: 636 [M+H]⁺.

### Compound 82: (5aS,6R,9S)-2-(5-(difluoromethyl)-3-methyl-2-(trifluoromethyl)phenyl)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

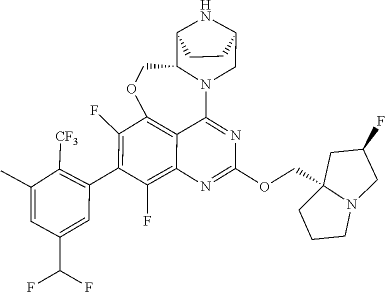 [SMILES: Cc1cc(C(F)F)cc(-c2c(F)c3c4c(nc(OC[C@@]56CCCN5C[C@H](F)C6)nc4c2F)N2C[C@H]4CC[C@H](N4)[C@H]2CO3)c1C(F)(F)F]

LC/MS (ESI) m/z: 670 [M+H]⁺.

### Compound 83: (5aS,6R,9S)-2-(3-chloro-2-cyclopropyl-5-(difluoromethyl)phenyl)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

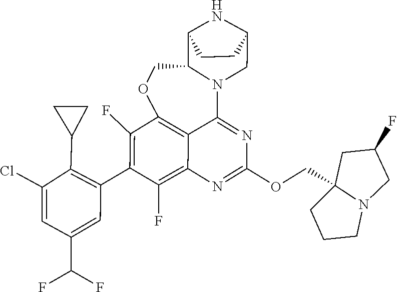 [SMILES: Fc1c(-c2cc(C(F)F)cc(Cl)c2C2CC2)c(F)c2nc(OC[C@@]34CCCN3C[C@H](F)C4)nc3c2c1OC[C@@H]1[C@@H]2CC[C@H](CN31)N2]

LC/MS (ESI) m/z: 662 [M+H]⁺.

### Compound 84: (5aS,6R,9S)-2-(5-chloro-4-cyclopropylpyridin-3-yl)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

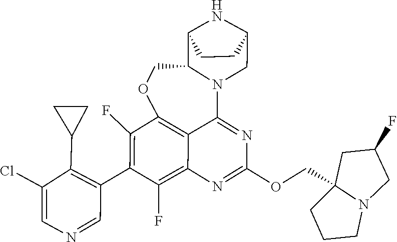 [SMILES: Fc1c(-c2cncc(Cl)c2C2CC2)c(F)c2nc(OC[C@@]34CCCN3C[C@H](F)C4)nc3c2c1OC[C@@H]1[C@@H]2CC[C@H](CN31)N2]

LC/MS (ESI) m/z: 613 [M+H]⁺.

### Compound 85:5-chloro-6-((5aS,6R,9S)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methylpyridin-2-amine

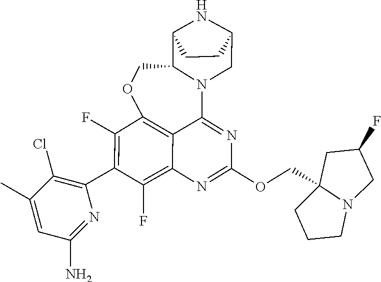 [SMILES: Cc1cc(N)nc(-c2c(F)c3c4c(nc(OC[C@@]56CCCN5C[C@H](F)C6)nc4c2F)N2C[C@H]4CC[C@H](N4)[C@H]2CO3)c1Cl]

LC/MS (ESI) m/z: 602 [M+H]⁺.

### Compound 86:6-((5aS,6R,9S)-1,3-difluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-(difluoromethyl)-4-methylpyridin-2-amine

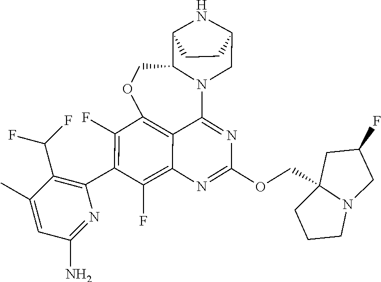 [SMILES: Cc1cc(N)nc(-c2c(F)c3c4c(nc(OC[C@@]56CCCN5C[C@H](F)C6)nc4c2F)N2C[C@H]4CC[C@H](N4)[C@H]2CO3)c1C(F)F]

LC/MS (ESI) m/z: 618 [M+H]⁺.

### Compound 87:6-((5aS,6R,9S)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl) pyridin-2-amine

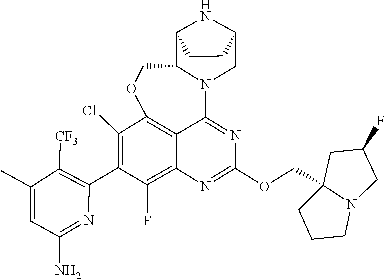 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1C(F)(F)F]

LC/MS (ESI) m/z: 652 [M+H]⁺.

### Compound 88: (5aS,6R,9S)-3-chloro-2-(5-(difluoromethyl)-3-methyl-2-(trifluoromethyl)phenyl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

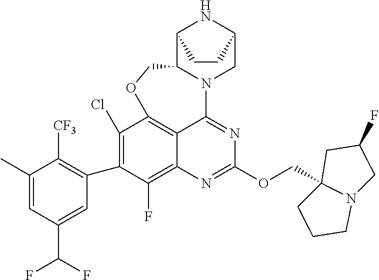 [SMILES: Cc1cc(C(F)F)cc(-c2c(Cl)c3c4c(nc(OC[C@@]56CCCN5C[C@H](F)C6)nc4c2F)N2C[C@H]4CC[C@H](N4)[C@H]2CO3)c1C(F)(F)F]

LC/MS (ESI) m/z: 686 [M+H]⁺.

### Compound 89: (5aS,6R,9S)-3-chloro-2-(3-chloro-2-cyclopropyl-5-(difluoromethyl)phenyl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

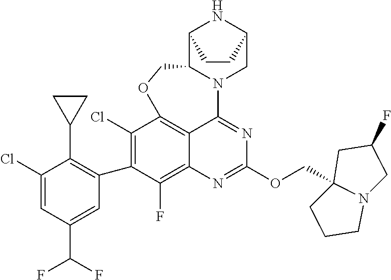 [SMILES: Fc1c(-c2cc(C(F)F)cc(Cl)c2C2CC2)c(Cl)c2c3c(nc(OC[C@@]45CCCN4C[C@H](F)C5)nc13)N1C[C@H]3CC[C@H](N3)[C@H]1CO2]

LC/MS (ESI) m/z: 678 [M+H]⁺.

### Compound 90: (5aS,6R,9S)-3-chloro-2-(5-chloro-4-cyclopropylpyridin-3-yl)-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazoline

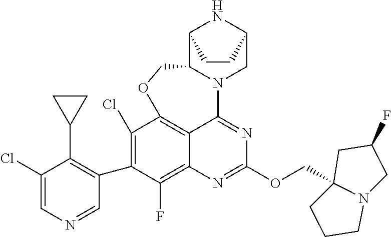 [SMILES: Fc1c(-c2cncc(Cl)c2C2CC2)c(Cl)c2c3c(nc(OC[C@@]45CCCN4C[C@H](F)C5)nc13)N1C[C@H]3CC[C@H](N3)[C@H]1CO2]

LC/MS (ESI) m/z: 629 [M+H]⁺.

### Compound 91: 5-chloro-6-((5aS,6R,9S)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-4-methylpyridin-2-amine

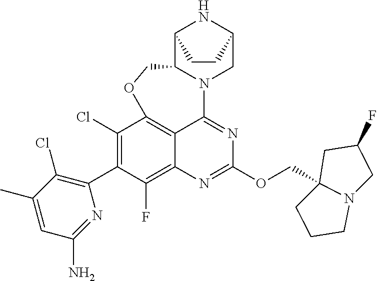 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1Cl]

LC/MS (ESI) m/z: 618 [M+H]⁺.

### Compound 92: 6-((5aS,6R,9S)-3-chloro-1-fluoro-13-(((2R,7aS)-2-fluorotetrahydro-1H-pyrrolizin-7a(5H)-yl)methoxy)-5a,6,7,8,9,10-hexahydro-5H-6,9-epiminoazepino[2′,1′:3,4][1,4]oxazepino[5,6,7-de]quinazolin-2-yl)-5-(difluoromethyl)-4-methylpyridin-2-amine

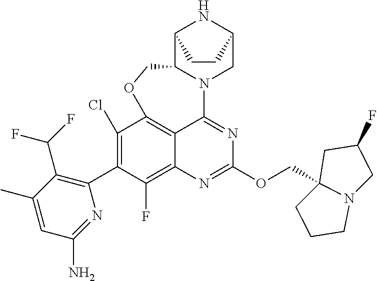 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1C(F)F]

LC/MS (ESI) m/z: 634 [M+H]⁺.

### Compound 99: (5aS,6R,9S)-2-(8-ethynyl-7-fluoronaphthalen-1-yl)-1,3-difluoro-13-((1-(morpholinomethyl)cyclopropyl)methoxy)-5a,6,7,8,9,10-hexahydro-5H-6,9-epiminoazepino[2′,1′:3,4][1,4]oxazepino[5,6,7-de]quinazoline

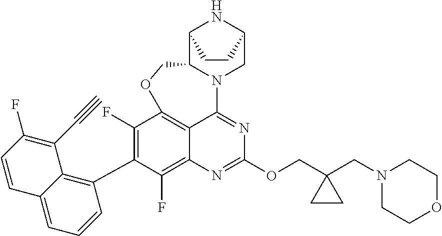 [SMILES: C#Cc1c(F)ccc2cccc(-c3c(F)c4c5c(nc(OCC6(CN7CCOCC7)CC6)nc5c3F)N3C[C@H]5CC[C@H](N5)[C@H]3CO4)c12]

LC/MS (ESI) m/z: 642 [M+H]⁺.

### Compound 100: 4-((5aS,6R,9S)-1,3-difluoro-13-((1-(morpholinomethyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethyl-6-fluoronaphthalen-2-ol

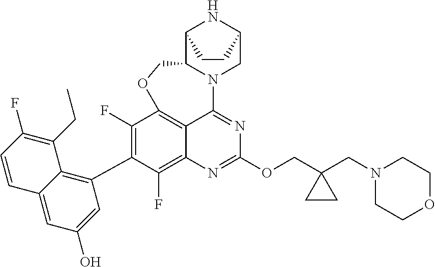 [SMILES: CCc1c(F)ccc2cc(O)cc(-c3c(F)c4c5c(nc(OCC6(CN7CCOCC7)CC6)nc5c3F)N3C[C@H]5CC[C@H](N5)[C@H]3CO4)c12]

LC/MS (ESI) m/z: 662 [M+H]⁺.

### Compound 101: 6-((5aS,6R,9S)-1,3-difluoro-13-((1-(morpholinomethyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl) pyridin-2-amine

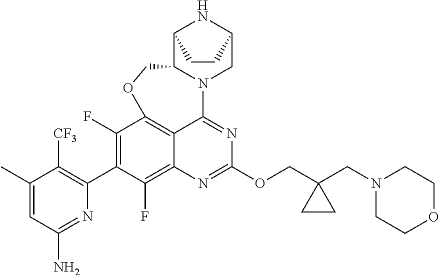 [SMILES: Cc1cc(N)nc(-c2c(F)c3c4c(nc(OCC5(CN6CCOCC6)CC5)nc4c2F)N2C[C@H]4CC[C@H](N4)[C@H]2CO3)c1C(F)(F)F]

LC/MS (ESI) m/z: 648 [M+H]⁺.

### Compound 102:4-((5aS,6R,9S)-1,3-difluoro-13-((1-(morpholinomethyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethynyl-6-fluoronaphthalen-2-ol

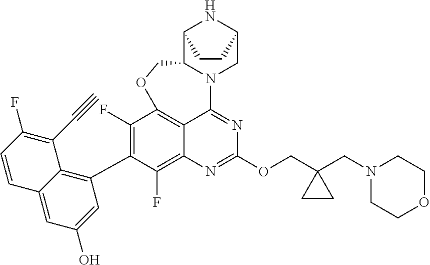 [SMILES: C#Cc1c(F)ccc2cc(O)cc(-c3c(F)c4c5c(nc(OCC6(CN7CCOCC7)CC6)nc5c3F)N3C[C@H]5CC[C@H](N5)[C@H]3CO4)c12]

LC/MS (ESI) m/z: 658 [M+H]⁺.

### Compound 103: 4-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethyl-6-fluoronaphthalen-2-ol

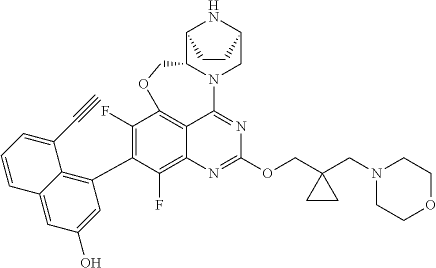 [SMILES: C#Cc1cccc2cc(O)cc(-c3c(F)c4c5c(nc(OCC6(CN7CCOCC7)CC6)nc5c3F)N3C[C@H]5CC[C@H](N5)[C@H]3CO4)c12]

LC/MS (ESI) m/z: 640 [M+H]⁺.

### Compound 104: 5-chloro-4-((5aS,6R,9S)-1,3-difluoro-13-((1-(morpholinomethyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-6-fluoronaphthalen-2-ol

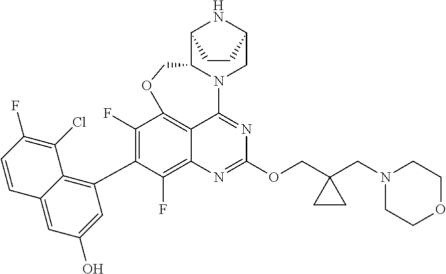 [SMILES: Oc1cc(-c2c(F)c3c4c(nc(OCC5(CN6CCOCC6)CC5)nc4c2F)N2C[C@H]4CC[C@H](N4)[C@H]2CO3)c2c(Cl)c(F)ccc2c1]

LC/MS (ESI) m/z: 668 [M+H]⁺.

### Compound 105: (5aS,6R,9S)-2-(8-ethynyl-7-fluoronaphthalen-1-yl)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazoline

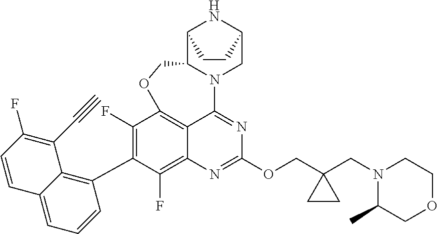 [SMILES: C#Cc1c(F)ccc2cccc(-c3c(F)c4c5c(nc(OCC6(CN7CCOC[C@H]7C)CC6)nc5c3F)N3C[C@H]5CC[C@H](N5)[C@H]3CO4)c12]

LC/MS (ESI) m/z: 656 [M+H]⁺.

### Compound 106: 4-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethyl-6-fluoronaphthalen-2-ol

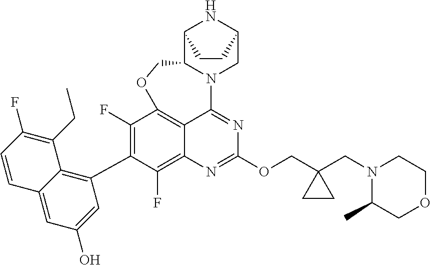 [SMILES: CCc1c(F)ccc2cc(O)cc(-c3c(F)c4c5c(nc(OCC6(CN7CCOC[C@H]7C)CC6)nc5c3F)N3C[C@H]5CC[C@H](N5)[C@H]3CO4)c12]

LC/MS (ESI) m/z: 676 [M+H]⁺.

### Compound 107: 6-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-4-methyl-5-(trifluoromethyl) pyridin-2-amine

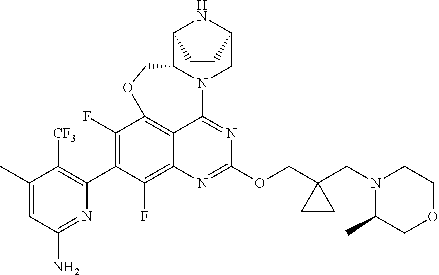 [SMILES: Cc1cc(N)nc(-c2c(F)c3c4c(nc(OCC5(CN6CCOC[C@H]6C)CC5)nc4c2F)N2C[C@H]4CC[C@H](N4)[C@H]2CO3)c1C(F)(F)F]

LC/MS (ESI) m/z: 662 [M+H]⁺.

### Compound 108: 4-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethynyl-6-fluoronaphthalen-2-ol

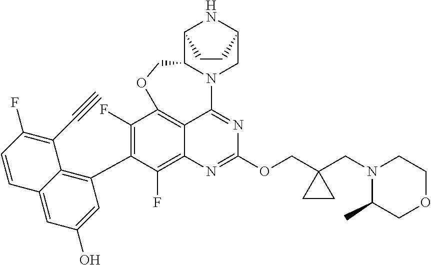 [SMILES: C#Cc1c(F)ccc2cc(O)cc(-c3c(F)c4c5c(nc(OCC6(CN7CCOC[C@H]7C)CC6)nc5c3F)N3C[C@H]5CC[C@H](N5)[C@H]3CO4)c12]

LC/MS (ESI) m/z: 672 [M+H]⁺.

### Compound 109:4-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-5-ethynylnaphthalen-2-ol

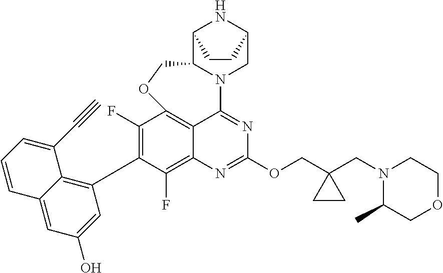 [SMILES: C#Cc1cccc2cc(O)cc(-c3c(F)c4c5c(nc(OCC6(CN7CCOC[C@H]7C)CC6)nc5c3F)N3C[C@H]5CC[C@H](N5)[C@H]3CO4)c12]

LC/MS (ESI) m/z: 654 [M+H]⁺.

### Compound 110: 5-chloro-4-((5aS,6R,9S)-1,3-difluoro-13-((1-(((R)-3-methylmorpholino)methyl)cyclopropyl)methoxy)-5a,6,7,8,9,10-hexahydro-5H-6,9-epiminoazepino[2′,1′:3,4][1,4]oxazepino[5,6,7-de]quinazolin-2-yl)-6-fluoronaphthalen-2-ol

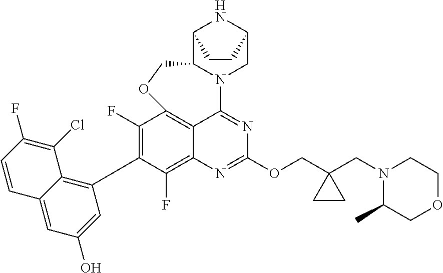 [SMILES: C[C@@H]1COCCN1CC1(COc2nc3c4c(c(F)c(-c5cc(O)cc6ccc(F)c(Cl)c56)c(F)c4n2)OC[C@@H]2[C@@H]4CC[C@H](CN32)N4)CC1]

LC/MS (ESI) m/z: 682 [M+H]⁺.

### Compound 111: 5-ethynyl-6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol

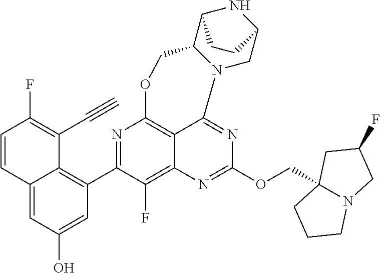 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12]

LC/MS (ESI) m/z: 629.4 [M+H]⁺.

¹H NMR (400 MHZ, Methanol-d₄) δ 8.35 (s, 2H), 7.87-7.82 (m, 1H), 7.35-7.29 (m, 2H), 7.20 (dd, J=30.3, 2.5 Hz, 1H), 5.49 (d, J=52.6 Hz, 1H), 5.12 (dd, J=13.9, 2.2 Hz, 1H), 4.67-4.57 (m, 2H), 4.54-4.47 (m, 2H), 4.23 (s, 1H), 3.90 (s, 1H), 3.87-3.67 (m, 4H), 3.51 (d, J=34.9 Hz, 1H), 3.34 (s, 2H), 2.63-2.47 (m, 2H), 2.35 (s, 1H), 2.26 (d, J=6.7 Hz, 2H), 2.09-1.87 (m, 5H).

### Example 10

### Preparation of (Decanoyloxy)methyl (5aS,6S,9R)-2-(8-ethyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (Compound 45)

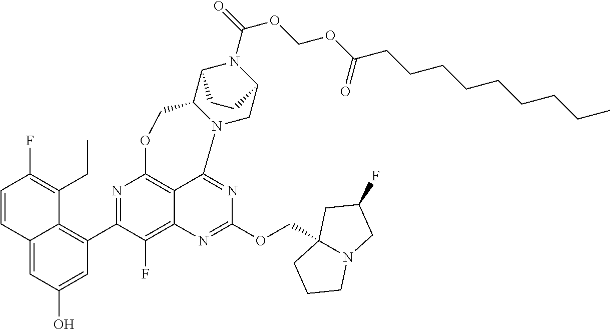 [SMILES: CCCCCCCCCC(=O)OCOC(=O)N1[C@@H]2CC[C@H]1[C@H]1COc3nc(-c4cc(O)cc5ccc(F)c(CC)c45)c(F)c4nc(OC[C@@]56CCCN5C[C@H](F)C6)nc(c34)N1C2]

To a solution of 5-ethyl-6-fluoro-4-((5aS,6S,9R)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalen-2-yl)naphthalen-2-ol (100 mg, 0.16 mmol) in DMF (1 mL) was added (((4-nitrophenoxy)carbonyl)oxy)methyl decanoate (46 mg, 0.13 mmol) and NaHCO₃ (28 mg, 0.26 mmol) at 0° C. and the mixture was stirred at rt for 6 hrs. The mixture was filtered. The filtrate was purified by prep-HPLC (C18, 30˜90% acetonitrile in H₂O with 0.1% formic acid) to give (decanoyloxy)methyl (5aS,6S,9R)-2-(8-ethyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate (8.1 mg, 6% yield) as a white solid.

LC/MS (ESI) m/z: 861 (M+H)⁺.

¹H NMR (400 MHZ, CDCl₃) 7.40-7.16 (m, 2H), 7.07-6.87 (m, 2H), 6.53-6.48 (m, 1H), 5.63 (s, 2H), 5.41-5.16 (m, 1H), 5.05-4.91 (m, 1H), 4.53-4.41 (m, 1H), 4.24-4.11 (m, 3H), 4.02-3.83 (m, 2H), 3.59-3.38 (m, 2H), 3.31-3.09 (m, 3H), 3.03-2.84 (m, 3H), 2.37-2.16 (m, 5H), 2.08-2.04 (m, 2H), 1.95-1.87 (m, 3H), 1.64-1.61 (m, 2H), 1.48-1.45 (m, 2H), 1.10 (s, 12H), 0.73-0.70 (m, 3H), 0.67-0.63 (m, 3H).

The following compounds can be prepared in a similar way to Compound 45, except for using other appropriate aryl boronic esters and alcohols.

### Compound 67: 1-(pivaloyloxy)ethyl (6R,9S)-2-(8-ethynyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

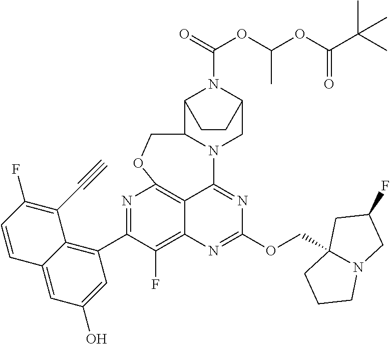 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)OC(=O)C(C)(C)C)c12]

LC/MS (ESI) m/z: 801 (M+H)⁺.

### Compound 68: 1-acetoxyethyl (6R,9S)-2-(8-ethynyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

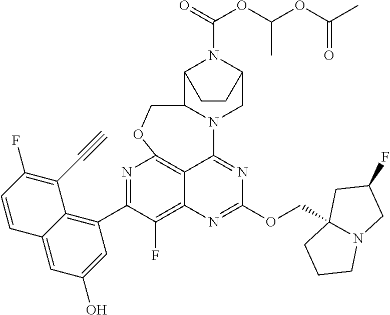 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)OC(C)=O)c12]

LC/MS (ESI) m/z: 759 (M+H)⁺.

### Compound 69: 1-(propionyloxy)ethyl (6R,9S)-2-(8-ethynyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

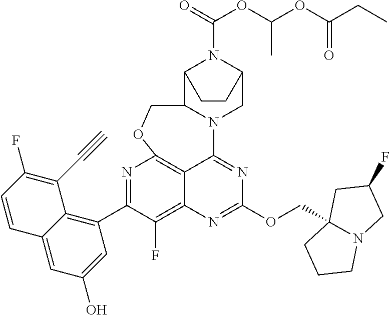 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)OC(=O)CC)c12]

LC/MS (ESI) m/z: 773 (M+H)⁺.

### Compound 70: 1-(isobutyryloxy)ethyl (6R,9S)-2-(8-ethynyl-7-fluoro-3-hydroxynaphthalen-1-yl)-1-fluoro-12-(((2R,7aS)-2-fluorotetrahydro-1H-pyrrolizin-7a(5H)-yl)methoxy)-5a,6,7,8,9,10-hexahydro-5H-4-oxa-3,10a,11,13,14-pentaaza-6,9-methanonaphtho[1,8-ab]heptalene-14-carboxylate

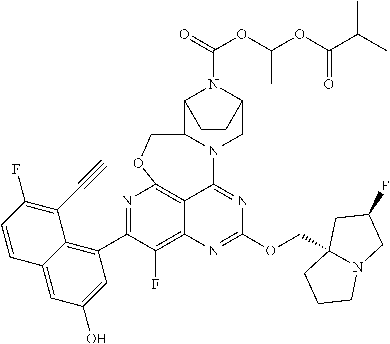 [SMILES: C#Cc1c(F)ccc2cc(O)cc(-c3nc4c5c(nc(OC[C@@]67CCCN6C[C@H](F)C7)nc5c3F)N3CC5CCC(C3CO4)N5C(=O)OC(C)OC(=O)C(C)C)c12]

LC/MS (ESI) m/z: 787 (M+H)⁺.

### Example 11

### Preparation of (9S,12R)-3-(8-chloro-7-fluoronaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one (Compound 93 and Compound 94)

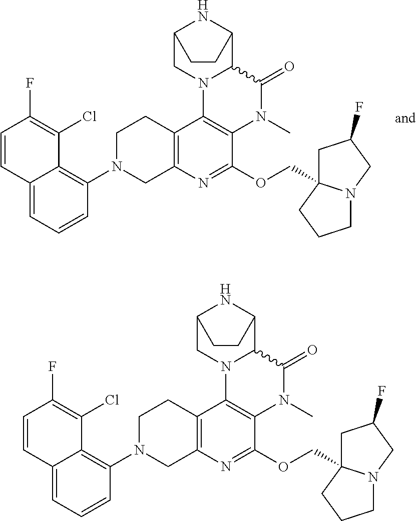 [SMILES: CN1C(=O)C2C3CCC(CN2c2c4c(nc(OC[C@@]56CCCN5C[C@H](F)C6)c21)CN(c1cccc2ccc(F)c(Cl)c12)CC4)N3 ; CN1C(=O)C2C3CCC(CN2c2c4c(nc(OC[C@@]56CCCN5C[C@H](F)C6)c21)CN(c1cccc2ccc(F)c(Cl)c12)CC4)N3]

### Ethyl (1S,5R)-8-(4-methoxybenzyl)-4-oxo-3,8-diazabicyclo[3.2.1]octane-2-carboxylate

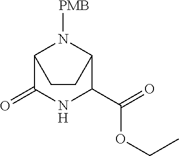

To a solution of ethyl (1S,2S,5R)-4-oxo-3,8-diazabicyclo[3.2.1]octane-2-carboxylate (4 g, 20 mmol) in DCE (100 mL) were added sodium bis(acetyloxy)boranyl acetate (6.38 g, 30 mmol), molecular sieve 4 Å (1 g, 1.0 mmol), and 4-methoxybenzaldehyde (3.2 mL, 26 mmol), AcOH (1.2 mL, 20 mmol) and the reaction was stirred at room temperature for 18 hours. The reaction was concentrated in vacuo. The residue was purified using silica gel column chromatography eluting with methanol in chloroform (1:10) to afford the title compound ethyl (1S,5R)-8-(4-methoxybenzyl)-4-oxo-3,8-diazabicyclo[3.2.1]octane-2-carboxylate (4 g, 62%) as s white solid.

MS (ESI) m/z: 319 [M+H]⁺.

### Ethyl (1S,5R)-8-(4-methoxybenzyl)-3,8-diazabicyclo[3.2.1]octane-2-carboxylate

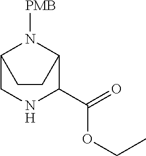

To a solution of ethyl (1S,5R)-8-(4-methoxybenzyl)-4-oxo-3,8-diazabicyclo[3.2.1]octane-2-carboxylate (4 g, 12.5 mmol) in THF (30 mL) were added Borane-methyl sulfide complex (31.4 mL, 2.5 M in Dimethyl sulfide), and the reaction was stirred at room temperature for 18 hours. LCMS showed the reaction was over, then MeOH (5 mL) was added and reflux at 60° C. for 18 hours. The reaction was concentrated in vacuo. The residue was purified using silica gel column chromatography eluting with methanol in chloroform (1:10) to afford the title compound ethyl (1S,5R)-8-(4-methoxybenzyl)-3,8-diazabicyclo[3.2.1]octane-2-carboxylate (1.3 g, 34%) as a yellow oil.

MS (ESI) m/z: 305 [M+H]⁺.

### Ethyl 3-aminoisonicotinate

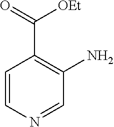 [SMILES: CCOC(=O)c1ccncc1N]

To a oven dried flask containing 3-aminoisonicotinic acid (30 g, 217 mmol) was added EtOH (300 mL) followed by the addition of H₂SO₄ (34 mL, 651 mmol). The mixture was stirred at 90° C. for 24 hours. The mixture was concentrated in vacuo. The reaction was diluted with EA and saturated Na₂CO₃ solution to PH=8. The organic layer was separated, washed with further saturated NaCl solution. The organic layer was collected, concentrated in vacuo, and dried to afford the title compound ethyl 3-aminoisonicotinate (30 g, 83%).

MS (ESI) m/z: 167 [M+H]⁺.

### Ethyl 2,4-dihydroxy-1,7-naphthyridine-3-carboxylate

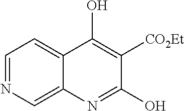 [SMILES: CCOC(=O)c1c(O)nc2cnccc2c1O]

To a solution of ethyl 3-aminoisonicotinate (20 g, 120 mmol) in EtOH (300 mL) was added EtONa (12 g, 180 mmol) and diethyl malonate (29.9 g, 180 mmol), and the reaction was stirred at 90° C. for 24 hours. The reaction was concentrated under reduced pressure and diluted with water, which was adjust pH=5 with 1N HCl. The mixture was filtration, the filtrate cake washed with water and concentrated under reduced pressure to afford the title compound ethyl 2,4-dihydroxy-1,7-naphthyridine-3-carboxylate (16 g, 56.7%).

MS (ESI) m/z: 235 [M+H]⁺.

### 1,7-Naphthyridine-2,4-diol

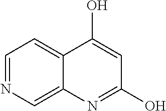 [SMILES: Oc1cc(O)c2ccncc2n1]

To a flask containing ethyl 2,4-dihydroxy-1,7-naphthyridine-3-carboxylate (15 g, 64 mmol) was added H₂O (50 mL) followed by the addition of HCl (150 mL). The mixture was stirred at 70° C. for overnight. The resulting mixture was concentrated under vacuum to provide the title compound 1,7-naphthyridine-2,4-diol (9.0 g, 86.6%).

MS (ESI) m/z: 163 [M+H]⁺.

### 3-Nitro-1,7-naphthyridine-2,4-diol

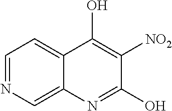 [SMILES: O=[N+]([O-])c1c(O)nc2cnccc2c1O]

To a flask containing 1,7-naphthyridine-2,4-diol (10 g, 61 mmol) was added H₂SO₄ (100 mL) followed by the addition of HNO₃ (15 mL) at 0° C. The mixture was stirred at 60° C. for 30 mins. The reaction mixture was poured into crushed ice and adjusted pH=6 by Na₂CO₃, the solid was filtered to afford the title compound 3-nitro-1,7-naphthyridine-2,4-diol (6.0 g, 46.9%).

MS (ESI) m/z: 208 [M+H]⁺.

### 4-Chloro-3-nitro-1,7-naphthyridin-2 (1H)-one

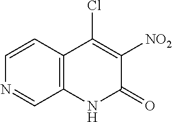 [SMILES: O=c1[nH]c2cnccc2c(Cl)c1[N+](=O)[O-]]

To a solution of DMF (0.48 mL, 6.27 mmol) in MeCN (15 mL) was added (COCl)₂ (0.61 mL, 7.24 mmol) at 0° C. After 10 mins, 3-nitro-1,7-naphthyridine-2,4-diol (1.0 g, 4.82 mmol) was added, and reaction was stirred for 1.5 hours at 0° C. The reaction was quenched by water, and the acetonitrile was evaporated under vacuum. The mixture was filtered and washed with water. The solid was collected, concentrated in vacuo to afford the title compound 4-chloro-3-nitro-1,7-naphthyridin-2-ol (900 mg, 82.6%).

MS (ESI) m/z: 226 [M+H]⁺.

### Ethyl (1R,5S)-8-(4-methoxybenzyl)-3-(3-nitro-2-oxo-1,2-dihydro-1,7-naphthyridin-4-yl)-3,8-diazabicyclo[3.2.1]octane-2-carboxylate

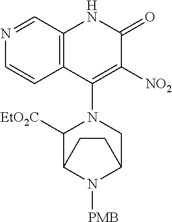

To a solution of 4-chloro-3-nitro-1,7-naphthyridin-2 (1H)-one (718 mg, 3.19 mmol) in MeCN (25 mL) were added NaHCO₃ (803 mg, 9.56 mmol), ethyl 8-(4-methoxybenzyl)-3,8-diazabicyclo[3.2.1]octane-2-carboxylate (970 mg, 3.19 mmol), and the reaction was stirred at 80° C. for 3 hours. The reaction was concentrated in vacuo. The residue was purified using silica gel column chromatography eluting with methanol in chloroform (1:15) to afford the title compound ethyl (1R,5S)-8-(4-methoxybenzyl)-3-(3-nitro-2-oxo-1,2-dihydro-1,7-naphthyridin-4-yl)-3,8-diazabicyclo[3.2.1]octane-2-carboxylate (375 mg, 23.8%) as a yellow solid.

MS (ESI) m/z: 494 [M+H]⁺.

### Ethyl (1R,5S)-3-(2-(((2R,7aS)-2-fluorotetrahydro-1H-pyrrolizin-7a(5H)-yl)methoxy)-3-nitro-1,7-naphthyridin-4-yl)-8-(4-methoxybenzyl)-3,8-diazabicyclo[3.2.1]octane-2-carboxylate

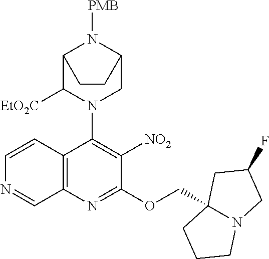

To a solution of ethyl (1R,5S)-8-(4-methoxybenzyl)-3-(3-nitro-2-oxo-1,2-dihydro-1,7-naphthyridin-4-yl)-3,8-diazabicyclo[3.2.1]octane-2-carboxylate (375 mg, 0.76 mmol) in Toluene (10 mL) were added ((2R,7aS)-2-fluorotetrahydro-1H-pyrrolizin-7a(5H)-yl) methanol (181 mg, 1.14 mmol), and 2-(tributyl-15-phosphanylidene) acetonitrile (550 mg, 2.28 mmol), and the reaction was stirred at 110° C. for 3 hours. The reaction was concentrated in vacuo. The residue was purified using silica gel column chromatography eluting with methanol in chloroform (1:25) to afford the title compound ethyl (1R,5S)-3-(2-(((2R,7aS)-2-fluorotetrahydro-1H-pyrrolizin-7a(5H)-yl)methoxy)-3-nitro-1,7-naphthyridin-4-yl)-8-(4-methoxybenzyl)-3,8-diazabicyclo[3.2.1]octane-2-carboxylate (400 mg, 82.9%).

MS (ESI) m/z: 635 [M+H]⁺.

### (9S,12R)-6-(((2R,7aS)-2-fluorotetrahydro-1H-pyrrolizin-7a(5H)-yl)methoxy)-15-(4-methoxybenzyl)-8a,9,10,11,12,13-hexahydro-9,12-epiminoazepino[1′,2′:4,5]pyrazino[2,3-c][1,7]naphthyridin-8(7H)-one

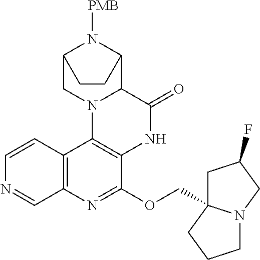

To a solution of ethyl (1R,5S)-3-(2-(((2R,7aS)-2-fluorotetrahydro-1H-pyrrolizin-7a(5H)-yl)methoxy)-3-nitro-1,7-naphthyridin-4-yl)-8-(4-methoxybenzyl)-3,8-diazabicyclo[3.2.1]octane-2-carboxylate (200 mg, 0.31 mmol) in EtOH (5 mL), H₂O (3 mL) were added Fe (105 mg, 1.89 mmol) and NH₄Cl (100 mg, 1.89 mmol), and the reaction was stirred at 60° C. for 3 hours. The reaction was concentrated in vacuo. The residue was purified using silica gel column chromatography eluting with DCM:MeOH=10:1 to afford the title compound (9S,12R)-6-(((2R,7aS)-2-fluorotetrahydro-1H-pyrrolizin-7a(5H)-yl)methoxy)-15-(4-methoxybenzyl)-8a,9,10,11,12,13-hexahydro-9,12-epiminoazepino[1′,2′:4,5]pyrazino[2,3-c][1,7]naphthyridin-8(7H)-one (100 mg, 56.8%).

MS (ESI) m/z: 559 [M+H]⁺.

### (9S,12R)-6-(((2R,7aS)-2-fluorotetrahydro-1H-pyrrolizin-7a(5H)-yl)methoxy)-15-(4-methoxybenzyl)-7-methyl-8a,9,10,11,12,13-hexahydro-9,12-epiminoazepino[1′,2′:4,5]pyrazino[2,3-c][1,7]naphthyridin-8(7H)-one

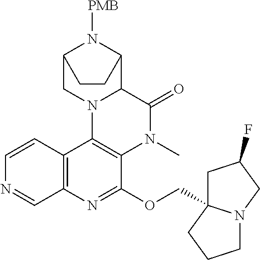

To a solution of (9S,12R)-6-(((2R,7aS)-2-fluorotetrahydro-1H-pyrrolizin-7a(5H)-yl)methoxy)-15-(4-methoxybenzyl)-8a,9,10,11,12,13-hexahydro-9,12-epiminoazepino[1′,2′:4,5]pyrazino[2,3-c][1,7]naphthyridin-8(7H)-one (100 mg, 0.17 mmol) in DMF (4 mL) were added Cs₂CO₃ (69 mg, 0.21 mmol) and CH₃I (4.4 mL, 0.04 mol/L in THF), and the reaction was stirred at room temperature for 1 hour. The reaction was concentrated in vacuo. The residue was purified using silica gel column chromatography eluting with DCM:MeOH=10:1 to afford the title compound (9S,12R)-6-(((2R,7aS)-2-fluorotetrahydro-1H-pyrrolizin-7a(5H)-yl)methoxy)-15-(4-methoxybenzyl)-7-methyl-8a,9,10,11,12,13-hexahydro-9,12-epiminoazepino[1′,2′:4,5]pyrazino[2,3-c][1,7]naphthyridin-8(7H)-one (15 mg, 14.6%).

MS (ESI) m/z: 573 [M+H]⁺.

### (9S,12R)-6-(((2R,7aS)-2-fluorotetrahydro-1H-pyrrolizin-7a(5H)-yl)methoxy)-15-(4-methoxybenzyl)-7-methyl-2,3,4,7,8a,9,10,11,12,13-decahydro-9,12-epiminoazepino[1′,2′:4,5]pyrazino[2,3-c][1,7]naphthyridin-8(1H)-one

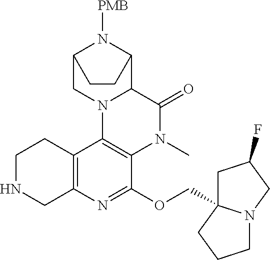

To a solution of (9S,12R)-6-(((2R,7aS)-2-fluorotetrahydro-1H-pyrrolizin-7a(5H)-yl)methoxy)-15-(4-methoxybenzyl)-7-methyl-8a,9,10,11,12,13-hexahydro-9,12-epiminoazepino[1′,2′:4,5]pyrazino[2,3-c][1,7]naphthyridin-8(7H)-one (25 mg, 0.044 mmol) in AcOH (2 mL) was added NaBH₄ (7 mg, 0.218 mmol) at rt. The mixture was stirred at rt for 1 hour. The reaction mixture was quenched with ice water and pH was adjusted to about 7 with solid NaHCO₃. The reaction was extracted with EA (3×3 mL). The combined organic layers were dried over anhydrous Na₂SO₄.

After filtration, the filtrate was concentrated under reduced pressure to give the desired product as a yellow solid (25 mg, yield: 99.3%), which was used to next step without further purification. MS (ESI) m/z: 577 [M+H]⁺.

### (9S,12R)-3-(8-chloro-7-fluoronaphthalen-1-yl)-6-(((2R,7aS)-2-fluorotetrahydro-1H-pyrrolizin-7a(5H)-yl)methoxy)-15-(4-methoxybenzyl)-7-methyl-2,3,4,7,8a,9,10,11,12,13-decahydro-9,12-epiminoazepino[1′,2′:4,5]pyrazino[2,3-c][1,7]naphthyridin-8(1H)-one

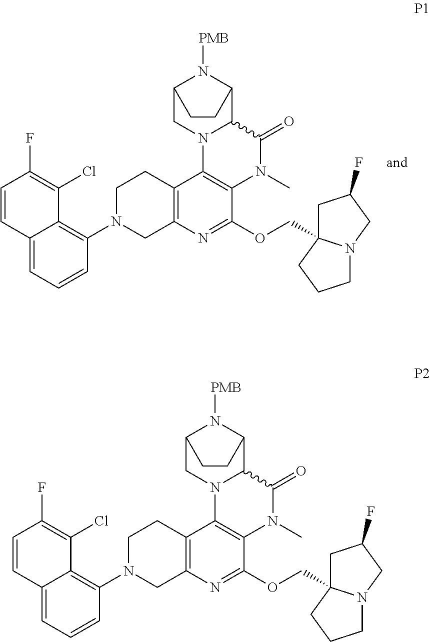

To a solution of (9S,12R)-6-(((2R,7aS)-2-fluorotetrahydro-1H-pyrrolizin-7a(5H)-yl)methoxy)-15-(4-methoxybenzyl)-7-methyl-2,3,4,7,8a,9,10,11,12,13-decahydro-9,12-epiminoazepino[1′,2′:4,5]pyrazino[2,3-c][1,7]naphthyridin-8(1H)-one (25 mg, 0.043 mmol) in Toluene (1.5 mL) were added 8-chloro-7-fluoronaphthalen-1-yl trifluoromethanesulfonate (28 mg, 0.087 mmol), RuPhos Pd G2 (3 mg, 0.004 mmol), RuPhos (2 mg, 0.004 mmol) and Cs₂CO₃ (42 mg, 0.13 mmol), and the reaction was stirred at 110° C. for overnight. The reaction was concentrated in vacuo. The residue was purified Pre-TLC eluting with DCM/MeOH=10:1 to afford the title compound (9S,12R)-3-(8-chloro-7-fluoronaphthalen-1-yl)-6-(((2R,7aS)-2-fluorotetrahydro-1H-pyrrolizin-7a(5H)-yl)methoxy)-15-(4-methoxybenzyl)-7-methyl-2,3,4,7,8a,9,10,11,12,13-decahydro-9,12-epiminoazepino[1′,2′:4,5]pyrazino[2,3-c][1,7]naphthyridin-8(1H)-one (10 mg, 30.5%).

The chiral separation of this product (10.0 mg) was performed by chiral-HPLC with the following condition: Equipment and Column: SHIMADZU PREP

SOLUTION SFC Column: ChiralPak IH, 250×21.2 mm I.D., 5 um; Mobile phase: A for CO₂ and B for MEOH+0.1% NH₃·H₂O; Gradient: B 40%; Flow rate: 40 mL/min; Back pressure: 100 bar; Column temperature: 35° C.; Wavelength: 220 nm; Cycle-time: 8.0 min; Eluted time: 2.2 H) to give two desired product as white solids.

P1: (Retention time: 4.241 min). LC-MS (ESI): m/z 755 [M+H]⁺.

¹H NMR (400 MHZ, MeOD) δ 7.89-7.82 (m, 1H), 7.66 (d, J=8.2 Hz, 1H), 7.46 (t, J=7.8 Hz, 1H), 7.37 (ddd, J=19.8, 12.5, 6.0 Hz, 4H), 6.89 (d, J=7.3 Hz, 2H), 5.32 (d, J=15.1 Hz, 1H), 5.17 (s, 1H), 4.25-4.13 (m, 2H), 4.07 (d, J=10.3 Hz, 1H), 3.83 (s, 2H), 3.78 (d, J=2.2 Hz, 3H), 3.74-3.68 (m, 1H), 3.61-3.43 (m, 6H), 3.36 (s, 2H), 3.23-2.94 (m, 7H), 2.28-2.03 (m, 7H), 1.96 (s, 3H).

P2: (Retention time: 5.621 min). LC-MS (ESI): m/z 755 [M+H]⁺.

¹H NMR (400 MHZ, MeOD) δ 7.89-7.83 (m, 1H), 7.66 (d, J=8.2 Hz, 1H), 7.49-7.43 (m, 1H), 7.41-7.30 (m, 4H), 6.93-6.86 (m, 2H), 5.34-5.28 (m, 1H), 5.16 (s, 1H), 4.20 (dd, J=14.3, 9.1 Hz, 1H), 4.05 (dd, J=21.1, 8.0 Hz, 2H), 3.85 (t, J=16.4 Hz, 2H), 3.78 (d, J=2.5 Hz, 3H), 3.71 (d, J=16.1 Hz, 1H), 3.53 (dd, J=30.0, 8.4 Hz, 6H), 3.37 (s, 2H), 3.27-2.88 (m, 7H), 2.26-1.88 (m, 10H).

### (9S,12R)-3-(8-chloro-7-fluoronaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one

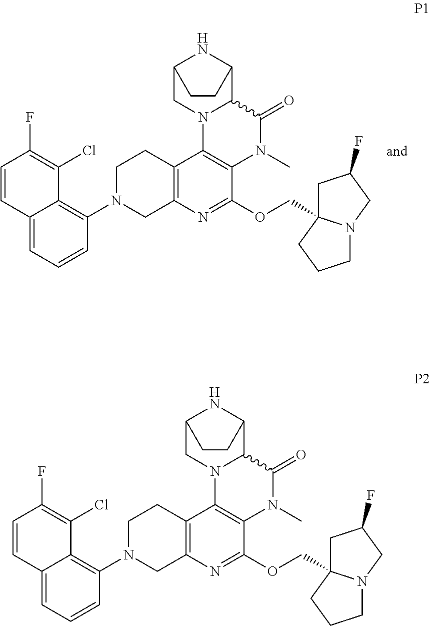 [SMILES: CN1C(=O)C2C3CCC(CN2c2c4c(nc(OC[C@@]56CCCN5C[C@H](F)C6)c21)CN(c1cccc2ccc(F)c(Cl)c12)CC4)N3 ; CN1C(=O)C2C3CCC(CN2c2c4c(nc(OC[C@@]56CCCN5C[C@H](F)C6)c21)CN(c1cccc2ccc(F)c(Cl)c12)CC4)N3]

LC-MS (ESI): m/z 635 [M+H]⁺.

### Compound 95: (9S,12R)-3-(8-ethynyl-7-fluoronaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one

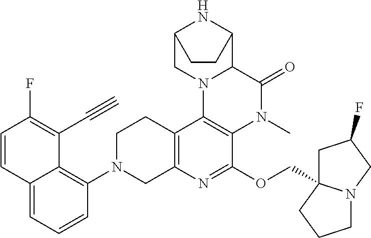 [SMILES: C#Cc1c(F)ccc2cccc(N3CCc4c(nc(OC[C@@]56CCCN5C[C@H](F)C6)c5c4N4CC6CCC(N6)C4C(=O)N5C)C3)c12]

LC-MS (ESI): m/z 625 [M+H]⁺.

### Compound 96: (9S,12R)-3-(8-ethyl-7-fluoro-3-hydroxynaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one

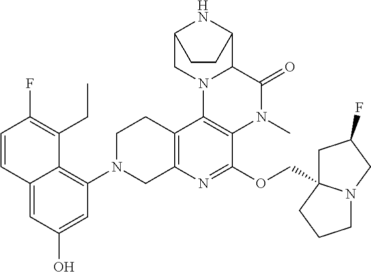 [SMILES: CCc1c(F)ccc2cc(O)cc(N3CCc4c(nc(OC[C@@]56CCCN5C[C@H](F)C6)c5c4N4CC6CCC(N6)C4C(=O)N5C)C3)c12]

LC-MS (ESI): m/z 645 [M+H]⁺.

### Compound 97: (9S,12R)-3-(8-chloro-7-fluoro-3-hydroxynaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one

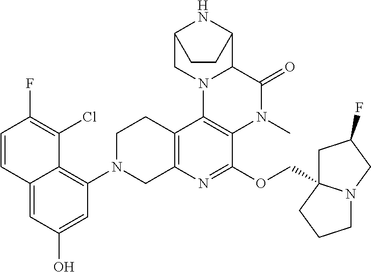 [SMILES: CN1C(=O)C2C3CCC(CN2c2c4c(nc(OC[C@@]56CCCN5C[C@H](F)C6)c21)CN(c1cc(O)cc2ccc(F)c(Cl)c12)CC4)N3]

LC-MS (ESI): m/z 651 [M+H]⁺.

### Compound 98: (9S,12R)-3-(7,8-difluoro-3-hydroxynaphthalen-1-yl)-6-(((2R,7aS)-2-fluorotetrahydro-1H-pyrrolizin-7a(5H)-yl)methoxy)-7-methyl-2,3,4,7,8a,9,10,11,12,13-decahydro-9,12-epiminoazepino[1′,2′:4,5]pyrazino[2,3-c][1,7]naphthyridin-8(1H)-one

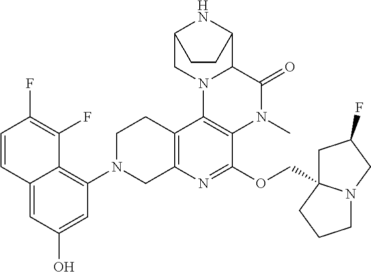 [SMILES: CN1C(=O)C2C3CCC(CN2c2c4c(nc(OC[C@@]56CCCN5C[C@H](F)C6)c21)CN(c1cc(O)cc2ccc(F)c(F)c12)CC4)N3]

LC-MS (ESI): m/z 635 [M+H]⁺.

### Biology Assay

The following assays were used to measure the effects of the compounds of the present disclosure.

### Phospho-ERK 1/2 Assay:

Allow PNAC-1/HPAC cell growth in a T75 flask in DMEM and 10% 1 fetal calf serum (FCS; Gibco), using standard tissue culture procedures until ˜80% confluency is achieved. Day 1, seed 6000 cells/well in 384 well plate and incubate at 37° C., 5% CO₂. Add diluted compound by Echo 550, final DMSO is 0.5%, incubate cells at 37° C., 5% CO₂ for 3 hours. Then, remove medium and fix cells with 3.7% formaldehyde in PBS (PFA) by Apricot. Wash with PBS once. Permeabilize cells with cold 100% methanol and repeat wash once with PBS once. Add Li-Cor blocking buffer to each well and incubate 1.5 hours at RT. Remove blocking buffer and add primary antibody mixture (rabbit anti pERK, mouse anti GAPDH). Incubate at 4° C. overnight. Day 2, wash with PBST (Tween-20 in PBS) with total 3 times and then add secondary antibody mixture (goat anti rabbit 800CW (1:800 dilution in the combined solution) and goat anti mouse 680RD (1:800 dilution in the combined solution)), incubate for 60 minutes at RT away from light. Repeat washing with PBST 3 times. After final wash, centrifuge plate up-side-down at 1000 rpm to remove wash solution completely from wells. Before plate scanning, clean the bottom plate surface and the Odyssey* Imager scanning bed (if applicable) with moist, lint-free tissue to avoid any obstructions during scanning. Scan plate with detection in both 700 and 800 nm channels.

The p-ERK IC₅₀ values of the exemplary compounds of the present disclosure are shown in Table 1.

TABLE 1
p-ERK IC₅₀ values of the exemplary compounds

| Compound No. | IC₅₀ (nM) |
|---|---|
| 1 | 33 |
| 2 | 730 |
| 3 | 26 |
| 4 | 121 |
| 5 | 77 |
| 6 | 86 |
| 7 | 34 |
| 8 | 9 |
| 9 | 308 |
| 10 | 2 |
| 11 | 53 |
| 12 | 68 |
| 13 | 237 |
| 14 | 141 |
| 15 | 221 |
| 16 | 355 |
| 17 | 1000 |
| 18 | 404 |
| 19 | 29 |
| 20 | 735 |
| 21 | 1234 |
| 22 | 493 |
| 23 | 375 |
| 24 | 7 |
| 25 | 10 |
| 26 | 148 |
| 27 | 5 |
| 28 | 0.76 |
| 30 | 17 |
| 31 | 8 |
| 32 | 140 |
| 33 | 75 |
| 34 | 5 |
| 37 | 50 |
| 38 | 70 |
| 40 | 155 |
| 41 | 390 |
| 42 | 159 |
| 44 | 131 |
| 45 | 3.5 |
| 46 | 328 |
| 47 | 105 |
| 49 | 851 |
| 53 | 457 |
| 111 | 12 |

Other compounds of the present disclosure show IC₅₀ value of 0.5 to 2000 nM. Some compounds of the present disclosure show IC₅₀ value of 1-1000 nM. Some compounds of the present disclosure show IC₅₀ value of 1-500 nM.

Table 2 describes the p-ERK IC₅₀ values and selectivity over WT-KRAS cell line MKN-1 of the exemplary compounds of the present disclosure and reference compound MRTX-1133.

TABLE 2
Selectivity over WT-KRAS cell line
MKN-1 of exemplary compounds

| | Panc-1 p-ERK | MKN-1 | Selectivity |
|---|---|---|---|
| Compound No. | IC₅₀ (nM) | IC₅₀ (nM) | Fold |
| MRTX-1133 | 2.3 | 83 | 36 |
| 10 | 2 | 343 | 171 |
| 25 | 10 | 754 | 75 |
| 28 | 0.76 | 76 | 100 |

### KRAS G12D 2D CellTiter-Glo® Proliferation Assay:

AsPC-1 (ATCC CRL-1682) and LS513 (ATCC CRL-2134) cells were purchased from ATCC, GP2D (Cobioer CBP60010) cells was purchased from Cobioer biosciences CO., LTD, and each cell was cultured in medium supplemented with 10% fetal bovine serum (FBS), according to the protocol recommended by the manufacture. Cells were seeded 800 cells/well in 384-well plates (Corning) and incubated at 37° C., 5% CO₂ for 18 hours. Serially diluted compound was added to the cells, and plates were incubated at 37° C., 5% CO₂ for 72 hours. Cell viability was measured using a CellTiter-Glo® Luminescent Cell Viability Assay kit (Promega) according to the manufacturer's protocol.

### KRAS G12D 3D CellTiter-Glo® Proliferation Assay:

HPAC (ATCC CRL-2119) and AsPC-1 (ATCC CRL-1682) cells were purchased from ATCC, and each cell was cultured in medium supplemented with 10% fetal bovine serum (FBS), according to the protocol recommended by the manufacture. Serially diluted compound was added to 384-well Ultra-Low Attachment Surface round bottom plate (Corning). 400 cells/well were seeded in plate and incubated at 37° C., 5% CO₂ for 7 days. Cell viability was measured using a CellTiter-Glo® 3D Cell Viability Assay kit (Promega) according to the manufacturer's protocol.

The 2D and 3D proliferation data of the exemplary compounds of the present disclosure in different cell lines are shown in Table 3.

TABLE 3
2D and 3D proliferation data of the exemplary compound

| | HPAC | AsPC-1 | AsPC-1 | GP2D | LS513 |
|---|---|---|---|---|---|
| | 3D | 3D | 2D | 2D | 2D |
| Compound | IC₅₀ | IC₅₀ | IC₅₀ | IC₅₀ | IC₅₀ |
| No. | (nM) | (nM) | (nM) | (nM) | (nM) |
| 10 | 28 | 48 | 490 | 15 | 238 |
| 24 | 7.1 | 8.7 | 71 | 2.2 | 93 |
| 25 | 76 | 54 | 516 | 23 | 80 |
| 28 | 3.3 | 4.7 | 35 | 1.2 | 25.2 |

### Caco-2 Cell Monolayers Permeability:

10 μM input concentration and pH 6.5/7.4 (apical/basolateral), in the presence of efflux inhibitors, Zosuquidar, Benzbromarone and KO-143. Incubations were performed at 37° C. with shaking at 480 rpm on a rotary shaker over the course of 120 minutes, and samples were collected at 45 and 120 minutes to assess recovery. All incubations were performed in singlet. Lucifer yellow was used as a marker to confirm the integrity of the cell monolayers following 120 min of incubation. UPLC-MS/MS was used to quantify compound concentrations in the incubation medium of donor and receiver compartments. The concentration data was used to calculate the apparent permeability after 120 min of incubation. Table 4 shows the apparent permeability data of exemplary compounds of the present disclosure and reference compound MRTX-1133.

TABLE 4
Apparent permeability data of the exemplary compounds

| Compound No. | Papp (×10⁻⁶ cm/s) |
|---|---|
| MRTX-1133 | 0.38 |
| 10 | 1.48 |
| 25 | 0.66 |
| 40 | 1.87 |
| 41 | 2.08 |
| 42 | 3.31 |

### In Vivo Oral Bioavailability Assay:

Single dose following IV bolus (1 mg/kg, 0.2 mg/mL in 1% DMSO, 99% SBE-β-CD (10% w/v) in water) and oral gavage (30 mg/kg, 3 mg/mL in 1% MC in DI water) administration of test compound in Balb/c Mice female. The blood samples were collected at 2 min, 5 min, 10 min, 30 min, 1 hr, 2 hr, 4 hr, 8 hr and 24 hr (additional 32 hr and 48 hr for MRTX1133) after IV bolus, at 15 min, 30 min, 1 hr, 1.5 hr, 2 hr, 3 hr, 4 hr, 8 hr and 24 hr (additional 32 hr and 48 hr for MRTX1133) after PO administration. The plasma concentrations of compounds were determined with UPLC-MS/MS. Table 5 shows the bioavailability data of exemplary compounds of the present disclosure and reference compound MRTX-1133.

TABLE 5
Bioavailability data of exemplary compounds

| Compound No. | Fa*Fg (%) | Bioavailability (%) |
|---|---|---|
| MRTX-1133 | 0.95 | 0.66 |
| 10 | 54.0 | 37.7 |
| 25 | 56.6 | 39.2 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1-97. (canceled)

98. A compound having Formula (IIb): 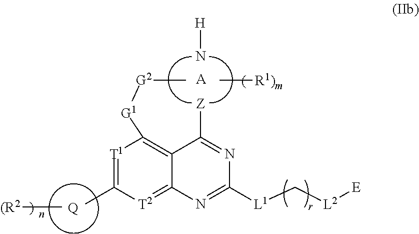
or a pharmaceutically acceptable salt thereof,
wherein
Ring A is heterocyclyl or heteroaryl,
each R¹ is independently selected from the group consisting of oxo, hydroxyl, halogen, cyano, alkyl, alkenyl, alkynyl, heteroalkyl, heteroaryl, —C(O)R*, —C(O)OR*, —C(O)N(R^{a})₂, —N(R^{a})₂, —P(O)OR*OR*, and —C(O)OC(R^{a})₂—Z¹—Z², wherein the alkyl, alkenyl, alkynyl and heteroaryl are optionally substituted with one or more groups independently selected from cyano, hydroxyl, halogen, —OR^{b}, or —N(R^{b})₂;
each R^{a} and R^{b} is independently hydrogen, alkyl, alkenyl or alkynyl;
R* is selected from hydrogen, alkyl, alkylaryl or aryl;
R** is selected from hydrogen, alkyl, alkenyl or alkynyl; or
R* and R** together with the oxygen atoms to which they are attached form a heterocyclyl optionally substituted with aryl or haloaryl;
Z¹ is —OC(O)-#, —OP(═O)(OR*)O-#, or —OP(═O)(OR*)N(R^{a})-#, wherein # end is connected to Z²;
Z² is hydrogen or alkyl optionally substituted with aryl or —C(O)OR^{a};
R*** is independently selected from hydrogen, alkyl, alkenyl or alkynyl; or
R*** and Z² together with the oxygen atoms to which they are attached form a heterocyclyl optionally substituted with aryl or haloaryl;
Ring Q is selected from cycloalkyl, heterocyclyl, aryl or heteroaryl;
each R² is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, heteroaryl and —C(O)R′, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
each R³ is independently selected from the group consisting of hydrogen, oxo, hydroxyl, halogen, cyano, amino, nitro, alkyl, alkenyl, alkynyl, alkoxy, heteroalkyl, heteroalkenyl, heteroalkynyl, cycloalkyl, heterocyclyl, aryl, and heteroaryl, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of hydroxyl, halogen, cyano, amino, nitro, alkyl, alkoxy, haloalkyl, hydroxyalkyl, cycloalkyl, heterocyclyl, aryl and heteroaryl;
G¹ is a bond, —O—, —S(O)ₚ—, —S—S—, —N(R^{c})—, or —C(R^{d})═C(R^{d})—;
G² is a bond, —[C(R^{d})₂]ᵤ—, —C(O)— or —C(O)C(R^{d})₂—;
R^{c} is selected from the group consisting of hydrogen, alkyl, haloalkyl, hydroxyalkyl, heteroalkyl, cycloalkyl and heterocyclyl;
each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of hydroxyl, halogen, cyano, amino, nitro, alkyl, alkoxy, haloalkyl, and hydroxyalkyl; or
two R^{d} together with the carbon atom to which they are both attached form cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
Z is C(R^{e}) or N;
R^{e} is absent or hydrogen;
L¹ is selected from a bond, —O—, —S—, —N(R^{a})—, —C(O)N(R^{a})—, alkenyl, alkynyl or cycloalkyl; 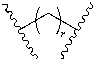
is optionally substituted with hydroxyl, halogen, cyano or amino;
L² is a bond, alkyl, cycloalkyl, heterocyclyl, aryl or heteroaryl, wherein the cycloalkyl, heterocyclyl, aryl and heteroaryl are optionally substituted with one or more groups independently selected from hydroxyl, halogen, cyano, amino, alkyl, hydroxyalkyl or heteroaryl;
E is selected from the group consisting of hydrogen, hydroxyl, halogen, —N(R^{a})₂, alkyl, haloalkyl, heteroalkyl, cycloalkyl, heterocyclyl, aryl, heteroaryl, —COOH, —CH₂OC(O)-heterocyclyl, —CH₂OC(O)N(R^{a})₂, —NHC(═NH)NH₂, —C(O)N(R^{a})₂, —OR^{a}, —(CH₂OR^{a})(CH₂)ₚOR^{a}, —N(R^{a})C(O)-aryl and —(CH₂)ᵤ-heterocyclyl, wherein the cycloalkyl, heterocyclyl, aryl, and heteroaryl are optionally substituted with one or more R″, and the aryl portion in —N(R^{a})C(O)-aryl and the heterocyclyl portion in —(CH₂)ᵤ-heterocyclyl and —CH₂OC(O)-heterocyclyl is optionally substituted with one or more R′″;
each R″ is independently selected from hydroxyl, halogen, —C(O)H, alkyl, alkoxy, haloalkyl, hydroxyalkyl, or —N(R^{a})₂;
each R′″ is independently selected from oxo, hydroxyl, halogen, alkyl, heteroalkyl, hydroxyalkyl, haloalkyl, alkoxy, -T-phenyl, -T-phenylSO₂F, —N(R^{a})₂, —SO₂F, —C(O)(alkyl), or —C(O)(haloalkyl), wherein the alkyl, heteroalkyl, hydroxyalkyl, haloalkyl, and alkoxy are optionally substituted with one or more groups independently selected from aryl, heteroaryl, or tert-butyldimethylsilyloxy;
T is a bond, —O—, or —NHC(O)—;
m is an integer from 0 to 6;
n is an integer from 0 to 5;
r is an integer from 0 to 4;
p is an integer from 0 to 2; and
u is an integer from 0 to 4.

99. The compound of claim 98, or a pharmaceutically acceptable salt thereof, wherein Ring A is heterocyclyl, preferably Ring A is selected from the group consisting of: 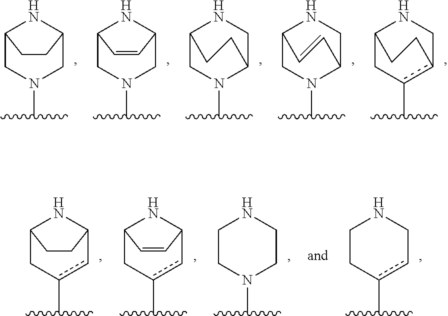
wherein [symbol] represents a single bond or a double bond.

100. The compound of claim 98, or a pharmaceutically acceptable salt thereof, wherein
(a) Ring Q is aryl, preferably Ring Q is phenyl or naphthalenyl; or
(b) Ring Q is heteroaryl, preferably Ring Q is selected from benzothiophenyl, benzoimidazolyl, quinazolinyl, benzotriazolyl, thiophenyl, thienopyridinyl, isoquinolinyl, indolyl, or indazolyl.

101. The compound of claim 98, or a pharmaceutically acceptable salt thereof, wherein G¹ is —O—.

102. The compound of claim 98, or a pharmaceutically acceptable salt thereof, wherein G² is —[C(R^{d})₂]ᵤ—, optionally each R^{d} is independently hydrogen or alkyl.

103. The compound of claim 98, or a pharmaceutically acceptable salt thereof, wherein:
(a) m is 0;
(b) m is an integer from 1 to 3, and each R¹ is independently alkyl;
(c) m is 1, and R¹ is —C(O)R* or —C(O)OR*, wherein R* is alkyl or alkylaryl; or
(d) m is 1, R¹ is —C(O)OC(R^{a})₂—Z¹—Z², Z¹ is —OC(O)-# and Z² is alkyl optionally substituted with aryl.

104. The compound of claim 98, or a pharmaceutically acceptable salt thereof, wherein n is an integer from 1 to 4, and each R² is independently selected from hydroxyl, halogen, cyano, amino, alkyl, alkenyl, alkynyl, or cycloalkyl, wherein alkyl, alkenyl, alkynyl, and cycloalkyl are optionally substituted with one or more groups independently selected from cyano, hydroxyl, halogen, or alkyl.

105. The compound of claim 98, or a pharmaceutically acceptable salt thereof, wherein L¹ is —O—.

106. The compound of claim 98, or a pharmaceutically acceptable salt thereof, wherein L² is alkyl, cycloalkyl, heterocyclyl, or heteroaryl, each optionally substituted with one or more of halogen or alkyl.

107. The compound of claim 98, or a pharmaceutically acceptable salt thereof, wherein L² is selected from hexahydro-1H-pyrrolizinyl, azetidinyl, pyrrolidinyl or pyridinyl.

108. The compound of claim 98, or a pharmaceutically acceptable salt thereof, wherein E is selected from hydrogen, hydroxyl, halogen, haloalkyl, heteroalkyl, —N(R^{a})₂, or —CH₂OC(O)-heterocyclyl.

109. The compound of claim 98, T¹ is N or C(R′); T² is N or C(R′); and v is an integer from 0 to 4.

110. The compound of claim 98 selected from the group consisting of: 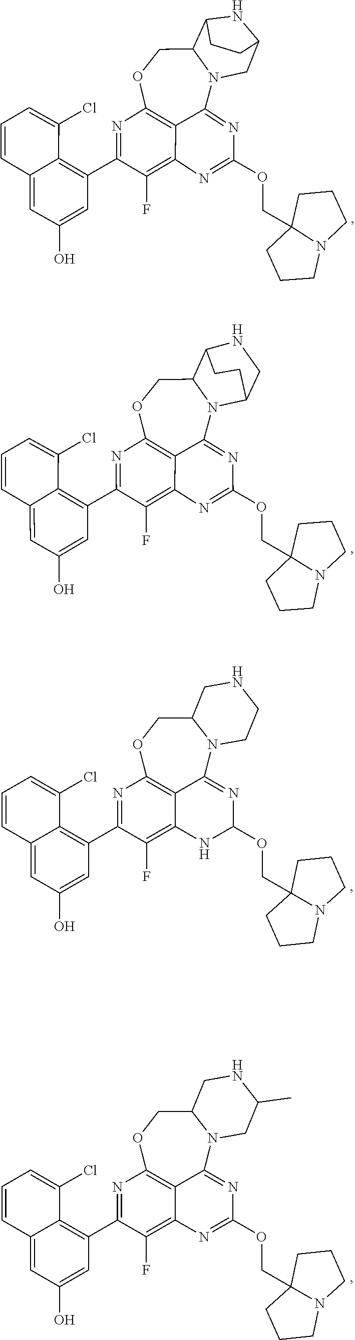 [SMILES: Oc1cc(-c2nc3c4c(nc(OCC56CCCN5CCC6)nc4c2F)N2CC4CCC(N4)C2CO3)c2c(Cl)cccc2c1 ; Oc1cc(-c2nc3c4c(nc(OCC56CCCN5CCC6)nc4c2F)N2C4CCC(NC4)C2CO3)c2c(Cl)cccc2c1 ; Oc1cc(-c2nc3c4c(c2F)NC(OCC25CCCN2CCC5)N=C4N2CCNCC2CO3)c2c(Cl)cccc2c1 ; CC1CN2c3nc(OCC45CCCN4CCC5)nc4c(F)c(-c5cc(O)cc6cccc(Cl)c56)nc(c34)OCC2CN1] 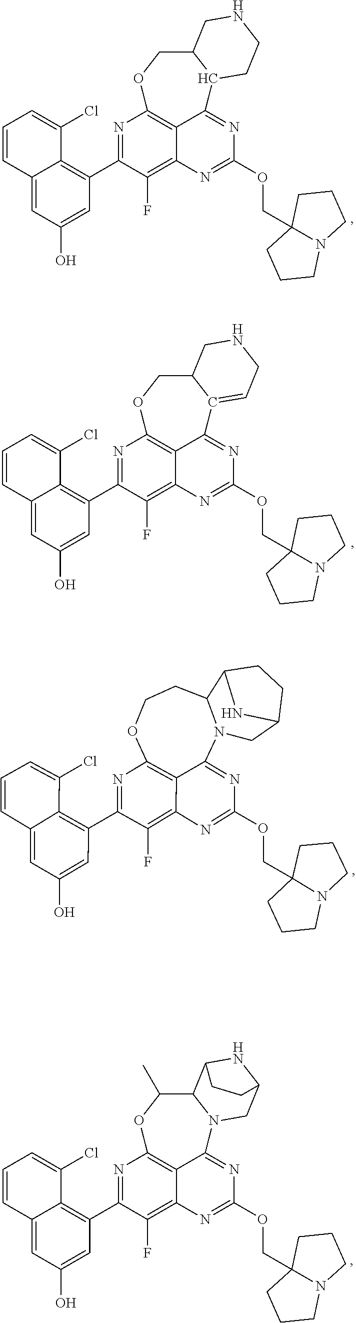 [SMILES: Oc1cc(-c2nc3c4c(nc(OCC56CCCN5CCC6)nc4c2F)C2CCNCC2CO3)c2c(Cl)cccc2c1 ; Oc1cc(-c2nc3c4c(nc(OCC56CCCN5CCC6)nc4c2F)C2=CCNCC2CO3)c2c(Cl)cccc2c1 ; Oc1cc(-c2nc3c4c(nc(OCC56CCCN5CCC6)nc4c2F)N2CC4CCC(N4)C2CCO3)c2c(Cl)cccc2c1 ; CC1Oc2nc(-c3cc(O)cc4cccc(Cl)c34)c(F)c3nc(OCC45CCCN4CCC5)nc(c23)N2CC3CCC(N3)C12] 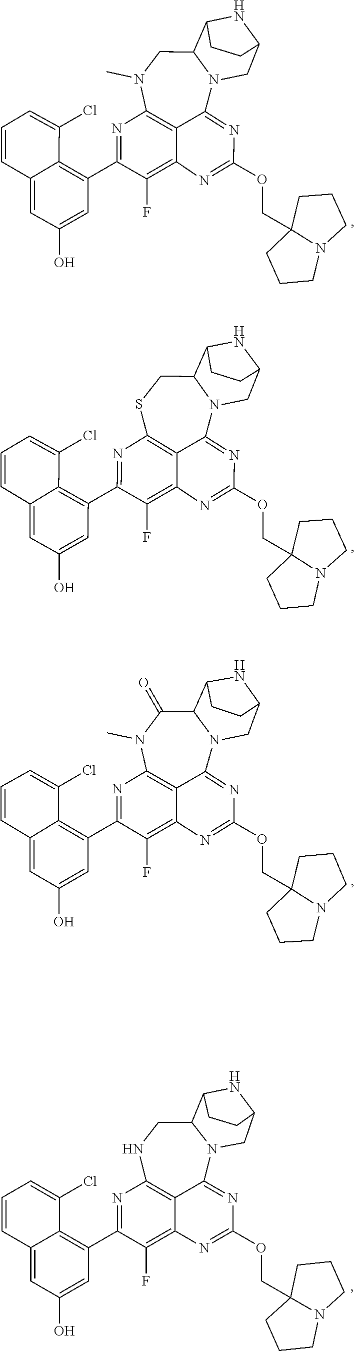 [SMILES: CN1CC2C3CCC(CN2c2nc(OCC45CCCN4CCC5)nc4c(F)c(-c5cc(O)cc6cccc(Cl)c56)nc1c24)N3 ; Oc1cc(-c2nc3c4c(nc(OCC56CCCN5CCC6)nc4c2F)N2CC4CCC(N4)C2CS3)c2c(Cl)cccc2c1 ; CN1C(=O)C2C3CCC(CN2c2nc(OCC45CCCN4CCC5)nc4c(F)c(-c5cc(O)cc6cccc(Cl)c56)nc1c24)N3 ; Oc1cc(-c2nc3c4c(nc(OCC56CCCN5CCC6)nc4c2F)N2CC4CCC(N4)C2CN3)c2c(Cl)cccc2c1] 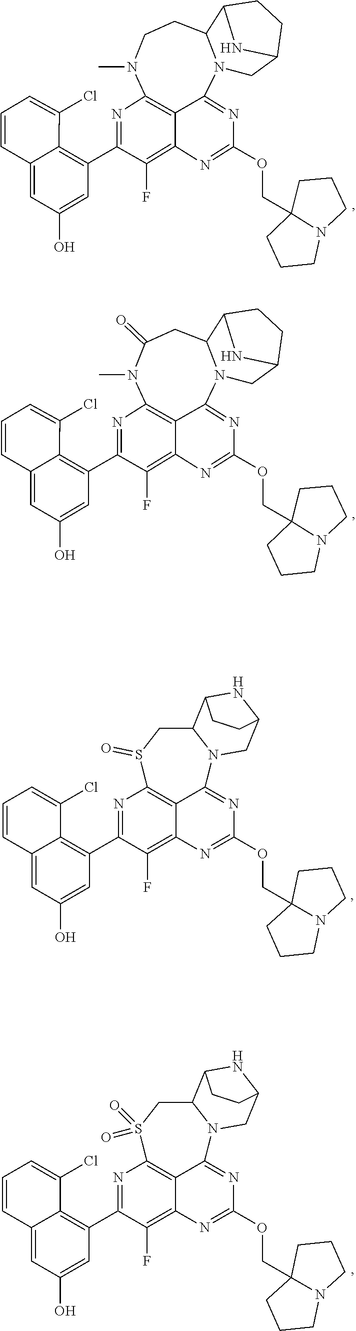 [SMILES: CN1CCC2C3CCC(CN2c2nc(OCC45CCCN4CCC5)nc4c(F)c(-c5cc(O)cc6cccc(Cl)c56)nc1c24)N3 ; CN1C(=O)CC2C3CCC(CN2c2nc(OCC45CCCN4CCC5)nc4c(F)c(-c5cc(O)cc6cccc(Cl)c56)nc1c24)N3 ; O=S1CC2C3CCC(CN2c2nc(OCC45CCCN4CCC5)nc4c(F)c(-c5cc(O)cc6cccc(Cl)c56)nc1c24)N3 ; O=S1(=O)CC2C3CCC(CN2c2nc(OCC45CCCN4CCC5)nc4c(F)c(-c5cc(O)cc6cccc(Cl)c56)nc1c24)N3] 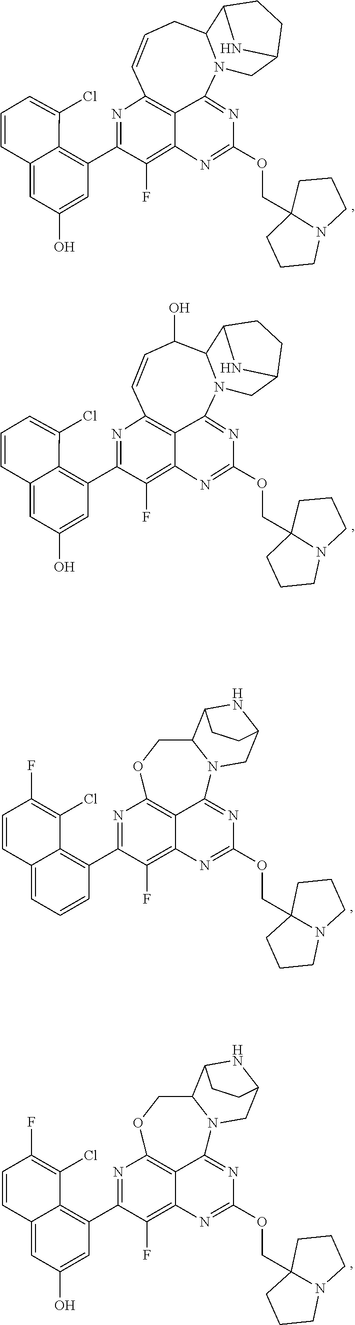 [SMILES: Oc1cc(-c2nc3c4c(nc(OCC56CCCN5CCC6)nc4c2F)N2CC4CCC(N4)C2C/C=C\3)c2c(Cl)cccc2c1 ; Oc1cc(-c2nc3c4c(nc(OCC56CCCN5CCC6)nc4c2F)N2CC4CCC(N4)C2C(O)/C=C\3)c2c(Cl)cccc2c1 ; Fc1ccc2cccc(-c3nc4c5c(nc(OCC67CCCN6CCC7)nc5c3F)N3CC5CCC(N5)C3CO4)c2c1Cl ; Oc1cc(-c2nc3c4c(nc(OCC56CCCN5CCC6)nc4c2F)N2CC4CCC(N4)C2CO3)c2c(Cl)c(F)ccc2c1] 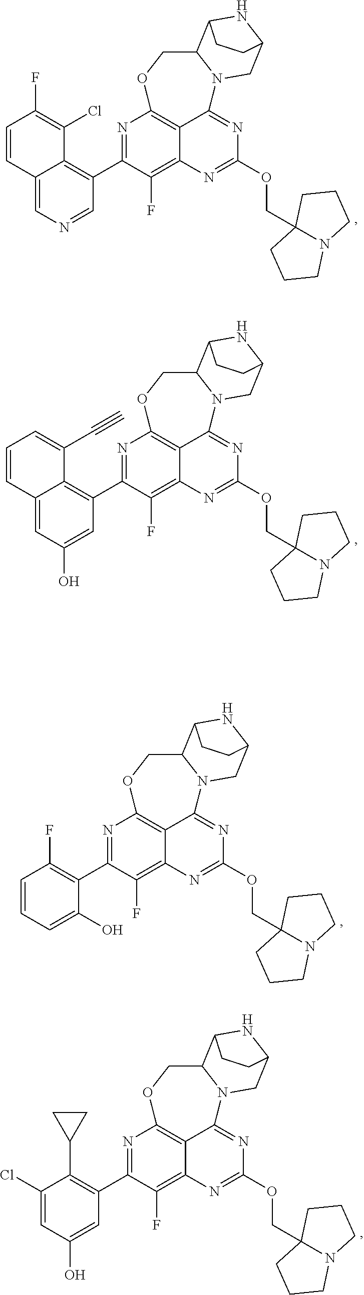 [SMILES: Fc1ccc2cncc(-c3nc4c5c(nc(OCC67CCCN6CCC7)nc5c3F)N3CC5CCC(N5)C3CO4)c2c1Cl ; C#Cc1cccc2cc(O)cc(-c3nc4c5c(nc(OCC67CCCN6CCC7)nc5c3F)N3CC5CCC(N5)C3CO4)c12 ; Oc1cccc(F)c1-c1nc2c3c(nc(OCC45CCCN4CCC5)nc3c1F)N1CC3CCC(N3)C1CO2 ; Oc1cc(Cl)c(C2CC2)c(-c2nc3c4c(nc(OCC56CCCN5CCC6)nc4c2F)N2CC4CCC(N4)C2CO3)c1] 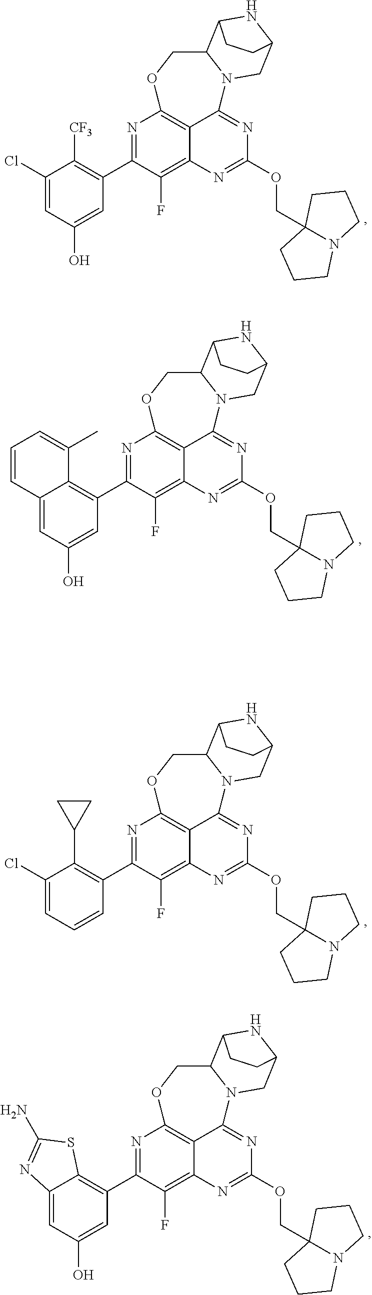 [SMILES: Oc1cc(Cl)c(C(F)(F)F)c(-c2nc3c4c(nc(OCC56CCCN5CCC6)nc4c2F)N2CC4CCC(N4)C2CO3)c1 ; Cc1cccc2cc(O)cc(-c3nc4c5c(nc(OCC67CCCN6CCC7)nc5c3F)N3CC5CCC(N5)C3CO4)c12 ; Fc1c(-c2cccc(Cl)c2C2CC2)nc2c3c(nc(OCC45CCCN4CCC5)nc13)N1CC3CCC(N3)C1CO2 ; Nc1nc2cc(O)cc(-c3nc4c5c(nc(OCC67CCCN6CCC7)nc5c3F)N3CC5CCC(N5)C3CO4)c2s1] 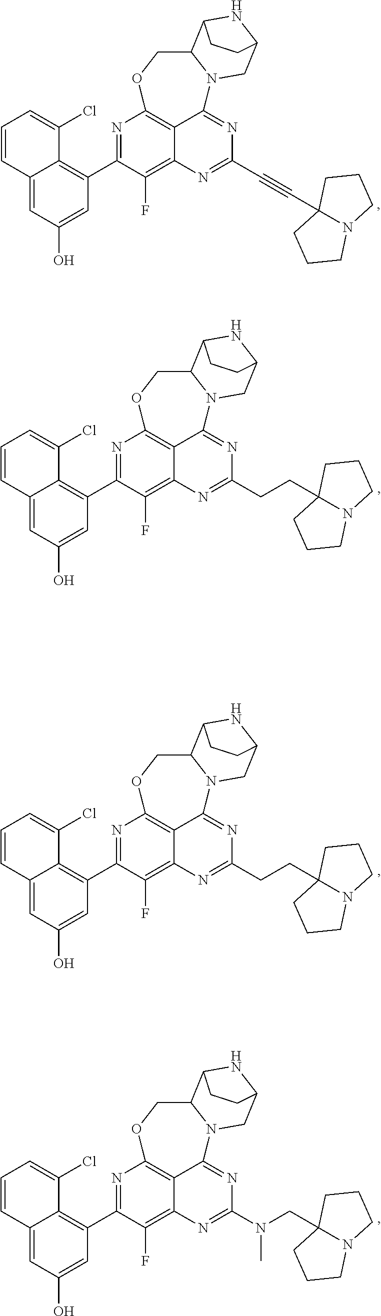 [SMILES: Oc1cc(-c2nc3c4c(nc(C#CC56CCCN5CCC6)nc4c2F)N2CC4CCC(N4)C2CO3)c2c(Cl)cccc2c1 ; Oc1cc(-c2nc3c4c(nc(CCC56CCCN5CCC6)nc4c2F)N2CC4CCC(N4)C2CO3)c2c(Cl)cccc2c1 ; Oc1cc(-c2nc3c4c(nc(CCC56CCCN5CCC6)nc4c2F)N2CC4CCC(N4)C2CO3)c2c(Cl)cccc2c1 ; CN(CC12CCCN1CCC2)c1nc2c3c(nc(-c4cc(O)cc5cccc(Cl)c45)c(F)c3n1)OCC1C3CCC(CN21)N3] 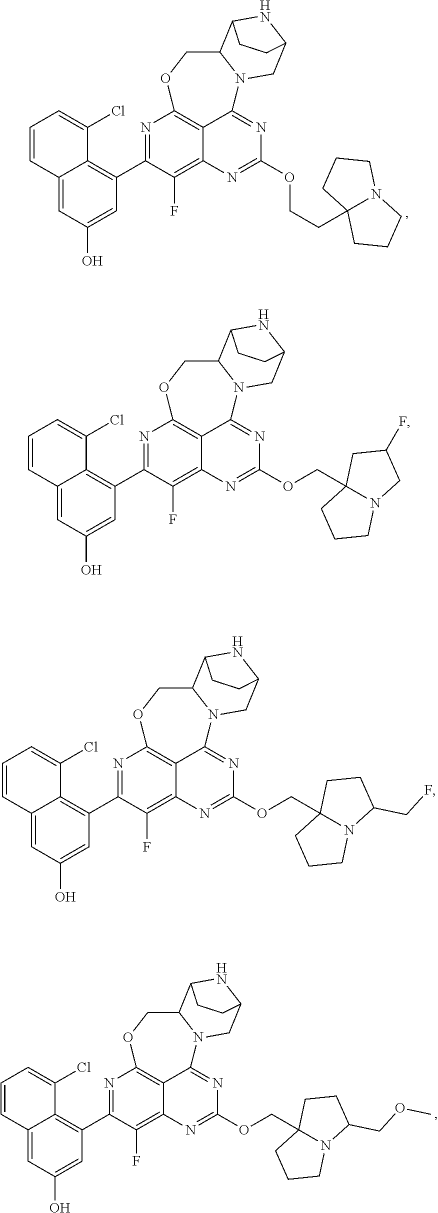 [SMILES: Oc1cc(-c2nc3c4c(nc(OCCC56CCCN5CCC6)nc4c2F)N2CC4CCC(N4)C2CO3)c2c(Cl)cccc2c1 ; Oc1cc(-c2nc3c4c(nc(OCC56CCCN5CC(F)C6)nc4c2F)N2CC4CCC(N4)C2CO3)c2c(Cl)cccc2c1 ; Oc1cc(-c2nc3c4c(nc(OCC56CCCN5C(CF)CC6)nc4c2F)N2CC4CCC(N4)C2CO3)c2c(Cl)cccc2c1 ; COCC1CCC2(COc3nc4c5c(nc(-c6cc(O)cc7cccc(Cl)c67)c(F)c5n3)OCC3C5CCC(CN43)N5)CCCN12] 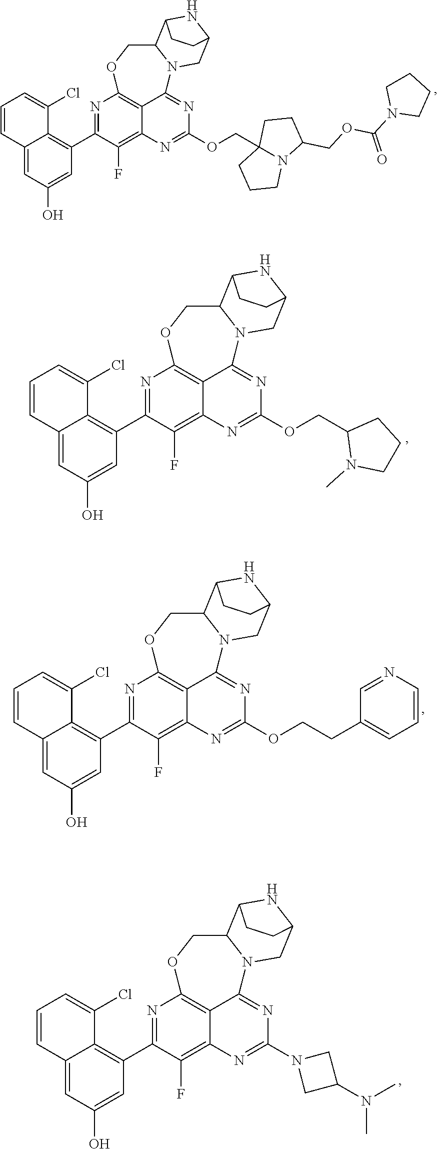 [SMILES: O=C(OCC1CCC2(COc3nc4c5c(nc(-c6cc(O)cc7cccc(Cl)c67)c(F)c5n3)OCC3C5CCC(CN43)N5)CCCN12)N1CCCC1 ; CN1CCCC1COc1nc2c3c(nc(-c4cc(O)cc5cccc(Cl)c45)c(F)c3n1)OCC1C3CCC(CN21)N3 ; Oc1cc(-c2nc3c4c(nc(OCCc5cccnc5)nc4c2F)N2CC4CCC(N4)C2CO3)c2c(Cl)cccc2c1 ; CN(C)C1CN(c2nc3c4c(nc(-c5cc(O)cc6cccc(Cl)c56)c(F)c4n2)OCC2C4CCC(CN32)N4)C1] 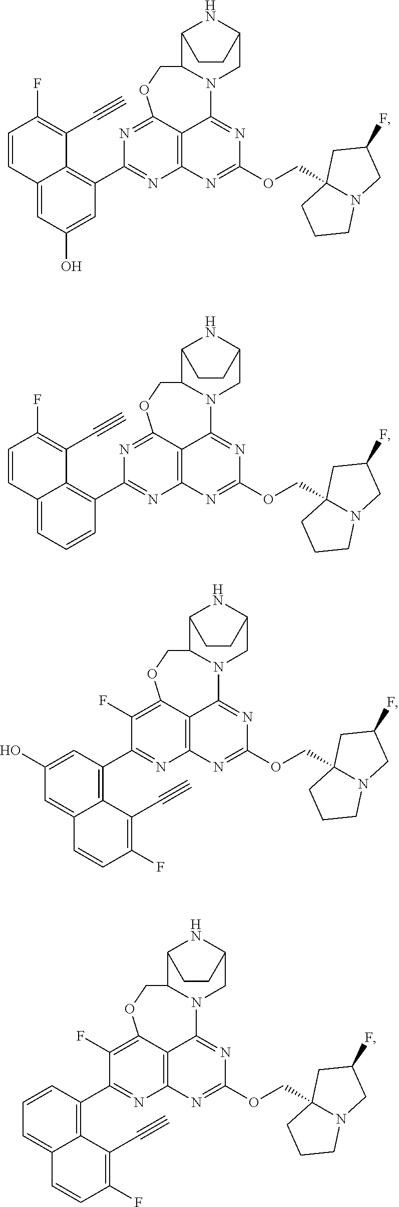 [SMILES: C#Cc1c(F)ccc2cc(O)cc(-c3nc4c5c(nc(OC[C@@]67CCCN6C[C@H](F)C7)nc5n3)N3CC5CCC(N5)C3CO4)c12 ; C#Cc1c(F)ccc2cccc(-c3nc4c5c(nc(OC[C@@]67CCCN6C[C@H](F)C7)nc5n3)N3CC5CCC(N5)C3CO4)c12 ; C#Cc1c(F)ccc2cc(O)cc(-c3nc4nc(OC[C@@]56CCCN5C[C@H](F)C6)nc5c4c(c3F)OCC3C4CCC(CN53)N4)c12 ; C#Cc1c(F)ccc2cccc(-c3nc4nc(OC[C@@]56CCCN5C[C@H](F)C6)nc5c4c(c3F)OCC3C4CCC(CN53)N4)c12] 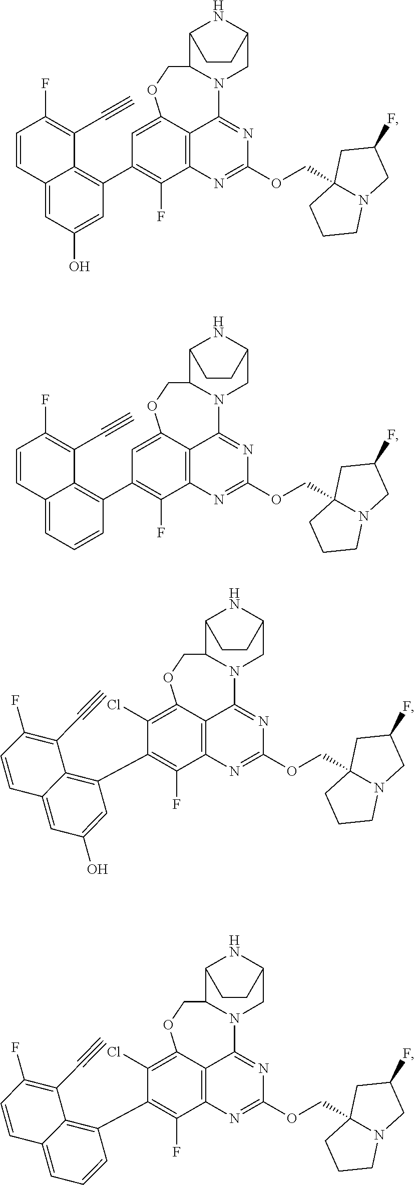 [SMILES: C#Cc1c(F)ccc2cc(O)cc(-c3cc4c5c(nc(OC[C@@]67CCCN6C[C@H](F)C7)nc5c3F)N3CC5CCC(N5)C3CO4)c12 ; C#Cc1c(F)ccc2cccc(-c3cc4c5c(nc(OC[C@@]67CCCN6C[C@H](F)C7)nc5c3F)N3CC5CCC(N5)C3CO4)c12 ; C#Cc1c(F)ccc2cc(O)cc(-c3c(Cl)c4c5c(nc(OC[C@@]67CCCN6C[C@H](F)C7)nc5c3F)N3CC5CCC(N5)C3CO4)c12 ; C#Cc1c(F)ccc2cccc(-c3c(Cl)c4c5c(nc(OC[C@@]67CCCN6C[C@H](F)C7)nc5c3F)N3CC5CCC(N5)C3CO4)c12] 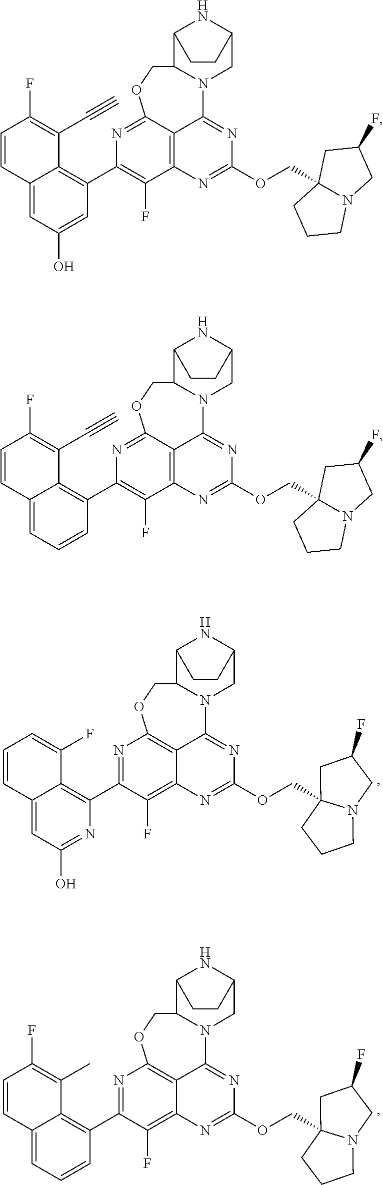 [SMILES: C#Cc1c(F)ccc2cc(O)cc(-c3nc4c5c(nc(OC[C@@]67CCCN6C[C@H](F)C7)nc5c3F)N3CC5CCC(N5)C3CO4)c12 ; C#Cc1c(F)ccc2cccc(-c3nc4c5c(nc(OC[C@@]67CCCN6C[C@H](F)C7)nc5c3F)N3CC5CCC(N5)C3CO4)c12 ; Oc1cc2cccc(F)c2c(-c2nc3c4c(nc(OC[C@@]56CCCN5C[C@H](F)C6)nc4c2F)N2CC4CCC(N4)C2CO3)n1 ; Cc1c(F)ccc2cccc(-c3nc4c5c(nc(OC[C@@]67CCCN6C[C@H](F)C7)nc5c3F)N3CC5CCC(N5)C3CO4)c12] 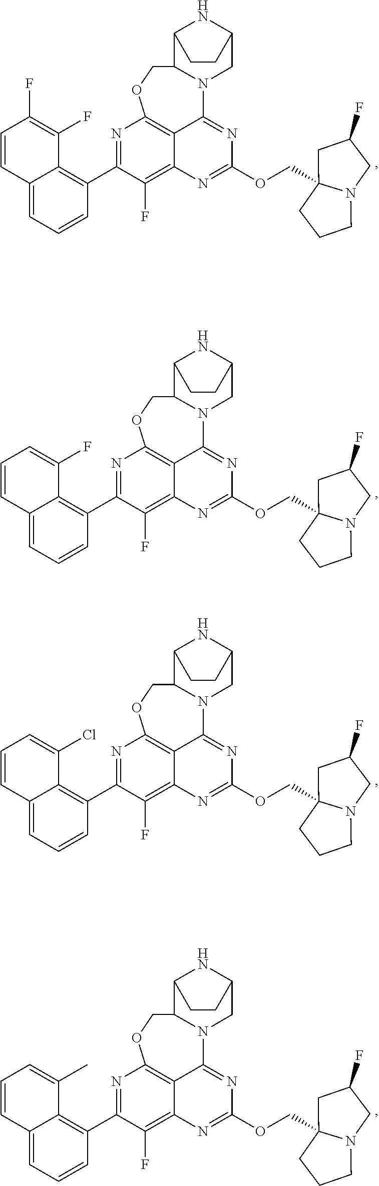 [SMILES: Fc1ccc2cccc(-c3nc4c5c(nc(OC[C@@]67CCCN6C[C@H](F)C7)nc5c3F)N3CC5CCC(N5)C3CO4)c2c1F ; Fc1c(-c2cccc3cccc(F)c23)nc2c3c(nc(OC[C@@]45CCCN4C[C@H](F)C5)nc13)N1CC3CCC(N3)C1CO2 ; Fc1c(-c2cccc3cccc(Cl)c23)nc2c3c(nc(OC[C@@]45CCCN4C[C@H](F)C5)nc13)N1CC3CCC(N3)C1CO2 ; Cc1cccc2cccc(-c3nc4c5c(nc(OC[C@@]67CCCN6C[C@H](F)C7)nc5c3F)N3CC5CCC(N5)C3CO4)c12] 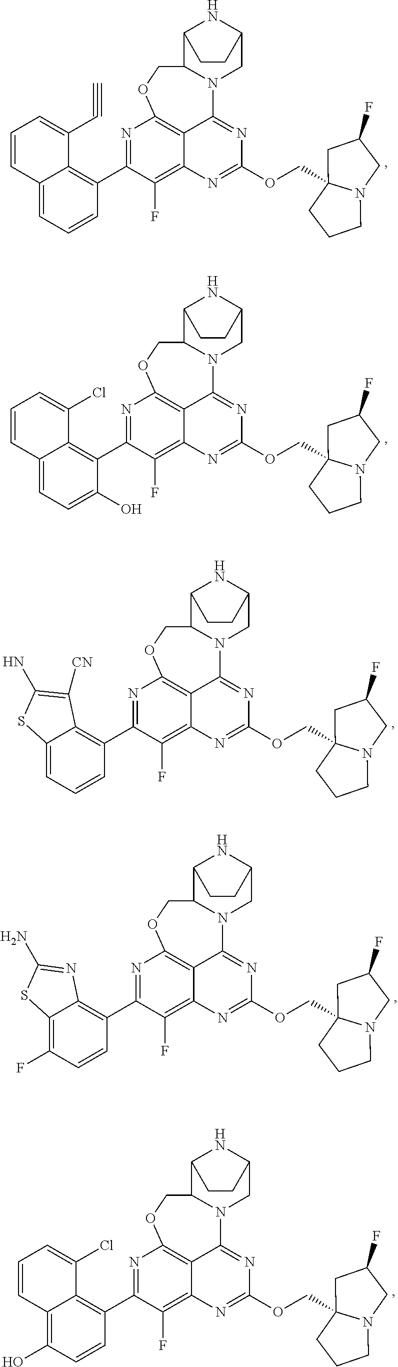 [SMILES: C#Cc1cccc2cccc(-c3nc4c5c(nc(OC[C@@]67CCCN6C[C@H](F)C7)nc5c3F)N3CC5CCC(N5)C3CO4)c12 ; Oc1ccc2cccc(Cl)c2c1-c1nc2c3c(nc(OC[C@@]45CCCN4C[C@H](F)C5)nc3c1F)N1CC3CCC(N3)C1CO2 ; N#Cc1c(N)sc2cccc(-c3nc4c5c(nc(OC[C@@]67CCCN6C[C@H](F)C7)nc5c3F)N3CC5CCC(N5)C3CO4)c12 ; Nc1nc2c(-c3nc4c5c(nc(OC[C@@]67CCCN6C[C@H](F)C7)nc5c3F)N3CC5CCC(N5)C3CO4)ccc(F)c2s1 ; Oc1ccc(-c2nc3c4c(nc(OC[C@@]56CCCN5C[C@H](F)C6)nc4c2F)N2CC4CCC(N4)C2CO3)c2c(Cl)cccc12] 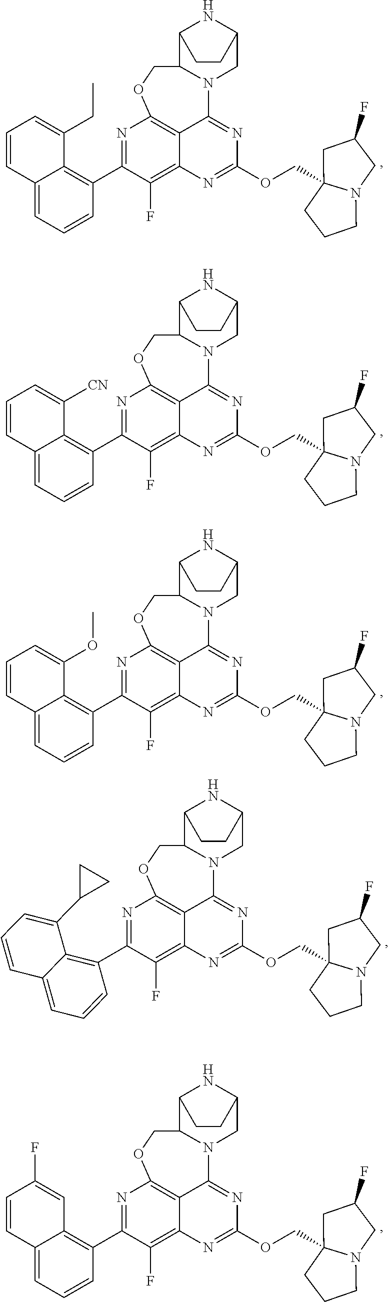 [SMILES: CCc1cccc2cccc(-c3nc4c5c(nc(OC[C@@]67CCCN6C[C@H](F)C7)nc5c3F)N3CC5CCC(N5)C3CO4)c12 ; N#Cc1cccc2cccc(-c3nc4c5c(nc(OC[C@@]67CCCN6C[C@H](F)C7)nc5c3F)N3CC5CCC(N5)C3CO4)c12 ; COc1cccc2cccc(-c3nc4c5c(nc(OC[C@@]67CCCN6C[C@H](F)C7)nc5c3F)N3CC5CCC(N5)C3CO4)c12 ; Fc1c(-c2cccc3cccc(C4CC4)c23)nc2c3c(nc(OC[C@@]45CCCN4C[C@H](F)C5)nc13)N1CC3CCC(N3)C1CO2 ; Fc1ccc2cccc(-c3nc4c5c(nc(OC[C@@]67CCCN6C[C@H](F)C7)nc5c3F)N3CC5CCC(N5)C3CO4)c2c1] 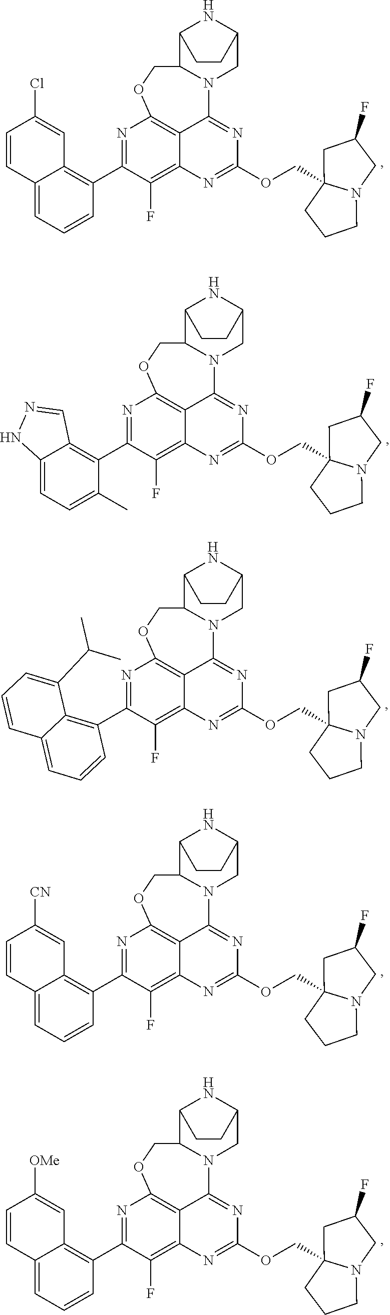 [SMILES: Fc1c(-c2cccc3ccc(Cl)cc23)nc2c3c(nc(OC[C@@]45CCCN4C[C@H](F)C5)nc13)N1CC3CCC(N3)C1CO2 ; Cc1ccc2[nH]ncc2c1-c1nc2c3c(nc(OC[C@@]45CCCN4C[C@H](F)C5)nc3c1F)N1CC3CCC(N3)C1CO2 ; CC(C)c1cccc2cccc(-c3nc4c5c(nc(OC[C@@]67CCCN6C[C@H](F)C7)nc5c3F)N3CC5CCC(N5)C3CO4)c12 ; N#Cc1ccc2cccc(-c3nc4c5c(nc(OC[C@@]67CCCN6C[C@H](F)C7)nc5c3F)N3CC5CCC(N5)C3CO4)c2c1 ; COc1ccc2cccc(-c3nc4c5c(nc(OC[C@@]67CCCN6C[C@H](F)C7)nc5c3F)N3CC5CCC(N5)C3CO4)c2c1] 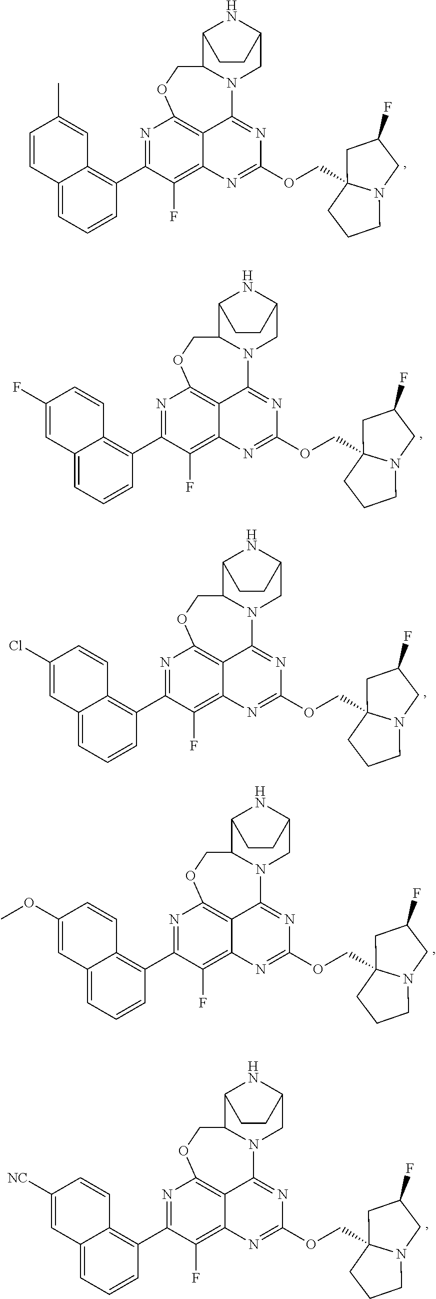 [SMILES: Cc1ccc2cccc(-c3nc4c5c(nc(OC[C@@]67CCCN6C[C@H](F)C7)nc5c3F)N3CC5CCC(N5)C3CO4)c2c1 ; Fc1ccc2c(-c3nc4c5c(nc(OC[C@@]67CCCN6C[C@H](F)C7)nc5c3F)N3CC5CCC(N5)C3CO4)cccc2c1 ; Fc1c(-c2cccc3cc(Cl)ccc23)nc2c3c(nc(OC[C@@]45CCCN4C[C@H](F)C5)nc13)N1CC3CCC(N3)C1CO2 ; COc1ccc2c(-c3nc4c5c(nc(OC[C@@]67CCCN6C[C@H](F)C7)nc5c3F)N3CC5CCC(N5)C3CO4)cccc2c1 ; N#Cc1ccc2c(-c3nc4c5c(nc(OC[C@@]67CCCN6C[C@H](F)C7)nc5c3F)N3CC5CCC(N5)C3CO4)cccc2c1] 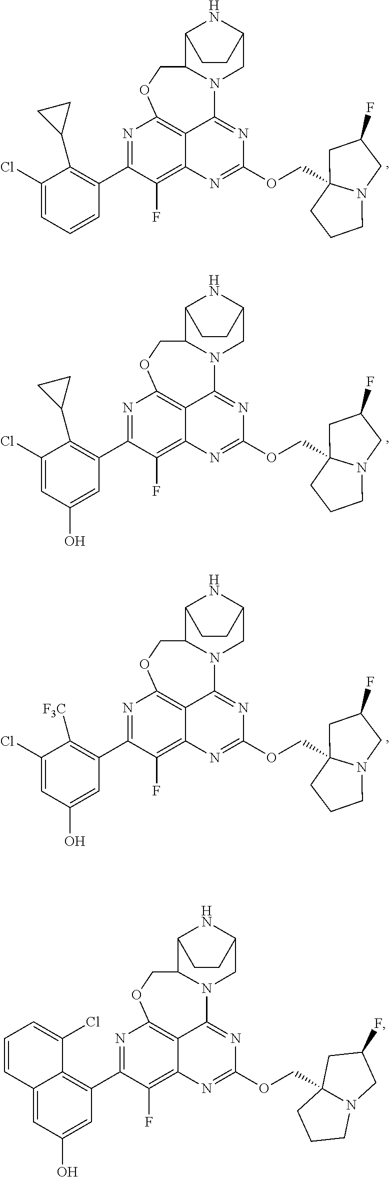 [SMILES: Fc1c(-c2cccc(Cl)c2C2CC2)nc2c3c(nc(OC[C@@]45CCCN4C[C@H](F)C5)nc13)N1CC3CCC(N3)C1CO2 ; Oc1cc(Cl)c(C2CC2)c(-c2nc3c4c(nc(OC[C@@]56CCCN5C[C@H](F)C6)nc4c2F)N2CC4CCC(N4)C2CO3)c1 ; Oc1cc(Cl)c(C(F)(F)F)c(-c2nc3c4c(nc(OC[C@@]56CCCN5C[C@H](F)C6)nc4c2F)N2CC4CCC(N4)C2CO3)c1 ; Oc1cc(-c2nc3c4c(nc(OC[C@@]56CCCN5C[C@H](F)C6)nc4c2F)N2CC4CCC(N4)C2CO3)c2c(Cl)cccc2c1] 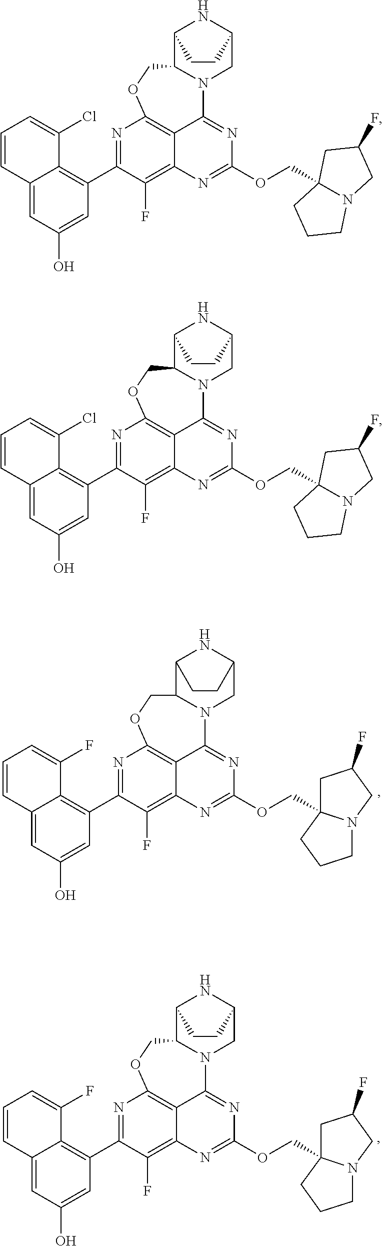 [SMILES: Oc1cc(-c2nc3c4c(nc(OC[C@@]56CCCN5C[C@H](F)C6)nc4c2F)N2C[C@H]4CC[C@H](N4)[C@H]2CO3)c2c(Cl)cccc2c1 ; Oc1cc(-c2nc3c4c(nc(OC[C@@]56CCCN5C[C@H](F)C6)nc4c2F)N2C[C@H]4CC[C@H](N4)[C@@H]2CO3)c2c(Cl)cccc2c1 ; Oc1cc(-c2nc3c4c(nc(OC[C@@]56CCCN5C[C@H](F)C6)nc4c2F)N2CC4CCC(N4)C2CO3)c2c(F)cccc2c1 ; Oc1cc(-c2nc3c4c(nc(OC[C@@]56CCCN5C[C@H](F)C6)nc4c2F)N2C[C@H]4CC[C@H](N4)[C@H]2CO3)c2c(F)cccc2c1] 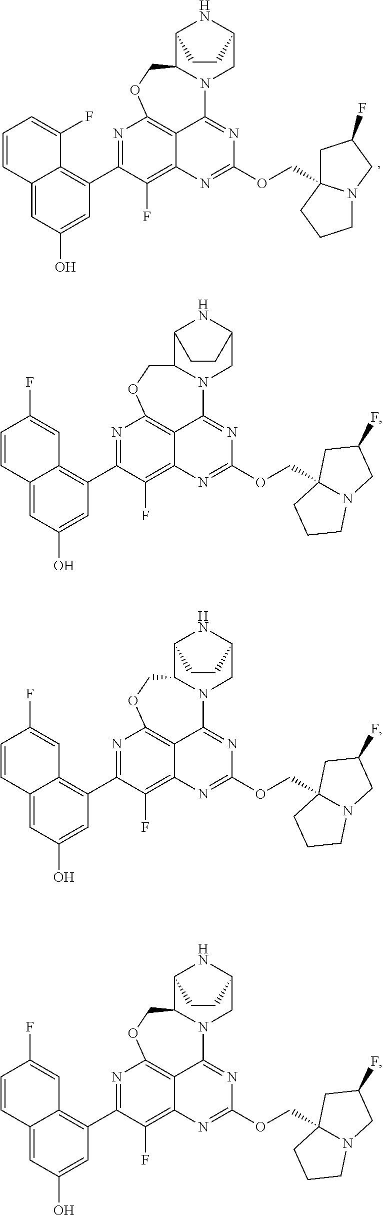 [SMILES: Oc1cc(-c2nc3c4c(nc(OC[C@@]56CCCN5C[C@H](F)C6)nc4c2F)N2C[C@H]4CC[C@H](N4)[C@@H]2CO3)c2c(F)cccc2c1 ; Oc1cc(-c2nc3c4c(nc(OC[C@@]56CCCN5C[C@H](F)C6)nc4c2F)N2CC4CCC(N4)C2CO3)c2cc(F)ccc2c1 ; Oc1cc(-c2nc3c4c(nc(OC[C@@]56CCCN5C[C@H](F)C6)nc4c2F)N2C[C@H]4CC[C@H](N4)[C@H]2CO3)c2cc(F)ccc2c1 ; Oc1cc(-c2nc3c4c(nc(OC[C@@]56CCCN5C[C@H](F)C6)nc4c2F)N2C[C@H]4CC[C@H](N4)[C@@H]2CO3)c2cc(F)ccc2c1] 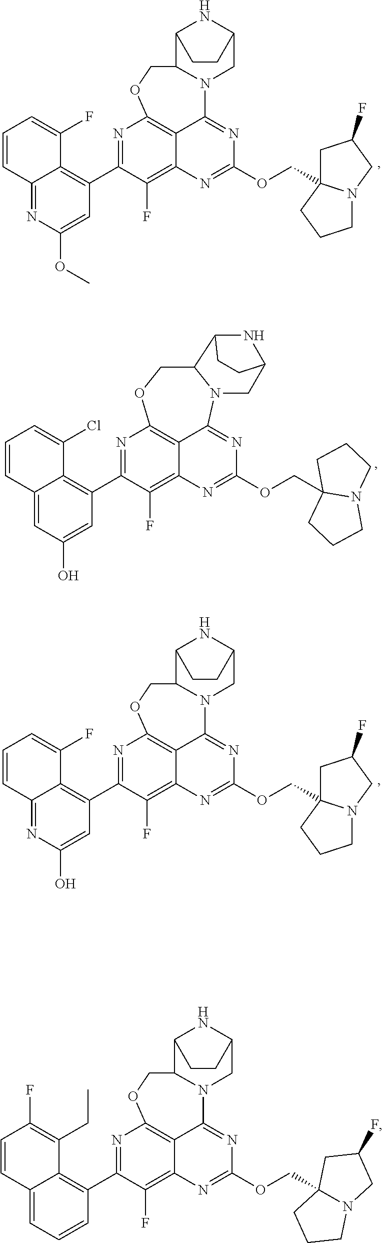 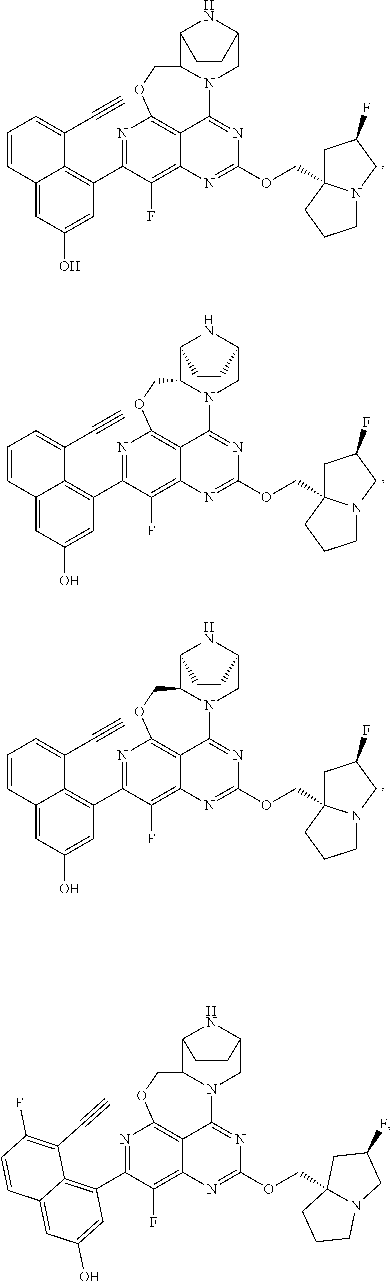 [SMILES: C#Cc1cccc2cc(O)cc(-c3nc4c5c(nc(OC[C@@]67CCCN6C[C@H](F)C7)nc5c3F)N3CC5CCC(N5)C3CO4)c12 ; C#Cc1cccc2cc(O)cc(-c3nc4c5c(nc(OC[C@@]67CCCN6C[C@H](F)C7)nc5c3F)N3C[C@H]5CC[C@H](N5)[C@H]3CO4)c12 ; C#Cc1cccc2cc(O)cc(-c3nc4c5c(nc(OC[C@@]67CCCN6C[C@H](F)C7)nc5c3F)N3C[C@H]5CC[C@H](N5)[C@@H]3CO4)c12 ; C#Cc1c(F)ccc2cc(O)cc(-c3nc4c5c(nc(OC[C@@]67CCCN6C[C@H](F)C7)nc5c3F)N3CC5CCC(N5)C3CO4)c12] 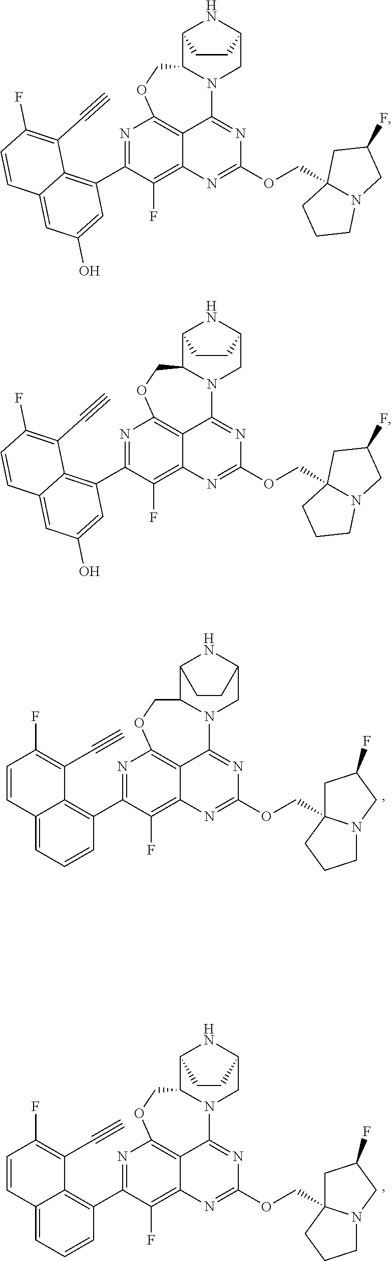 [SMILES: C#Cc1c(F)ccc2cc(O)cc(-c3nc4c5c(nc(OC[C@@]67CCCN6C[C@H](F)C7)nc5c3F)N3C[C@H]5CC[C@H](N5)[C@H]3CO4)c12 ; C#Cc1c(F)ccc2cc(O)cc(-c3nc4c5c(nc(OC[C@@]67CCCN6C[C@H](F)C7)nc5c3F)N3C[C@H]5CC[C@H](N5)[C@@H]3CO4)c12 ; C#Cc1c(F)ccc2cccc(-c3nc4c5c(nc(OC[C@@]67CCCN6C[C@H](F)C7)nc5c3F)N3CC5CCC(N5)C3CO4)c12 ; C#Cc1c(F)ccc2cccc(-c3nc4c5c(nc(OC[C@@]67CCCN6C[C@H](F)C7)nc5c3F)N3C[C@H]5CC[C@H](N5)[C@H]3CO4)c12] 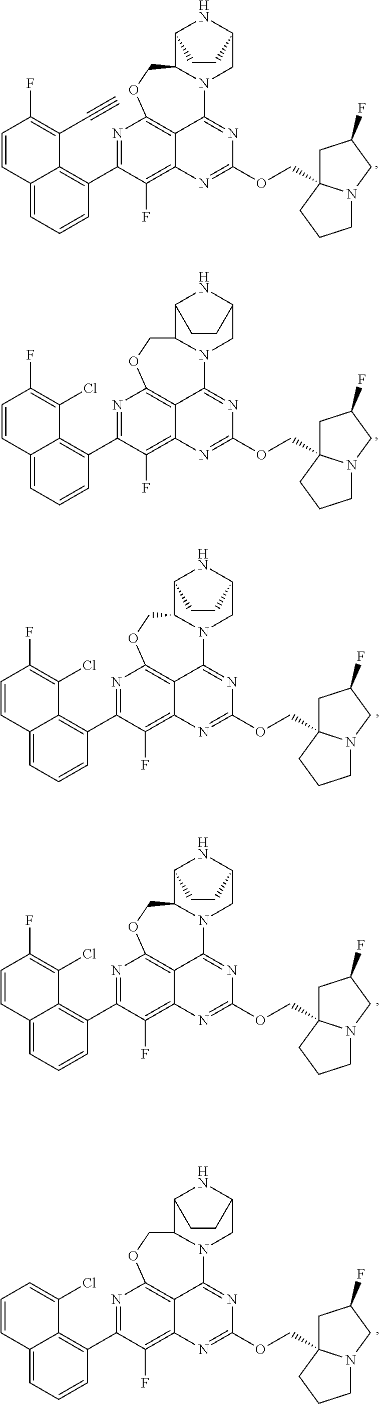 [SMILES: C#Cc1c(F)ccc2cccc(-c3nc4c5c(nc(OC[C@@]67CCCN6C[C@H](F)C7)nc5c3F)N3C[C@H]5CC[C@H](N5)[C@@H]3CO4)c12 ; Fc1ccc2cccc(-c3nc4c5c(nc(OC[C@@]67CCCN6C[C@H](F)C7)nc5c3F)N3CC5CCC(N5)C3CO4)c2c1Cl ; Fc1ccc2cccc(-c3nc4c5c(nc(OC[C@@]67CCCN6C[C@H](F)C7)nc5c3F)N3C[C@H]5CC[C@H](N5)[C@H]3CO4)c2c1Cl ; Fc1ccc2cccc(-c3nc4c5c(nc(OC[C@@]67CCCN6C[C@H](F)C7)nc5c3F)N3C[C@H]5CC[C@H](N5)[C@@H]3CO4)c2c1Cl ; Fc1c(-c2cccc3cccc(Cl)c23)nc2c3c(nc(OC[C@@]45CCCN4C[C@H](F)C5)nc13)N1CC3CCC(N3)C1CO2] 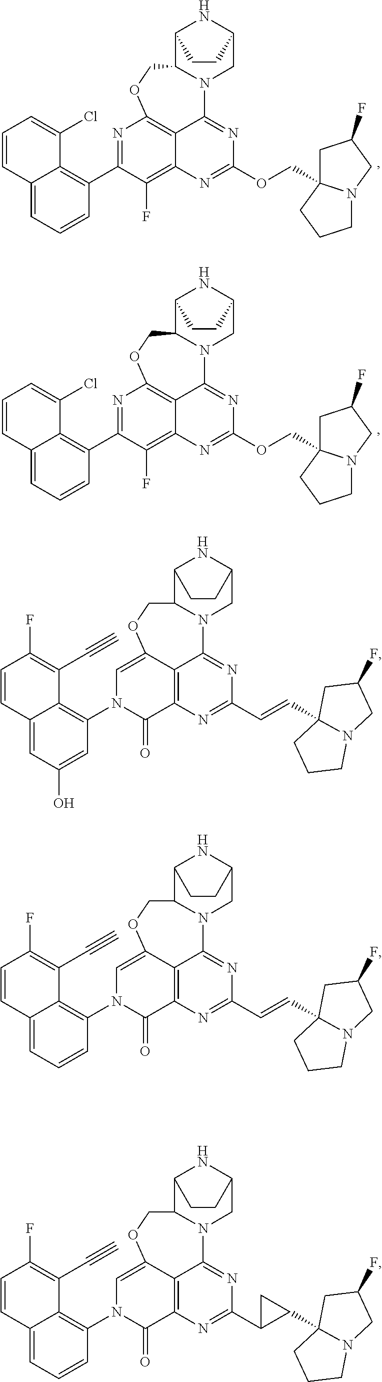 [SMILES: Fc1c(-c2cccc3cccc(Cl)c23)nc2c3c(nc(OC[C@@]45CCCN4C[C@H](F)C5)nc13)N1C[C@H]3CC[C@H](N3)[C@H]1CO2 ; Fc1c(-c2cccc3cccc(Cl)c23)nc2c3c(nc(OC[C@@]45CCCN4C[C@H](F)C5)nc13)N1C[C@H]3CC[C@H](N3)[C@@H]1CO2 ; C#Cc1c(F)ccc2cc(O)cc(-n3cc4c5c(nc(/C=C/[C@@]67CCCN6C[C@H](F)C7)nc5c3=O)N3CC5CCC(N5)C3CO4)c12 ; C#Cc1c(F)ccc2cccc(-n3cc4c5c(nc(/C=C/[C@@]67CCCN6C[C@H](F)C7)nc5c3=O)N3CC5CCC(N5)C3CO4)c12 ; C#Cc1c(F)ccc2cccc(-n3cc4c5c(nc(C6CC6[C@@]67CCCN6C[C@H](F)C7)nc5c3=O)N3CC5CCC(N5)C3CO4)c12] 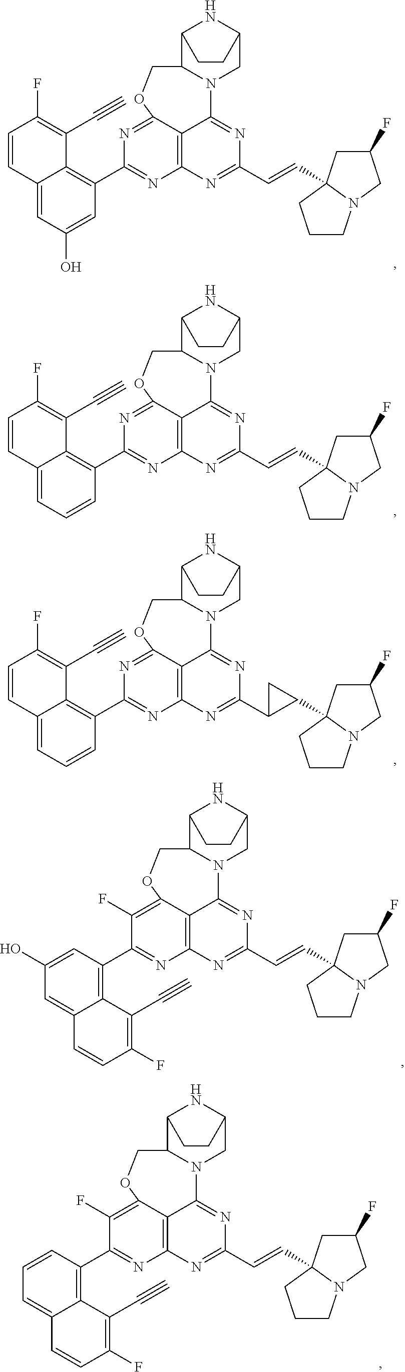 [SMILES: C#Cc1c(F)ccc2cc(O)cc(-c3nc4c5c(nc(/C=C/[C@@]67CCCN6C[C@H](F)C7)nc5n3)N3CC5CCC(N5)C3CO4)c12 ; C#Cc1c(F)ccc2cccc(-c3nc4c5c(nc(/C=C/[C@@]67CCCN6C[C@H](F)C7)nc5n3)N3CC5CCC(N5)C3CO4)c12 ; C#Cc1c(F)ccc2cccc(-c3nc4c5c(nc(C6CC6[C@@]67CCCN6C[C@H](F)C7)nc5n3)N3CC5CCC(N5)C3CO4)c12 ; C#Cc1c(F)ccc2cc(O)cc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(/C=C/[C@@]56CCCN5C[C@H](F)C6)nc5c4c(c3F)OCC3C4CCC(CN53)N4)c12] 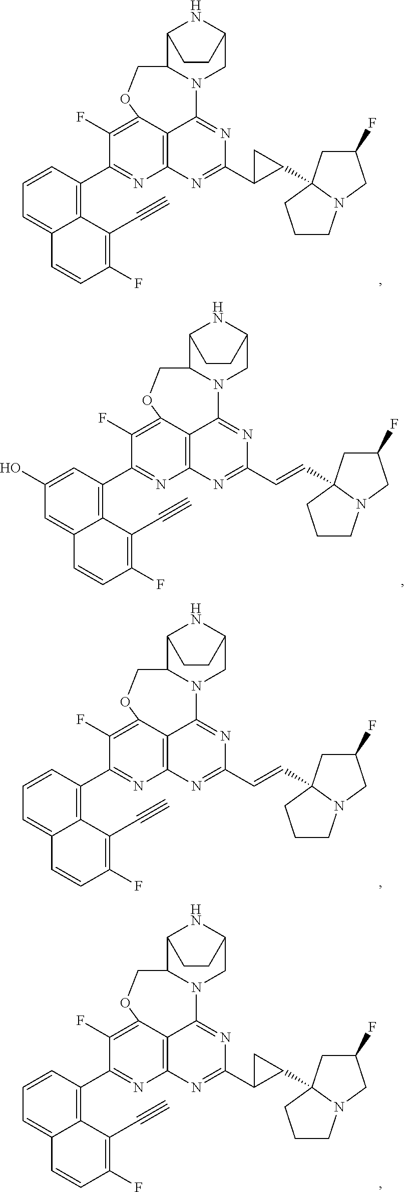 [SMILES: C#Cc1c(F)ccc2cccc(-c3nc4nc(C5CC5[C@@]56CCCN5C[C@H](F)C6)nc5c4c(c3F)OCC3C4CCC(CN53)N4)c12 ; C#Cc1c(F)ccc2cc(O)cc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(/C=C/[C@@]56CCCN5C[C@H](F)C6)nc5c4c(c3F)OCC3C4CCC(CN53)N4)c12 ; C#Cc1c(F)ccc2cccc(-c3nc4nc(C5CC5[C@@]56CCCN5C[C@H](F)C6)nc5c4c(c3F)OCC3C4CCC(CN53)N4)c12] 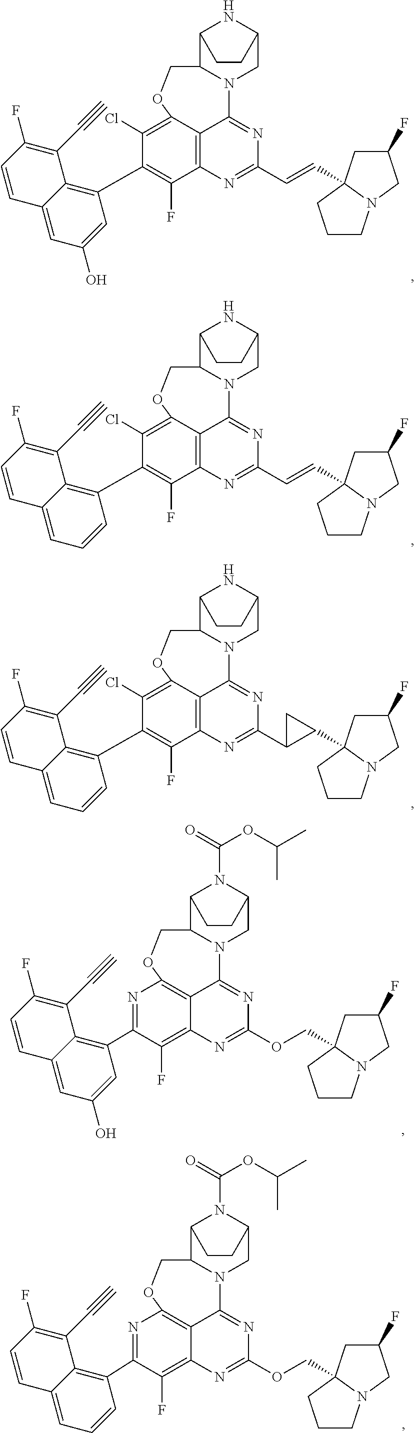 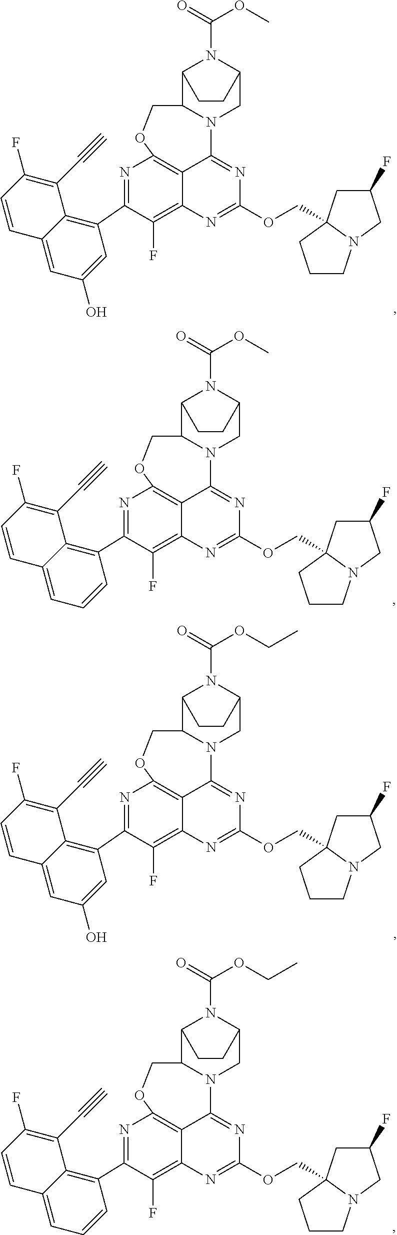 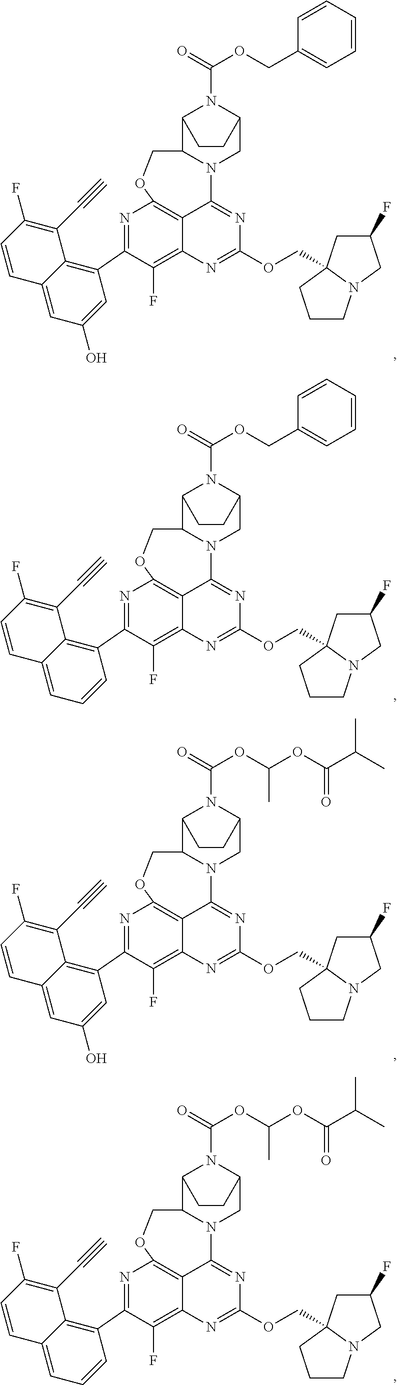 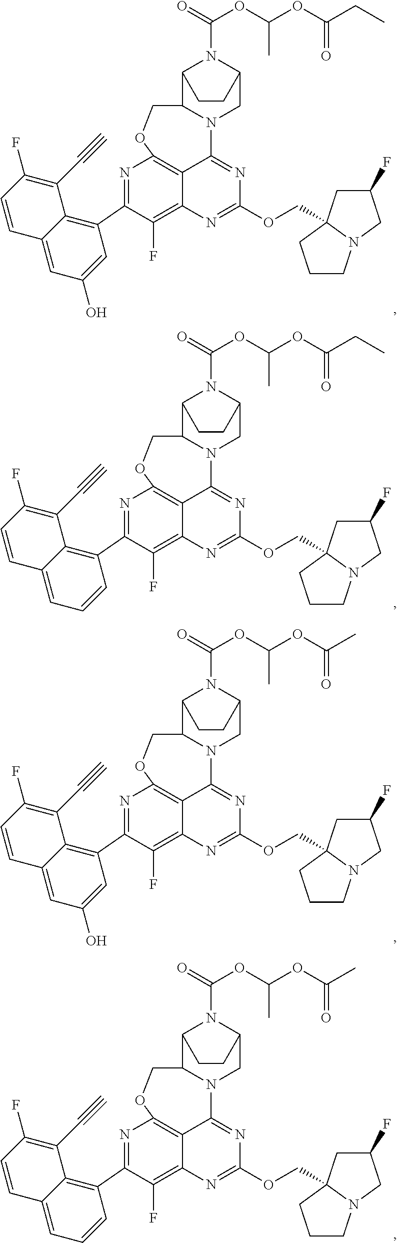 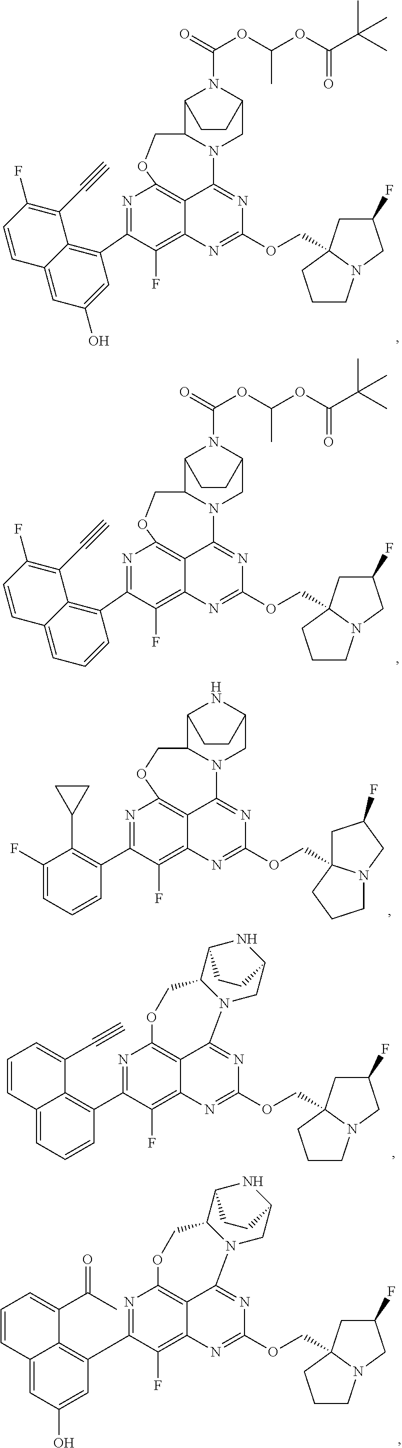 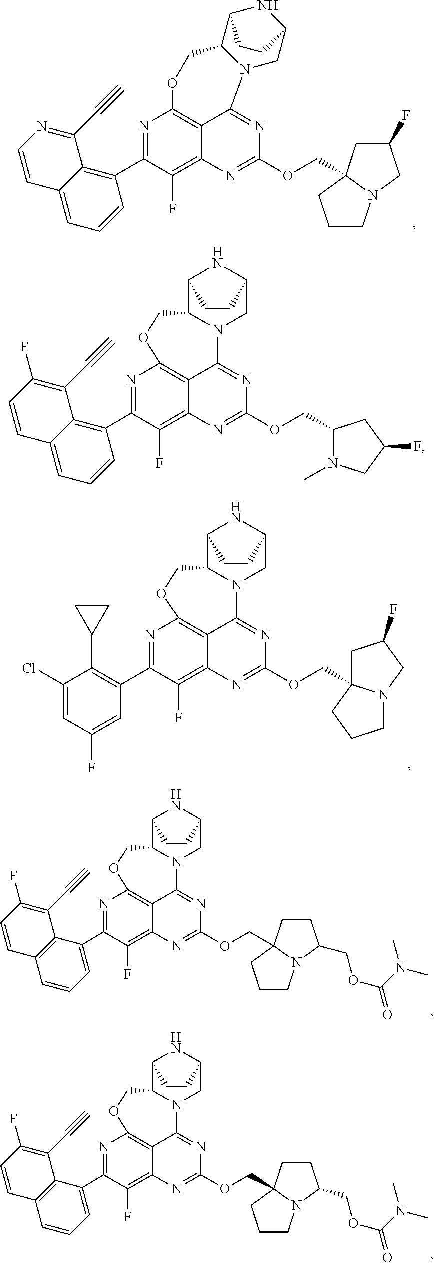 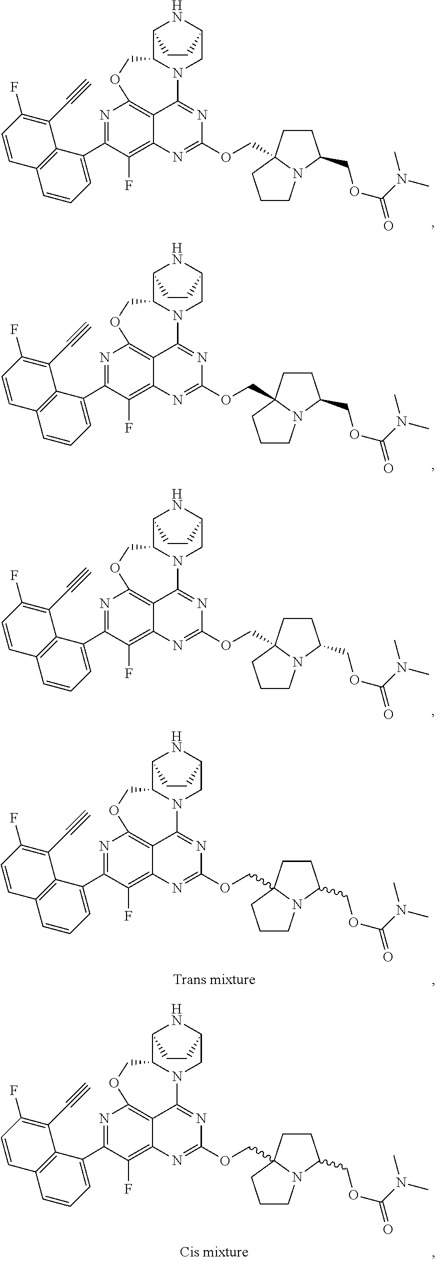 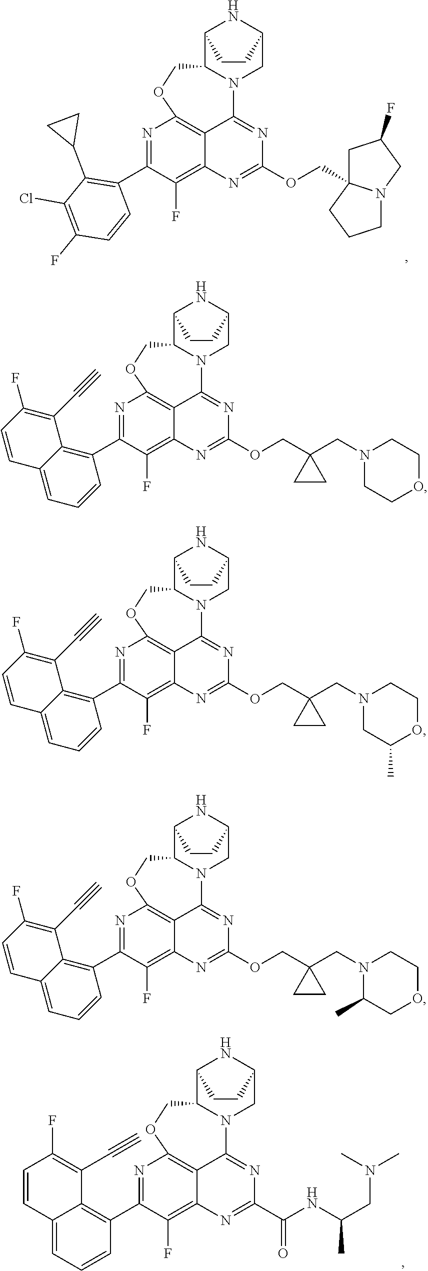 [SMILES: Fc1ccc(-c2nc3c4c(nc(OC[C@@]56CCCN5C[C@H](F)C6)nc4c2F)N2C[C@H]4CC[C@H](N4)[C@H]2CO3)c(C2CC2)c1Cl ; C#Cc1c(F)ccc2cccc(-c3nc4c5c(nc(OCC6(CN7CCOCC7)CC6)nc5c3F)N3C[C@H]5CC[C@H](N5)[C@H]3CO4)c12 ; C#Cc1c(F)ccc2cccc(-c3nc4c5c(nc(OCC6(CN7CCO[C@H](C)C7)CC6)nc5c3F)N3C[C@H]5CC[C@H](N5)[C@H]3CO4)c12 ; C#Cc1c(F)ccc2cccc(-c3nc4c5c(nc(OCC6(CN7CCOC[C@H]7C)CC6)nc5c3F)N3C[C@H]5CC[C@H](N5)[C@H]3CO4)c12 ; C#Cc1c(F)ccc2cccc(-c3nc4c5c(nc(C(=O)N[C@H](C)CN(C)C)nc5c3F)N3C[C@H]5CC[C@H](N5)[C@H]3CO4)c12] 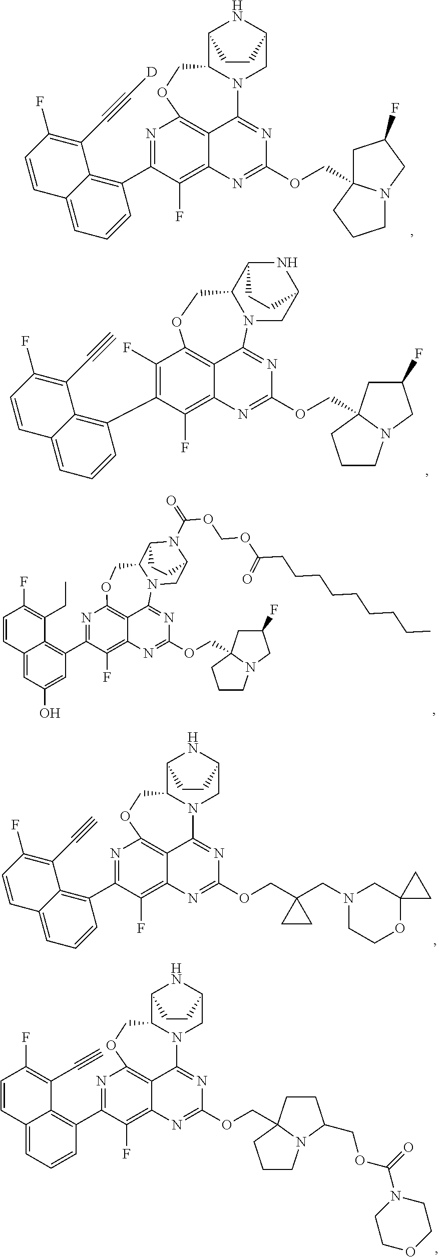 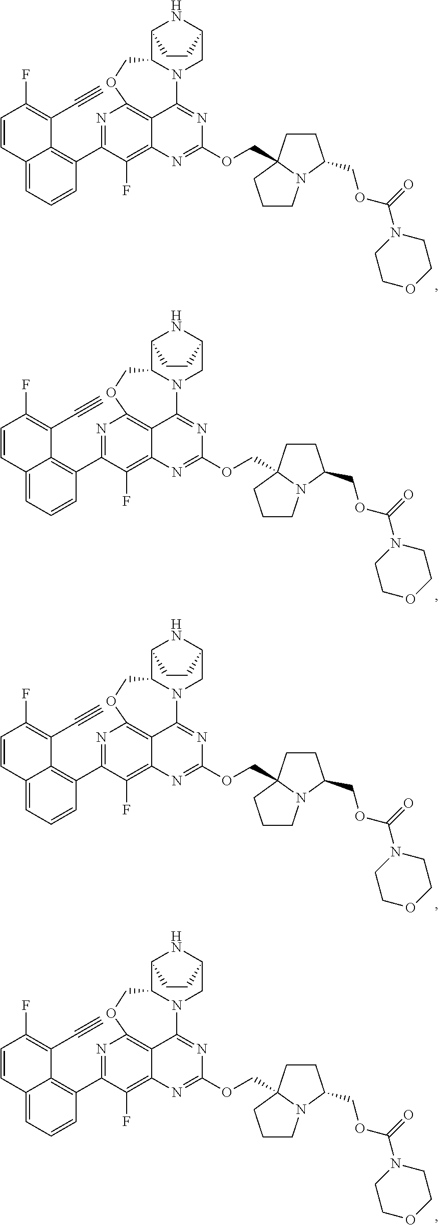 [SMILES: C#Cc1c(F)ccc2cccc(-c3nc4c5c(nc(OC[C@]67CCCN6[C@@H](COC(=O)N6CCOCC6)CC7)nc5c3F)N3C[C@H]5CC[C@H](N5)[C@H]3CO4)c12 ; C#Cc1c(F)ccc2cccc(-c3nc4c5c(nc(OC[C@@]67CCCN6[C@H](COC(=O)N6CCOCC6)CC7)nc5c3F)N3C[C@H]5CC[C@H](N5)[C@H]3CO4)c12 ; C#Cc1c(F)ccc2cccc(-c3nc4c5c(nc(OC[C@]67CCCN6[C@H](COC(=O)N6CCOCC6)CC7)nc5c3F)N3C[C@H]5CC[C@H](N5)[C@H]3CO4)c12 ; C#Cc1c(F)ccc2cccc(-c3nc4c5c(nc(OC[C@@]67CCCN6[C@@H](COC(=O)N6CCOCC6)CC7)nc5c3F)N3C[C@H]5CC[C@H](N5)[C@H]3CO4)c12] 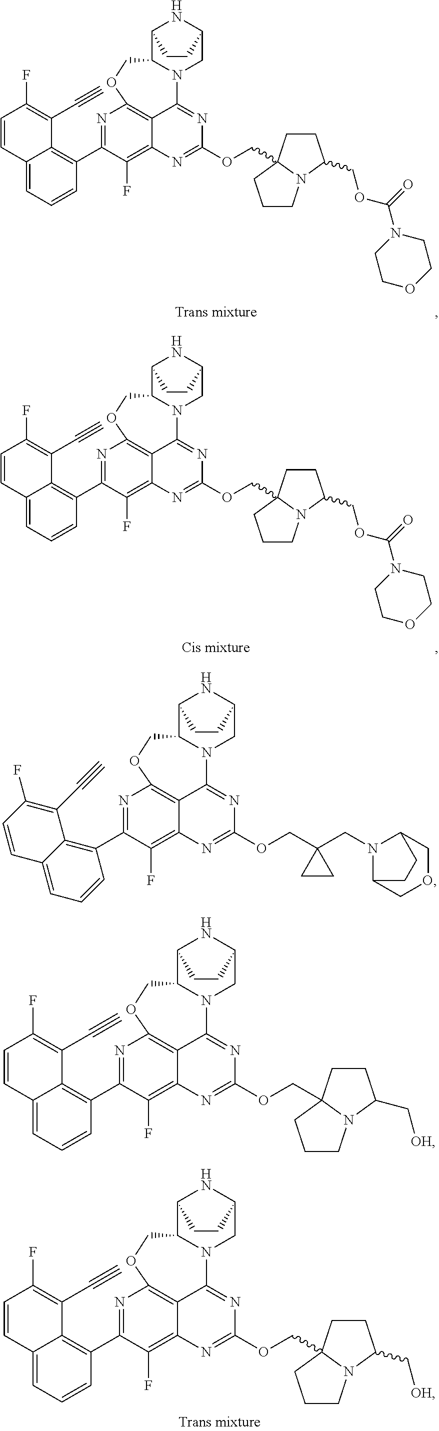 [SMILES: C#Cc1c(F)ccc2cccc(-c3nc4c5c(nc(OCC67CCCN6C(COC(=O)N6CCOCC6)CC7)nc5c3F)N3C[C@H]5CC[C@H](N5)[C@H]3CO4)c12 ; C#Cc1c(F)ccc2cccc(-c3nc4c5c(nc(OCC67CCCN6C(COC(=O)N6CCOCC6)CC7)nc5c3F)N3C[C@H]5CC[C@H](N5)[C@H]3CO4)c12 ; C#Cc1c(F)ccc2cccc(-c3nc4c5c(nc(OCC6(CN7C8CCC7COC8)CC6)nc5c3F)N3C[C@H]5CC[C@H](N5)[C@H]3CO4)c12 ; C#Cc1c(F)ccc2cccc(-c3nc4c5c(nc(OCC67CCCN6C(CO)CC7)nc5c3F)N3C[C@H]5CC[C@H](N5)[C@H]3CO4)c12 ; C#Cc1c(F)ccc2cccc(-c3nc4c5c(nc(OCC67CCCN6C(CO)CC7)nc5c3F)N3C[C@H]5CC[C@H](N5)[C@H]3CO4)c12] 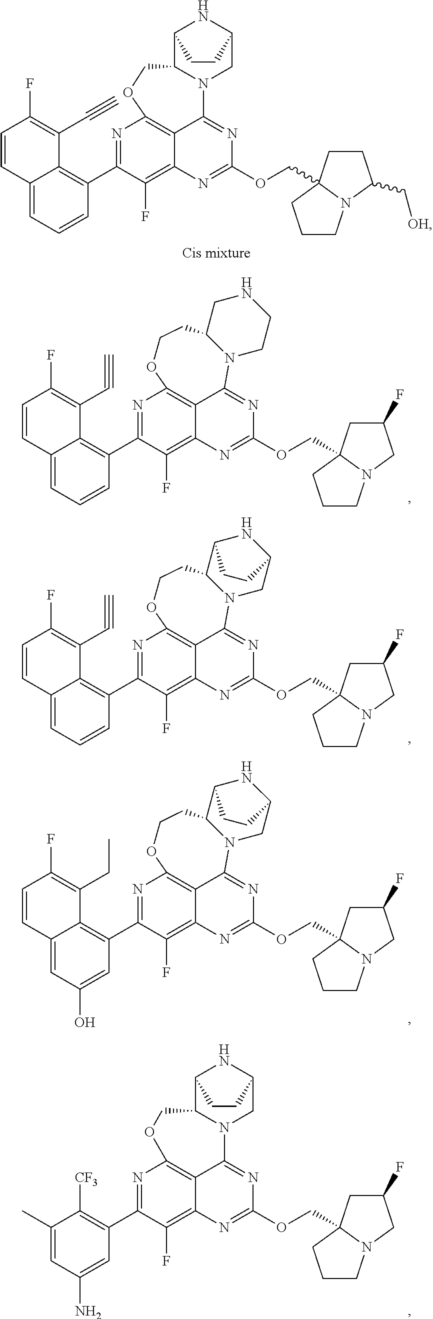 [SMILES: C#Cc1c(F)ccc2cccc(-c3nc4c5c(nc(OCC67CCCN6C(CO)CC7)nc5c3F)N3C[C@H]5CC[C@H](N5)[C@H]3CO4)c12 ; C#Cc1c(F)ccc2cccc(-c3nc4c5c(nc(OC[C@@]67CCCN6C[C@H](F)C7)nc5c3F)N3CCNC[C@H]3CCO4)c12 ; C#Cc1c(F)ccc2cccc(-c3nc4c5c(nc(OC[C@@]67CCCN6C[C@H](F)C7)nc5c3F)N3C[C@H]5CC[C@H](N5)[C@H]3CCO4)c12 ; CCc1c(F)ccc2cc(O)cc(-c3nc4c5c(nc(OC[C@@]67CCCN6C[C@H](F)C7)nc5c3F)N3C[C@H]5CC[C@H](N5)[C@H]3CCO4)c12 ; Cc1cc(N)cc(-c2nc3c4c(nc(OC[C@@]56CCCN5C[C@H](F)C6)nc4c2F)N2C[C@H]4CC[C@H](N4)[C@H]2CO3)c1C(F)(F)F] 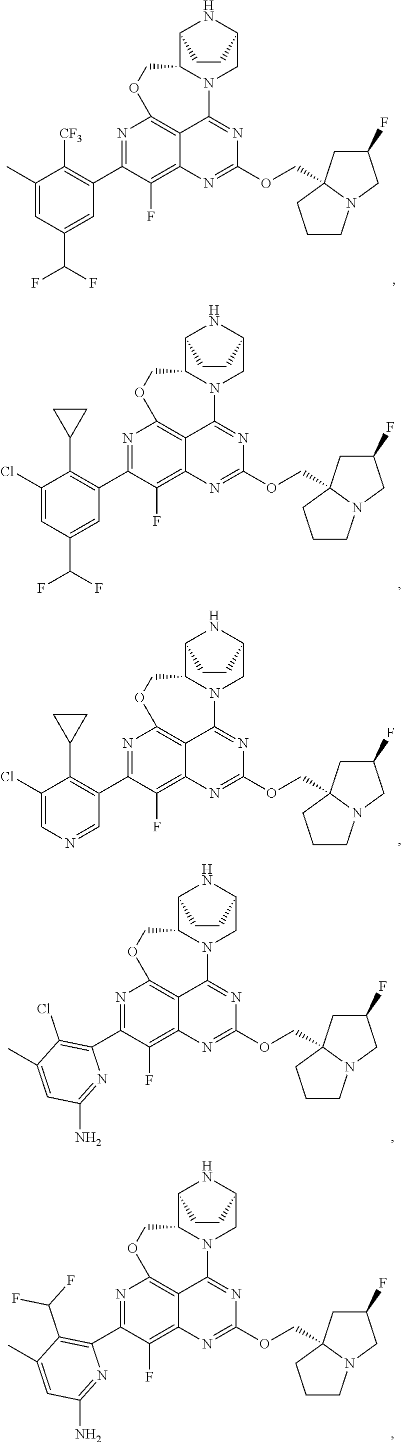 [SMILES: Cc1cc(C(F)F)cc(-c2nc3c4c(nc(OC[C@@]56CCCN5C[C@H](F)C6)nc4c2F)N2C[C@H]4CC[C@H](N4)[C@H]2CO3)c1C(F)(F)F ; Fc1c(-c2cc(C(F)F)cc(Cl)c2C2CC2)nc2c3c(nc(OC[C@@]45CCCN4C[C@H](F)C5)nc13)N1C[C@H]3CC[C@H](N3)[C@H]1CO2 ; Fc1c(-c2cncc(Cl)c2C2CC2)nc2c3c(nc(OC[C@@]45CCCN4C[C@H](F)C5)nc13)N1C[C@H]3CC[C@H](N3)[C@H]1CO2 ; Cc1cc(N)nc(-c2nc3c4c(nc(OC[C@@]56CCCN5C[C@H](F)C6)nc4c2F)N2C[C@H]4CC[C@H](N4)[C@H]2CO3)c1Cl ; Cc1cc(N)nc(-c2nc3c4c(nc(OC[C@@]56CCCN5C[C@H](F)C6)nc4c2F)N2C[C@H]4CC[C@H](N4)[C@H]2CO3)c1C(F)F] 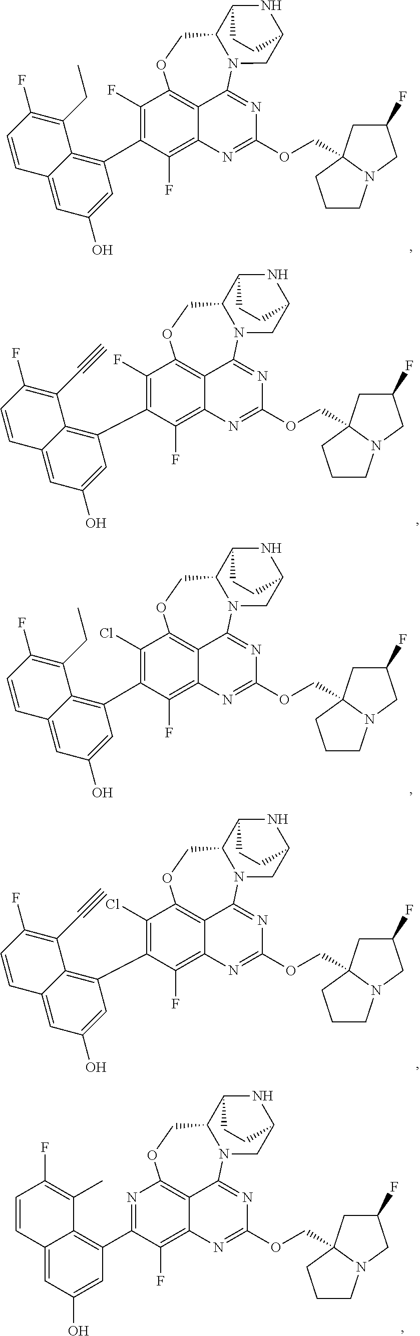 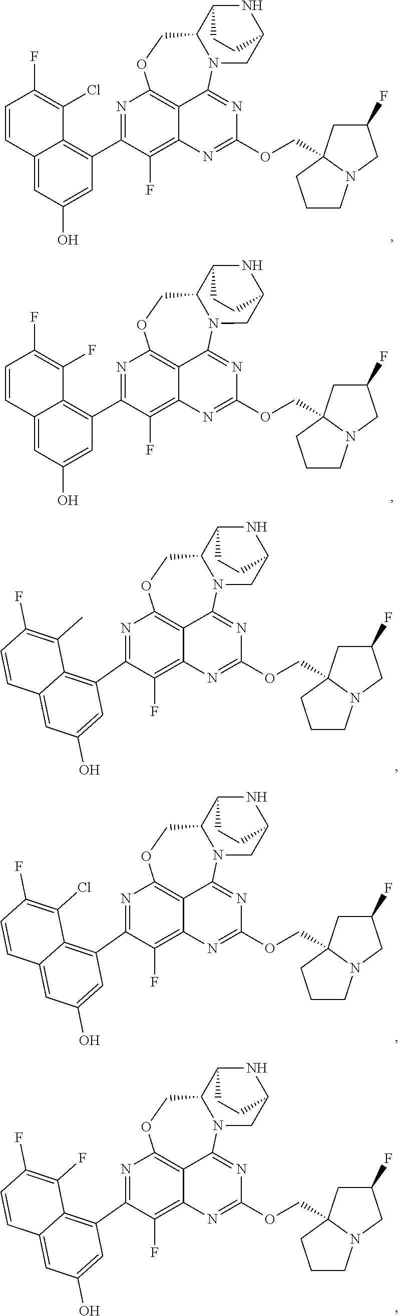 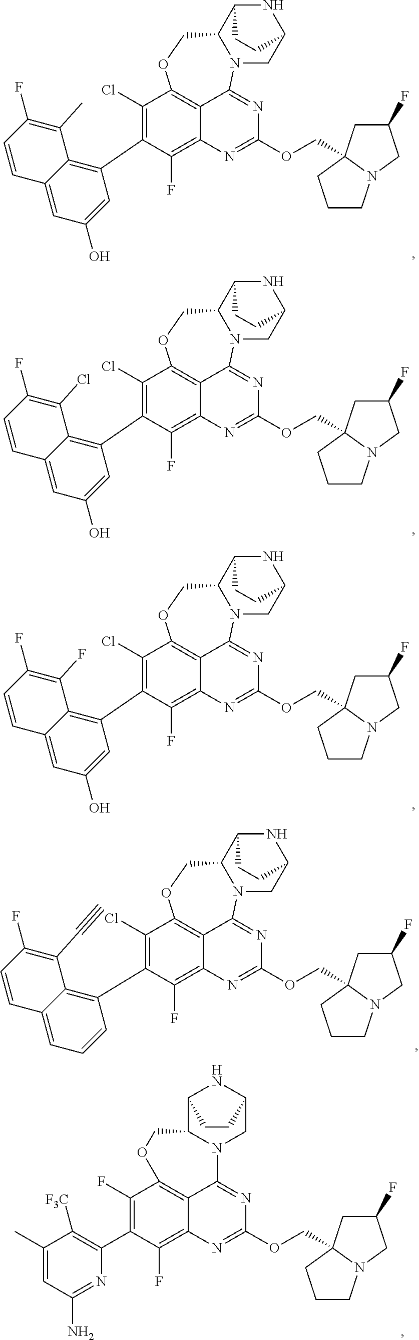 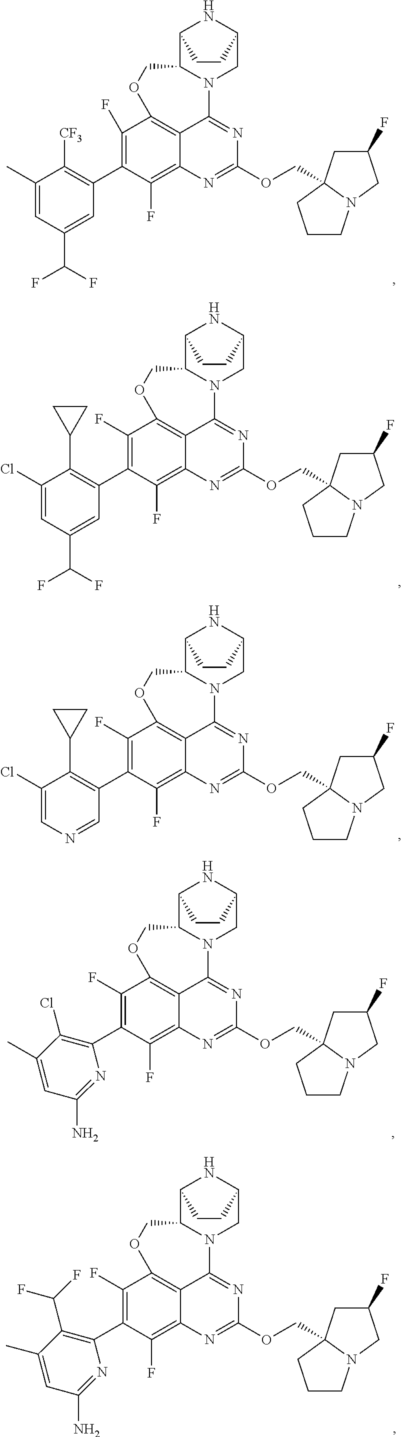 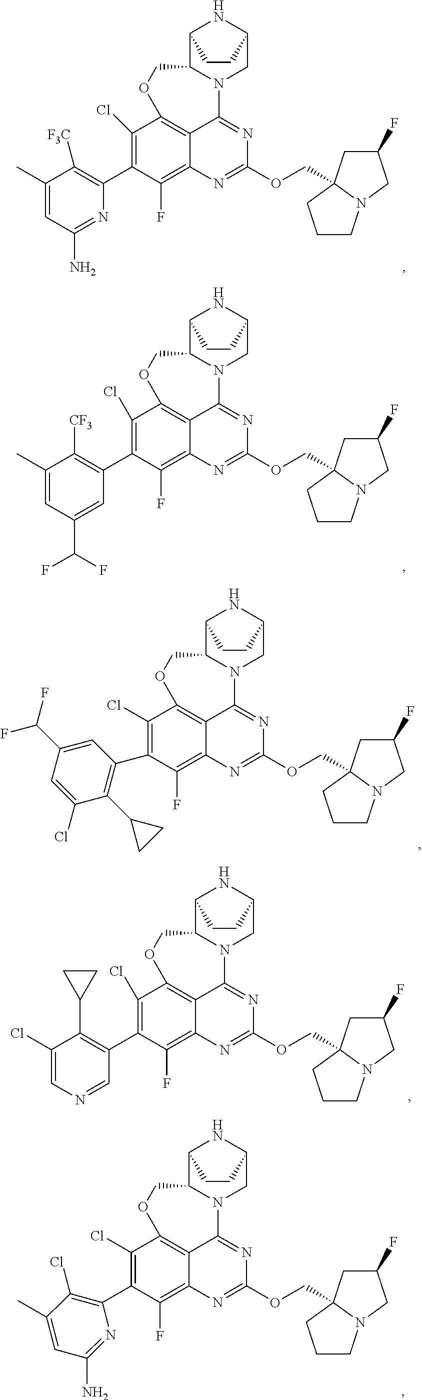 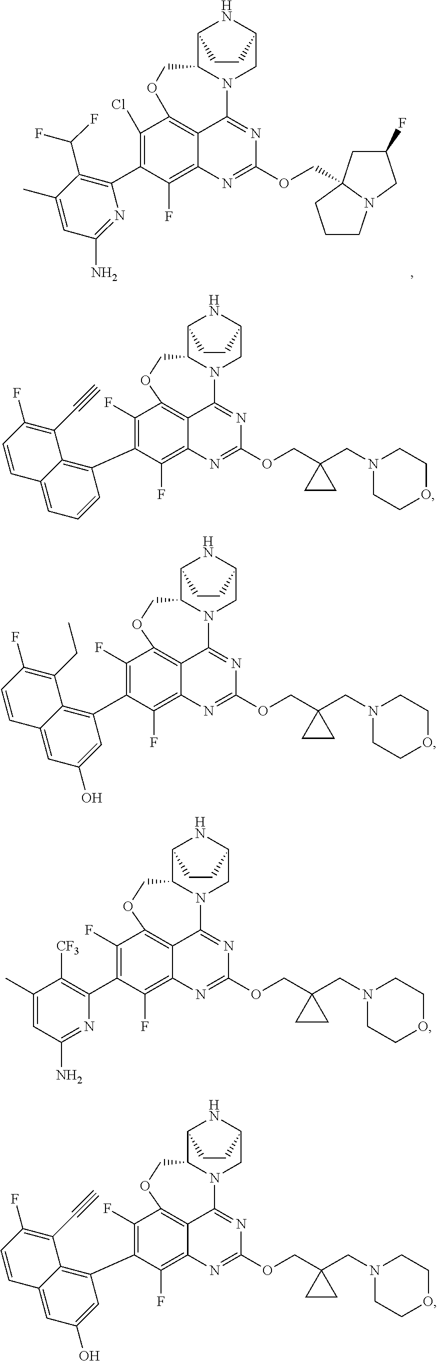 [SMILES: Cc1cc(N)nc(-c2c(Cl)c3c4c(nc(OC[C@@]56CCCN5C[C@H](F)C6)nc4c2F)N2C[C@H]4CC[C@H](N4)[C@H]2CO3)c1C(F)F ; C#Cc1c(F)ccc2cccc(-c3c(F)c4c5c(nc(OCC6(CN7CCOCC7)CC6)nc5c3F)N3C[C@H]5CC[C@H](N5)[C@H]3CO4)c12 ; CCc1c(F)ccc2cc(O)cc(-c3c(F)c4c5c(nc(OCC6(CN7CCOCC7)CC6)nc5c3F)N3C[C@H]5CC[C@H](N5)[C@H]3CO4)c12 ; Cc1cc(N)nc(-c2c(F)c3c4c(nc(OCC5(CN6CCOCC6)CC5)nc4c2F)N2C[C@H]4CC[C@H](N4)[C@H]2CO3)c1C(F)(F)F ; C#Cc1c(F)ccc2cc(O)cc(-c3c(F)c4c5c(nc(OCC6(CN7CCOCC7)CC6)nc5c3F)N3C[C@H]5CC[C@H](N5)[C@H]3CO4)c12] 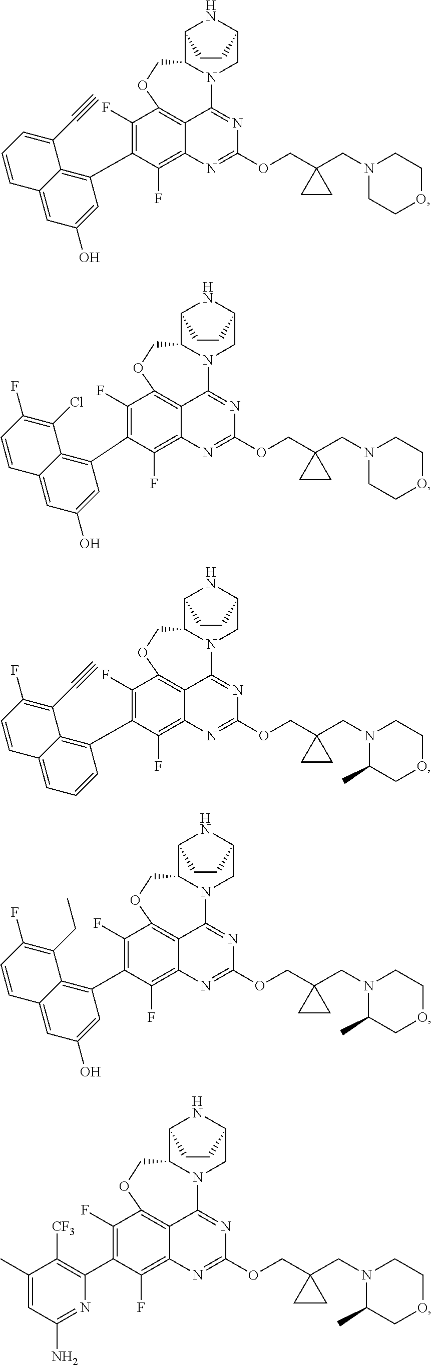 [SMILES: C#Cc1cccc2cc(O)cc(-c3c(F)c4c5c(nc(OCC6(CN7CCOCC7)CC6)nc5c3F)N3C[C@H]5CC[C@H](N5)[C@H]3CO4)c12 ; Oc1cc(-c2c(F)c3c4c(nc(OCC5(CN6CCOCC6)CC5)nc4c2F)N2C[C@H]4CC[C@H](N4)[C@H]2CO3)c2c(Cl)c(F)ccc2c1 ; C#Cc1c(F)ccc2cccc(-c3c(F)c4c5c(nc(OCC6(CN7CCOC[C@H]7C)CC6)nc5c3F)N3C[C@H]5CC[C@H](N5)[C@H]3CO4)c12 ; CCc1c(F)ccc2cc(O)cc(-c3c(F)c4c5c(nc(OCC6(CN7CCOC[C@H]7C)CC6)nc5c3F)N3C[C@H]5CC[C@H](N5)[C@H]3CO4)c12 ; Cc1cc(N)nc(-c2c(F)c3c4c(nc(OCC5(CN6CCOC[C@H]6C)CC5)nc4c2F)N2C[C@H]4CC[C@H](N4)[C@H]2CO3)c1C(F)(F)F] 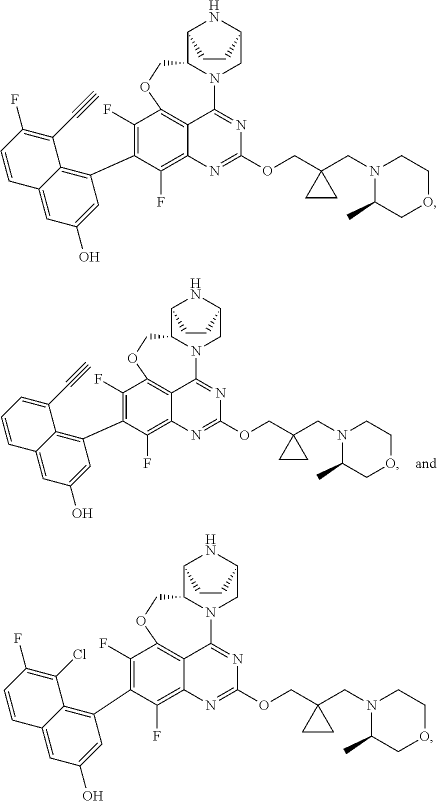 [SMILES: C#Cc1c(F)ccc2cc(O)cc(-c3c(F)c4c5c(nc(OCC6(CN7CCOC[C@H]7C)CC6)nc5c3F)N3C[C@H]5CC[C@H](N5)[C@H]3CO4)c12 ; C#Cc1cccc2cc(O)cc(-c3c(F)c4c5c(nc(OCC6(CN7CCOC[C@H]7C)CC6)nc5c3F)N3C[C@H]5CC[C@H](N5)[C@H]3CO4)c12 ; C[C@@H]1COCCN1CC1(COc2nc3c4c(c(F)c(-c5cc(O)cc6ccc(F)c(Cl)c56)c(F)c4n2)OC[C@@H]2[C@@H]4CC[C@H](CN32)N4)CC1]
or a pharmaceutically acceptable salt thereof.

111. A pharmaceutical composition comprising the compound of claim 98 or a pharmaceutically acceptable salt thereof, and a pharmaceutically acceptable carrier.

112. A method for treating a KRas G12D-associated cancer comprising administering an effective amount of a compound of claim 98 or a pharmaceutically acceptable salt thereof.

113. The method of claim 112, wherein the KRas G12D-associated cancer is selected from the group consisting of:
(i)Cardiac: sarcoma (angiosarcoma, fibrosarcoma, rhabdomyosarcoma, liposarcoma), myxoma, rhabdomyoma, fibroma, lipoma and teratoma;
(ii) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
(iii) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
(iv) Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
(v) Liver: hepatoma (hepatocellular carcinoma), cholangiocarcinoma, hepatoblastoma, angiosarcoma, hepatocellular adenoma, hemangioma;
(vi) Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
(vii) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);
(viii) Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
(ix) Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
(x) Skin: malignant melanoma, basal cell carcinoma, squamous cell carcinoma, Kaposi's sarcoma, moles dysplastic nevi, lipoma, angioma, dermatofibroma, keloids, psoriasis; and
(xi) Adrenal glands: neuroblastoma.

114. The method of claim 112, wherein the KRas G12D-associated cancer is non-small cell lung cancer, small cell lung cancer, colorectal cancer, rectal cancer or pancreatic cancer.

115. The method of claim 112, wherein the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

116. The method of claim 112, wherein the compound is administered simultaneously, separately or sequentially with one or more additional therapeutic agents.

117. The method of claim 116, wherein the one or more additional therapeutic agents are selected from an anti-PD-1 or PD-L1 antagonist, an MEK inhibitor, a CDK4/CDK6 inhibitor, an EGFR inhibitor, ERK inhibitor, a SHP2 inhibitor, a platinum agent or pemetrexed.
